(19) United States
(12) Patent Application Publication
(10) Pub. No.: US 2024/0093169 A1
(43) Pub. Date: Mar. 21, 2024

(54) SYNTHETIC TRANSCRIPTION FACTORS

(71) Applicant: THE REGENTS OF THE UNIVERSITY OF CALIFORNIA, Oakland, CA (US)

(72) Inventors: Niklas F. C. HUMMEL, San Francisco, CA (US); Patrick M. SHIH, Berkeley, CA (US)

(21) Appl. No.: 18/298,942

(22) Filed: Apr. 11, 2023

Related U.S. Application Data
(60) Provisional application No. 63/330,243, filed on Apr. 12, 2022.

(51) Int. Cl. C12N 9/22; C12N 9/96; C12N 15/11

(52) U.S. Cl. CPC C12N 9/22; C12N 9/96; C12N 15/111; C12N 2310/20

(57) ABSTRACT

The present invention provides for a synthetic transcription factor (TF) comprising (a) a DNA-binding domain of a transcription factor linked to (b) an effector domain, and (c) optionanlly a nuclear localization sequence (NLS). The present invention provides for a nucleic acid encoding an effector domain of the present invention. The DNA-binding domain can be a deactivated RNA-guided nuclease variant of Cas9 (dCas9).

### CROSS REFERENCE TO RELATED APPLICATIONS

This application claims priority to U.S. Provisional Patent Application Ser. No. 63/330,243, filed Apr. 12, 2022, which is incorporated by reference in its entirety.

### STATEMENT OF GOVERNMENTAL SUPPORT

The invention was made with government support under Contract Nos. DE-AC02-05CH11231 awarded by the U.S. Department of Energy. The government has certain rights in the invention.

### REFERENCE TO SEQUENCE LISTING

The instant application contains a Sequence Listing which has been submitted electronically in XML format and is hereby incorporated by reference in its entirety. Said XML copy, created on Jul. 17, 2023, is named 2021-082-02 Sequence Listing 17 Jul. 2023 .xml and is 413,000 bytes in size.

### FIELD OF THE INVENTION

The present invention is in the field of regulating gene expression in plants.

### BACKGROUND OF THE INVENTION

Biological systems are predicated on transcriptional networks, which are largely regulated by transcription factors (TFs). At their core, TFs are defined by two broad functions: 1) specifically binding target regulatory DNA sequences through DNA-binding domains (DBDs) and 2) regulating transcription (i.e., gene activation or repression) through effector domains. Recent technical advances and large consortium efforts have dramatically expanded our understanding of TF binding sites across full genomes ((1), (2)). However, the nature of these interactions has remained elusive, as the characterization of effector domains has not been as readily scalable. As a result, our knowledge of trans-effector domains has not kept pace with our characterization of cis-regulatory elements (3). Therefore, elucidating the activity of effector domains represents a key missing piece to comprehensively understanding transcriptional networks described in gene regulatory networks (GRNs).

The regulatory role of each TF defines the functional nature of its interactions with its downstream genes. Incorrect predictions of up- or down-regulation (activation or repression, respectively) can dramatically alter the anticipated output of genetic circuits, highlighting our largely incomplete understanding of GRNs. Moreover, due to the lack of information on effector domains, GRNs are largely limited to DNA binding information, limiting the scope of analyses, specifically on genes associated with multiple regulators of unknown activity (4, 5). Effector domains can serve as biochemical beacons recruiting or inhibiting transcriptional machinery; however, the mechanisms underlying these processes are not well understood and have primarily been studied in eukaryotic families distant from plants (6). Identification and characterization of these domains in plants is an important first step towards elucidating the design principles that govern gene regulation in order to ultimately enable more refined approaches to engineer and fine-tune transcription.

### SUMMARY OF THE INVENTION

The present invention provides for a synthetic transcription factor (TF) comprising (a) a DNA-binding domain of a transcription factor linked to (b) an effector domain, and (c) optionally a nuclear localization sequence (NLS).

In some embodiments, the DNA-binding domain is a DNA-binding domain of a eukaryotic TF or a prokaryotic TF. In some embodiments, the DNA-binding domain is a DNA-binding domain of a eukaryotic TF. In some embodiments, the DNA-binding domain is a deactivated RNA-guided nuclease variant of Cas9 (dCas9). In some embodiments, the DNA-binding domain is about 8, 10, 20, 30, 40, 50, 60, 70, 80, 90, 100, 110, 120, 130, 140, 146, or 150 amino acid residues long, or within a range of any two preceding values.

In some embodiments, the eukaryotic TF is a yeast TF. In some embodiments, the yeast TF is a Saccharomyces TF. In some embodiments, the Saccharomyces TF is a Saccharomyces cerevisiae TF.

In some embodiments, the S. cerevisiae TF is Ga14, YAP1, GAT1, MATAL1, MATAL2, MCM1, Abf1, Adr1, Ash1, Gcn4, Gcr1, Hap4, Hsf1, Ime1, Ino2/Ino4, Leu3, Lys14, Mata2, Mga2, Met4, Mig1, Rap1, Rgt1, Rlm1, Smp1, Rme1, Rox1, Rtg3, Spt23, Teal, Ume6, or Zap1. In some embodiments, the S. cerevisiae TF is Ga14, YAP1, GAT1, MATAL1, MATAL2, or MCM1.

In some embodiments, the S. cerevisiae TF is Ga14. In some embodiments, the DNA-binding domain comprises the amino acid sequence of Ga14 or MKLLSSIEQA CDICRLKKLK CSKEKPKCAK CLKNNWECRY SPKTKRSPLT RAHLTEVESR LERLEQLFLL IFPREDLDMI LKMDSLQDIK ALLTGLFVQD NVNKDAVTDR LASVETDMPL TLRQHRISAT SSSEESSNKG QRQLTV (SEQ ID NO:404).

In some embodiments, the S. cervisiae TF is YAP1. In some embodiments, the DNA-binding domain comprises the amino acid sequence of YAP1, PETKQKR TAQNRAAQRA FRERKERKMK ELEKKVQSLE SIQQQNEVEA TFLRDQLITL VNELKKY (SEQ ID NO:405) or KQ DLDPETKQKR TAQNRAAQRA FRERKERKMK ELEKKVQSLE SIQQQNEVEA TFLRDQLITL VNELKKYRPE TRNDSKVLEY LARRDPNL (SEQ ID NO:406).

In some embodiments, the S. cervisiae TF is GAT1. In some embodiments, the DNA-binding domain comprises the amino acid sequence of GAT1, IFTNNLP FLNNNSINNN HSHNSSHNNN SPSIANNTNA NTNTNTSAST NTNSPLL (SEQ ID NO:407) or D DHFIFTNNLP FLNNNSINNN HSHNSSHNNN SPSIANNTNA NTNTNTSAST NTNSPLLRRN PSP (SEQ ID NO:408).

In some embodiments, the S. cervisiae TF is MATAL1. In some embodiments, the DNA-binding domain comprises the amino acid sequence of MATAL1 or KKEKS PKGKSSISPQ ARAFLEQVFR RKQSLNSKEK EEVAKKCGIT PLQVRVWFIN KRMRSK (SEQ ID NO:409).

In some embodiments, the S. cerevisiae TF is MATAL2. In some embodiments, the DNA-binding domain comprises the amino acid sequence of MATAL2 or STKP YRGHRFTKEN VRILESWFAK NIENPYLDTK GLENLMKNTS LSRIQIKNWV SNRRRKEKTI TIAP (SEQ ID NO:410).

In some embodiments, the S. cerevisiae TF is MCM1. In some embodiments, the DNA-binding domain comprises the amino acid sequence of MCM1, RRK IEIKFIENKT RRHVTFSKRK HGIMKKAFEL SVLTGTQVLL LVVSETGLVY TF (SEQ ID NO:411) or KERRK IEIKFIENKT RRHVTFSKRK HGIMKKAFEL SVLTGTQVLL LVVSETGLVY TFSTPKFEPI VTQQEGRNLI QACLNA (SEQ ID NO:412).

In some embodiments, the S. cerevisiae TF is Rap1. In some embodiments, the DNA-binding domain comprises the amino acid sequence of Rap1, or GXXIRXRF (wherein X is any amino acid) (SEQ ID NO:413), G(G, P, A or R)(S or A)IRXRF (wherein X is any amino acid) (SEQ ID NO:414), or GNSIRHRFRV(SEQ ID NO:415).

In some embodiments, the effector domain is an activator domain, inactive domain, or repressor domain. In some embodiments, the repressor domain comprises the amino acid sequence of one of SEQ ID NO:1 to SEQ ID NO:72. In some embodiments, the repressor domain has the capability to effect a “log2_GFP foldchange” (using the conditions as described herein) of equal to or less than about −0.7, −0.8, −0.9, −1.0, −1.1, −1.2, −1.3, −1.4, −1.5, −1.6, −1.7, −1.8, −1.9, −2.0, −2.1, −2.2, or −2.3, or any value within any two preceding values. In some embodiments, the repressor domain comprises an amino acid sequence having equal to or more than 70%, 75%, 80%, 85%, 90%, 95%, or 99% amino acid identity to any one of SEQ ID NO:1 to SEQ ID NO:72, and optionally (a) comprises at least about one, two, three. four, five, six, seven, eight, nine, ten, 11, 12, 13, 14, 15, 16, 17, 18, 19, or 20, and/or equal to or more than 10%, 20%, 30%, 40%, 50%, 60%, 70%, 80%, 90%, or 100% of the Arg of the corresponding SEQ ID NO:1 to SEQ ID NO:72.

In some embodiments, the inactive domain comprises the amino acid sequence of one of SEQ ID NO:73 to SEQ ID NO:335. In some embodiments, the inactive domain has the capability to effect a “log2 GFP foldchange” (using the conditions as described herein) of equal to about −0.7, −0.6, −0.5, −0.4, −0.3, −0.2, −0.1, 0.0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, or 1.9, or any value within any two preceding values.

In some embodiments, the activator domain comprises the amino acid sequence of one of SEQ ID NO:336 to SEQ ID NO:403. In some embodiments, the activator domain has the capability to effect a “log2 GFP foldchange” (using the conditions as described herein) of equal to or more than about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.00, or any value within any two preceding values. In some embodiments, the activator domain comprises an amino acid sequence having equal to or more than 70%, 75%, 80%, 85%, 90%, 95%, or 99% amino acid identity to any one of SEQ ID NO:336 to SEQ ID NO:403, and optionally (a) comprises at least about one, two, three. four, five, six, seven, eight, nine, ten, 11, 12, 13, 14, 15, 16, 17, 18, 19, or 20, and/or equal to or more than 10%, 20%, 30%, 40%, 50%, 60%, 70%, 80%, 90%, or 100% of the acidic and/or hydrophobic amino acid residues, and/or comprises equal to or fewer basic amino acid residues, of the corresponding SEQ ID NO:336 to SEQ ID NO:403.

In some embodiments, the acidic amino acid residue is Glu and/or Asp. In some embodiments, the hydrophobic amino acid residue is Ala, Val, Iso, Leu, Met, Phe, Tyr and/or Trp. In some embodiments, the basic amino acid residue is Arg, Lys and/or His.

In some embodiments, the NLS is monopartite. In some embodiments, the NLS comprises the amino acid sequence K-K/R-X-K/R (SEQ ID NO:416), PKKKRKV (SV40 Large T-antigen) (SEQ ID NO:417), PAAKRVKLD (c-Myc) (SEQ ID NO:418) or KLKIKRPVK (TUS-protein) (SEQ ID NO:419).

In some embodiments, the NLS is bipartite. In some embodiments, the NLS comprises the amino acid sequence KRXioKKKK (SEQ ID NO:420), KRPAATKKAGQAKKKK (SEQ ID NO:421) or AVKRPAATKKAGQAKKKKLD (nucleoplasmin NLS) (SEQ ID NO:422) or MSRRRKANPTKLSENAKKLAKEVEN (EGL-13) (SEQ ID NO:423).

In some embodiments, the NLS comprises a M9 domain or PY-NLS motif. In some embodiments, the NLS comprises the M9 domain comprising the amino acid sequence (a) one or more of YNDFGNYN (SEQ ID NO:424) or FGNYN (SEQ ID NO:425), SN-F/Y-GPMK (SEQ ID NO:426), N-F/Y-GG (SEQ ID NO:427), GPYGGG (SEQ ID NO:428), (b) GNYNNQS SNFGPMKGGN FGGRSSGPYG GGGQYFAKPR NQGGY (hnRNP A1) (SEQ ID NO:429), (c) FGNYNQQPSN YGPMKSGNFG GSRNMGGPYG GGNYGPGGSG GSGGY(hnRNP A2/B1) (SEQ ID NO:430), (d) FGNYNSQSSS NFGPMKGGNY GGRNSGPYGG GYGGGSASSS SGY (Xenopus RNP A1) (SEQ ID NO:431), or (e) FGNYNQQSSN YGPMKSGGNF GGNRSMGGGP YGGGNYGPGN ASGGNGGGY (Xenopus RNP A2) (SEQ ID NO:432).

In some embodiments, the NLS comprises the amino acid sequence KIPIK (yeast Matα2) (SEQ ID NO:433). In some embodiments, the NLS is about 5, 10, 20, 30, 40, 50, 55, or 60 amino acid residues long, or within a range of any two preceding values.

In some embodiments, wherein any two, or all, of the DNA-binding domain, the effector domain, and the NLS are heterologous to each other.

In some embodiments, wherein one or more, or all, of the DNA-binding domain, the effector domain, and the NLS are obtained or derived from a non-viral organism.

In some embodiments, the DNA-binding domain, the NLS, and the effector domain are linked in this order from N- to C-terminus. Exemplary synthetic TF include, but are not limited to, the following:

The amino acid sequence of MCM1 is as follows:

(SEQ ID NO: 434)
MSDIEEGTPTNNGQQKERRKIEIKFIENKTRRHVTFSKRKHGIMKKAFE
LSVLTGTQVLLLVVSETGLVYTFSTPKFEPIVTQQEGRNLIQACLNAPD
DEEEDEEEDGDDDDDDDDDGNDMQRQQPQQQQPQQQQQVLNAHANSLGH
LNQDQVPAGALKQEVKSQLLGGANPNQNSMIQQQQHHTQNSQPQQQQQQ
QPQQQMSQQQMSQHPRPQQGIPHPQQSQPQQQQQQQQQLQQQQQQQQQQ
PLTGIHQPHQQAFANAASPYLNAEQNAAYQQYFQEPQQGQY.

The amino acid sequence of MATAL1 is as follows:

(SEQ ID NO: 435)
MDDICSMAENINRTLFNILGTEIDEINLNTNNLYNFIMESNLTKVEQHT
LHKNISNNRLEIYHHIKKEKSPKGKSSISPQARAFLEQVFRRKQSLNSK
EKEEVAKKCGITPLQVRVWFINKRMRSK.

The amino acid sequence of MATAL2 is as follows:

(SEQ ID NO: 436)
MNKIPIKDLLNPQITDEFKSSILDINKKLFSICCNLPKLPESVTTEEEV
ELRDILGFLSRANKNRKISDEEKKLLQTTSQLTTTITVLLKEMRSIEND
RSNYQLTQKNKSADGLVFNVVTQDMINKSTKPYRGHRFTKENVRILESW
FAKNIENPYLDTKGLENLMKNTSLSRIQIKNWVSNRRRKEKTITIAPEL
ADLLSGEPLAKKKE.

The amino acid sequence of Yap1 is as follows:

(SEQ ID NO: 437)
MSVSTAKRSLDVVSPGSLAEFEGSKSRHDEIENEHRRTGTRDGEDSEQP
KKKGSKTSKKQDLDPETKQKRTAQNRAAQRAFRERKERKMKELEKKVQS
LESIQQQNEVEATFLRDQLITLVNELKKYRPETRNDSKVLEYLARRDPN
LHFSKNNVNHSNSEPIDTPNDDIQENVKQKMNFTFQYPLDNDNDNDNSK
NVGKQLPSPNDPSHSAPMPINQTQKKLSDATDSSSATLDSLSNSNDVLN
NTPNSSTSMDWLDNVIYTNRFVSGDDGSNSKTKNLDSNMFSNDFNFENQ
FDEQVSEFCSKMNQVCGTRQCPIPKKPISALDKEVFASSSILSSNSPAL
TNTWESHSNITDNTPANVIATDATKYENSFSGFGRLGFDMSANHYVVND
NSTGSTDSTGSTGNKNKKNNNNSDDVLPFISESPFDMNQVTNFFSPGST
GIGNNAASNTNPSLLQSSKEDIPFINANLAFPDDNSTNIQLQPFSESQS
QNKFDYDMFFRDSSKEGNNLFGEFLEDDDDDKKAANMSDDESSLIKNQL
INEEPELPKQYLQSVPGNESEISQKNGSSLQNADKINNGNDNDNDNDVV
PSKEGSLLRCSEIWDRITTHPKYSDIDVDGLCSELMAKAKCSERGVVIN
AEDVQLALNKHMN.

The amino acid sequence of Gat1 is as follows:

(SEQ ID NO: 438)
MHVFFPLLFRPSPVLFIACAYIYIDIYIHCTRCTVVNITMSTNRVPNLD
PDLNLNKEIWDLYSSAQKILPDSNRILNLSWRLHNRTSFHRINRIMQHS
NSIMDFSASPFASGVNAAGPGNNDLDDTDTDNQQFFLSDMNLNGSSVFE
NVFDDDDDDDDVETHSIVHSDLLNDMDSASQRASHNASGFPNFLDTSCS
SSFDDHFIFTNNLPFLNNNSINNNHSHNSSHNNNSPSIANNTNANTNTN
TSASTNTNSPLLRRNPSPSIVKPGSRRNSSVRKKKPALKKIKSSTSVQS
SATPPSNTSSNPDIKCSNCTTSTTPLWRKDPKGLPLCNACGLFLKLHGV
TRPLSLKTDIIKKRQRSSTKINNNITPPPSSSLNPGAAGKKKNYTASVA
ASKRKNSLNIVAPLKSQDIPIPKIASPSIPQYLRSNTRHHLSSSVPIEA
ETFSSFRPDMNMTMNMNLHNASTSSFNNEAFWKPLDSAIDHHSGDTNPN
SNMNTTPNGNLSLDWLNLNL.

The present invention also provides for a nucleic acid encoding any one of the synthetic TF of the present invention operatively linked to a promoter capable of expressing the synthetic TF in vitro or in vivo.

The present invention provides for a nucleic acid encoding an effector domain of the present invention. In some embodiments, the effector domain comprises an amino acid sequence of SEQ ID NO:1-403. In some embodiments, the effector domain is about 27, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 572, 580, 590, or 600 amino acid residues long, or within a range of any two preceding values.

The present invention also provides for a vector comprising the nucleic acid of the present invention. In some embodiments, the vector is capable of stably integrating into a chromosome of a host cell or stably residing in a host cell. In some embodiments, the vector is an expression vector.

The present invention also provides for a host cell comprising the vector of the present invention, wherein the host cell is capable of expressing the synthetic TF or effector domain.

The present invention also provides for a system comprising a nucleic acid of the present invention and a second nucleic acid, or the nucleic acid, encodes a gene of interest (GOI) operatively linked to a promoter and one or more activator/repressor binding domains, or combination thereof, wherein the synthetic TF binds at least one of the one or more activator/repressor binding domain such that the synthetic TF modulates the expression of the GOI.

The present invention also provides for a genetically modified eukaryotic cell or organism, such as a plant cell or plant, comprising: (a) (i) one or more nucleic acids each encoding one or more transcription activators operatively linked to a first promoter, (ii) one or more nucleic acids each encoding one or more transcription repressors each operatively linked to a second promoter, or (iii) combinations thereof; and (b) one or more nucleic acids each encoding one or more independent genes of interest (GOI) each operatively linked to a promoter that is activated by the one or more transcription activators, repressed by the one or more transcription repressors, or a combination of both; wherein at least one transcription activator or transcription repressor is a synthetic transcription factor (TF) of the present invention

In some embodiments, the first promoter, the second promoter, or both, is a tissue-specific or inducible promoter.

In some embodiments, the transcription activator is the synthetic TF. In some embodiments, the transcription repressor is the synthetic TF.

In some embodiments, any domain of the synthetic TF is heterologous to the plant cell or plant, one or more of the GOI, any other transcription activator or transcription repressor, and/or any of the promoters.

In some embodiments, the transcription activator is heterologous to the eukaryotic cell or organism, such as a plant cell or plant, one or more of the GOI, any other or transcription activator, transcription repressor, and/or any of the promoters. In some embodiments, the transcription repressor is heterologous to the eukaryotic cell or organism, such as a plant cell or plant, one or more of the GOI, any other transcription activator, and/or any of the promoters.

In some embodiments, the genetically modified eukaryotic cell or organism, such as a plant cell or plant comprises: (a) a first nucleic acid encoding a transcription activator operatively linked to a first tissue-specific or inducible promoter, (b) optionally a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription activators, repressed by the transcription repressors, or a combination of both.

In some embodiments, the genetically modified eukaryotic cell or organism, such as a plant cell or plant comprises: (a) optionally a first nucleic acid encoding a transcription activator operatively linked to a first tissue-specific or inducible promoter, (b) a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription activators, repressed by the transcription repressors, or a combination of both.

In some embodiments, the promoter is a tissue-specific promoter. Examples of tissue-specific promoters under developmental control include promoters that initiate transcription only (or primarily only) in certain tissues, such as vegetative tissues, cell walls, including e.g., roots or leaves. A variety of promoters specifically active in vegetative tissues, such as leaves, stems, roots and tubers are known. For example, promoters controlling patatin, the major storage protein of the potato tuber, can be used (see, e.g., Kim, Plant Mol. Biol. 26:603-615, 1994; Martin, Plant J. 11:53-62, 1997). The ORF13 promoter from Agrobacterium rhizogenes that exhibits high activity in roots can also be used (Hansen, Mol. Gen. Genet. 254:337-343, 1997). Other useful vegetative tissue-specific promoters include: the tarn promoter of the gene encoding a globulin from a major taro (Colocasia esculenta L. Schott) corm protein family, tarin (Bezerra, Plant Mol. Biol. 28:137-144, 1995); the curculin promoter active during taro corm development (de Castro, Plant Cell 4:1549-1559, 1992) and the promoter for the tobacco root-specific gene TobRB7, whose expression is localized to root meristem and immature central cylinder regions (Yamamoto, Plant Cell 3:371-382, 1991).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier, FEBS Lett. 415:91-95, 1997). A ribulose bisphosphate carboxylase promoters expressed almost exclusively in mesophyll cells in leaf blades and leaf sheaths at high levels (e.g., Matsuoka, Plant J. 6:311-319, 1994), can be used. Another leaf-specific promoter is the light harvesting chlorophyll a/b binding protein gene promoter (see, e.g., Shiina, Plant Physiol. 115:477-483, 1997; Casal, Plant Physiol. 116:1533-1538, 1998). The Arabidopsis thaliana myb-related gene promoter (Atmyb5) (Li, et al., FEBS Lett. 379:117-121 1996), is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize (e.g., Busk et al., Plant J. 11:1285-1295, 1997) can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, (e.g., Di Laurenzio, et al., Cell 86:423-433, 1996; and, Long, et al., Nature 379:66-69, 1996); can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto, Plant Cell. 7:517-527, 1995). Also useful are knl-related genes from maize and other species which show meristem-specific expression, (see, e.g., Granger, Plant Mol. Biol. 31:373-378, 1996; Kerstetter, Plant Cell 6:1877-1887, 1994; Hake, Philos. Trans. R. Soc. Lond. B. Biol. Sci. 350:45-51, 1995). For example, the Arabidopsis thaliana KNAT1 promoter (see, e.g., Lincoln, Plant Cell 6:1859-1876, 1994) can be used.

In some embodiments, the promoter is substantially identical to the native promoter of a promoter that drives expression of a gene involved in secondary wall deposition. Examples of such promoters are promoters from IRX1, IRX3, IRX5, IRX8, IRX9, IRX14, IRX7, IRX10, GAUT13, or GAUT14 genes. Specific expression in fiber cells can be accomplished by using a promoter such as the NST1 promoter and specific expression in vessels can be accomplished by using a promoter such as VND6 or VND7. (See, e.g., PCT/US2012/023182 for illustrative promoter sequences). In some embodiments, the promoter is a secondary cell wall-specific promoter or a fiber cell-specific promoter. In some embodiments, the promoter is from a gene that is co-expressed in the lignin biosynthesis pathway (phenylpropanoid pathway). In some embodiments, the promoter is a C4H, C3H, HCT, CCR1, CAD4, CADS, FSH, PALL PAL2, 4CL1, or CCoAMT promoter. In some embodiments, the tissue-specific secondary wall promoter is an IRX1, IRX3, IRX5, IRX8, IRX9, IRX14, IRX7, IRX10, GAUT13, GAUT14, or CESA4 promoter. Suitable tissue-specific secondary wall promoters, and other transcription factors, promoters, regulatory systems, and the like, suitable for this present invention are taught in U.S. Patent Application Pub. Nos. 2014/0298539, 2015/0051376, and 2016/0017355.

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In some embodiments, each GOI is operatively linked to a promoter that is activated by the transcription activator, repressed by the transcription repressors, or a combination of both.

In some embodiments, the promoter comprises one or more DNA-binding sites specific for the transcription activator, one or more DNA-binding sites specific for the transcription repressor, or a combination of both.

In some embodiments, the promoter comprises 1, 2, 3, 4, 5, 6, 7, 8, 9, or 10 DNA-binding sites specific for the transcription activator), 1, 2, 3, 4, 5, 6, 7, 8, 9, or 10 DNA-binding sites specific for the transcription repressor, or a combination of both.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Genome-wide screen identifying hundreds of novel transcriptional effectors gives insight into regulatory dynamics and structural features of plant transcription factors. (A) Truncated putative effector domains are fused to the yeast Ga14-DBD to generate a library of synthetic TFs and targeted to a fluorescent reporter to observe modulation of gene expression. (B) GFP expression of 403 synthetic TFs in relation to background reporter expression in N. benthamiana leaves 3 days post infiltration (n=16 biological replicates). Arrow indicates positions of Ga14-VP16 as a strong activator control. (C) Left: Effector domains characterized as repressors are more likely to auto-regulate their own expression than activators. Sliding window analysis (window size n=25) of DNA binding behavior based on autoregulation of TF sorted by performance in the effector screen. Right: Fractions of TF populations showing the potential for auto-regulation (asterisks indicate Kruskal-Wallis significance values **P<5×10⁻³). (D) Genomic targets of strong activators link strong activation to response to environmental cues. GO ontology enrichment for genomic targets of strong activators, clustered by overarching biological processes. Non boxed GO terms were not linked to an overarching GO parent. (E) Fraction of protein in amino acid groups for every effector candidate in the respective population (asterisks indicate Mann-Whitney U significance test *P≤5×10⁻², **P≤5×10⁻³, ***P≤5×10⁻⁴, ****P≤5×10⁻⁵, ns non significant). (F) Isoelectric point of effector domains mapped to performance in effector screen.

FIG. 2. Effector activity allows to study GRNs in new depth. A) GRN describing TFs and target genes responsive to nitrate in A. thaliana. Edges are annotated with effector activity data (color) and the predicted influence of a TF to its target (edge width) (4). Green nodes indicate core nitrogen metabolism genes. (B) Expression profiles for genes targeted by TFs overexpressed at 10 min and 15 min. (C) Distributions for the rate of expression change between timepoints for the genes in (B). (D) Counts showing time step with largest rate of gene expression increase for the genes in (B).

FIG. 3. Strong plant activators outperform VP16 in different gene expression setups. (A) Fusion of strong activators to the anthocyanin master regulator PAP1 promotes production of anthocyanins. (B) Visual representation of anthocyanin extracts quantified in C. (C) Quantification of anthocyanins extracted from N. benthamiana leaf tissue expressing PAP1-fusion constructs. (D) Activator fusion to dCas9 to modulate target gene expression. (E) Quantification of relative change of transcript numbers for dCas9-activator fusions using the ΔΔC_{q}-method.

FIG. 4. Plant effector activity is conserved in fungi and predictable using machine learning. (A) Plant activators can induce a native yeast promoter when fused to the GAL4-DBD. Fractions of cells showing fluorescence in the repressed state of the GAL1 promoter grown in glucose. (B) Fluorescence intensity distributions of activator and control populations. (C) Plant activators are enriched in activation domains predicted by a fungal machine learning model. (D) ADpred scores for effector domains of three strong activators. (E) ADpred predicted activator motifs can perform similar to full length effectors. Distribution of fluorescence of

FIG. 5. Effector activity can be linked to multiple biochemical properties. (A) Fraction of protein sequence predicted to be disordered by VSL2 in relation to GFP fold change (B) Box plot representing distribution of individual amino acid frequency for each effector in respective population.

FIG. 6. Combining effector activity with DBD-data suggests network properties. (A) Fully annotated FIG. 1D. (B) There is no observable trend for feedback loops between effector populations. Sum of effector TF targeted TFs binding the initial effectors promoter region.

FIG. 7. Integration of effector information decodes network behavior in nitrogen response and cold response GRNs. A) Subnetwork of FIG. 2a 10 min post induction with nitrate. B) Repressor activity 10 min post nitrate induction leads to temporal repression of genes in the nitrogen response GRN. Each dot represents the fold change in expression of a single gene present in GRN at time point 10 and 15 min. C) Activating Single input modules lead to increased expression compared to repressing single input modules and duo HHO-repressed genes. D) Simplified overview of CBF-regulon dependent cold response in A. thaliana.

FIG. 8. ADpred predicts putative activation domains in plant TFs. A) ADpred evaluation of the top 20 activators in this study. ADpred scores were calculated for every 30 amino acid stretch slided along the protein sequence with window size=5.

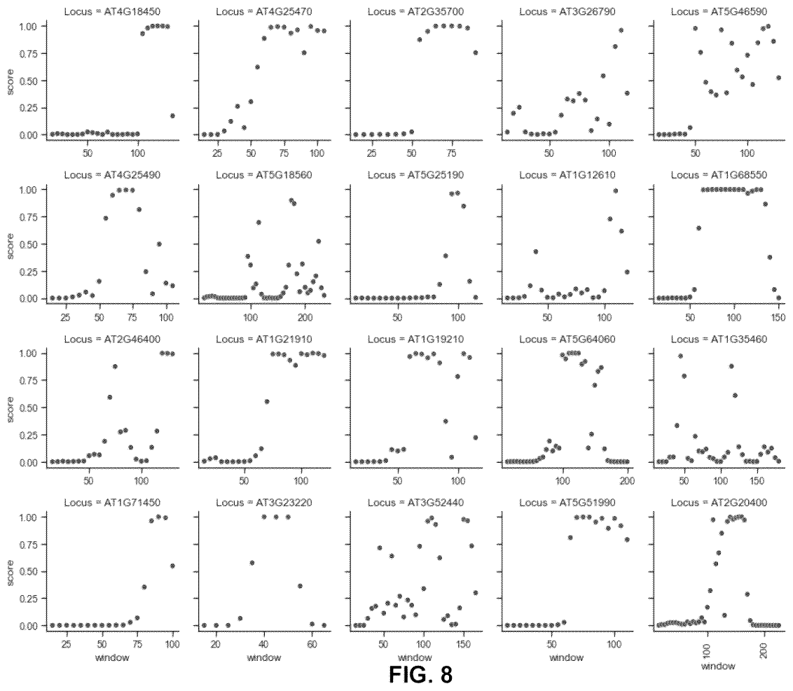

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

As used herein, the term “promoter” refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon.

A “constitutive promoter” is one that is capable of initiating transcription in nearly all cell types, whereas a “cell type-specific promoter” initiates transcription only in one or a few particular cell types or groups of cells forming a tissue. In some embodiments, the promoter is secondary cell wall-specific and/or fiber cell-specific. A “fiber cell-specific promoter” refers to a promoter that initiates substantially higher levels of transcription in fiber cells as compared to other non-fiber cells of the plant. A “secondary cell wall-specific promoter” refers to a promoter that initiates substantially higher levels of transcription in cell types that have secondary cell walls, e.g., lignified tissues such as vessels and fibers, which may be found in wood and bark cells of a tree, as well as other parts of plants such as the leaf stalk. In some embodiments, a promoter is fiber cell-specific or secondary cell wall-specific if the transcription levels initiated by the promoter in fiber cells or secondary cell walls, respectively, are at least 3-fold, 4-fold, 5-fold, 6-fold, 7-fold, 8-fold, 9-fold, 10-fold, 50-fold, 100-fold, 500-fold, 000-fold higher or more as compared to the transcription levels initiated by the promoter in other tissues, resulting in the encoded protein substantially localized in plant cells that possess fiber cells or secondary cell wall, e.g., the stem of a plant. Non-limiting examples of fiber cell and/or secondary cell wall specific promoters include the promoters directing expression of the genes IRX1, IRX3, IRX5, IRX7, IRX8, IRX9, IRX10, IRX14, NST1, NST2, NST3, MYB46, MYB58, MYB63, MYB83, MYB85, MYB103, PALL PAL2, C3H, CcOAMT, CCR1, FSH, LAC4, LAC17, CADc, and CADd. See, e.g., Turner et al 1997; Meyer et al 1998; Jones et al 2001; Franke et al 2002; Ha et al 2002;Rohde et al 2004; Chen et al 2005; Stobout et al 2005; Brown et al 2005; Mitsuda et al 2005; Zhong et al 2006; Mitsuda et al 2007; Zhong et al 2007a, 2007b; Zhou et al 2009; Brown et al 2009; McCarthy et al 2009; Ko et al 2009; Wu et al 2010; Berthet et al 2011. In some embodiments, a promoter is substantially identical to a promoter from the lignin biosynthesis pathway. A promoter originated from one plant species may be used to direct gene expression in another plant species.

A polynucleotide or amino acid sequence is “heterologous” to an organism or a second polynucleotide or amino acid sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety, or a gene that is not naturally expressed in the target tissue).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “host cell” of “host organism” is used herein to refer to a living biological cell that can be transformed via insertion of an expression vector.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The present invention provides for a toolbox or library of strong plant transcriptional activators that enable us strong upregulation of gene expression in plants. The library enables us to modulate transcription specifically and is easy to implement into different expression systems as well as fusion proteins.

In some embodiments, the toolbox or library of plant transcription factor based regulatory domains that enable strong enhancement of gene expression in plants. The parts work by being tethering to a DNA binding domain of any one of interest and allow strong activation at any locus the transcription factor can be targeted to.

The present invention provides for a method for fast throughput characterization of plant regulatory domains while excluding native DNA binding activity. The method comprises: scanning a library of transcription factors, such as plant transcription factors, such as Arabidopsis thaliana transcription factors, for their DNA binding domains; generating a truncation library excluding the native DNA binding activity or native DNA binding domain; and characterizing of the regulatory domains of the transcription factors. In some embodiments, the characterizing step is parallel to the other steps.

The present invention can be useful for: controlling gene expression in plants; inclusion in a known or novel expression systems, such as for increasing yields in protein expression using our technology.

In some embodiments, the synthetic TF of the present invention do not contain any viral or mammalian parts, or nucleic acid sequence of a viral or mammalian origin.

The synthetic TF of the present invention can be used in the invention taught in PCT International Patent Application No. PCT/US2018/050514 (Publication No. WO 2019/051503 A2), which is hereby incorporated by reference.

The present invention can be used in new or non-model organisms for the controlled expression of multiple genes in a certain manner, including expressing multiple genes simultaneously. The expression of these genes can be regulated in a temporal and/or spatial manner.

The present invention can be used in a strategy to design system utilizing synthetic promoters for the ultimate purpose of controlling expression strength, tissue-specificity, and environmentally-responsive promoters and associated downstream products (e.g. RNA, protein). This method utilizes the synthetic TF of the present invention with its corresponding DNA binding sequence (cis-element), where multiple slightly varying nucleotide sequences of cis-elements are concatenated to provide variability in the binding strength of the transcriptional regulator. The cis-elements are fused to varying minimal promoter sequences (minimal promoter or minimal promoter +UTR upstream sequence of ATG) of the eukaryote host organism of interest to enable the synthetic TF the ability to control expression of the target downstream gene. This invention provides a strategy for engineering an entirely orthogonal transcriptional network into any eukaryotic host for controlling expression strengths of multiple genes through the heterologous expression of the synthetic TF.

The present invention enables one skilled in the art to control the expression of a single or multiple genes simultaneously in any eukaryote organism with only one endogenous promoter using the synthetic TF. Many times, such as in plants, reuse of the same promoter to drive heterologous expression of multiple genes may increase the likelihood of gene silencing and even creates genome instability. Moreover, use of one endogenous promoter may offer the desired expression level required to express a gene of interest. The present invention offers the capacity of retaining expression specificity while offering a dynamic range of expression of the transgene using the synthetic TF. For example, there are many promoters that display tissue-specific expression in one specific tissue (e.g., plant roots, seeds, leaves, or the like). By utilizing a promoter of interest to drive expression of the synthetic TF, one can generate a library of synthetic promoters that are turned on by the synthetic TF at varying expression strengths. This is an efficient and productive way in controlling the exact expression strength of a single or multiple genes in a tissue-specific or environmentally-responsive manner.

The present invention can be applied to any host eukaryotic organism of interest, such as fungi, plant, and animal cells., using the synthetic TF. This invention offers the ability to perform various permutations and test multiple expression profiles. For example, one set of plants could be generated with different promoters driving the synthetic TF (set A) and another set of plants would be transformed with different combination of synthetic promoters driving one or a multiple transgene of interests (set B). Plants from set A could be crossed with those of set B, this would great a 2D matrix of new plants expressing transgene of interests in different tissues and at different strength. This approach has the capacity to reduce number of transformations. For example, generation of 50 plants for each set (A and B) will require 100 transformations and will be used to generate 2500 combinations that would normally require 2500 independent transformations without the use of matrix as presented above. Such matrix approach is applicable to any eukaryotic host that can be crossed such as crops and yeast.

The present invention provides for a strategy to repress genes of interest using the synthetic TF. The invention described here provides an additional layer of control and regulation by utilizing synthetic TF to repress expression of genes. The synthetic TF would comprise a DNA-binding domain which binds the synthetic promoter cis elements and a repressor domain. There are varying strategies to control the level of repression. Various derivatives of the synthetic TF (N- or C-terminus) can result in varying levels of repression. Furthermore, repressors could also either be degrade, sequestered, or change in protein conformation to control spatial and temporal changes in repression of genes of interest.

With the synthetic TF of this present invention, one skilled in the art is able to subtract out certain tissues for where one or more genes of interest (GOI) are expressed. For example, one can use a constitutive promoter to activate expression of GOIs in all tissue and express a repressor specifically in the roots; thus, only expression will be found in the shoots. This is useful for those who may want to avoid the length and laborious process of discovering, characterizing, and validating promoters that have properties they want. Furthermore, within the context of the synthetic promoters system, this provides an additional level of regulation which other strategies and technologies do not have. A further application of this invention is in the context of an environmental response. For example, if one desires a GO1 to be repressed in response to an abiotic or biotic stress for optimal growth, the present invention can provide for a repression system to effect a gradual decrease in expression of the GOIs.

This invention can be used by nearly any biotechnology industry. This invention can easily be utilized for any eukaryotic host, such as plant, yeast or animal hosts.

The present invention provides for the following embodiments of the invention:

TABLE 1
Effector Domains

| | | | | | | log2_ |
|---|---|---|---|---|---|---|
| SEQ | | Locus | | | | GFP |
| ID | | (Common | | | aa | fold- |
| NO: | ID | Name) | Family | amino_acid_seq | length | change |
| 1 | 189 | AT2 | G2- | DEPNEGDQGFSFEHGAGYTYNLSQLPMLQSFDQRPSSSLGYGGGSWTDH | 271 | − |
| | | G40 | like | RRQIYRSPWRGLTTRENTRTRQTMFSSQPGERYHGVSNSILNDKNKTIS | | 2.355 |
| | | 260 | | FRINSHEGVHDNNGVAGAVPRIHRSFLEGMKTENKSWGQSLSSNLKSST | | 57669 |
| | | | | ATIPQDHIATTLNSYQWENAGVAEGSENVLKRKRLLFSDDCNKSDQDLD | | |
| | | | | LSLSLKVPRTHDNLGECLLEDEVKEHDDHQDIKSLSLSLSSSGSSKLDR | | |
| | | | | TIRKEDQTDHKKRKISVLASPLDLTL | | |
| 2 | 127 | AT3 | C2C2- | KKRRTLISNRSEDKKKKSHNRNPKFGDSLKQRLMELGREVMMQRSTAEN | 76 | - |
| | | G06 | GATA | QRRNKLGEEEQAAVLLMALSYASSVYA | | 2.262 |
| | | 740 | | | | 09036 |
| 3 | 138 | AT3 | C2H2 | MALDTLNSPTSTTTTTAPPPFLRCLDETEPENLESWTKRKRTKRHRIDQ | 83 | - |
| | | G49 | | PNPPPSEEEYLALCLLMLARGSSDHHSPPSDHHS | | 2.133 |
| | | 930 | | | | 08504 |
| 4 | 130 | AT2 | C2C2- | MMGYQTNSNFSMFFSSENDDQNHHNYDPYNNFSSSTSVDCTLSLGTPST | 87 | - |
| | | G18 | GATA | RLDDHHRFSSANSNNISGDFYIHGGNAKTSSYKKGGVA | | 2.096 |
| | | 380 | | | | 27875 |
| 5 | 108 | AT1 | C2C2- | RSGSSPSSNLKNQTVAEKPDHHGSGSEEKEERVSGQEMNPTRMLYGLPV | 107 | − |
| | | G51 | DOF | GDPNGASFSSLLASNMQMGGLVYESGSRWLPGMDLGLGSVRRSDDTWTD | | 2.086 |
| | | 700 | | LAMNRMEKN | | 31238 |
| 6 | 234 | AT4 | Homeo | MMMGKEDLGLSLSLGFAQNHPLQLNLKPTSSPMSNLQMFPWNQTLVSSS | 129 | - |
| | | G17 | box | DQQKQQFLRKIDVNSLPTTVDLEEETGVSSPNSTISSTVSGKRRSTERE | | 2.081 |
| | | 460 | | GTSGGGCGDDLDITLDRSSSRGTSDEEEDYG | | 62461 |
| 7 | 338 | AT5 | MYB- | MVSHKCVEEFGYASYLVPSNARAPRSARKRRSIEKRISKEDDNMCAIDL | 292 | - |
| | | G59 | related | LATVAGHLSFESGSSLMSIDKLIEDHRVKEEFPEEEKPLMPVALSPYRG | | 1.975 |
| | | 430 | | SLSPCGFSSVINGKVENEVDGFSYSGGSDACQVGNFSQDVKPDIDGDAV | | 33535 |
| | | | | VLDARPNVVVSLGSSSRTEVPSIGNCVSHGVRDDVNLFSRDDDENESKY | | |
| | | | | IHPRVTKHSPRTVPRIGDRRIRKILASRHWKGGSRHSDTKPWRNYYLHQ | | |
| | | | | QRSYPIKKRKNFDHISDSVTDDYRMRTKMHRGSRKGQGASFVASDSH | | |
| 8 | 155 | AT1 | C2H2 | NLPWKLKQRTSKEVRKRVYVCPEKSCVHHHPTRALGDLTGIKKHFCRKH | 414 | - |
| | | G03 | | GEKKWKCEKCAKRYAVQSDWKAHSKTCGTREYRCDCGTIFSRRDSFITH | | 1.975 |
| | | 840 | | RAFCDALAEETARLNAASHLKSFAATAGSNLNYHYLMGTLIPSPSLPQP | | 24748 |
| | | | | PSFPFGPPQPQHHHHHQFPITTNNFDHQDVMKPASTLSLWSGGNINHHQ | | |
| | | | | QVTIEDRMAPQPHSPQEDYNWVFGNANNHGELITTSDSLITHDNNINIV | | |
| | | | | QSKENANGATSLSVPSLFSSVDQITQDANAASVAVANMSATALLQKAAQ | | |
| | | | | MGATSSTSPTTTITTDQSAYLQSFASKSNQIVEDGGSDRFFASFGSNSV | | |
| | | | | ELMSNNNNGLHEIGNPRNGVTVVSGMGELQNYPWKRRRVDIGNAGGGGQ | | |
| | | | | TRDFLGVGVQTICHSSSINGWI | | |
| 9 | 145 | AT5 | C2H2 | MEAFEEATKEQSLILKGKRTKRQRPQSPIPFSISPPIVSTPENNMEEEY | 152 | - |
| | | G04 | | TDLDSKDNALGNDEGNHKKDGVITSSSSSASWSSQNNHTLKAAEDEEDQ | | 1.961 |
| | | 390 | | DIANCLILLAQGHSLPHNNHHLPNSNNNNTYRFTSRRFLETSSSNSGGK | | 8251 |
| | | | | AGYYV | | |
| 10 | 235 | AT5 | Homeo | MMMGKEDLGLSLSLGFSQNHNPLQMNLNPNSSLSNNLQRLPWNQTFDPT | 124 | - |
| | | G47 | box | SDLRKIDVNSFPSTVNCEEDTGVSSPNSTISSTISGKRSEREGISGTGV | | 1.892 |
| | | 370 | | GSGDDHDEITPDRGYSRGTSDEEEDG | | 76128 |
| 11 | 133 | AT4 | C2C2- | MFGRHSIIPNNQIGTASASAGEDHVSASATSGHIPYDDMEEIPHPDSIY | 207 | - |
| | | G24 | GATA | GAASDLIPDGSQLVAHRSDGSELLVSRPPEGANQLTISFRGQVYVEDAV | | 1.835 |
| | | 470 | | GADKVDAVLSLLGGSTELAPGPQVMELAQQQNHMPVVEYQSRCSLPQRA | | 4667 |
| | | | | QSLDRFRKKRNARCFEKKVRYGVRQEVALRMARNKGQFTSSKMTDGAYN | | |
| | | | | SGTDQDSAQDD | | |
| 12 | 144 | AT3 | C2H2 | EEEQRPSQLSYETESDVSSSDPKFAFTSSVLLEDGESESESSRNVINLT | 141 | - |
| | | G60 | | RKRSKRTRKLDSFVTKKVKTSQLGYKPESDQEPPHSSASDTTTEEDLAF | | 1.808 |
| | | 580 | | CLMMLSRDKWKKNKSNKEVVEEIETEEESEGYNKINRATTKGR | | 38676 |
| 13 | 240 | AT2 | Homeo | KRFNGTNMTTPSSSPNSVMMAANDHYHPLLHHHHGVPMQRPANSVNVKL | 194 | - |
| | | G17 | box | NQDHHLYHHNKPYPSFNNGNLNHASSGTECGVVNASNGYMSSHVYGSME | | 1.798 |
| | | 950 | | QDCSMNYNNVGGGWANMDHHYSSAPYNFFDRAKPLFGLEGHQEEEECGG | | 50715 |
| | | | | DAYLEHRRTLPLFPMHGEDHINGGSGAIWKYGQSEVRPCASLELRLN | | |
| 14 | 128 | AT5 | C2C2- | KKRRGGTEDNKKLKKSSSGGGNRKFGESLKQSLMDLGIRKRSTVEKQRQ | 71 | − |
| | | G49 | GATA | KLGEEEQAAVLLMALSYGSVYA | | 1.762 |
| | | 300 | | | | 50735 |
| 15 | 508 | AT5 | bZIP | ARQQGVFISGTGDQAHSTGGNGALAFDAEHSRWLEEKNKQMNELRSALN | 242 | - |
| | | G06 | | AHAGDSELRIIVDGVMAHYEELFRIKSNAAKNDVFHLLSGMWKTPAERC | | 1.756 |
| | | 950 | | FLWLGGFRSSELLKLLANQLEPMTERQLMGINNLQQTSQQAEDALSQGM | | 34582 |
| | | | | ESLQQSLADTLSSGTLGSSSSGNVASYMGQMAMAMGKLGTLEGFIRQAD | | |
| | | | | NLRLQTLQQMIRVLTTRQSARALLAIHDYESRLRALSSLWLARPRE | | |
| 16 | 152 | AT1 | C2H2 | NLPWKLKQRSNKDVVRKKVYVCPEPGCVHHHPSRALGDLTGIKKHFFRK | 341 | - |
| | | G55 | | HGEKKWKCEKCSKKYAVQSDWKAHAKTCGTKEYKCDCGTLFSRRDSFIT | | 1.741 |
| | | 110 | | HRAFCDALAEESARAMPNPIMIQASNSPHHHHHQTQQNIGFSSSSQNII | | 87114 |
| | | | | SNSNLHGPMKQEESQHHYQNIPPWLISSNPNPNGNNGNLFPPVASSVNT | | |
| | | | | GRSSFPHPSPAMSATALLQKAAQMGSTKSTTPEEEERSSRSSYNNLITT | | |
| | | | | TMAAMMTSPPEPGFGFQDYYMMNHQHHGGGEAFNGGFVPGEEKNDVVDD | | |
| | | | | GGGETRDFLGLRSLMSHNEILSFANNLGNCLNTSATEQQQQQHSHQD | | |
| 17 | 208 | AT1 | HB | SVNGWGRRPAALRALSQRLSRGFNEAVNGFTDEGWSVIGDSMDDVTITV | 458 | - |
| | | G52 | | NSSPDKLMGLNLTFANGFAPVSNVVLCAKASMLLQNVPPAILLRFLREH | | 1.729 |
| | | 150 | | RSEWADNNIDAYLAAAVKVGPCSARVGGFGGQVILPLAHTIEHEEFMEV | | 00452 |
| | | | | IKLEGLGHSPEDAIVPRDIFLLQLCSGMDENAVGTCAELIFAPIDASFA | | |
| | | | | DDAPLLPSGFRIIPLDSAKQEVSSPNRTLDLASALEIGSAGTKASTDQS | | |
| | | | | GNSTCARSVMTIAFEFGIESHMQEHVASMARQYVRGIISSVQRVALALS | | |
| | | | | PSHISSQVGLRTPLGTPEAQTLARWICQSYRGYMGVELLKSNSDGNESI | | |
| | | | | LKNLWHHTDAIICCSMKALPVFTFANQAGLDMLETTLVALQDISLEKIF | | |
| | | | | DDNGRKTLCSEFPQIMQQGFACLQGGICLSSMGRPVSYERAVAWKVLNE | | |
| | | | | EENAHCICFVFINWSFV | | |
| 18 | 171 | AT2 | CCAA | QKEKRKTVNGDDLLWAMATLGFEDYLEPLKIYLARYREVFETNSVLFIP | 92 | - |
| | | G38 | T- | WDWLLTHHLLMQLEGDNKGSGKSGDGSNRDAGGGVSGEEMPSW | | 1.679 |
| | | 880 | HAP3 | | | 67349 |
| 19 | 162 | AT5 | C3H | MSKPEETSDPNPTGPDPSRSSSDEVTVTVADRAPSDLNHVSEELSDQLR | 100 | - |
| | | G63 | | NVGLDDSAKELSVPISVPQGNVETDSRALFGSDQKEEEEGSEKRMMMVY | | 1.675 |
| | | 260 | | PV | | 93209 |
| 20 | 137 | AT3 | C2H2 | REKASNVLVTHSFMPETTTVTTLKKSSSGKRVACLDEDLTSVESFVNTE | 57 | - |
| | | G46 | | LELGRTMY | | 1.639 |
| | | 070 | | | | 19306 |
| 21 | 156 | AT5 | C2H2 | NLPWKLKQRTSKEVRKRVYVCPEKTCVHHHSSRALGDLTGIKKHFCRKH | 378 | - |
| | | G44 | | GEKKWTCEKCAKRYAVQSDWKAHSKTCGTREYRCDCGTIFSRRDSFITH | | 1.598 |
| | | 160 | | RAFCDALAEETAKINAVSHLNGLAAAGAPGSVNLNYQYLMGTFIPPLQP | | 88718 |
| | | | | FVPQPQTNPNHHHQHFQPPTSSSLSLWMGQDIAPPQPQPDYDWVFGNAK | | |
| | | | | AASACIDNNNTHDEQITQNANASLTTTTTLSAPSLFSSDQPQNANANSN | | |
| | | | | VNMSATALLQKAAEIGATSTTTAATNDPSTFLQSFPLKSTDQTTSYDSG | | |
| | | | | EKFFALFGSNNNIGLMSRSHDHQEIENARNDVTVASALDELQNYPWKRR | | |
| | | | | RVDGGGEVGGGGQTRDFLGVGVQTLCHPSSINGWI | | |
| 22 | 168 | AT5 | C3H | ISRELRRKLFGRYRRSYRRGSRSRSRSISPRRKREHSRERERGDVRDRD | 108 | - |
| | | G42 | | RHGNGKRSSDRSERHDRDGGGRRRHGSPKRSRSPRNVREGSEERRARIE | | 1.572 |
| | | 820 | | QWNRERDEGV | | 72079 |
| 23 | 163 | AT1 | C3H | MSEIEELVCIEASVTRKSTSNTVEIRESRRNKVTLGSSDSPAFPTPHLF | 113 | - |
| | | G70 | | LKNIVSFDEQSMYNLLYPRLQDPNLCSILSFKIAFEAKRVPGPLYISYD | | 1.567 |
| | | 910 | | VTLTPQIFEEPDMET | | 15058 |
| 24 | 303 | AT4 | MYB | LKMGIDPVTHTPRLDLLDISSILSSSIYNSSHHHHHHHQQHMNMSRLMM | 207 | - |
| | | G05 | | SDGNHQPLVNPEILKLATSLFSNQNHPNNTHENNTVNQTEVNQYQTGYN | | 1.529 |
| | | 100 | | MPGNEELQSWFPIMDQFTNFQDLMPMKTTVQNSLSYDDDCSKSNFVLEP | | 32787 |
| | | | | YYSDFASVLTTPSSSPTPLNSSSSTYINSSTCSTEDEKESYYSDNITNY | | |
| | | | | SFDVNGFLQFQ | | |
| 25 | 444 | AT2 | WRKY | MAVELMTRNYISGVGADSFAVQEAAASGLKSIENFIGLMSRDSFNSDQP | 233 | − |
| | | G23 | | SSSSASASASAAADLESARNTTADAAVSKFKRVISLLDRTRTGHARFRR | | 1.528 |
| | | 320 | | APVHVISPVLLQEEPKTTPFQSPLPPPPQMIRKGSFSSSMKTIDFSSLS | | 49439 |
| | | | | SVTTESDNQKKIHHHQRPSETAPFASQTQSLSTTVSSFSKSTKRKCNSE | | |
| | | | | NLLTGKCASASSSGRCHCSKKRKIKQRRIIRVPAISA | | |
| 26 | 149 | AT3 | C2H2 | NLPWKLRQKSNKEVKKKVYVCPEVSCVHHDPSRALGDLTGIKKHFCRKH | 367 | - |
| | | G50 | | GEKKWKCDKCSKKYAVQSDWKAHSKICGTKEYKCDCGTLFSRRDSFITH | | 1.525 |
| | | 700 | | RAFCDALAEENARSHHSQSKKQNPEILTRKNPVPNPVPAPVDTESAKIK | | 45828 |
| | | | | SSSTLTIKQSESPKTPPEIVQEAPKPTSLNVVTSNGVFAGLFESSSASP | | |
| | | | | SIYTTSSSSKSLFASSSSIEPISLGLSTSHGSSFLGSNRFHAQPAMSAT | | |
| | | | | ALLQKAAQMGAASSGGSLLHGLGIVSSTSTSIDAIVPHGLGLGLPCGGE | | |
| | | | | SSSGLKELMMGNSSVFGPKQTTLDFLGLGRAVGNGNGPSNGLSTLVGGG | | |
| | | | | TGIDMATTFGSGEFSGKDISRRKS | | |
| 27 | 220 | AT5 | HSF | VPDRWEFSNDFFKRGEKRLLREIQRRKITTTHQTVVAPSSEQRNQTMVV | 212 | - |
| | | G62 | | SPSNSGEDNNNNQVMSSSPSSWYCHQTKTTGNGGLSVELLEENEKLRSQ | | 1.522 |
| | | 020 | | NIQLNRELTQMKSICDNIYSLMSNYVGSQPTDRSYSPGGSSSQPMEFLP | | 94456 |
| | | | | AKRFSEMEIEEEEEASPRLFGVPIGLKRTRSEGVQVKTTAVVGENSDEE | | |
| | | | | TPWLRHYNRTNQRVCN | | |
| 28 | 359 | AT3 | NAC | EELVLGEEDSKSDEVEEPAVSSPTVEVTKSEVSEVIKTEDVKRHDIAES | 305 | - |
| | | G49 | | SLVISGDSHSDACDEATTAELVDFKWYPELESLDFTLFSPLHSQVQSEL | | 1.519 |
| | | 530 | | GSSYNTFQPGSSNFSGNNNNSFQIQTQYGTNEVDTYISDFLDSILKSPD | | 95807 |
| | | | | EDPEKHKYVLQSGFDVVAPDQIAQVCQQGSAVDMSNDVSVTGIQIKSRQ | | |
| | | | | AQPSGYTNDYIAQGNGPRRLRLQSNENGINTKNPELQAIKREAEDTVGE | | |
| | | | | SIKKRCGKLMRSKNVTGFVFKKITSVKCSYGGLFRAAVVAVVFLMSVCS | | |
| | | | | LTVDFRASAVS | | |
| 29 | 190 | AT4 | G2- | MVQTETDQRMGLNLNLSIYSLPKPLSQFLDEVSRIKDNHSKLSEIDGYV | 213 | - |
| | | G37 | like | GKLEEERNKIDVFKRELPLCMLLLNEEIVELCVAIGALKDEARKGLSLM | | 1.445 |
| | | 180 | | ASNGKFDDVERAKPETDKKSWMSSAQLWISNPNSQFRSTNEEEEDRCVS | | 80364 |
| | | | | QNPFQTCNYPNQGGVFMPFNRPPPPPPPAPLSLMTPTSEMMMDYSRIEQ | | |
| | | | | SHHHHQFNKPSSQSHHI | | |
| 30 | 307 | AT2 | MYB | KHEAMAKENRIACCVNSDNKRLLFPDGISTPLKAESESPLTKKMRRSHI | 353 | − |
| | | G02 | | PNLTEIKSYGDRSHIKVESTMNQQRRHPFSVVAHNATSSDGTEEQKQIG | | 1.390 |
| | | 820 | | NVKESDGEDKSNQEVFLKKDDSKVTALMQQAELLSSLAQKVNADNTDQS | | 37827 |
| | | | | MENAWKVLQDFLNKSKENDLFRYGIPDIDFQLDEFKDLVEDLRSSNEDS | | |
| | | | | QSSWRQPDLHDSPASSEYSSGSGSGSTIMTHPSGDKTQQLMSDTQTTSH | | |
| | | | | QQNGGELLQDNGIVSDATVEQVGLLSTGHDVLKNSNETVPIPGEEEENS | | |
| | | | | PVQVTPLERSLAAGIPSPQFSESERNFLLKTLGVESPSPYPSANPSQPP | | |
| | | | | PCKRVLLDSL | | |
| 31 | 375 | AT1 | NAC | TGDRKNVGLIHNQISYLHNHSLSTTHHHHHEALPLLIEPSNKTLTNFPS | 163 | - |
| | | G76 | | LLYDDPHQNYNNNNFLHGSSGHNIDELKALINPVVSQLNGIIFPSGNNN | | 1.388 |
| | | 420 | | NDEDDFDFNLGVKTEQSSNGNEIDVRDYLENPLFQEASYGLLGFSSSPG | | 79104 |
| | | | | PLHMLLDSPCPLGFQL | | |
| 32 | 159 | AT1 | C2H2 | MALEALTSPRLASPIPPLFEDSSVFHGVEHWTKGKRSKRSRSDFHHQNL | 79 | - |
| | | G27 | | TEEEYLAFCLMLLARDNRQPPPPPAVEKLS | | 1.351 |
| | | 730 | | | | 56052 |
| 33 | 126 | AT3 | C2C2- | MSGREDEEEDLGTAMQKIPIPVNVFDKEPMDLDTVFGFADGVREIIEDS | 110 | - |
| | | G45 | GATA | NLLLEESREFDTNDSKPSRNFSNLPTATRGRLHAPKRSGNKRGRQKRLS | | 1.348 |
| | | 170 | | FKSPSDLFDSKF | | 2749 |
| 34 | 44 | AT2 | AP2- | ELLAGLTVSNGGGRGGDLSAAYIRRKAAEVGAQVDALGATVVVNTGGEN | 92 | - |
| | | G23 | EREB | RGDYEKIENCRKSGNGSLERVDLNKLPDPENSDGDDDECVKRR | | 1.333 |
| | | 340 | P | | | 70617 |
| 35 | 161 | AT1 | C2H2 | MSNPACSNLENNGCDHNSFNYSTSLSYIYNSHGSYYYSNTTNPNYINHT | 177 | - |
| | | G51 | | HTTSTSPNSPPLREALPLLSLSPIRHQEQQDQHYFMDTHQISSSNELDD | | 1.302 |
| | | 220 | | PLVTVDLHLGLPNYGVGESIRSNIAPDATTDEQDQDHDRGVEVTVESHL | | 18052 |
| | | | | DDDDDHHGDLHRGHHYWIPTPSQILIGPTQ | | |
| 36 | 471 | AT4 | WRKY | MTVELMMSSYSGGGGGGDGFPAIAAAAKMEDTALREAASAGIHGVEEFL | 274 | − |
| | | G24 | | KLIGQSQQPTEKSQTEITAVTDVAVNSFKKVISLLGRSRTGHARFRRAP | | 1.294 |
| | | 240 | | ASTQTPFKQTPVVEEEVEVEEKKPETSSVLTKQKTEQYHGGGSAFRVYC | | 15365 |
| | | | | PTPIHRRPPLSHNNNNNQNQTKNGSSSSSPPMLANGAPSTINFAPSPPV | | |
| | | | | SATNSFMSSHRCDTDSTHMSSGFEFTNPSQLSGSRGKPPLSSASLKRRC | | |
| | | | | NSSPSSRCHCSKKRKSRVKRVIRVPAVSS | | |
| 37 | 374 | AT5 | NAC | TTLASTGAVSEGGGGGGATVSVSSGTGPSKKTKVPSTISRNYQEQPSSP | 206 | - |
| | | G53 | | SSVSLPPLLDPTTTLGYTDSSCSYDSRSTNTTVTASAITEHVSCFSTVP | | 1.292 |
| | | 950 | | TTTTALGLDVNSFSRLPPPLGEDEDPFPRFVSRNVSTQSNFRSFQENEN | | 1397 |
| | | | | QFPYFGSSSASTMTSAVNLPSFQGGGGVSGMNYWLPATAEENESKVGVL | | |
| | | | | HAGLDCIWNY | | |
| 38 | 290 | AT1 | MYB | RERSKLRPRGLGHDGTVAATGMIGNYKDCDKERRLATTTAINFPYQFSH | 143 | - |
| | | G17 | | INHFQVLKEFLTGKIGFRNSTTPIQEGAIDQTKRPMEFYNFLQVNTDSK | | 1.278 |
| | | 950 | | IHELIDNSRKDEEEDVDQNNRIPNENCVPFFDFLSVGNSASQGLC | | 84299 |
| 39 | 319 | AT5 | MYB- | TTLHHKRRRTSLFDMVSAGNVEENSTTKRICNDHIGSSSKVVWKQGLLN | 92 | - |
| | | G56 | related | PRLGYPDPKVSVSGSGNSGGLDLELKLASIQSPESNIRPISVT | | 1.211 |
| | | 840 | | | | 03834 |
| 40 | 140 | AT5 | C2H2 | MTSIPNGLNSYVDDTVNICGFTPIEMSSNLRNHESKMVHSMENTSDHTN | 245 | - |
| | | G22 | | HHGLFSSSRVFNFYQDSHVSSSSFGFNNSHMAYHMRKNMVSTFGMPCIT | | 1.204 |
| | | 990 | | QNSNNPHLSQISITQTITNSYSAIVPTYNLITSQNEYQRAKEPNIENPP | | 33665 |
| | | | | FYPPNFVDKNVGNQCQILNPTPLNTIFPHQASIFPRNVDKESFSPKQNP | | |
| | | | | HQYVSYRQPLKRHCRPTKKFENTFSDFDSGKDIEYDGRTHSLPYEKYGP | | |
| 41 | 304 | AT3 | MYB | SGGVAVTTVTETEEDQDRPKKRRSVSFDSAFAPVDTGLYMSPESPNGID | 194 | - |
| | | G50 | | VSDSSTIPSPSSPVAQLFKPMPISGGFTVVPQPLPVEMSSSSEDPPTSL | | 1.177 |
| | | 060 | | SLSLPGAENTSSSHNNNNNALMFPRFESQMKINVEERGEGRRGEFMTVV | | 66466 |
| | | | | QEMIKAEVRSYMAEMQKTSGGFVVGGLYESGGNGGFRDCGVITPKVE | | |
| 42 | 202 | AT1 | G2- | MELFPAQPDLSLQISPPNSKPSSTWQRRRSTTDQEDHEELDLGFWRRAL | 208 | - |
| | | G32 | like | DSRTSSLVSNSTSKTINHPFQDLSLSNISHHQQQQQHHHPQLLPNCNSS | | 1.134 |
| | | 240 | | NILTSFQFPTQQQQQHLQGFLAHDLNTHLRPIRGIPLYHNPPPHHHPHR | | 97586 |
| | | | | PPPPCFPFDPSSLIPSSSTSSPALTGNNNSENTSSVSNPNYHNHHHQTL | | |
| | | | | NRARFMPRFPAK | | |
| 43 | 122 | AT4 | C2C2- | RVNQPSVARMVSVETQRGNNQPFSNVQENVHLVGSFGASSSSSVGAVGN | 170 | - |
| | | G21 | DOF | LFGSLYDIHGGMVTNLHPTRTVRPNHRLAFHDGSFEQDYYDVGSDNLLV | | 1.124 |
| | | 080 | | NQQVGGYGYHMNPVDQFKWNQSFNNTMNMNYNNDSTSGSSRGSDMNVNH | | 80267 |
| | | | | DNKKIRYRNSVIMHPCHLEKDGP | | |
| 44 | 283 | AT4 | MYB | INRGIDPTSHRPIQESSASQDSKPTQLEPVTSNTINISFTSAPKVETFH | 166 | - |
| | | G38 | | ESISFPGKSEKISMLTFKEEKDECPVQEKFPDLNLELRISLPDDVDRLQ | | 1.113 |
| | | 620 | | GHGKSTTPRCFKCSLGMINGMECRCGRMRCDVVGGSSKGSDMSNGEDFL | | 13341 |
| | | | | GLAKKETTSLLGFRSLEMK | | |
| 45 | 132 | AT3 | C2C2- | MESVELTLKNSNMKDKTLTGGAQNGDDFSVDDLLDFSKEEEDDDVLVED | 216 | − |
| | | G51 | GATA | EAELKVQRKRGVSDENTLHRSNDESTADFHTSGLSVPMDDIAELEWLSN | | 1.111 |
| | | 080 | | FVDDSSFTPYSAPTNKPVWLTGNRRHLVQPVKEETCFKSQHPAVKTRPK | | 18695 |
| | | | | RARTGVRVWSHGSQSLTDSSSSSTTSSSSSPRPSSPLWLASGQFLDEPM | | |
| | | | | TKTQKKKKVWKNAGQTQTQT | | |
| 46 | 330 | AT5 | MYB- | NVSRRKRRSSLFDMVPDEVGDIPMDLQEPEEDNIPVETEMQGADSIHQT | 219 | - |
| | | G47 | related | LAPSSLHAPSILEIEECESMDSTNSTTGEPTATAAAASSSSRLEETTQL | | 1.105 |
| | | 390 | | QSQLQPQPQLPGSFPILYPTYFSPYYPFPFPIWPAGYVPEPPKKEETHE | | 32589 |
| | | | | ILRPTAVHSKAPINVDELLGMSKLSLAESNKHGESDQSLSLKLGGGSSS | | |
| | | | | RQSAFHPNPSSDSSDIKSVIHAL | | |
| 47 | 229 | AT1 | Homeo | HTEMECEYLKRWFGSLKEQNRRLQIEVEELRALKPSSTSALTMCPRCER | 82 | - |
| | | G70 | box | VTDAVDNDSNAVQEGAVLSSRSRMTISSSSSLC | | 1.096 |
| | | 920 | | | | 09828 |
| 48 | 244 | AT2 | LOBAS2 | LRHKYQEATTITSLQNNENSTTTTSSVSCDQHALASAILLPPPPPPPPT | 116 | - |
| | | G30 | | PRPPRLLSSQPAPPPTPPVSLPSPSMVVSSSSSSNSSATNSMYNPPPSS | | 1.079 |
| | | 340 | | TAGYSNSLSSDNNVHYFD | | 33364 |
| 49 | 251 | AT5 | MADS | MKQTLSRYGNHQSSSASKAEEDCAEVDILKDQLSKLQEKHLQLQGKGLN | 207 | - |
| | | G13 | | PLTFKELQSLEQQLYHALITVRERKERLLTNQLEESRLKEQRAELENET | | 1.077 |
| | | 790 | | LRRQVQELRSFLPSFTHYVPSYIKCFAIDPKNALINHDSKCSLQNTDSD | | 89686 |
| | | | | TTLQLGLPGEAHDRRTNEGERESPSSDSVTTNTSSETAERGDQSSLANS | | |
| | | | | PPEAKRQRFSV | | |
| 50 | 88 | AT1 | ARID | FTARGPLLHPIATFHANPSTSKEMALVEYTPPSIRYHNTHPPSQGSSSE | 125 | - |
| | | G76 | | TAIGTIEGKFDCGYLVKVKLGSEILNGVLYHSAQPGPSSSPTAVLNNAV | | 1.061 |
| | | 110 | | VPYVETGRRRRRLGKRRRSRRREDPNY | | 08216 |
| 51 | 147 | AT5 | C2H2 | NLPWKLRQRSTKEVRKKVYVCPVSGCVHHDPSRALGDLTGIKKHFCRKH | 417 | - |
| | | G66 | | GEKKWKCEKCSKKYAVQSDWKAHSKICGTKEYKCDCGTLFSRRDSFITH | | 1.047 |
| | | 730 | | RAFCDALAEESAKNHTQSKKLYPETVTRKNPEIEQKSPAAVESSPSLPP | | 87583 |
| | | | | SSPPSVAIAPAPAISVETESVKIISSSVLPIQNSPESQENNNHPEVIIE | | |
| | | | | EASRTIGENVSSSDLSNDHSNNNGGYAGLFVSSTASPSLYASSTASPSL | | |
| | | | | FAPSSSMEPISLCLSTNPSLFGPTIRDPPHELTPLPPQPAMSATALLQK | | |
| | | | | AAQMGSTGSGGSLLRGLGIVSTTSSSMELSNHDALSLAPGLGLGLPCSS | | |
| | | | | GGSGSGLKELMMGNSSVFGPKQTTLDFLGLGRAVGNGGNTGGGLSALLT | | |
| | | | | SIGGGGGIDLFGSGEFSGKDIGRSS | | |
| 52 | 507 | AT5 | bZIP | ARSQGVFFGGSLIGGDQQQGGLPIGPGNISSEAAVEDMEYARWLEEQQR | 257 | − |
| | | G06 | | LLNELRVATQEHLSENELRMFVDTCLAHYDHLINLKAMVAKTDVFHLIS | | 1.035 |
| | | 839 | | GAWKTPAERCFLWMGGFRPSEIIKVIVNQIEPLTEQQIVGICGLQQSTQ | | 89525 |
| | | | | EAEEALSQGLEALNQSLSDSIVSDSLPPASAPLPPHLSNFMSHMSLALN | | |
| | | | | KLSALEGFVLQADNLRHQTIHRLNQLLTTRQEARCLLAVAEYFHRLQAL | | |
| | | | | SSLWLARPRQDG | | |
| 53 | 335 | AT1 | MYB- | KEAEVKGIPVCQALDIEIPPPRPKRKPNTPYPRKPGNNGTSSSQVSSAK | 572 | − |
| | | G01 | related | DAKLVSSASSSQLNQAFLDLEKMPFSEKTSTGKENQDENCSGVSTVNKY | | 1.034 |
| | | 060 | | PLPTKQVSGDIETSKTSTVDNAVQDVPKKNKDKDGNDGTTVHSMQNYPW | | 03155 |
| | | | | HFHADIVNGNIAKCPQNHPSGMVSQDFMFHPMREETHGHANLQATTASA | | |
| | | | | TTTASHQAFPACHSQDDYRSFLQISSTFSNLIMSTLLQNPAAHAAATFA | | |
| | | | | A54SVWPYASVGNSGDSSTPMSSSPPSITAIAAATVAAATAWWASHGLL | | |
| | | | | PVCAPAPITCVPFSTVAVPTPAMTEMDTVENTQPFEKQNTALQDQNLAS | | |
| | | | | KSPASSSDDSDETGVTKLNADSKTNDDKIEEVVVTAAVHDSNTAQKKNL | | |
| | | | | VDRSSCGSNTPSGSDAETDALDKMEKDKEDVKETDENQPDVIELNNRKI | | |
| | | | | KMRDNNSNNNATTDSWKEVSEEGRIAFQALFARERLPQSFSPPQVAENV | | |
| | | | | NRKQSDTSMPLAPNFKSQDSCAADQEGVVMIGVGTCKSLKTRQTGFKPY | | |
| | | | | KRCSMEVKESQVGNINNQSDEKVCKRLRLEGEAST | | |
| 54 | 134 | AT3 | C2C2- | MDDLHGRNGRMHIGVAQNPMHVQYEDHGLHHIDNENSMMDDHADGGMDE | 212 | - |
| | | G21 | GATA | GVETDIPSHPGNSADNRGEVVDRGIENGDQLTLSFQGQVYVEDRVSPEK | | 1.029 |
| | | 175 | | VQAVLLLLGGREVPHTLPTTLGSPHQNNRVLGLSGTPQRLSVPQRLASL | | 9277 |
| | | | | LRFREKRKGRNFDKTIRYTVRKEVALRMQRKKGQFTSAKSSNDDSGSTG | | |
| | | | | SDWGSNQSWAVEGTET | | |
| 55 | 58 | AT1 | AP2- | IDSSSPPPPNLRENQIRNQNQNQVDPFMDHRLFTDHQQQFPIVNRPTSS | 141 | - |
| | | G50 | EREBP | SMSSTVESFSGPRPTTMKPATTKRYPRTPPVVPEDCHSDCDSSSSVIDD | | 1.023 |
| | | 640 | | DDDIASSSRRRNPPFQFDLNFPPLDCVDLENGADDLHCTDLRL | | 82487 |
| 56 | 511 | AT5 | bZIP | ARQQGVFISSSGDQAHSTAGDGAMAFDVEYRRWQEDKNRQMKELSSAID | 242 | - |
| | | G06 | | SHATDSELRIIVDGVIAHYEELYRIKGNAAKSDVFHLLSGMWKTPAERC | | 0.996 |
| | | 960 | | FLWLGGFRSSELLKLIASQLEPLTEQQSLDINNLQQSSQQAEDALSQGM | | 32295 |
| | | | | DNLQQSLADTLSSGTLGSSSSGNVASYMGQMAMAMGKLGTLEGFIRQAD | | |
| | | | | NLRLQTYQQMVRLLTTRQSARALLAVHNYTLRLRALSSLWLARPRE | | |
| 57 | 139 | AT4 | C2H2 | MVSPFSMPFIAQTSGFVNYSQVFITQTIAKRYHALIPTSNMVIVQNDND | 157 | - |
| | | G26 | | RVNRFMTSYPPILKSTVNPPNDFDKQYETFTPKPIDFFCSQQDYACRQH | | 0.995 |
| | | 030 | | LDIFSSSPKHYHEQYVHKNGRSVKYICKPTEVLEEIHDEIDYEKDGGWI | | 04235 |
| | | | | YSLPFEKDSS | | |
| 58 | 236 | AT4 | Homeo | MGLDDSCNTGLVLGLGLSPTPNNYNHAIKKSSSTVDHRFIRLDPSLTLS | 120 | - |
| | | G37 | box | LSGESYKIKTGAGAGDQICRQTSSHSGISSFSSGRVKREREISGGDGEE | | 0.967 |
| | | 790 | | EAEETTERVVCSRVSDDHDDEE | | 67879 |
| 59 | 238 | AT3 | Homeo | LMSSTVSTSTNPSPINCNGRKSMLKLAKRMTDNFCGGVCASSLQKWSKL | 267 | - |
| | | G61 | box | NVGNVDEDVRIMTRKSVNNPGEPPGIILNAATSVWMPVSPRRLFDFLGN | | 0.965 |
| | | 150 | | ERLRSEWDILSNGGPMKEMAHIAKGHDRSNSVSLLRASAINANQSSMLI | | 89465 |
| | | | | LQETSIDAAGAVVVYAPVDIPAMQAVMNGGDSAYVALLPSGFAILPNGQ | | |
| | | | | AGTQRCAAEERNSIGNGGCMEEGGSLLTVAFQILVNSLPTAKLTVESVE | | |
| | | | | TVNNLISCTVQKIKAALHCDST | | |
| 60 | 165 | AT5 | C3H | RTVDFNKVVIALKDYAALRERTADGDPNPVVVNNNTSSSGIDPDAVAAI | 200 | - |
| | | G08 | | RRQRLSEISLWFGPHCSTNNNNSSNSAAAGTASSQVTSEQPVGIVNEDI | | 0.946 |
| | | 750 | | LPMESRATKWAVEGTGILLATGLLTVTLAWLIAPRVGKRTAKSGLHILL | | 71795 |
| | | | | GGLCALTVVIFFRFVVLTRIRYGPARYWAILFVFWFLVFGIWASRSHAS | | |
| | | | | HSST | | |
| 61 | 215 | AT1 | HB | YSGGRQPAVLRTFSQRLCRGENDAVNGFVDDGWSPMSSDGGEDITIMIN | 453 | − |
| | | G30 | | SSSAKFAGSQYGSSFLPSFGSGVLCAKASMLLQNVPPLVLIRFLREHRA | | 0.943 |
| | | 490 | | EWADYGVDAYSAASLRATPYAVPCVRTGGFPSNQVILPLAQTLEHEEFL | | 20642 |
| | | | | EVVRLGGHAYSPEDMGLSRDMYLLQLCSGVDENVVGGCAQLVFAPIDES | | |
| | | | | FADDAPLLPSGFRVIPLDQKTNPNDHQSASRTRDLASSLDGSTKTDSET | | |
| | | | | NSRLVLTIAFQFTFDNHSRDNVATMARQYVRNVVGSIQRVALAITPRPG | | |
| | | | | SMQLPTSPEALTLVRWITRSYSIHTGADLFGADSQSCGGDTLLKQLWDH | | |
| | | | | SDAILCCSLKTNASPVFTFANQAGLDMLETTLVALQDIMLDKTLDDSGR | | |
| | | | | RALCSEFAKIMQQGYANLPAGICVSSMGRPVSYEQATVWKVVDDNESNH | | |
| | | | | CLAFTLVSWSFV | | |
| 62 | 247 | AT1 | LOBAS2 | GWDNNQRVENNNSNNKNGLAMTNSSGSGGFSVNNNGVGVNREIVNGGYA | 83 | - |
| | | G06 | | SRNVQGGWENLKHDQRQQCYAVINNGFKQHYLPL | | 0.939 |
| | | 280 | | | | 83042 |
| 63 | 241 | AT2 | LIM | KGSYNHLIKSASIKRATAAATAAAAAVAAVPES | 33 | - |
| | | G39 | | | | 0.934 |
| | | 900 | | | | 34777 |
| 64 | 225 | AT2 | HSF | TTIRWEFSNEMFRKGQRELMSNIRRRKSQHWSHNKSNHQVVPTTTMVNQ | 140 | - |
| | | G41 | | EGHQRIGIDHHHEDQQSSATSSSFVYTALLDENKCLKNENELLSCELGK | | 0.932 |
| | | 690 | | TKKKCKQLMELVERYRGEDEDATDESDDEEDEGLKLFGVKLE | | 47166 |
| 65 | 255 | AT1 | MADS | SPGTQIAILATPLSSHSHASFYSFGHSSVDHVVSSLLHNQHPSLPTNQD | 151 | - |
| | | G60 | | NRSGLGFWWEDQAFDRLENVDELKEAVDAVSRMLNNVRLRLDDAVKSNQ | | 0.909 |
| | | 920 | | RDGSLVIHQEDEEVLQLGYKDTNQITKLEGETSASASLLKNVVDNLHID | | 86825 |
| | | | | DRYY | | |
| 66 | 442 | AT4 | WRKY | MAVDLMRFPKIDDQTAIQEAASQGLQSMEHLIRVLSNRPEQQHNVDCSE | 239 | - |
| | | G31 | | ITDFTVSKFKTVISLLNRTGHARFRRGPVHSTSSAASQKLQSQIVKNTQ | | 0.888 |
| | | 550 | | PEAPIVRTTTNHPQIVPPPSSVTLDFSKPSIFGTKAKSAELEFSKENFS | | 72558 |
| | | | | VSLNSSFMSSAITGDGSVSNGKIFLASAPLQPVNSSGKPPLAGHPYRKR | | |
| | | | | CLEHEHSESFSGKVSGSAYGKCHCKKSRKNRMKRTVRVPAISA | | |
| 67 | 259 | AT2 | MADS | MKEVLERHNLQSKNLEKLDQPSLELQLVENSDHARMSKEIADKSHRLRQ | 179 | - |
| | | G22 | | MRGEELQGLDIEELQQLEKALETGLTRVIETKSDKIMSEISELQKKGMQ | | 0.878 |
| | | 540 | | LMDENKRLRQQGTQLTEENERLGMQICNNVHAHGGAESENAAVYEEGQS | | 10122 |
| | | | | SESITNAGNSTGAPVDSESSDTSLRLGLPYGG | | |
| 68 | 5 | AT4 | ABI3- | AEINFVHNINNHNFVFGSPTYPTARFYPVTPEYSMPYRSFPPFYQNQFQ | 188 | - |
| | | G01 | VP1 | EREYLGYGYGRVVNGNGVRYYAGSPLDQHHQWNLGRSEPLVYDSVPVFP | | 0.876 |
| | | 500 | | AGRVPPSAPPQPSTTKKLRLFGVDVEESSSSGDTRGEMGVAGYSSSSPV | | 61283 |
| | | | | VIRDDDQSFWRSPRGEMASSSSAMQLSDDEEYKRKGKSLEL | | |
| 69 | 193 | AT1 | G2- | MMMFKSGDMDYTQKMKRCHEYVEALEEEQKKIQVFQRELPLCLELVTQA | 205 | - |
| | | G25 | like | IESCRKELSESSEHVGGQSECSERTTSECGGAVFEEFMPIKWSSASSDE | | 0.866 |
| | | 550 | | TDKDEEAEKTEMMTNENNDGDKKKSDWLRSVQLWNQSPDPQPNNKKPMV | | 1177 |
| | | | | IEVKRSAGAFQPFQKEKPKAADSQPLIKAITPTSTTTTSSTAETVGGGK | | |
| | | | | EFEEQKQSH | | |
| 70 | 86 | AT1 | ARID | NNGELNLPGSTLILSSSVEKEPSSHQGSGSGRARRDSAARAMQGWHAQR | 117 | - |
| | | G20 | | LVGSGEVTAPAVKDKGLISTPKHKKLKSIGLQKHKQQTSMDHVVTNEAD | | 0.834 |
| | | 910 | | KQLAAEVVDVGPVADWVKI | | 09023 |
| 71 | 264 | AT3 | MYB | IDFEKAKNIGTGSLVVDDSGEDRTTTVASSEETLSSGGGCHVTTPIVSP | 237 | - |
| | | G09 | | EGKEATTSMEMSEEQCVEKTNGEGISRQDDKDPPTLFRPVPRLSSFNAC | | 0.774 |
| | | 230 | | NHMEGSPSPHIQDQNQLQSSKQDAAMLRLLEGAYSERFVPQTCGGGCCS | | 18221 |
| | | | | NNPDGSFQQESLLGPEFVDYLDSPTFPSSELAAIATEIGSLAWLRSGLE | | |
| | | | | SSSVRVMEDAVGRLRPQGSRGHRDHYLVSEQGTNITNVLST | | |
| 72 | 223 | AT5 | HSF | DTERWEFANEHFLKGERHLLKNIKRRKTSSQTQTQSLEGEIHELRRDRM | 199 | − |
| | | G43 | | ALEVELVRLRRKQESVKTYLHLMEEKLKVTEVKQEMMMNFLLKKIKKPS | | 0.765 |
| | | 840 | | FLQSLRKRNLQGIKNREQKQEVISSHGVEDNGKFVKAEPEEYGDDIDDQ | | 78374 |
| | | | | CGGVFDYGDELHIASMEHQGQGEDEIEMDSEGIWKGFVLSEEEMCDLVE | | |
| | | | | HFI | | |
| 73 | 402 | AT1 | REM | MQMDSAQNQFNKRARLFEDPELKDAKVIYPSNPESTEPVNKGYGGSTAI | 128 | - |
| | | G49 | | QSFFKESKAEETPKVLKKRGRKKKNPNPEEVNSSTPGGDDSENRSKFYE | | 0.718 |
| | | 480 | | SASARKRTVTAEERERAVNAAKTFEPTNPY | | 33073 |
| 74 | 340 | AT1 | NAC | GEETEISSSSTGSEIEQIHSLIPLVNSSGGSEGSSFHSQELQNSSQSGV | 208 | - |
| | | G02 | | FANVQGESQIDDATTPIEEEWKTWLNNDGDEQRNIMFMQDHRSDYTPLK | | 0.686 |
| | | 230 | | SLTGVFSDDSSDDNDSDLISPKTNSIGTSSTCASFASSNHQIDQTQHSP | | 6819 |
| | | | | DSTVQLVSLTQEVSQGPGQVTVIREHKLGEESVKKKRASFVYRMIHRLV | | |
| | | | | KKIHQCYSISRT | | |
| 75 | 261 | AT3 | MYB | EDYQPAKPKTSNKKKGTKPKSESVITSSNSTRSESELADSSNPSGESLF | 169 | - |
| | | G23 | | STSPSTSEVSSMTLISHDGYSNEINMDNKPGDISTIDQECVSFETFGAD | | 0.664 |
| | | 250 | | IDESFWKETLYSQDEHNYVSNDLEVAGLVEIQQEFQNLGSANNEMIFDS | | 86023 |
| | | | | EMDFWFDVLARTGGEQDLLAGL | | |
| 76 | 326 | AT3 | MYB- | METLHPFSHLPISDHRFVVQEMVSLHSSSSGSWTKEENKMFERALAIYA | 120 | - |
| | | G11 | related | EDSPDRWFKVASMIPGKTVFDVMKQYSKLEEDVEDIEAGRVPIPGYPAA | | 0.652 |
| | | 280 | | SSPLGFDTDMCRKRPSGARGSD | | 31617 |
| 77 | 172 | AT1 | CCAA | HRENRKTVNGDDIWWALSTLGLDNYADAVGRHLHKYREAERERTEHNKG | 85 | - |
| | | G09 | T- | SNDSGNEKETNTRSDVQNQSTKFIRVVEKGSSSSAR | | 0.630 |
| | | 030 | HAP3 | | | 49693 |
| 78 | 125 | AT5 | C2C2- | MEDEAHEFFHTSDFAVDDLLVDESNDDDEENDVVADSTTTTTITDSSNF | 214 | - |
| | | G25 | GATA | SAADLPSFHGDVQDGTSFSGDLCIPSDDLADELEWLSNIVDESLSPEDV | | 0.612 |
| | | 830 | | HKLELISGFKSRPDPKSDTGSPENPNSSSPIFTTDVSVPAKARSKRSRA | | 41872 |
| | | | | AACNWASRGLLKETFYDSPFTGETILSSQQHLSPPTSPPLLMAPLGKKQ | | |
| | | | | AVDGGHRRKKDVSSPESG | | |
| 79 | 221 | AT4 | HSF | VPDRWEFSNDCFKRGEKILLRDIQRRKISQPAMAAAAAAAAAAVAASAV | 254 | - |
| | | G11 | | TVAAVPVVAHIVSPSNSGEEQVISSNSSPAAAAAAIGGVVGGGSLQRTT | | 0.612 |
| | | 660 | | SCTTAPELVEENERLRKDNERLRKEMTKLKGLYANIYTLMANFTPGQED | | 05438 |
| | | | | CAHLLPEGKPLDLLPERQEMSEAIMASEIETGIGLKLGEDLTPRLFGVS | | |
| | | | | IGVKRARREEELGAAEEEDDDRREAAAQEGEQSSDVKAEPMEENNSGNH | | |
| | | | | NGSWLELGK | | |
| 80 | 337 | AT5 | MYB- | WGSRKKAKLALKRTPPGTKQDDNNTALTIVALTNDDERAKPTSPGGSGG | 238 | - |
| | | G67 | related | GSPRTCASKRSITSLDKIIFEAITNLRELRGSDRTSIFLYIEENFKTPP | | 0.579 |
| | | 580 | | NMKRHVAVRLKHLSSNGTLVKIKHKYRFSSNFIPAGARQKAPQLFLEGN | | 86361 |
| | | | | NKKDPTKPEENGANSLTKFRVDGELYMIKGMTAQEAAEAAARAVAEAEF | | |
| | | | | AITEAEQAAKEAERAEAEAEAAQIFAKAAMKALKFRIRNHPW | | |
| 81 | 207 | AT4 | HB | LISSSVTSHDNTSITPGGRKSMLKLAQRMTFNFCSGISAPSVHNWSKLT | 256 | − |
| | | G00 | | VGNVDPDVRVMTRKSVDDPGEPPGIVLSAATSVWLPAAPQRLYDELRNE | | 0.572 |
| | | 730 | | RMRCEWDILSNGGPMQEMAHITKGQDQGVSLLRSNAMNANQSSMLILQE | | 74295 |
| | | | | TCIDASGALVVYAPVDIPAMHVVMNGGDSSYVALLPSGFAVLPDGGIDG | | |
| | | | | GGSGDGDQRPVGGGSLLTVAFQILVNNLPTAKLTVESVETVNNLISCTV | | |
| | | | | QKIRAALQCES | | |
| 82 | 195 | AT2 | G2- | LPDSSSEGKKTDKKESGDMLSGLDGSSGMQITEALKLQMEVQKRLHEQL | 214 | - |
| | | G01 | like | EVQRQLQLRIEAQGKYLKKIIEEQQRLSGVLGEPSAPVTGDSDPATPAP | | 0.551 |
| | | 060 | | TSESPLQDKSGKDCGPDKSLSVDESLSSYREPLTPDSGCNIGSPDESTG | | 96733 |
| | | | | EERLSKKPRLVRGAAGYTPDIVVGHPILESGLNTSYHQSDHVLAFDQPS | | |
| | | | | TSLLGAEEQLDKVSGDNL | | |
| 83 | 339 | AT3 | MYB- | MVSHKVLEFGDDGYKLPAQARAPRSLRKKRIYEKKIPGDDKMCAIDLLA | 284 | - |
| | | G46 | related | TVAGSLLLESKSPVNACLVVQNTVKNEYPADENPVKAVPYSESPSLEDN | | 0.545 |
| | | 590 | | GKCGFSSVITNPNHLLVGDKVGKEVEGFSSLGVSGDVKPDVVASIGSNS | | 85295 |
| | | | | STEVGACGNGSPNESRDDVNLFSRNDDDENFSGYIRTRMTRPVPRIGDR | | |
| | | | | RIRKILASRHWKGGSKNNTDAKPWYCSKRSYYLHHHQRSYPIKKRKYFD | | |
| | | | | SVYDSNSDDYRLQGKTHKGSRTISSMKSRNASFVSRDHH | | |
| 84 | 186 | AT1 | G2- | MGSLGDELSLGSIFGRGVSMNVVAVEKVDEHVKKLEEEKRKLESCQLEL | 188 | - |
| | | G49 | like | PLSLQILNDAILYLKDKRCSEMETQPLLKDFISVNKPIQGERGIELLKR | | 0.529 |
| | | 560 | | EELMREKKFQQWKANDDHTSKIKSKLEIKRNEEKSPMLLIPKVETGLGL | | 3219 |
| | | | | GLSSSSIRRKGIVASCGFTSNSMPQPPTPAVPQQPAFLKQQ | | |
| 85 | 64 | AT3 | AP2- | IDCSPSSPLQPLTYLHNQNLCSPPVIQNQIDPFMDHRLYGGGNFQEQQQ | 161 | - |
| | | G20 | EREBP | QQIISRPASSSMSSTVKSCSGPRPMEAAAASSSVAKPLHAIKRYPRTPP | | 0.520 |
| | | 310 | | VAPEDCHSDCDSSSSVIDDGDDIASSSSRRKTPFQFDLNFPPLDGVDLF | | 53968 |
| | | | | AGGIDDLHCTDLRL | | |
| 86 | 114 | AT1 | C2C2- | MATQDSQGIKLFGKTITFNANITQTIKKEEQQQQQQPELQATTAVRSPS | 61 | - |
| | | G29 | DOF | SDLTAEKRPDKI | | 0.506 |
| | | 160 | | | | 53019 |
| 87 | 154 | AT5 | C2H2 | NLPWKLKQRSKQEVIKKKVYICPIKTCVHHDASRALGDLTGIKKHYSRK | 399 | − |
| | | G03 | | HGEKKWKCEKCSKKYAVQSDWKAHAKTCGTREYKCDCGTLFSRKDSFIT | | 0.482 |
| | | 150 | | HRAFCDALTEEGARMSSLSNNNPVISTTNLNFGNESNVMNNPNLPHGFV | | 34955 |
| | | | | HRGVHHPDINAAISQFGLGFGHDLSAMHAQGLSEMVQMASTGNHHLFPS | | |
| | | | | SSSSLPDFSGHHQFQIPMTSTNPSLTLSSSSTSQQTSASLQHQTLKDSS | | |
| | | | | FSPLFSSSSENKQNKPLSPMSATALLQKAAQMGSTRSNSSTAPSFFAGP | | |
| | | | | TMTSSSATASPPPRSSSPMMIQQQLNNENTNVLRENHNRAPPPLSGVST | | |
| | | | | SSVDNNPFQSNRSGLNPAQQMGLTRDFLGVSNEHHPHQTGRRPFLPQEL | | |
| | | | | ARFAPLG | | |
| 88 | 179 | AT5 | E2F- | IFENRFIDGSASLCDRNVPKKRAFGTELTNVNAKRNKSGCSKEDSKRNG | 139 | - |
| | | G14 | DP | NQNTSIVIKQEQCDDVKPDVKNFASGSSTPAGTSESNDMGNNIRPRGRL | | 0.476 |
| | | 960 | | GVIEALSTLYQPSYCNPELLGLFAHYNETFRSYQEEFGREK | | 76792 |
| 89 | 43 | AT5 | AP2- | ELLPGEKFSDEDMSAATIRKKATEVGAQVDALGTAVQNNRHRVFGQNRD | 107 | - |
| | | G67 | EREBP | SDVDNKNFHRNYQNGEREEEEEDEDDKRLRSGGRLLDRVDLNKLPDPES | | 0.467 |
| | | 190 | | SDEEWESKH | | 13498 |
| 90 | 266 | AT2 | MYB | RAGLPLYPHEIQHQGIDIDDEFEFDLTSFQFQNQDLDHNHQNMIQYTNS | 368 | - |
| | | G32 | | SNTSSSSSSFSSSSSQPSKRLRPDPLVSTNPGLNPIPDSSMDFQMFSLY | | 0.467 |
| | | 460 | | NNSLENDNNQFGFSVPLSSSSSSNEVCNPNHILEYISENSDTRNTNKKD | | 06633 |
| | | | | IDAMSYSSLLMGDLEIRSSSFPLGLDNSVLELPSNQRPTHSFSSSPIID | | |
| | | | | NGVHLEPPSGNSGLLDALLEESQALSRGGLFKDVRVSSSDLCEVQDKRV | | |
| | | | | KMDFENLLIDHLNSSNHSSLGANPNIHNKYNEPTMVKVTVDDDDELLTS | | |
| | | | | LLNNFPSTTTPLPDWYRVTEMQNEASYLAPPSGILMGNHQGNGRVEPPT | | |
| | | | | VPPSSSVDPMASLGSCYWSNMPSIC | | |
| 91 | 196 | AT2 | G2- | MASSSELSLDCKPQSYSMLLKSFGDNFQSDPTTHKLEDLLSRLEQERLK | 229 | - |
| | | G03 | like | IDAFKRELPLCMQLLNNAVEVYKQQLEAYRANSNNNNQSVGTRPVLEEF | | 0.460 |
| | | 500 | | IPLRNQPEKTNNKGSNWMTTAQLWSQSETKPKNIDSTTDQSLPKDEINS | | 01485 |
| | | | | SPKLGHFDAKQRNGSGAFLPFSKEQSLPELALSTEVKRVSPTNEHTNGQ | | |
| | | | | DGNDESMINNDNNYNNNNNNNSNSNGVSSTTSQ | | |
| 92 | 253 | AT5 | MADS | NLVKILDRYGKQHADDLKALDHQSKALNYGSHYELLELVDSKLVGSNVK | 135 | - |
| | | G10 | | NVSIDALVQLEEHLETALSVTRAKKTELMLKLVENLKEKEKMLKEENQV | | 0.455 |
| | | 140 | | LASQMENNHHVGAEAEMEMSPAGQISDNLPVTLPLLN | | 58866 |
| 93 | 214 | AT5 | HB | QLEQLYDSLRQEYDVVSREKQMLHDEVKKLRALLRDQGLIKKQISAGTI | 103 | - |
| | | G03 | | KVSGEEDTVEISSVVVAHPRTENMNANQITGGNQVYGQYNNPMLVASSG | | 0.451 |
| | | 790 | | WPSYP | | 76504 |
| 94 | 74 | AT1 | AP2- | DLLLQEEDHLSAATTADMPAALIREKAAEVGARVDALLASAAPSMAHST | 66 | − |
| | | G46 | EREBP | PPVIKPDLNQIPESGDI | | 0.427 |
| | | 768 | | | | 49972 |
| 95 | 53 | AT1 | AP2- | CYNINAHCLSLTQSLSQSSTVESSFPNLNLGSDSVSSRFPFPKIQVKAG | 90 | - |
| | | G28 | EREBP | MMVFDERSESDSSSVVMDVVRYEGRRVVLDLDLNFPPPPEN | | 0.422 |
| | | 370 | | | | 98671 |
| 96 | 60 | AT3 | AP2- | TFLELSDQKVPTGFARSPSQSSTLDCASPPTLVVPSATAGNVPPQLELS | 141 | - |
| | | G15 | EREBP | LGGGGGGSCYQIPMSRPVYFLDLMGIGNVGRGQPPPVTSAFRSPVVHVA | | 0.413 |
| | | 210 | | TKMACGAQSDSDSSSVVDFEGGMEKRSQLLDLDLNLPPPSEQA | | 67205 |
| 97 | 115 | AT2 | C2C2- | SSSSSSSNILQTIPSSLPDLNPPILFSNQIHNKSKGSSQDLNLLSFPVM | 235 | - |
| | | G46 | DOF | QDQHHHHVHMSQFLQMPKMEGNGNITHQQQPSSSSSVYGSSSSPVSALE | | 0.408 |
| | | 590 | | LLRTGVNVSSRSGINSSFMPSGSMMDSNTVLYTSSGFPTMVDYKPSNLS | | 99102 |
| | | | | FSTDHQGLGHNSNNRSEALHSDHHQQGRVLFPFGDQMKELSSSITQEVD | | |
| | | | | HDDNQQQKSHGNNNNNNNSSPNNGYWSGMFSTTGGGSSW | | |
| 98 | 272 | AT5 | MYB | SKRKHKRESNADNNDRDASPSAKRPCILQDYIKSIERNNINKDNDEKKN | 224 | - |
| | | G58 | | ENTISVISTPNLDQIYSDGDSASSILGGPYDEELDYFQNIFANHPISLE | | 0.374 |
| | | 850 | | NLGLSQTSDEVTQSSSSGFMIKNPNPNLHDSVGIHHQEATITAPANTPH | | 84044 |
| | | | | LASDIYLSYLLNGTTSSYSDTHFPSSSSSTSSTTVEHGGHNEFLEPQAN | | |
| | | | | STSERREMDLIEMLSGSIQGSNICFPLV | | |
| 99 | 67 | AT5 | AP2 | LPGESTTVNDGGENDSYVNRTTVTTAREMTRQRFPFACHRERKVVGGYA | 111 | - |
| | | G44 | EREBP | SAGFFFDPSRAASLRAELSRVCPVREDPVNIELSIGIRETVKVEPRREL | | 0.329 |
| | | 210 | | NLDLNLAPPVVDV | | 20229 |
| 100 | 482 | AT5 | bHLH | MELPQPRPFKTQEFRTGRKPTHDFLSLCSHSTVHPDPKPTPPPSSQGSH | 280 | - |
| | | G08 | | LKTHDFLQPLECVGAKEDVSRINSTTTASEKPPPPAPPPPLQHVLPGGI | | 0.328 |
| | | 130 | | GTYTISPIPYFHHHHQRIPKPELSPPMMENANERNVLDENSNSNCSSYA | | 58898 |
| | | | | AASSGFTLWDESASGKKGQTRKENSVGERVNMRADVAATVGQWPVAERR | | |
| | | | | SQSLTNNHMSGFSSLSSSQGSVLKSQSFMDMIRSAKGSSQEDDLDDEED | | |
| | | | | FIMKKESSSTSQSHRVDLRVKADVRGSPNDQKLNT | | |
| 101 | 321 | AT1 | MYB- | NLNRRRRRSSLFDITTETVTEMAMEQDPTQENSPLPETNISSGQQAMQV | 133 | - |
| | | G19 | related | FTDVPTKTENAPETFHLNDPYLVPVTFQAKPTENLNTDAAPLSLNLCLA | | 0.325 |
| | | 000 | | SSFNLNEQPNSRHSAFTMMPSFSDGDSNSSIIRVA | | 19448 |
| 102 | 331 | AT5 | MYB- | KSGTGEHLPPPRPKRKAAHPYPQKAHKNVQLQVPGSFKSTSEPNDPSFM | 210 | - |
| | | G52 | related | FRPESSSMLMTSPTTAAAAPWTNNAQTISFTPLPKAGAGANNNCSSSSE | | 0.322 |
| | | 660 | | NTPRPRSNRDARDHGNVGHSLRVLPDFAQVYGFIGSVFDPYASNHLQKL | | 35527 |
| | | | | KKMDPIDVETVLLLMRNLSINLSSPDFEDHRRLLSSYDIGSETATDHGG | | |
| | | | | VNKTLNKDPPEIST | | |
| 103 | 121 | AT4 | C2C2- | RINQPSVAQMVSVGIQPGSHKPFFNVQENNDFVGSFGASSSSFVAAVGN | 153 | - |
| | | G21 | DOF | RFSSLSHIHGGMVTNVHPTQTFRPNHRLAFHNGSFEQDYYDVGSDNLLV | | 0.320 |
| | | 040 | | NQQVGGYVDNHNGYHMNQVDQYNWNQSFNNAMNMNYNNASTSGRMHPSH | | 71913 |
| | | | | LEKGGP | | |
| 104 | 354 | AT3 | NAC | NNIGPPSGNRYAPFMEEEWADGGGALIPGIDVRVRVEALPQANGNNQMD | 315 | - |
| | | G10 | | QWADLLKLHNSIKFAITFCRTQLNLTALSNERCSTREIFIVFWLICKEM | | 0.305 |
| | | 480 | | HSASKDLININELPRDATPMDIEPNQQNHHESAFKPQESNNHSGYEEDE | | 60613 |
| | | | | DTLKREHAEEDERPPSLCILNKEAPLPLLQYKRRRQNESNNNSSRNTQD | | |
| | | | | HCSSTITTVDNTTTLISSSAAAATNTAISALLEFSLMGISDKKENQQKE | | |
| | | | | ETSPPSPIASPEEKVNDLQKEVHQMSVERETFKLEMMSAEAMISILQSR | | |
| | | | | IDALRQENEELKKKNASGQAS | | |
| 105 | 254 | AT5 | MADS | MQKTIERYRKYTKDHETSNHDSQIHLQQLKQEASHMITKIELLEFHKRK | 149 | - |
| | | G62 | | LLGQGIASCSLEELQEIDSQLQRSLGKVRERKAQLFKEQLEKLKAKEKQ | | 0.297 |
| | | 165 | | LLEENVKLHQKNVINPWRGSSTDQQQEKYKVIDLNLEVETDLFIGLPNR | | 35578 |
| | | | | NC | | |
| 106 | 443 | AT1 | WRKY | MCSVSELLDMENFQGDLTDVVRGIGGHVLSPETPPSNIWPLPLSHPTPS | 210 | - |
| | | G30 | | PSDLNINPFGDPFVSMDDPLLQELNSITNSGYFSTVGDNNNNIHNNNGF | | 0.296 |
| | | 650 | | LVPKVFEEDHIKSQCSIFPRIRISHSNIIHDSSPCNSPAMSAHVVAAAA | | 84329 |
| | | | | AASPRGIINVDTNSPRNCLLVDGTTFSSQIQISSPRNLGLKRRKSQAKK | | |
| | | | | VVCIPAPAAMNSRS | | |
| 107 | 178 | AT3 | E2F- | IPGALKELQEEGVKDTFHRFYVNENVKGSDDEDDDEESSQPHSSSQTDS | 301 | - |
| | | G48 | DP | SKPGSLPQSSDPSKIDNRREKSLGLLTQNFIKLFICSEAIRIISLDDAA | | 0.284 |
| | | 160 | | KLLLGDAHNTSIMRTKVRRLYDIANVLSSMNLIEKTHTLDSRKPAFKWL | | 84674 |
| | | | | GYNGEPTFTLSSDLLQLESRKRAFGTDITNVNVKRSKSSSSSQENATER | | |
| | | | | RLKMKKHSTPESSYNKSFDVHESRHGSRGGYHFGPFAPGTGTYPTAGLE | | |
| | | | | DNSRRAFDVENLDSDYRPSYQNQVLKDLFSHYMDAWKTWFSEVTQENPL | | |
| | | | | PNTSQHR | | |
| 108 | 300 | AT3 | MYB | LSQGLDPSTHNLMPSHKRSSSSNNNNIPKPNKTTSIMKNPTDLDQSTTA | 181 | - |
| | | G12 | | FSITNINPPTSTKPNKLKSPNQTTIPSQTVIPINDNMSSTQTMIPINDP | | 0.278 |
| | | 720 | | MSSLLDDENMIPHWSDVDGMAIHEAPMLPSDKAVVGVDDDDLNMDILEN | | 25974 |
| | | | | TPSSSAFDPDFASIFSSAMSIDFNPMDDLGSWTF | | |
| 109 | 231 | AT2 | Homeo | QLEKDYGVLKTQYDSLRHNFDSLRRDNESLLQEISKLKTKLNGGGGEEE | 194 | - |
| | | G22 | box | EEENNAAVTTESDISVKEEEVSLPEKITEAPSSPPQFLEHSDGLNYRSF | | 0.253 |
| | | 430 | | TDLRDLLPLKAAASSFAAAAGSSDSSDSSALLNEESSSNVTVAAPVTVP | | 71934 |
| | | | | GGNFFQFVKMEQTEDHEDFLSGEEACEFFSDEQPPSLHWYSTVDHWN | | |
| 110 | 256 | AT2 | MADS | IESTIERYNRCYNCSLSNNKPEETTQSWCQEVTKLKSKYESLVRTNRNL | 191 | - |
| | | G45 | | LGEDLGEMGVKELQALERQLEAALTATRQRKTQVMMEEMEDLRKKERQL | | 0.253 |
| | | 650 | | GDINKQLKIKFETEGHAFKTFQDLWANSAASVAGDPNNSEFPVEPSHPN | | 16786 |
| | | | | VLDCNTEPFLQIGFQQHYYVQGEGSSVSKSNVAGETNFVQGWVL | | |
| 111 | 450 | AT5 | WRKY | MDREDINPMLSRLDVENNNTFSSFVDKTLMMMPPSTFSGEVEPSSSSSW | 91 | - |
| | | G41 | | YPESFHVHAPPLPPENDQIGEKGKELKEKRSRKVPRIAFHTR | | 0.238 |
| | | 570 | | | | 38244 |
| 112 | 420 | AT3 | TCP | PPLPISPENFSIFNHHQSFLNLGQRPGQDPTQLGFKINGCVQKSTTTSR | 223 | - |
| | | G02 | | EENDREKGENDVVYTNNHHVGSYGTYHNLEHHHHHHQHLSLQADYHSHQ | | 0.235 |
| | | 150 | | LHSLVPFPSQILVCPMTTSPTTTTIQSLFPSSSSAGSGTMETLDPRQMV | | 84824 |
| | | | | SHFQMPLMGNSSSSSSQNISTLYSLLHGSSSNNGGRDIDNRMSSVQENR | | |
| | | | | TNSTTTANMSRHLGSERCTSRGSDHHM | | |
| 113 | 103 | AT1 | C2C2- | WPSSNHYLQVTSEDCDNNNSGTILSFGSSESSVTETGKHQSGDTAKISA | 213 | - |
| | | G69 | DOF | DSVSQENKSYQGFLPPQVMLPNNSSPWPYQWSPTGPNASFYPVPFYWGC | | 0.233 |
| | | 570 | | TVPIYPTSETSSCLGKRSRDQTEGRINDTNTTITTTRARLVSESLRMNI | | 77776 |
| | | | | EASKSAVWSKLPTKPEKKTQGFSLFNGFDTKGNSNRSSLVSETSHSLQA | | |
| | | | | NPAAMSRAMNFRESMQQ | | |
| 114 | 320 | AT5 | MYB- | VNDKRKRRASLFDISLEDQKEKERNSQDASTKTPPKQPITGIQQPVVQG | 159 | - |
| | | G61 | related | HTQTEISNRFQNLSMEYMPIYQPIPPYYNFPPIMYHPNYPMYYANPQVP | | 0.227 |
| | | 620 | | VRFVHPSGIPVPRHIPIGLPLSQPSEASNMTNKDGLDLHIGLPPQATGA | | 57923 |
| | | | | SDLTGHGVIHVK | | |
| 115 | 85 | AT1 | ARID | FRSNGQIPPDSMQSPSARPCFIQGAIRPSQELQALTFTPQPKINTAEFL | 142 | - |
| | | G04 | | GGSLAGSNVVGVIDGKFESGYLVTVTIGSEQLKGVLYQLLPQNTVSYQT | | 0.226 |
| | | 880 | | PQQSHGVLPNTLNISANPQGVAGGVTKRRRRRKKSEIKRRDPDH | | 26197 |
| 116 | 412 | AT2 | SBP | MSMRRSKAEGKRSLRELSEEEEEEEETEDEDTFEEEEALEKKQKGKATS | 50 | - |
| | | G33 | | S | | 0.224 |
| | | 810 | | | | 13365 |
| 117 | 433 | AT3 | Tri- | KEFKKAKQHEDKATSGGSTKMSYYNEIEDIFRERKKKVAFYKSPATTTP | 261 | - |
| | | G25 | helix | SSAKVDSFMQFTDKGFEDTGISFTSVEANGRPTLNLETELDHDGLPLPI | | 0.215 |
| | | 990 | | AADPITANGVPPWNWRDTPGNGVDGQPFAGRIITVKFGDYTRRVGIDGT | | 71602 |
| | | | | AEAIKEAIRSAFRLRTRRAFWLEDEEQVIRSLDRDMPLGNYILRIDEGI | | |
| | | | | AVRVCHYDESDPLPVHQEEKIFYTEEDYRDFLARRGWTCLREFDAFQNI | | |
| | | | | DNMDELQSGRLYRGMR | | |
| 118 | 370 | AT1 | NAC | ESYMPWSHGFLNMLDLLFTRTVNGTTL | 27 | - |
| | | G19 | | | | 0.212 |
| | | 040 | | | | 79153 |
| 119 | 141 | AT1 | C2H2 | NLPWKLKQKSNKEVRRKVYLCPEPSCVHHDPARALGDLTGIKKHYYRKH | 363 | − |
| | | G14 | | GEKKWKCDKCSKRYAVQSDWKAHSKTCGTKEYRCDCGTIFSRRDSYITH | | 0.206 |
| | | 580 | | RAFCDALIQESARNPTVSFTAMAAGGGGGARHGFYGGASSALSHNHFGN | | 52379 |
| | | | | NPNSGFTPLAAAGYNLNRSSSDKFEDFVPQATNPNPGPTNFLMQCSPNQ | | |
| | | | | GLLAQNNQSLMNHHGLISLGDNNNNNHNFENLAYFQDTKNSDQTGVPSL | | |
| | | | | FTNGADNNGPSALLRGLTSSSSSSVVVNDFGDCDHGNLQGLMNSLAATT | | |
| | | | | DQQGRSPSLFDLHFANNLSMGGSDRLTLDFLGVNGGIVSTVNGRGGRSG | | |
| | | | | GPPLDAEMKFSHPNHPYGKA | | |
| 120 | 45 | AT4 | AP2- | EEVFKDGNGGEGLGGDMSPTLIRKKAAEVGARVDAELRLENRMVENLDM | 58 | - |
| | | G06 | EREBP | NKLPEAYGL | | 0.192 |
| | | 746 | | | | 0977 |
| 121 | 142 | AT2 | C2H2 | FLSSSTTRKEAKTTRPNKAHPSTSSSSSSSRWSNLLSSAEAGISRLGND | 67 | - |
| | | G41 | | ISQKLQFSSSKDNGIVEV | | 0.166 |
| | | 835 | | | | 95289 |
| 122 | 460 | AT1 | WRKY | MDQYSSSLVDTSLDLTIGVTRMRVEEDPPTSALVEELNRVSAENKKLSE | 139 | - |
| | | G80 | | MLTLMCDNYNVLRKQLMEYVNKSNITERDQISPPKKRKSPAREDAFSCA | | 0.166 |
| | | 840 | | VIGGVSESSSTDQDEYLCKKQREETVVKEKVSRVYYKTEAS | | 41409 |
| 123 | 479 | AT1 | ZF-HD | MDMRSHEMIERRREDNGNNNGGVVISNIISTNIDDNCNGNNNNTRVSCN | 223 | - |
| | | G75 | | SQTLDHHQSKSPSSFSISAAAKPTVRYRECLKNHAASVGGSVHDGCGEF | | 0.163 |
| | | 240 | | MPSGEEGTIEALRCAACDCHRNFHRKEMDGVGSSDLISHHRHHHYHHNQ | | 84919 |
| | | | | YGGGGGRRPPPPNMMLNPLMLPPPPNYQPIHHHKYGMSPPGGGGMVTPM | | |
| | | | | SVAYGGGGGGAESSSEDLNLYGQSSGE | | |
| 124 | 6 | AT4 | ABI3- | TLCEKPTSYFVRKCGHAEKTKASHTGYEQEEHINSDIDTASAQLPVISP | 106 | - |
| | | G33 | VP1 | TSTVRVSEGKYPLSGFKKMRRELSNDNLDQKADVEMISAGSNKKALSLA | | 0.163 |
| | | 280 | | KRAISPDG | | 60667 |
| 125 | 441 | AT1 | Tri- | MEQGGGGGGNEVVEEASPISSRPPANNLEELMRFSAAADDGGLGGGGGG | 433 | - |
| | | G33 | helix | GGGGSASSSSGNRWPREETLALLRIRSDMDSTFRDATLKAPLWEHVSRK | | 0.152 |
| | | 240 | | LLELGYKRSSKKCKEKFENVQKYYKRTKETRGGRHDGKAYKFFSQLEAL | | 07721 |
| | | | | NTTPPSSSLDVTPLSVANPILMPSSSSSPFPVFSQPQPQTQTQPPQTHN | | |
| | | | | VSFTPTPPPLPLPSMGPIFTGVTFSSHSSSTASGMGSDDDDDDMDVDQA | | |
| | | | | NIAGSSSRKRKRGNRGGGGKMMELFEGLVRQVMQKQAAMQRSFLEALEK | | |
| | | | | REQERLDREEAWKRQEMARLAREHEVMSQERAASASRDAAIISLIQKIT | | |
| | | | | GHTIQLPPSLSSQPPPPYQPPPAVTKRVAEPPLSTAQSQSQQPIMAIPQ | | |
| | | | | QQILPPPPPSHPHAHQPEQKQQQQPQQEMVMSSEQSSLPSS | | |
| 126 | 3 | AT5 | ABI3- | TMCKKIRRSSDQSEEIKVESDSDEQNQASDDVLSLDEDDDDSDYNCGED | 216 | - |
| | | G60 | VP1 | NDSDDYADEAAVEKDDNDADDEDVDNVADDVPVEDDDYVEAFDSRDHAK | | 0.147 |
| | | 130 | | ADDDDEDERQYLDDRENPSFTLILNPKKKSQLLIPARVIKDYDLHFPES | | 14093 |
| | | | | ITLVDPLVKKFGTLEKQIKIQTNGSVFVKGFGSIIRRNKVKTTDKMIFE | | |
| | | | | IKKTGDNNLVQTIKIHIISG | | |
| 127 | 317 | AT3 | MYB- | NKKGKRFSIHDMTLGDAENVTVPVSNLNSMGQQPHFDDQSPPDHYQDYF | 142 | - |
| | | G10 | related | SQSNVTIPGCNMHFMGQQPRFGDQIPPGEYHPYSRDNVTVTGSNLNSIG | | 0.146 |
| | | 580 | | QQPHFNDQISPDQYGRYLQENFGFFDDDGEDDGSLASFQQLYKA | | 37314 |
| 128 | 250 | AT3 | MADS | GFQDLLLNPVLTAGCSTDFSLQSTHQNYISDCNLGYFLQIGFQQHYEQG | 69 | - |
| | | G61 | | EGSSVTKSNARSDAETNFVQ | | 0.115 |
| | | 120 | | | | 84727 |
| 129 | 135 | AT1 | C2C2- | MDDLHGSNARMHIREAQDPMHVQFEHHALHHIHNGSGMVDDQADDGNAG | 216 | − |
| | | G51 | GATA | GMSEGVETDIPSHPGNVTDNRGEVVDRGSEQGDQLTLSFQGQVYVEDSV | | 0.101 |
| | | 600 | | LPEKVQAVLLLLGGRELPQAAPPGLGSPHQNNRVSSLPGTPQRFSIPQR | | 10322 |
| | | | | LASLVRFREKRKGRNFDKKIRYTVRKEVALRMQRNKGQFTSAKSNNDEA | | |
| | | | | ASAGSSWGSNQTWAIESSEA | | |
| 130 | 268 | AT3 | MYB | IQMGIDPVTHRPRTDHLNVLAALPQLLAAANENNLLNLNQNIQLDATSV | 205 | - |
| | | G02 | | AKAQLLHSMIQVLSNNNTSSSFDIHHTTNNLFGQSSFLENLPNIENPYD | | 0.086 |
| | | 940 | | QTQGLSHIDDQPLDSFSSPIRVVAYQHDQNFIPPLISTSPDESKETQMM | | 43891 |
| | | | | VKNKEIMKYNDHTSNPSSTSTFTQDHQPWCDIIDDEASDSYWKEIIEQT | | |
| | | | | CSEPWPFRE | | |
| 131 | 458 | AT4 | WRKY | MFRFPVSLGGSRDEDRHDQITPLDDHRVVVDEVDFFSEKRDRVSRENIN | 290 | - |
| | | G22 | | DDDDEGNKVLIKMEGSRVEENDRSRDVNIGLNLLTANTGSDESTVDDGL | | 0.081 |
| | | 070 | | SMDMEDKRAKIENAQLQEELKKMKIENQRLRDMLSQATTNFNALQMQLV | | 38204 |
| | | | | AVMRQQEQRNSSQDHLLAQESKAEGRKRQELQIMVPRQFMDLGPSSGAA | | |
| | | | | EHGAEVSSEERTTVRSGSPPSLLESSNPRENGKRLLGREESSEESESNA | | |
| | | | | WGNPNKVPKHNPSSSNSNGNRNGNVIDQSAAEATMRKARVSVRAR | | |
| 132 | 413 | AT3 | SBP | MEGQRTQRRGYLKDKATVSNLVEEEMENGMDGEEEDGGDEDKRKKVMER | 59 | - |
| | | G15 | | VRGPSTDRVP | | 0.059 |
| | | 270 | | | | 09685 |
| 133 | 213 | AT5 | HB | LLSSEDHTGLSHAGTKSILKLAQRMKLNFYSGITASCIHKWEKLLAENV | 253 | - |
| | | G52 | | GQDTRILTRKSLEPSGIVLSAATSLWLPVTQQRLFEFLCDGKCRNQWDI | | 0.014 |
| | | 170 | | LSNGASMENTLLVPKGQQEGSCVSLLRAAGNDQNESSMLILQETWNDVS | | 6853 |
| | | | | GALVVYAPVDIPSMNTVMSGGDSAYVALLPSGFSILPDGSSSSSDQFDT | | |
| | | | | DGGLVNQESKGCLLTVGFQILVNSLPTAKLNVESVETVNNLIACTIHKI | | |
| | | | | RAALRIPA | | |
| 134 | 472 | AT3 | WRKY | MDTNKAKKLKVMNQLVEGHDLTTQLQQLLSQPGSGLEDLVAKILVCENN | 113 | - |
| | | G56 | | TISVLDTFEPISSSSSLAAVEGSQNASCDNDGKFEDSGDSRKRLGPVKG | | 0.012 |
| | | 400 | | KRGCYKRKKRSETCT | | 07828 |
| 135 | 131 | AT3 | C2C2- | MDVYGMSSPDLLRIDDLLDFSNDEIFSSSSTVTSSAASSAASSENPFSF | 153 | - |
| | | G60 | GATA | PSSTYTSPTLLTDFTHDLCVPSDDAAHLEWLSRFVDDSFSDFPANPLTM | | 0.007 |
| | | 530 | | TVRPEISFTGKPRSRRSRAPAPSVAGTWAPMSESELCHSVAKPKPKKVY | | 04007 |
| | | | | NAESVT | | |
| 136 | 391 | AT2 | ND | VHEQFMKTQRKHMDHVTDQLMVELHRGRRLDDLDLSEINALISFSRENI | 161 | - |
| | | G15 | | ILLRKELEFVQHSPLGDPRVPPFEAQFEELTTIANDVFVRGGQVDERAW | | 0.006 |
| | | 660 | | KNYEATKRVSIGNALRGNQSHYLVDKWLFASPKPREPTNQSRLTYQTIF | | 11432 |
| | | | | YTKEAVATDALIWI | | |
| 137 | 423 | AT3 | TCP | TGHGVTTTSNEDIQPNRNFPSYTENGDNISNNVFPCTVVNTGHRQMVEP | 94 | - |
| | | G45 | | VSTMTDHAPSTNYSTISDNYNSTFNGNATASDTTSAATTTATTTV | | 0.005 |
| | | 150 | | | | 73704 |
| 138 | 197 | AT3 | G2- | LNGQANNSENKIGIMTMMEEKTPDADEIQSENLSIGPQPNKNSPIGEAL | 292 | 0.002 |
| | | G04 | like | QMQIEVQRRLHEQLEVQRHLQLRIEAQGKYLQSVLEKAQETLGRQNLGA | | 67371 |
| | | 030 | | AGIEAAKVQLSELVSKVSAEYPNSSFLEPKELQNLCSQQMQTNYPPDCS | | 9 |
| | | | | LESCLTSSEGTQKNSKMLENNRLGLRTYIGDSTSEQKEIMEEPLFQRME | | |
| | | | | LTWTEGLRGNPYLSTMVSEAEQRISYSERSPGRLSIGVGLHGHKSQHQQ | | |
| | | | | GNNEDHKLETRNRKGMDSTTELDLNTHVENYCTTRTKQFDLNGFSWN | | |
| 139 | 446 | AT4 | WRKY | MDGSSFLDISLDLNTNPFSAKLPKKEVSVLASTHLKRKWLEQDESASEL | 176 | 0.004 |
| | | G31 | | REELNRVNSENKKLTEMLARVCESYNELHNHLEKLQSRQSPEIEQTDIP | | 82815 |
| | | 800 | | IKKRKQDPDEFLGFPIGLSSGKTENSSSNEDHHHHHQQHEQKNQLLSCK | | 9 |
| | | | | RPVTDSFNKAKVSTVYVPTETSDTSLTVK | | |
| 140 | 329 | AT5 | MYB- | SMNKDRRRSSIHDITSVGNADVSTPQGPITGQNNSNNNNNNNNNNSSPA | 130 | 0.017 |
| | | G08 | related | VAGGGNKSAKQAVSQAPPGPPMYGTPAIGQPAVGTPVNLPAPPHMAYGV | | 53887 |
| | | 520 | | HAAPVPGSVVPGAAMNIGQMPYTMPRTPTAHR | | |
| 141 | 459 | AT2 | WRKY | MAASFLTMDNSRTRQNMNGSANWSQQSGRTSTSSLEDLEIPKFRSFAPS | 355 | 0.057 |
| | | G38 | | SISISPSLVSPSTCFSPSLFLDSPAFVSSSANVLASPTTGALITNVTNQ | | 30509 |
| | | 470 | | KGINEGDKSNNNNFNLFDFSFHTQSSGVSAPTTTTTTTTTTTTTNSSIF | | 4 |
| | | | | QSQEQQKKNQSEQWSQTETRPNNQAVSYNGREQRKGEDGYNWRKYGQKQ | | |
| | | | | VKGSENPRSYYKCTFPNCPTKKKVERSLEGQITEIVYKGSHNHPKPQST | | |
| | | | | RRSSSSSSTFHSAVYNASLDHNRQASSDQPNSNNSFHQSDSFGMQQEDN | | |
| | | | | TTSDSVGDDEFEQGSSIVSRDEEDCGSEPEAKRWKGDNETNGGNGGGSK | | |
| | | | | TVREPRIVVQTT | | |
| 142 | 516 | AT2 | bZIP | KLRLQVMEQQAKLRDALNEQLKKEVERLKFATGEVSPADAYNLGMAHMQ | 156 | 0.060 |
| | | G40 | | YQQQPQQSFFQHHHQQQTDAQNLQQMTHQFHLFQPNNNQNQSSRTNPPT | | 13385 |
| | | 620 | | AHQLMHHATSNAPAQSHSYSEAMHEDHLGRLQGLDISSCGRGSNFGRSD | | 7 |
| | | | | TVSESSSTM | | |
| 143 | 527 | AT1 | bZIP | MDKEKSPAPPPSGGLPPPSGRYSAFSPNGSSFAMKAESSFPPLTPSGSN | 272 | 0.061 |
| | | G06 | | SSDANRFSHDISRMPDNPPKNLGHRRAHSEILTLPDDLSFDSDLGVVGA | | 79356 |
| | | 070 | | ADGPSFSDDTDEDLLYMYLDMEKENSSATSTSQMGEPSEPTWRNELAST | | 9 |
| | | | | SNLQSTPGSSSERPRIRHQHSQSMDGSTTIKPEMLMSGNEDVSGVDSKK | | |
| | | | | AISAAKLSELALIDPKRAKRIWANRQSAARSKERKMRYIAELERKVQTL | | |
| | | | | QTEATSLSAQLTLLQRDTNGLGVENNE | | |
| 144 | 305 | AT2 | MYB | RLGLPVYPDEVREHAMNAATHSGLNTDSLDGHHSQEYMEADTVEIPEVD | 303 | 0.064 |
| | | G26 | | FEHLPLNRSSSYYQSMLRHVPPTNVFVRQKPCFFQPPNVYNLIPPSPYM | | 58189 |
| | | 960 | | STGKRPREPETAFPCPGGYTMNEQSPRLWNYPFVENVSEQLPDSHLLGN | | 9 |
| | | | | AAYSSPPGPLVHGVENFEFPSFQYHEEPGGWGADQPNPMPEHESDNTLV | | |
| | | | | QSPLTAQTPSDCPSSSLYDGLLESVVYGSSGEKPATDTDSESSLFQSFT | | |
| | | | | PANENITGKTCFLTLYALHALHCLCNQFKKSPLLHLHDKLNWCNKFREN | | |
| | | | | SFKSGTHIL | | |
| 145 | 277 | AT3 | MYB | NKVNQDSHQELDRSSLSSSPSSSSANSNSNISRGQWERRLQTDIHLAKK | 207 | 0.064 |
| | | G28 | | ALSEALSPAVAPIITSTVTTTSSSAESRRSTSSASGFLRTQETSTTYAS | | 76390 |
| | | 910 | | STENIAKLLKGWVKNSPKTQNSADQIASTEVKEVIKSDDGKECAGAFQS | | 4 |
| | | | | FSEFDHSYQQAGVSPDHETKPDITGCCSNQSQWSLFEKWLFEDSGGQIG | | |
| | | | | DILLDENTNFF | | |
| 146 | 113 | AT3 | C2C2- | SSSHYRHITISEALEAARLDPGLQANTRVLSFGLEAQQQHVAAPMTPVM | 284 | 0.072 |
| | | G47 | DOF | KLQEDQKVSNGARNRFHGLADQRLVARVENGDDCSSGSSVTTSNNHSVD | | 47244 |
| | | 500 | | ESRAQSGSVVEAQMNNNNNNNMNGYACIPGVPWPYTWNPAMPPPGFYPP | | 2 |
| | | | | PGYPMPFYPYWTIPMLPPHQSSSPISQKCSNTNSPTLGKHPRDEGSSKK | | |
| | | | | DNETERKQKAGCVLVPKTLRIDDPNEAAKSSIWTTLGIKNEAMCKAGGM | | |
| | | | | FKGFDHKTKMYNNDKAENSPVLSANPAALSRSHNFHEQI | | |
| 147 | 440 | AT5 | Tri- | TRYKACETTEPDAIRQQFPFYNEIQSIFEARMQRMLWSEATEPSTSSKR | 215 | 0.078 |
| | | G01 | helix | KHHQFSSDDEEEEVDEPNQDINEELLSLVETQKRETEVITTSTSTNPRK | | 85472 |
| | | 380 | | RAKKGKGVASGTKAETAGNTLKDILEEFMRQTVKMEKEWRDAWEMKEIE | | 2 |
| | | | | REKREKEWRRRMAELEEERAATERRWMEREEERRLREEARAQKRDSLID | | |
| | | | | ALLNRLNRDHNDDHHNQGF | | |
| 148 | 498 | AT3 | bHLH | MNMDKETEQTLNYLPLGQSDPFGNGNEGTIGDFLGRYCNNPQEISPLTL | 190 | 0.080 |
| | | G23 | | QSFSLNSQISENFPISGGIRFPPYPGQFGSDREFGSQPTTQESNKSSLL | | 56270 |
| | | 690 | | DPDSVSDRVHTTKSNSRKRKSIPSGNGKESPASSSLTASNSKVSGENGG | | 7 |
| | | | | SKGGKRSKQDVAGSSKNGVEKCDSKGDNKDDAKPPEAPKDYIH | | |
| 149 | 448 | AT2 | WRKY | MEEIEGTNRAAVESCHRVLNLLHRSQQQDHVGFEKNLVSETREAVIRFK | 306 | 0.089 |
| | | G30 | | RVGSLLSSSVGHARFRRAKKLQSHVSQSLLLDPCQQRTTEVPSSSSQKT | | 82564 |
| | | 590 | | PVLRSGFQELSLRQPSDSLTLGTRSFSLNSNAKAPLLQLNQQTMPPSNY | | 8 |
| | | | | PTLFPVQQQQQQQQQQQQQEQQQQQQQQQQQFHERLQAHHLHQQQQLQK | | |
| | | | | HQAELMLRKCNGGISLSFDNSSCTPTMSSTRSFVSSLSIDGSVANIEGK | | |
| | | | | NSFHFGVPSSTDQNSLHSKRKCPLKGDEHGSLKCGSSSRCHCAKKRKHR | | |
| | | | | VRRSIRVPAISN | | |
| 150 | 116 | AT3 | C2C2- | RSRTCSNSSSSSVSGVVSNSNGVPLQTTPVLFPQSSISNGVTHTVTESD | 169 | 0.096 |
| | | G50 | DOF | GKGSALSLCGSFTSTLLNHNAAATATHGSGSVIGIGGFGIGLGSGEDDV | | 00144 |
| | | 410 | | SFGLGRAMWPFSTVGTATTTNVGSNGGHHAVPMPATWQFEGLESNAGGG | | 8 |
| | | | | FVSGEYFAWPDLSITTPGNSLK | | |
| 151 | 510 | AT5 | bZIP | DRARQQGFYVGNGVDTNALSFSDNMSSGIVAFEMEYGHWVEEQNRQICE | 244 | 0.102 |
| | | G10 | | LRTVLHGQVSDIELRSLVENAMKHYFQLFRMKSAAAKIDVFYVMSGMWK | | 77558 |
| | | 030 | | TSAERFFLWIGGFRPSELLKVLLPHFDPLTDQQLLDVCNLRQSCQQAED | | 2 |
| | | | | ALSQGMEKLQHTLAESVAAGKLGEGSYIPQMTCAMERLEALVSFVNQAD | | |
| | | | | HLRHETLQQMHRILTTRQAARGLLALGEYFQRLRALSSSWAARQREPT | | |
| 152 | 462 | AT2 | WRKY | MNGLVDSSRDKKMKNPRFSFRTKSDADILDDGYRWRKYGQKSVKNSLYP | 109 | 0.121 |
| | | G46 | | RSYYRCTQHMCNVKKQVQRLSKETSIVETTYEGIHNHPCEELMQTLTPL | | 02084 |
| | | 130 | | LHQLQFLSKFT | | 2 |
| 153 | 243 | AT2 | LOBAS2 | AGHQTSAAGDLRHSSESTNQFMTWQQTSVSPIGSAYSTPYNHHQPYYGH | 129 | 0.136 |
| | | G42 | | VNPNNPVSPQSSLEESFSNTSSDVTTTANVRETHHQTGGGVYGHDGIGF | | 24421 |
| | | 430 | | HEGYPNKKRSVSYCSSDLGELQALALRMMKN | | 2 |
| 154 | 328 | AT5 | MYB- | SGAKDKRRPSIHDITTVNLLNANLSRPSSDHGCLVSKQAEPKLGFTDRD | 96 | 0.153 |
| | | G05 | related | NAEEGVMFLGQNLSSVFSSYDPAIKFSGANVYGEGGYCISQDLETRK | | 28801 |
| | | 790 | | | | 2 |
| 155 | 117 | AT3 | C2C2- | SKSRSKSTVVVSTDNTTSTSSLTSRPSYSNPSKFHSYGQIPEFNSNLPI | 193 | 0.185 |
| | | G55 | DOF | LPPLQSLGDYNSSNTGLDFGGTQISNMISGMSSSGGILDAWRIPPSQQA | | 43742 |
| | | 370 | | QQFPFLINTTGLVQSSNALYPLLEGGVSATQTRNVKAEENDQDRGRDGD | | 4 |
| | | | | GVNNLSRNFLGNININSGRNEEYTSWGGNSSWTGFTSNNSTGHLSF | | |
| 156 | 52 | AT5 | AP2- | LEAGKHEDLGDNKKTISLKAKRKRQVTEDESQLISRKAVKREEAQVQAD | 92 | 0.190 |
| | | G51 | EREBP | ACPLTPSSWKGFWDGADSKDMGIFSVPLLSPCPSLGHSQLVVT | | 12254 |
| | | 190 | | | | 1 |
| 157 | 38 | AT3 | AP2- | ELLPCTSAEDMSAATIRKKATEVGAQVDAIGATVVQNNKRRRVFSQKRD | 76 | 0.195 |
| | | G50 | EREBP | FGGGLLELVDLNKLPDPENLDDDLVGK | | 71267 |
| | | 260 | | | | 6 |
| 158 | 278 | AT5 | MYB | RAGLPLYPPEMHVEALEWSQEYAKSRVMGEDRRHQDFLQLGSCESNVFF | 384 | 0.196 |
| | | G06 | | DTLNFTDMVPGTFDLADMTAYKNMGNCASSPRYENEMTPTIPSSKRLWE | | 18374 |
| | | 100 | | SELLYPGCSSTIKQEFSSPEQFRNTSPQTISKTCSFSVPCDVEHPLYGN | | 5 |
| | | | | RHSPVMIPDSHTPTDGIVPYSKPLYGAVKLELPSFQYSETTFDQWKKSS | | |
| | | | | SPPHSDLLDPFDTYIQSPPPPTGGEESDLYSNFDTGLLDMLLLEAKIRN | | |
| | | | | NSTKNNLYRSCASTIPSADLGQVTVSQTKSEEFDNSLKSFLVHSEMSTQ | | |
| | | | | NADETPPRQREKKRKPLLDITRPDVLLASSWLDHGLGIVKETGSMSDAL | | |
| | | | | AVLLGDDIGNDYMNMSVGASSGVGSCSWSNMPPVCQMTELP | | |
| 159 | 218 | AT4 | HSF | KPVHSHSLPNLQAQLNPLTDSERVRMNNQIERLTKEKEGLLEELHKQDE | 296 | 0.196 |
| | | G18 | | EREVFEMQVKELKERLQHMEKRQKTMVSFVSQVLEKPGLALNLSPCVPE | | 28198 |
| | | 880 | | TNERKRRFPRIEFFPDEPMLEENKTCVVVREEGSTSPSSHTREHQVEQL | | 6 |
| | | | | ESSIAIWENLVSDSCESMLQSRSMMTLDVDESSTFPESPPLSCIQLSVD | | |
| | | | | SRLKSPPSPRIIDMNCEPDGSKEQNTVAAPPPPPVAGANDGFWQQFFSE | | |
| | | | | NPGSTEQREVQLERKDDKDKAGVRTEKCWWNSRNVNAITEQLGHLTSSE | | |
| | | | | RS | | |
| 160 | 192 | AT1 | G2- | MIKKFSNMDYNQKRERCGQYIEALEEERRKIHVFQRELPLCLDLVTQAI | 177 | 0.198 |
| | | G13 | like | EACKRELPEMTTENMYGQPECSEQTTGECGPVLEQFLTIKDSSTSNEEE | | 98674 |
| | | 300 | | DEEFDDEHGNHDPDNDSEDKNTKSDWLKSVQLWNQPDHPLLPKEERLQQ | | 5 |
| | | | | ETMTRDESMRKDPMVNGGEGRKREAEKDGG | | |
| 161 | 107 | AT5 | C2C2- | RSRTYSSAATTSVVGSRNFPLQATPVLFPQSSSNGGITTAKGSASSFYG | 139 | 0.199 |
| | | G66 | DOF | GFSSLINYNAAVSRNGPGGGFNGPDAFGLGLGHGSYYEDVRYGQGITVW | | 95037 |
| | | 940 | | PFSSGATDAATTTSHIAQIPATWQFEGQESKVGFVSGDYVA | | 1 |
| 162 | 485 | AT5 | bHLH | MSNYGVKELTWENGQLTVHGLGDEVEPTTSNNPIWTQSLNGCETLESVV | 162 | 0.208 |
| | | G61 | | HQAALQQPSKFQLQSPNGPNHNYESKDGSCSRKRGYPQEMDRWFAVQEE | | 56622 |
| | | 270 | | SHRVGHSVTASASGTNMSWASFESGRSLKTARTGDRDYFRSGSETQDTE | | 9 |
| | | | | GDEQETRGEAGRSNG | | |
| 163 | 336 | AT5 | MYB- | REATGGDGSSVEPIVIPPPRPKRKPAHPYPRKFGNEADQTSRSVSPSER | 283 | 0.209 |
| | | G17 | related | DTQSPTSVLSTVGSEALCSLDSSSPNRSLSPVSSASPPAALTTTANAPE | | 22570 |
| | | 300 | | ELETLKLELFPSERLLNRESSIKEPTKQSLKLFGKTVLVSDSGMSSSLT | | 4 |
| | | | | TSTYCKSPIQPLPRKLSSSKTLPIIRNSQEELLSCWIQVPLKQEDVENR | | |
| | | | | CLDSGKAVQNEGSSTGSNTGSVDDTGHTEKTTEPETMLCQWEFKPSERS | | |
| | | | | AFSELRRTNSESNSRGFGPYKKRKMVTEEEEHEIHLHL | | |
| 164 | 310 | AT3 | MYB | KKMNDSCDSTINNGLDNKDFSISNKNTTSHQSSNSSKGQWERRLQTDIN | 217 | 0.215 |
| | | G47 | | MAKQALCDALSIDKPQNPTNFSIPDLGYGPSSSSSSTTTTTTTTRNTNP | | 67850 |
| | | 600 | | YPSGVYASSAENIARLLQNFMKDTPKTSVPLPVAATEMAITTAASSPST | | 5 |
| | | | | TEGDGEGIDHSLESENSIDEAEEKPKLIDHDINGLITQGSLSLFEKWLF | | |
| | | | | DEQSHDMIINNMSLEGQEVLF | | |
| 165 | 2 | AT5 | ABI3- | GVEIIDVPLGVEPETEPFHPTPKKPHKETTPASSFASGSGCSANGGING | 168 | 0.222 |
| | | G25 | VP1 | RGKQRSSDVKNPERYLLNPENPYFVQAVTKRNDVLYVSRPVVQSYRLKF | | 57929 |
| | | 475 | | GPVKSTITYLLPGEKKEEGENRIYNGKPCFSGWSVLCRRHNLNIGDSVV | | |
| | | | | CELERSGGVVTAVRVHFVKKD | | |
| 166 | 228 | AT1 | Homeo | TKQLEKDYDTLKRQFDTLKAENDLLQTHNQKLQAEIMGLKNREQTESIN | 156 | 0.253 |
| | | G69 | box | LNKETEGSCSNRSDNSSDNLRLDISTAPPSNDSTLTGGHPPPPQTVGRH | | 05853 |
| | | 780 | | FFPPSPATATTTTTTMQFFQNSSSGQSMVKEENSISNMFCAMDDHSGFW | | |
| | | | | PWLDQQQYN | | |
| 167 | 451 | AT2 | WRKY | MSSTSFTDLLGSSGVDCYEDDEDLRVSGSSFGGYYPERTGSGLPKFKTA | 165 | 0.262 |
| | | G30 | | QPPPLPISQSSHNFTFSDYLDSPLLLSSSHSLISPTTGTFPLQGENGTT | | 24534 |
| | | 250 | | NNHSDFPWQLQSQPSNASSALQETYGVQDHEKKQEMIPNEIATQNNNQS | | 5 |
| | | | | FGTERQIKIPAYMVSRNS | | |
| 168 | 431 | AT2 | Tri- | GDYKKIKEWESQIKEETESYWVMRNDVRREKKLPGFFDKEVYDIVDGGV | 210 | 0.264 |
| | | G33 | helix | IPPAVPVLSLGLAPASDEGLLSDLDRRESPEKLNSTPVAKSVTDVIDKE | | 83484 |
| | | 550 | | KQEACVADQGRVKEKQPEAANVEGGSTSQEERKRKRTSFGEKEEEEEEG | | |
| | | | | ETKKMQNQLIEILERNGQLLAAQLEVQNLNLKLDREQRKDHGDSLVAVL | | |
| | | | | NKLADAVAKIADKM | | |
| 169 | 50 | AT1 | AP2- | LIGYYGISSATPVNNNLSETVSDGNANLPLVGDDGNALASPVNNTLSET | 136 | 0.287 |
| | | G03 | EREBP | ARDGTLPSDCHDMLSPGVAEAVAGFFLDLPEVIALKEELDRVCPDQFES | | 65621 |
| | | 800 | | IDMGLTIGPQTAVEEPETSSAVDCKLRMEPDLDLNASP | | 1 |
| 170 | 355 | AT3 | NAC | SGSGPKNGEQYGAPFVEEEWEEEDDMTFVPDQEDLGSEDHVYVHMDDID | 390 | 0.322 |
| | | G10 | | QKSENFVVYDAIPIPLNFIHGESSNNVETNYSDSINYIQQTGNYMDSGG | | 03755 |
| | | 500 | | YFEQPAESYEKDQKPIIRDRDGSLQNEGIGCGVQDKHSETLQSSDNIFG | | 5 |
| | | | | TDTSCYNDFPVESNYLIGEAFLDPNSNLLENDGLYLETNDLSSTQQDGF | | |
| | | | | DFEDYLTFFDETFDPSQLMGNEDVFFDQEELFQEVETKELEKEETSRSK | | |
| | | | | HVVEEKEKDEASCSKQVDADATEFEPDYKYPLLKKASHMLGAIPAPLAN | | |
| | | | | ASEFPTKDAAIRLHAAQSSGSVHVTAGMITISDSNMGWSYGKNENLDLI | | |
| | | | | LSLGLVQGNTAPEKSGNSSAWAMLIFMCFWVLLLSVSFKVSILVSSR | | |
| 171 | 55 | AT2 | AP2- | MSSSDSVNNGVNSRMYFRNPSFSNVILNDNWSDLPLSVDDSQDMAIYNT | 90 | 0.329 |
| | | G44 | EREBP | LRDAVSSGWTPSVPPVTSPAEENKPPATKASGSHAPRQKGM | | 58350 |
| | | 840 | | | | 7 |
| 172 | 160 | AT1 | C2H2 | DKDNTGLGDGDKDNTCKGDDDKEKSGSGGCEKENEGNGGSGKDNNGNGD | 61 | 0.344 |
| | | G72 | | SQPAECSTGQKQ | | 98357 |
| | | 050 | | | | 4 |
| 173 | 271 | AT3 | MYB | MEFESVFKMHYPYLAAVIYDDSSTLKDFHPSLTDDFSCVHNVHHKPSMP | 183 | 0.370 |
| | | G27 | | HTYEIPSKETIRGITPSPCTEAFEACFHGTSNDHVFFGMAYTTPPTIEP | | 30552 |
| | | 785 | | NVSHVSHDNTMWENDQNQGFIFGTESTLNQAMADSNQFNMPKPLLSANE | | 5 |
| | | | | DTIMNRRQNNQVMIKTEQIKKKNKRFQMRRICKPTK | | |
| 174 | 373 | AT3 | NAC | SGVVSRETNLISSSSSSAVTGEFSSAGSAIAPIINTFATEHVSCFSNNS | 138 | 0.372 |
| | | G15 | | AAHTDASFHTFLPAPPPSLPPRQPRHVGDGVAFGQFLDLGSSGQIDFDA | | 51078 |
| | | 170 | | AAAAFFPNLPSLPPTVLPPPPSFAMYGGGSPAVSVWPFTL | | 2 |
| 175 | 299 | AT3 | MYB | RAGLPLYPPEIYVDDLHWSEEYTKSNIIRVDRRRRHQDFLQLGNSKDNV | 408 | 0.373 |
| | | G11 | | LFDDLNFAASLLPAASDLSDLVACNMLGTGASSSRYESYMPPILPSPKQ | | 11029 |
| | | 440 | | IWESGSRFPMCSSNIKHEFQSPEHFQNTAVQKNPRSCSISPCDVDHHPY | | 5 |
| | | | | ENQHSSHMMMVPDSHTVTYGMHPTSKPLFGAVKLELPSFQYSETSAFDQ | | |
| | | | | WKTTPSPPHSDLLDSVDAYIQSPPPSQVEESDCFSSCDTGLLDMLLHEA | | |
| | | | | KIKTSAKHSLLMSSPQKSFSSTTCTTNVTQNVPRGSENLIKSGEYEDSQ | | |
| | | | | KYLGRSEITSPSQLSAGGFSSAFAGNVVKTEELDQVWEPKRVDITRPDV | | |
| | | | | LLASSWLDQGCYGIVSDTSSMSDALALLGGDDIGNSYVTVGSSSGQAPR | | |
| | | | | GVGSYGWTNMPPVWSL | | |
| 176 | 263 | AT5 | MYB | SGMGIDPVTHKPFSHLMAEITTTLNPPQVSHLAEAALGCFKDEMLHLLT | 204 | 0.383 |
| | | G56 | | KKRVDLNQINFSNHNPNPNNFHEIADNEAGKIKMDGLDHGNGIMKLWDM | | 72721 |
| | | 110 | | GNGFSYGSSSSSFGNEERNDGSASPAVAAWRGHGGIRTAVAETAAAEEE | | 4 |
| | | | | ERRKLKGEVVDQEEIGSEGGRGDGMTMMRNHHHHQHVFNVDNVLWDLQA | | |
| | | | | DDLINHMV | | |
| 177 | 491 | AT2 | bHLH | ESVKEYEEQKKEKTMESVVLVKKSSLVLDENHQPSSSSSSDGNRNSSSS | 133 | 0.399 |
| | | G22 | | NLPEIEVRVSGKDVLIKILCEKQKGNVIKIMGEIEKLGLSITNSNVLPF | | 80112 |
| | | 750 | | GPTFDISIIAQKNNNFDMKIEDVVKNLSFGLSKLT | | 3 |
| 178 | 347 | AT1 | NAC | SALANKIEEQHHGTKKNKGTTNSEQSTSSTCLYSDGMYENLENSGYPVS | 475 | 0.404 |
| | | G65 | | PETGGLTQLGNNSSSDMETIENKWSQFMSHDTSFNFPPQSQYGTISYPP | | 78138 |
| | | 910 | | SKVDIALECARLQNRMLPPVPPLYVEGLTHNEYFGNNVANDTDEMLSKI | | 3 |
| | | | | IALAQASHEPRNSLDSWDGGSASGNFHGDENYSGEKVSCLEANVEAVDM | | |
| | | | | QEHHVNFKEERLVENLRWVGVSSKELEKSFVEEHSTVIPIEDIWRYHND | | |
| | | | | NQEQEHHDQDGMDVNNNNGDVDDAFTLEFSENEHNENLLDKNDHETTSS | | |
| | | | | SCFEVVKKVEVSHGLFVTTRQVTNTFFQQIVPSQTVIVYINPTDGNECC | | |
| | | | | HSMTSKEEVHVRKKINPRINGVSSTVLGQWRKFAHVIGFIPMLLLMRCV | | |
| | | | | HRGNSNKNRGSEGYSRQPTRGDCNNRGTILMMENAVVRRKIWKKKKEKN | | |
| | | | | MVDEQGFRFQDSFVLKKLGLSLAIILAVSTISLI | | |
| 179 | 522 | AT2 | bZIP | MIPAEINGYFQYLSPEYNVINMPSSPTSSLNYLNDLIINNNNYSSSSNS | 71 | 0.423 |
| | | G04 | | QDLMISNNSTSDEDHHQSIMVL | | 87725 |
| | | 038 | | | | 1 |
| 180 | 40 | AT4 | AP2- | VQPEPEPVQEQEQEPESNMSVSISESMDDSQHLSSPTSVLNYQTYVSEE | 161 | 0.425 |
| | | G27 | EREBP | PIDSLIKPVKQEFLEPEQEPISWHLGEGNTNTNDDSFPLDITELDNYEN | | 13581 |
| | | 950 | | ESLPDISIFDQPMSPIQPTENDFFNDLMLFDSNAEEYYSSEIKEIGSSF | | 9 |
| | | | | NDLDDSLISDLLLV | | |
| 181 | 54 | AT5 | AP2- | ERAQLASNTSTTTGPPNYYSSNNQIYYSNPQTNPQTIPYFNQYYYNQYL | 115 | 0.432 |
| | | G07 | EREBP | HQGGNSNDALSYSLAGGETGGSMYNHQTLSTTNSSSSGGSSRQQDDEQD | | 21228 |
| | | 310 | | YARYLRFGDSSPPNSGF | | 5 |
| 182 | 158 | AT1 | C2H2 | METEDDLCNTNWGSSSSKSREPGSSDCGNSTFAGFTSQQKWEDASILDY | 245 | 0.440 |
| | | G34 | | EMGVEPGLQESIQANVDFLQGVRAQAWDPRTMLSNLSFMEQKIHQLQDL | | 24328 |
| | | 370 | | VHLLVGRGGQLQGRQDELAAQQQQLITTDLTSIIIQLISTAGSLLPSVK | | 3 |
| | | | | HNMSTAPGPFTGQPGSAVFPYVREANNVASQSQNNNNCGAREFDLPKPV | | |
| | | | | LVDEREGHVVEEHEMKDEDDVEEGENLPPGSYEILQLEKEEILAPHTHF | | |
| 183 | 119 | AT2 | C2C2- | TKSNSNNNNNSTATSNNTSFSSGNASTISTILSSHYGGNQESILSQILS | 187 | 0.469 |
| | | G37 | DOF | PARLMNPTYNHLGDLTSNTKTDNNMSLLNYGGLSQDLRSIHMGASGGSL | | 16694 |
| | | 590 | | MSCVDEWRSASYHQQSSMGGGNLEDSSNPNPSANGFYSFESPRITSASI | | 2 |
| | | | | SSALASQFSSVKVEDNPYKWVNVNGNCSSWNDLSAFGSSR | | |
| 184 | 392 | AT2 | ND | ITISYIETAGSTLTRQKSLKEQYLFHCQCARCSNFGKPHDIEESAILEG | 238 | 0.471 |
| | | G17 | | YRCANEKCTGFLLRDPEEKGFVCQKCLLLRSKEEVKKLASDLKTVSEKA | | 18624 |
| | | 900 | | PTSPSAEDKQAAIELYKTIEKLQVKLYHSFSIPLMRTREKLLKMLMDVE | | 9 |
| | | | | IWREALNYCRLIVPVYQRVYPATHPLIGLQFYTQGKLEWLLGETKEAVS | | |
| | | | | SLIKAFDILRISHGISTPFMKELSAKLEEARAEASYKQLALH | | |
| 185 | 9 | AT5 | AP2- | MAPPMTNCLTFSLSPMEMLKSTDQSHFSSSYDDSSTPYLIDNFYAFKEE | 230 | 0.477 |
| | | G65 | EREBP | AEIEAAAASMADSTTLSTFFDHSQTQIPKLEDFLGDSFVRYSDNQTETQ | | 13035 |
| | | 510 | | DSSSLTPFYDPRHRTVAEGVTGFFSDHHQPDFKTINSGPEIFDDSTTSN | | 9 |
| | | | | IGGTHLSSHVVESSTTAKLGFNGDCTTTGGVLSLGVNNTSDQPLSCNNG | | |
| | | | | ERGGNSNKKKTVSKKETSDDSKKKIVETLGQRTS | | |
| 186 | 63 | AT4 | AP2- | MATPNEVSALFLIKKYLLDELSPLPTTATTNRWMNDFTSFDQTGFEFSE | 135 | 0.480 |
| | | G17 | EREBP | FETKPEIIDLVTPKPEIFDFDVKSEIPSESNDSFTFQSNPPRVTVQSNR | | 38010 |
| | | 490 | | KPPLKIAPPNRTKWIQFATGNPKPELPVPVVAAEEKR | | 4 |
| 187 | 380 | AT2 | NAC | ADFRASSTQKMEDGVVQDDGYVGQRGGLEKEDKSYYESEHQIPNGDIAE | 179 | 0.486 |
| | | G27 | | SSNVVEDQADTDDDCYAEILNDDIIKLDEEALKASQAFRPTNPTHQETI | | 05314 |
| | | 300 | | SSESSSKRSKCGIKKESTETMNCYALFRIKNVAGTDSSWRFPNPFKIKK | | 9 |
| | | | | DDSQRLMKNVLATTVFLAILFSFFWTVLIARN | | |
| 188 | 267 | AT1 | MYB | REQSSSYRRRKTMVSLKPLINPNPHIENDEDPTRLALTHLASSDHKQLM | 122 | 0.494 |
| | | G69 | | LPVPCFPGYDHENESPLMVDMFETQMMVGDYIAWTQEATTFDFLNQTGK | | 41184 |
| | | 560 | | SEIFERINEEKKPPFFDFLGLGTV | | |
| 189 | 436 | AT5 | Tri- | MELLAGDCRKRVGDDFEEDINPFDGSDGGCGWMYGTRQMGSNGNDDALA | 302 | 0.497 |
| | | G47 | helix | TLADLASPPQKLKPIRCGVKLPSSSEDRHPLDILAGTLDRLPEMGFGCF | | 27001 |
| | | 660 | | EAPLGSKIADVEESGQLTRGFSKEEDDSLPPLQMEFQARNRISWDGLSL | | 5 |
| | | | | SSSVDSSDSDSSPDVRKTVTGKRKRETRVKLEHFLEKLVGSMMKRQEKM | | |
| | | | | HNQLINVMEKMEVERIRREEAWRQQETERMTQNEEARKQEMARNLSLIS | | |
| | | | | FIRSVTGDEIEIPKQCEFPQPLQQILPEQCKDEKCESAQREREIKFRYS | | |
| | | | | SGSGSSGR | | |
| 190 | 350 | AT2 | NAC | VTSQRNPTILPPNRKPVITLTDTCSKTSSLDSDHTSHRTVDSMSHEPPL | 108 | 0.524 |
| | | G43 | | PQPQNPYWNQHIVGFNQPTYTGNDNNLLMSFWNGNGGDFIGDSASWDEL | | 84472 |
| | | 000 | | RSVIDGNTKP | | 5 |
| 191 | 71 | AT3 | AP2- | INRYDVKAILESSTLPIGGGAAKRLKEAQALESSRKREAEMIALGSSFQ | 233 | 0.525 |
| | | G20 | EREBP | YGGGSSTGSGSTSSRLQLQPYPLSIQQPLEPFLSLQNNDISHYNNNNAH | | 19556 |
| | | 840 | | DSSSFNHHSYIQTQLHLHQQTNNYLQQQSSQNSQQLYNAYLHSNPALLH | | 2 |
| | | | | GLVSTSIVDNNNNNGGSSGSYNTAAFLGNHGIGIGSSSTVGSTEEFPTV | | |
| | | | | KTDYDMPSSDGTGGYSGWTSESVQGSNPGGVFTMWNE | | |
| 192 | 461 | AT4 | WRKY | MFRFPVSLGGGPRENLKPSDEQHQRAVVNEVDFFRSAEKRDRVSREEQN | 285 | 0.549 |
| | | G04 | | IIADETHRVHVKRENSRVDDHDDRSTDHINIGLNLLTANTGSDESMVDD | | 48519 |
| | | 450 | | GLSVDMEEKRTKCENAQLREELKKASEDNQRLKQMLSQTTNNENSLQMQ | | 4 |
| | | | | LVAVMRQQEDHHHLATTENNDNVKNRHEVPEMVPRQFIDLGPHSDEVSS | | |
| | | | | EERTTVRSGSPPSLLEKSSSRQNGKRVLVREESPETESNGWRNPNKVPK | | |
| | | | | HHASSSICGGNGSENASSKVIEQAAAEATMRKARVSVRAR | | |
| 193 | 32 | AT5 | AP2- | DIVRQGHYKQILSPSINAKIESICNSSDLPLPQIEKQNKTEEVLSGFSK | 110 | 0.556 |
| | | G65 | EREBP | PEKEPEFGEIYGCGYSGSSPESDITLLDESSDCVKEDESFLMGLHKYPS | | 52508 |
| | | 130 | | LEIDWDAIEKLF | | 6 |
| 194 | 182 | AT1 | EIL | NSNVTETHRRGNNADRRKPVVNSDSDYDVDGTEEASGSVSSKDSRRNQI | 273 | 0.558 |
| | | G73 | | QKEQPTAISHSVRDQDKAEKHRRRKRPRIRSGTVNRQEEEQPEAQQRNI | | 61407 |
| | | 730 | | LPDMNHVDAPLLEYNINGTHQEDDVVDPNIALGPEDNGLELVVPEENNN | | 7 |
| | | | | YTYLPLVNEQTMMPVDERPMLYGPNPNQELQFGSGYNFYNPSAVFVHNQ | | |
| | | | | EDDILHTQIEMNTQAPPHNSGFEEAPGGVLQPLGLLGNEDGVTGSELPQ | | |
| | | | | YQSGILSPLTDLDEDYGGFGDDFSWFGA | | |
| 195 | 281 | AT5 | MYB | DSYMSSGLLDQYQAMPLAPYERSSTLQSTFMQSNIDGNGCLNGQAENEI | 779 | 0.567 |
| | | G11 | | DSRQNSSMVGCSLSARDFQNGTINIGHDFHPCGNSQENEQTAYHSEQFY | | 50463 |
| | | 510 | | YPELEDISVSISEVSYDMEDCSQFPDHNVSTSPSQDYQFDFQELSDISL | | 2 |
| | | | | EMRHNMSEIPMPYTKESKESTLGAPNSTLNIDVATYTNSANVLTPETEC | | |
| | | | | CRVLFPDQESEGHSVSRSLTQEPNEFNQVDRRDPILYSSASDRQISEAT | | |
| | | | | KSPTQSSSSRFTATAASGKGTLRPAPLIISPDKYSKKSSGLICHPFEVE | | |
| | | | | PKCTTNGNGSFICIGDPSSSTCVDEGTNNSSEEDQSYHVNDPKKLVPVN | | |
| | | | | DEASLAEDRPHSLPKHEPNMTNEQHHEDMGASSSLGFPSFDLPVENCDL | | |
| | | | | LQSKNDPLHDYSPLGIRKLLMSTMTCMSPLRLWESPTGKKTLVGAQSIL | | |
| | | | | RKRTRDLLTPLSEKRSDKKLEIDIAASLAKDESRLDVMFDETENRQSNF | | |
| | | | | GNSTGVIHGDRENHFHILNGDGEEWSGKPSSLFSHRMPEETMHIRKSLE | | |
| | | | | KVDQICMEANVREKDDSEQDVENVEFFSGILSEHNTGKPVLSTPGQSVT | | |
| | | | | KAEKAQVSTPRNQLQRTLMATSNKEHHSPSSVCLVINSPSRARNKEGHL | | |
| | | | | VDNGTSNENFSIFCGTPFRRGLESPSAWKSPFYINSLLPSPREDTDLTI | | |
| | | | | EDMGYIFSPGERSYESIGVMTQINEHTSAFAAFADAMEVSISPTNDDAR | | |
| | | | | QKKELDKENNDPLLAERRVLDENDCESPIKATEEVSSYLLKGCR | | |
| 196 | 521 | AT1 | bZIP | RAQVLELNHRLQSLNEIVDFVESSSSGFGMETGQGLFDGGLFDGVMNPM | 71 | 0.568 |
| | | G75 | | NLGFYNQPIMASASTAGDVENC | | 95699 |
| | | 390 | | | | 7 |
| 197 | 325 | AT3 | MYB- | KNGTLAHVPPPRPKRKAAHPYPQKASKNAQMPLQVSTSFTTTRNGDMPG | 206 | 0.573 |
| | | G09 | related | YASWDDASMLLNRVISPQHELATLRGAEADIGSKGLLNVSSPSTSGMGS | | 54616 |
| | | 600 | | SSRTVSGSEIVRKAKQPPVLHGVPDFAEVYNFIGSVEDPETRGHVEKLK | | |
| | | | | EMDPINFETVLLLMRNLTVNLSNPDLESTRKVLLSYDNVTTELPSVVSL | | |
| | | | | VKNSTSDKSA | | |
| 198 | 194 | AT1 | G2- | MMVEMDYAKKMQKCHEYVEALEEEQKKIQVFQRELPLCLELVTQAIEAC | 211 | 0.579 |
| | | G68 | like | RKELSGTTTTTSEQCSEQTTSVCGGPVFEEFIPIKKISSLCEEVQEEEE | | 76018 |
| | | 670 | | EDGEHESSPELVNNKKSDWLRSVQLWNHSPDLNPKEERVAKKAKVVEVK | | 8 |
| | | | | PKSGAFQPFQKRVLETDLQPAVKVASSMPATTTSSTTETCGGKSDLIKA | | |
| | | | | GDEERRIEQQQSQSH | | |
| 199 | 385 | AT1 | NAC | TQPRQCGSMEPKPKNLVNLNRFSYENIQAGFGYEHGGKSEETTQVIREL | 78 | 0.597 |
| | | G28 | | VVREGDGSCSFLSFTCDASKGKESFMKNQ | | 12378 |
| | | 470 | | | | 7 |
| 200 | 351 | AT3 | NAC | IVIEAKPRDQHRSYVHAMSNVSGNCSSSFDTCSDLEISSTTHQVQNTFQ | 322 | 0.601 |
| | | G03 | | PREGNERFNSNAISNEDWSQYYGSSYRPFPTPYKVNTEIECSMLQHNIY | | 23489 |
| | | 200 | | LPPLRVENSAFSDSDFFTSMTHNNDHGVEDDFTFAASNSNHNNSVGDQV | | 1 |
| | | | | IHVGNYDEQLITSNRHMNQTGYIKEQKIRSSLDNTDEDPGFHGNNTNDN | | |
| | | | | IDIDDFLSFDIYNEDNVNQIEDNEDVNTNETLDSSGFEVVEEETRENNQ | | |
| | | | | MLISTYQTTKILYHQVVPCHTLKVHVNPISHNVEERTLFIEEDKDSWLQ | | |
| | | | | RAEKITKTKLTLFSLMAQQYYKCLAIFF | | |
| 201 | 89 | AT3 | ARID | LEKPVSSLQSTDEALKSLANESPNPEEGIDEPQVGYEVQGFIDGKFDSG | 106 | 0.620 |
| | | G13 | | YLVTMKLGSQELKGVLYHIPQTPSQSQQTMETPSAIVQSSQRRHRKKSK | | 27463 |
| | | 350 | | LAVVDTQK | | 2 |
| 202 | 291 | AT4 | MYB | KNLWNSCLKKKLRLRGIDPVTHKLLTEIETGTDDKTKPVEKSQQTYLVE | 232 | 0.627 |
| | | G01 | | TDGSSSTTTCSTNQNNNTDHLYTGNFGFQRLSLENGSRIAAGSDLGIWI | | 40821 |
| | | 680 | | PQTGRNHHHHVDETIPSAVVLPGSMFSSGLTGYRSSNLGLIELENSEST | | |
| | | | | GPMMTEHQQIQESNYNNSTFFGNGNLNWGLTMEENQNPFTISNHSNSSL | | |
| | | | | YSDIKSETNFFGTEATNVGMWPCNQLQPQQHAYGHI | | |
| 203 | 343 | AT1 | NAC | SGSGPKNGEQYGAPFIEEEWAEDDDDDVDEPANQLVVSASVDNSLWGKG | 368 | 0.647 |
| | | G32 | | LNQSELDDNDIEELMSQVRDQSGPTLQQNGVSGLNSHVDTYNLENLEED | | 12525 |
| | | 870 | | MYLEINDLMEPEPEPTSVEVMENNWNEDGSGLLNDDDFVGADSYFLDLG | | 2 |
| | | | | VTNPQLDFVSGDLKNGFAQSLQVNTSLMTYQANNNQFQQQSGKNQASNW | | |
| | | | | PLRNSYTRQINNGSSWVQELNNDGLTVTRFGEAPGTGDSSEFLNPVPSG | | |
| | | | | ISTTNEDDPSKDESSKFASSVWTFLESIPAKPAYASENPFVKLNLVRMS | | |
| | | | | TSGGRFRFTSKSTGNNVVVMDSDSAVKRNKSGGNNDKKKKKNKGFFCLS | | |
| | | | | IIGALCALFWVIIGTMGGSGRPLLW | | |
| 204 | 143 | AT2 | C2H2 | LSPPRPLGTSTQRNPSSSLAGSRLKAMALDCEMVGGGADGTIDQCASVC | 238 | 0.657 |
| | | G48 | | LVDDDENVIFSTHVQPLLPVTDYRHEITGLTKEDLKDGMPLEHVRERVF | | 31819 |
| | | 100 | | SFLCGGQNDGAGRLLLVGHDLRHDMSCLKLEYPSHLLRDTAKYVPLMKT | | 1 |
| | | | | NLVSQSLKYLTKSYLGYKIQCGKHEVYEDCVSAMRLYKRMRDQEHVCSG | | |
| | | | | KAEGNGLNSRKQSDLEKMNAEELYQKSTSEYRCWCLDRLSNP | | |
| 205 | 525 | AT3 | bZIP | RAQASELTDRLRSLNSVLEMVEEISGQALDIPEIPESMQNPWQMPCPMQ | 60 | 0.664 |
| | | G62 | | PIRASADMEDC | | 49564 |
| | | 420 | | | | 7 |
| 206 | 496 | AT4 | bHLH | LQVKVLSMSRLGGAASASSQISEDAGGSHENTSSSGEAKMTEHQVAKLM | 124 | 0.665 |
| | | G30 | | EEDMGSAMQYLQGKGLCLMPISLATTISTATCPSRSPFVKDTGVPLSPN | | 05214 |
| | | 980 | | LSTTIVANGNGSSLVTVKDAPSVSKP | | 4 |
| 207 | 422 | AT3 | TCP | TGTGTIPANFTSLNISLRSSGSSMSLPSHFRSAASTESPNNIFSPAMLQ | 318 | 0.682 |
| | | G47 | | QQQQQQRGGGVGFHHPHLQGRAPTSSLFPGIDNFTPTTSFLNFHNPTKQ | | 05249 |
| | | 620 | | EGDQDSEELNSEKKRRIQTTSDLHQQQQQHQHDQIGGYTLQSSNSGSTA | | 8 |
| | | | | TAAAAQQIPGNFWMVAAAAAAGGGGGNNNQTGGLMTASIGTGGGGGEPV | | |
| | | | | WTFPSINTAAAALYRSGVSGVPSGAVSSGLHFMNFAAPMAFLTGQQQLA | | |
| | | | | TTSNHEINEDSNNNEGGRSDGGGDHHNTQRHHHHQQQHHHNILSGLNQY | | |
| | | | | GRQVSGDSQASGSLGGGDEEDQQD | | |
| 208 | 129 | AT4 | C2C2- | KEERRASTARNSTSGGGSTAAGVPTLDHQASANYYYNNNNQYASSSPWH | 104 | 0.685 |
| | | G36 | GATA | HQHNTQRVPYYSPANNEYSYVDDVRVVDHDVTTDPFLSWRLNVADRTGL | | 55741 |
| | | 620 | | VHDFTM | | 4 |
| 209 | 465 | AT4 | WRKY | MEEHIQDRREIAFLHSGEFLHGDSDSKDHQPNESPVERHHESSIKEVDE | 233 | 0.691 |
| | | G01 | | FAAKSQPFDLGHVRTTTIVGSSGFNDGLGLVNSCHGTSSNDGDDKTKTQ | | 95995 |
| | | 720 | | ISRLKLELERLHEENHKLKHLLDEVSESYNDLQRRVLLARQTQVEGLHH | | |
| | | | | KQHEDVPQAGSSQALENRRPKDMNHETPATTLKRRSPDDVDGRDMHRGS | | |
| | | | | PKTPRIDQNKSTNHEEQQNPHDQLPYRKARVSVRARS | | |
| 210 | 7 | AT3 | ABI3- | HSEINYHSTGLMDSAHNHFKRARLFEDLEDEDAEVIFPSSVYPSPLPES | 145 | 0.696 |
| | | G18 | VP1 | TVPANKGYASSAIQTLFTGPVKAEEPTPTPKIPKKRGRKKKNADPEEIN | | 90078 |
| | | 990 | | SSAPRDDDPENRSKFYESASARKRTVTAEERERAINAAKTFEPTNPF | | 7 |
| 211 | 211 | AT5 | HB | QLERDYGVLKSNFDALKRNRDSLQRDNDSLLGQIKELKAKLNVEGVKGI | 185 | 0.709 |
| | | G65 | | EENGALKAVEANQSVMANNEVLELSHRSPSPPPHIPTDAPTSELAFEMF | | 47672 |
| | | 310 | | SIFPRTENERDDPADSSDSSAVLNEEYSPNTVEAAGAVAATTVEMSTMG | | 6 |
| | | | | CFSQFVKMEEHEDLFSGEEACKLFADNEQWYCSDQWNS | | |
| 212 | 66 | AT1 | AP2- | VIVGSSPTQSSTVVDSPTAARFITPPHLELSLGGGGACRRKIPLVHPVY | 98 | 0.723 |
| | | G53 | EREBP | YYNMATYPKMTTCGVQSESETSSVVDFEGGAGKISPPLDLDLNLAPPAE | | 44146 |
| | | 170 | | | | 6 |
| 213 | 233 | AT1 | Homeo | LSVPASSSRDLGGVILSPEGKRSMMRLAQRMISNYCLSVSRSNNTRSTV | 262 | 0.745 |
| | | G73 | box | VSELNEVGIRVTAHKSPEPNGTVLCAATTFWLPNSPQNVENFLKDERTR | | 59076 |
| | | 360 | | PQWDVLSNGNAVQEVAHISNGSHPGNCISVLRGSNATHSNNMLILQESS | | 8 |
| | | | | TDSSGAFVVYSPVDLAALNIAMSGEDPSYIPLLSSGFTISPDGNGSNSE | | |
| | | | | QGGASTSSGRASASGSLITVGFQIMVSNLPTAKLNMESVETVNNLIGTT | | |
| | | | | VHQIKTALSGPTASTTA | | |
| 214 | 219 | AT5 | HSF | DPDRWEFANEGFLRGRKQLLKSIVRRKPSHVQQNQQQTQVQSSSVGACV | 390 | 0.745 |
| | | G16 | | EVGKFGIEEEVERLKRDKNVLMQELVRLRQQQQATENQLQNVGQKVQVM | | 88295 |
| | | 820 | | EQRQQQMMSFLAKAVQSPGFLNQLVQQNNNDGNRQIPGSNKKRRLPVDE | | 1 |
| | | | | QENRGDNVANGLNRQIVRYQPSINEAAQNMLRQFLNTSTSPRYESVSNN | | |
| | | | | PDSFLLGDVPSSTSVDNGNPSSRVSGVTLAEFSPNTVQSATNQVPEASL | | |
| | | | | AHHPQAGLVQPNIGQSPAQGAAPADSWSPEFDLVGCETDSGECFDPIMA | | |
| | | | | VLDESEGDAISPEGEGKMNELLEGVPKLPGIQDPFWEQFFSVELPAIAD | | |
| | | | | TDDILSGSVENNDLVLEQEPNEWTRNEQQMKYLTEQMGLLSSEAQRK | | |
| 215 | 438 | AT1 | Tri- | KEFKKAKHHDRGNGSAKMSYYKEIEDILRERSKKVTPPQYNKSPNTPPT | 263 | 0.760 |
| | | G13 | helix | SAKVDSFMQFTDKGFDDTSISFGSVEANGRPALNLERRLDHDGHPLAIT | | 60673 |
| | | 450 | | TAVDAVAANGVTPWNWRETPGNGDDSHGQPFGGRVITVKFGDYTRRIGV | | 2 |
| | | | | DGSAEAIKEVIRSAFGLRTRRAFWLEDEDQIIRCLDRDMPLGNYLLRLD | | |
| | | | | DGLAIRVCHYDESNQLPVHSEEKIFYTEEDYREFLARQGWSSLQVDGER | | |
| | | | | NIENMDDLQPGAVYRGVR | | |
| 216 | 334 | AT5 | MYB- | KNGTLAHVPPPRPKRKAAHPYPQKASKNAQMSLHVSMSFPTQINNLPGY | 196 | 0.772 |
| | | G02 | related | TPWDDDTSALLNIAVSGVIPPEDELDTLCGAEVDVGSNDMISETSPSAS | | 36141 |
| | | 840 | | GIGSSSRTLSDSKGLRLAKQAPSMHGLPDFAEVYNFIGSVEDPDSKGRM | | 7 |
| | | | | KKLKEMDPINFETVLLLMRNLTVNLSNPDFEPTSEYVDAAEEGHEHLSS | | |
| 217 | 426 | AT1 | TCP | PLLNTNFDHLDQNQNQTKSACSSGTSESSLLSLSRTEIRGKARERARER | 216 | 0.781 |
| | | G30 | | TAKDRDKDLQNAHSSFTQLLTGGFDQQPSNRNWTGGSDCFNPVQLQIPN | | 04294 |
| | | 210 | | SSSQEPMNHPFSFVPDYNFGISSSSSAINGGYSSRGTLQSNSQSLFLNN | | 2 |
| | | | | NNNITQRSSISSSSSSSSPMDSQSISFFMATPPPLDHHNHQLPETFDGR | | |
| | | | | LYLYYGEGNRSSDDKAKERR | | |
| 218 | 153 | AT1 | C2H2 | TESLNKARELVLRNDSFPPHQGPPSFSYHQGDVHIGDLTQFKPMMYPPR | 130 | 0.793 |
| | | G13 | | HFSLPGSSSILQLQPPYLYPPLSSPFPQHNTNIGNNGTRHQTLTNSVCG | | 95075 |
| | | 400 | | GRALPDSSYTFIGAPVANGSRVAPHLPPHHGL | | 2 |
| 219 | 209 | AT2 | HB | RVEDEYTKLKNAYETTVVEKCRLDSEVIHLKEQLYEAEREIQRLAKRVE | 104 | 0.795 |
| | | G18 | | GTLSNSPISSSVTIEANHTTPFFGDYDIGEDGEADENLLYSPDYIDGLD | | 50601 |
| | | 550 | | WMSQFM | | 4 |
| 220 | 416 | AT1 | TCP | TGTGTIPANFTSLNISLRSSRSSLSAAHLRTTPSSYYFHSPHQSMTHHL | 219 | 0.797 |
| | | G69 | | QHQHQVRPKNESHSSSSSSSQLLDHNQMGNYLVQSTAGSLPTSQSPATA | | 85671 |
| | | 690 | | PFWSSGDNTQNLWAFNINPHHSGVVAGDVYNPNSGGSGGGSGVHLMNFA | | 3 |
| | | | | APIALFSGQPLASGYGGGGGGGGEHSHYGVLAALNAAYRPVAETGNHNN | | |
| | | | | NQQNRDGDHHHNHQEDGSTSHHS | | |
| 221 | 430 | AT | Tri- | KYHKRTKEGRTGKSEGKTYRFFDQLEALESQSTTSLHHHQQQTPLRPQQ | 282 | 0.806 |
| | | G76 | helix | NNNNNNNNNNNSSIFSTPPPVTTVMPTLPSSSIPPYTQQINVPSFPNIS | | 08205 |
| | | 880 | | GDFLSDNSTSSSSSYSTSSDMEMGGGTATTRKKRKRKWKVFFERLMKQV | | 6 |
| | | | | VDKQEELQRKFLEAVEKREHERLVREESWRVQEIARINREHEILAQERS | | |
| | | | | MSAAKDAAVMAFLQKLSEKQPNQPQPQPQPQQVRPSMQLNNNNQQQPPQ | | |
| | | | | RSPPPQPPAPLPQPIQAVVSTLDTTKTDNGGDQNMTP | | |
| 222 | 466 | AT5 | WRKY | MNDADTNLGSSFSDDTHSVFEFPELDLSDEWMDDDLVSAVSGMNQSYGY | 106 | 0.809 |
| | | G26 | | QTSDVAGALFSGSSSCFSHPESPSTKTYVAATATASADNQNKKEKKKIK | | 63998 |
| | | 170 | | GRVAFKTR | | 1 |
| 223 | 360 | AT4 | NAC | KNLFKVVNEGSSSINSLDQHNHDASNNNHALQARSFMHRDSPYQLVRNH | 181 | 0.816 |
| | | G10 | | GAMTFELNKPDLALHQYPPIFHKPPSLGFDYSSGLARDSESAASEGLQY | | 43334 |
| | | 350 | | QQACEPGLDVGTCETVASHNHQQGLGEWAMMDRLVTCHMGNEDSSRGIT | | |
| | | | | YEDGNNNSSSVVQPVPATNQLTLRSEMDFWGYSK | | |
| 224 | 198 | AT3 | G2- | PHKEHSQNHSICIRDTNRASMLDLRRNAVFTTSPLIIGRNMNEMQMEVQ | 155 | 0.819 |
| | | G12 | like | RRIEEEVVIERQVNQRIAAQGKYMESMLEKACETQEASLTKDYSTLFED | | 73026 |
| | | 730 | | RTNICNNTSSIPIPWFEDHFPSSSSMDSTLILPDINSNFSLQDSRSSIT | | 7 |
| | | | | KGRTVCLG | | |
| 225 | 94 | AT1 | BSD | MFSNFLESLYDGIGDDDAADDDEDNNNDEKTPKASTERHDFSRNAVRLS | 203 | 0.847 |
| | | G10 | | PEEEAQARGVKDDLTELGHTLTRQFRGVANFLAPLPDGSSSSSSDLSNH | | 72965 |
| | | 720 | | PRENQSRSSDPGLNQSRSSDRDESCVGSDTPETGIRFRSWDLEEKLAEG | | 7 |
| | | | | NDPEDEEEEEEETDEEEEEEEEIAAVALTDEVLAFARNIAMHPETWLDF | | |
| | | | | PLDPDED | | |
| 226 | 120 | AT4 | C2C2- | PKIDQSSVSQMILAEIQQGNHQPFKKFQENISVSVSSSSDVSIVGNHED | 119 | 0.848 |
| | | G21 | DOF | DLSELHGITNSTPIRSFTMDRLDFGEESFQQDLYDVGSNDLIGNPLINQ | | 74146 |
| | | 030 | | SIGGYVDNHKDEHKLQFEYES | | 7 |
| 227 | 439 | AT1 | Tri- | KYHKRTKEGRTGKSEGKTYRFFEELEAFETLSSYQPEPESQPAKSSAVI | 291 | 0.878 |
| | | G76 | helix | TNAPATSSLIPWISSSNPSTEKSSSPLKHHHQVSVQPITTNPTFLAKQP | | 80731 |
| | | 890 | | SSTTPFPFYSSNNTTTVSQPPISNDLMNNVSSLNLFSSSTSSSTASDEE | | 3 |
| | | | | EDHHQVKSSRKKRKYWKGLFTKLTKELMEKQEKMQKRFLETLEYREKER | | |
| | | | | ISREEAWRVQEIGRINREHETLIHERSNAAAKDAAIISFLHKISGGQPQ | | |
| | | | | QPQQHNHKPSQRKQYQSDHSITFESKEPRAVLLDTTIKMGNYDNNH | | |
| 228 | 363 | AT5 | NAC | TAGGKKIPISTLIRIGSYGTGSSLPPLTDSSPYNDKTKTEPVYVPCFSN | 162 | 0.894 |
| | | G07 | | QAETRGTILNCFSNPSLSSIQPDFLQMIPLYQPQSLNISESSNPVLTQE | | 33264 |
| | | 680 | | QSVLQAMMENNRRQNFKTLSISQETGVSNTDNSSVFEFGRKRFDHQEVP | | 5 |
| | | | | SPSSGPVDLEPEWNY | | |
| 229 | 26 | AT2 | AP2- | KLAGELPRPVTNSPKDIQAAASLAAVNWQDSVNDVSNSEVAEIVEAEPS | 139 | 0.901 |
| | | G44 | EREBP | RAVVAQLFSSDTSTTTTTQSQEYSEASCASTSACTDKDSEEEKLEDLPD | | 71951 |
| | | 940 | | LFTDENEMMIRNDAFCYYSSTWQLCGADAGFRLEEPFFLSE | | 3 |
| 230 | 519 | AT3 | bZIP | MQPQTDVFSLHNYLNSSILQSPYPSNFPISTPFPTNGQNPYLLYGFQSP | 78 | 0.908 |
| | | G30 | | TNNPQSMSLSSNNSTSDEAEEQQTNNNII | | 60944 |
| | | 530 | | | | 4 |
| 231 | 405 | AT1 | RWP- | MADHTTKEQKSFSFLAHSPSFDHSSLSYPLFDWEEDLLALQENSGSQAF | 120 | 0.928 |
| | | G74 | RK | PFTTTSLPLPDLEPLSEDVLNSYSSASWNETEQNRGDGASSEKKRENGT | | 88359 |
| | | 480 | | VKETTKKRKINERHREHSVRII | | 7 |
| 232 | 447 | AT4 | WRKY | MNPQANDRKEFQGDCSATGDLTAKHDSAGGNGGGGARYKLMSPAKLPIS | 132 | 0.933 |
| | | G26 | | RSTDITIPPGLSPTSFLESPVFISNIKPEPSPTTGSLFKPRPVHISASS | | 73290 |
| | | 640 | | SSYTGRGFHQNTFTEQKSSEFEFRPPASNMVYAE | | 4 |
| 233 | 381 | AT4 | NAC | GEAAEISYEPSPSLVSDSHTVIAITGEPEPELQVEQPGKENLLGMSVDD | 319 | 0.936 |
| | | G01 | | LIEPMNQQEEPQGPHLAPNDDEFIRGLRHVDRGTVEYLFANEENMDGLS | | 69461 |
| | | 540 | | MNDLRIPMIVQQEDLSEWEGFNADTFFSDNNNNYNLNVHHQLTPYGDGY | | 1 |
| | | | | LNAFSGYNEGNPPDHELVMQENRNDHMPRKPVTGTIDYSSDSGSDAGSI | | |
| | | | | STTSYQGTSSPNISVGSSSRHLSSCSSTDSCKDLQTCTDPSIISREIRE | | |
| | | | | LTQEVKQEIPRAVDAPMNNESSLVKTEKKGLFIVEDAMERNRKKPRFIY | | |
| | | | | LMKMIIGNIISVLLPVKRLIPVKKL | | |
| 234 | 62 | AT5 | AP2- | MATPNEVSALWFIEKHLLDEASPVATDPWMKHESSSATESSSDSSSIIF | 154 | 0.949 |
| | | G47 | EREBP | GSSSSSFAPIDFSESVCKPEIIDLDTPRSMEFLSIPFEFDSEVSVSDED | | 41430 |
| | | 230 | | FKPSNQNQNQFEPELKSQIRKPPLKISLPAKTEWIQFAAENTKPEVTKP | | 7 |
| | | | | VSEEEKK | | |
| 235 | 487 | AT4 | bHLH | MYPSLDDDFVSDLFCFDQSNGAELDDYTQFGVNLQTDQEDTFPDFVSYG | 120 | 0.952 |
| | | G14 | | VNLQQEPDEVESIGASQLDLSSYNGVLSLEPEQVGQQDCEVVQEEEVEI | | 23581 |
| | | 410 | | NSGSSGGAVKEEQEHLDDDCSR | | 6 |
| 236 | 379 | AT2 | NAC | KNLHKTLNSPVGGASLSGGGDTPKTTSSQIFNEDTLDQFLELMGRSCKE | 185 | 0.957 |
| | | G46 | | ELNLDPFMKLPNLESPNSQAINNCHVSSPDTNHNIHVSNVVDTSFVTSW | | 43961 |
| | | 770 | | AALDRLVASQLNGPTSYSITAVNESHVGHDHLALPSVRSPYPSLNRSAS | | 4 |
| | | | | YHAGLTQEYTPEMELWNTTTSSLSSSPGPFCHVSNGSG | | |
| 237 | 456 | AT2 | WRKY | MAEKEEKEPSKLKSSTGVSRPTISLPPRPFGEMFFSGGVGFSPGPMTLV | 243 | 0.967 |
| | | G03 | | SNLFSDPDEFKSFSQLLAGAMASPAAAAVAAAAVVATAHHQTPVSSVGD | | 82847 |
| | | 340 | | GGGSGGDVDPRFKQSRPTGLMITQPPGMFTVPPGLSPATLLDSPSFFGL | | 2 |
| | | | | FSPLQGTFGMTHQQALAQVTAQAVQGNNVHMQQSQQSEYPSSTQQQQQQ | | |
| | | | | QQQASLTEIPSFSSAPRSQIRASVQETSQGQRETSEISVFEHRSQPQ | | |
| 238 | 51 | AT5 | AP2- | LDVRVTSETCSGEGVIGLGKRKRDKGSPPEEEKAARVKVEEEESNTSET | 96 | 1.006 |
| | | G61 | EREBP | TEAEVEPVVPLTPSSWMGFWDVGAGDGIFSIPPLSPTSPNFSVISVT | | 56422 |
| | | 600 | | | | 6 |
| 239 | 401 | AT1 | RAV | DVKMDEDEVDFLNSHSKSEIVDMLRKHTYNEELEQSKRRRNGNGNMTRT | 71 | 1.038 |
| | | G13 | | LLTSGLSNDGVSTTGFRSAEAL | | 29378 |
| | | 260 | | | | |
| 240 | 483 | AT1 | bHLH | EKVQKYEGSYPGWSQEPTKLTPWRNNHWRVQSLGNHPVAINNGSGPGIP | 215 | 1.039 |
| | | G69 | | FPGKFEDNTVTSTPAIIAEPQIPIESDKARAITGISIESQPELDDKGLP | | 11518 |
| | | 010 | | PLQPILPMVQGEQANECPATSDGLGQSNDLVIEGGTISISSAYSHELLS | | 9 |
| | | | | SLTQALQNAGIDLSQAKLSVQIDLGKRANQGLTHEEPSSKNPLSYDTQG | | |
| | | | | RDSSVEEESEHSHKRMKTL | | |
| 241 | 411 | AT3 | SBP | QPTTALFTSHYSRIAPSLYGNPNAAMIKSVLGDPTAWSTARSVMQRPGP | 221 | 1.047 |
| | | G57 | | WQINPVRETHPHMNVLSHGSSSFTTCPEMINNNSTDSSCALSLLSNSYP | | 49545 |
| | | 920 | | IHQQQLQTPTNTWRPSSGFDSMISFSDKVTMAQPPPISTHQPPISTHQQ | | 5 |
| | | | | YLSQTWEVIAGEKSNSHYMSPVSQISEPADFQISNGTTMGGFELYLHQQ | | |
| | | | | VLKQYMEPENTRAYDSSPQHFNWSL | | |
| 242 | 31 | AT5 | AP2- | HPQQQQQVVVNRNLSFSGHGSGSWAYNKKLDMVHGLDLGLGQASCSRGS | 217 | 1.055 |
| | | G18 | EREBP | CSERSSFLQEDDDHSHNRCSSSSGSNLCWLLPKQSDSQDQETVNATTSY | | 12610 |
| | | 450 | | GGEGGGGSTLTFSTNLKPKNLMSQNYGLYNGAWSRFLVGQEKKTEHDVS | | 7 |
| | | | | SSCGSSDNKESMLVPSCGGERMHRPELEERTGYLEMDDLLEIDDLGLLI | | |
| | | | | GKNGDFKNWCCEEFQHPWNWF | | |
| 243 | 106 | AT5 | C2C2- | TKNSSGGGGGSTSSGNSKSQDSATSNDQYHHRAMANNQMGPPSSSSSLS | 250 | 1.055 |
| | | G02 | DOF | SLLSSYNAGLIPGHDHNSNNNNILGLGSSLPPLKLMPPLDFTDNFTLQY | | 20456 |
| | | 460 | | GAVSAPSYHIGGGSSGGAAALLNGFDQWRFPATNQLPLGGLDPFDQQHQ | | 8 |
| | | | | MEQQNPGYGLVTGSGQYRPKNIFHNLISSSSSASSAMVTATASQLASVK | | |
| | | | | MEDSNNQLNLSRQLFGDEQQLWNIHGAAAASTAAATSSWSEVSNNESSS | | |
| | | | | STSNI | | |
| 244 | 341 | AT1 | NAC | GERREFSVATGSGIKHTHSLIPPTNNSGVLSVETEGSLFHSQESQNPSQ | 211 | 1.060 |
| | | G02 | | FSGFLDVDALDRDFCNILSDDFKGFENDDDEQSKIVSMQDDRNNHTPQK | | 15846 |
| | | 250 | | PLTGVFSDHSTDGSDSDPISATTISIQTLSTCPSFGSSNPLYQITDLQE | | 5 |
| | | | | SPNSIKLVSLAQEVSKTPGTGIDNDAQGTEIGEHKLGQETIKNKRAGFF | | |
| | | | | HRMIQKFVKKIHLRT | | |
| 245 | 232 | AT2 | Homeo | QLETEYNILRQNYDNLASQFESLKKEKQALVSELQRLKEATQKKTQEEE | 171 | 1.063 |
| | | G46 | box | RQCSGDQAVVALSSTHHESENEENRRRKPEEVRPEMEMKDDKGHHGVMC | | 37692 |
| | | 680 | | DHHDYEDDDNGYSNNIKREYFGGFEEEPDHLMNIVEPADSCLTSSDDWR | | 4 |
| | | | | GFKSDTTTLLDQSSNNYPWRDFWS | | |
| 246 | 293 | AT3 | MYB | KVSSENMMNHQHHCSGNSQSSGMTTQGSSGKAIDTAESFSQAKTTTENV | 77 | 1.063 |
| | | G01 | | VEQQSNENYWNVEDLWPVHLLNGDHHVI | | 66400 |
| | | 530 | | | | 9 |
| 247 | 473 | AT1 | WRKY | STLRGTVAAEHLLVHRGGGGSLLHSFPRHHQDFLMMKHSPANYQSVGSL | 87 | 1.065 |
| | | G29 | | SYEHGHGTSSYNFNNNQPVVDYGLLQDIVPSMFSKNES | | 03370 |
| | | 860 | | | | 3 |
| 248 | 102 | AT1 | C2C2- | KRHRSFSTTATSSSSSSSVITTTTQEPATTEASQTKVINLISGHGSFAS | 126 | 1.081 |
| | | G47 | DOF | LLGLGSGNGGLDYGFGYGYGLEEMSIGYLGDSSVGEIPVVDGCGGDTWQ | | 48125 |
| | | 655 | | IGEIEGKSGGDSLIWPGLEISMQTNDVK | | 3 |
| 249 | 311 | AT5 | MYB | KKINESGEEDNDGVSSSNTSSQKNHQSTNKGQWERRLQTDINMAKQALC | 236 | 1.082 |
| | | G62 | | EALSLDKPSSTLSSSSSLPTPVITQQNIRNFSSALLDRCYDPSSSSSST | | 14828 |
| | | 470 | | TTTTTSNTTNPYPSGVYASSAENIARLLQDEMKDTPKALTLSSSSPVSE | | |
| | | | | TGPLTAAVSEEGGEGFEQSFFSENSMDETQNLTQETSFFHDQVIKPEIT | | |
| | | | | MDQDHGLISQGSLSLFEKWLFDEQSHEMVGMALAGQEGMF | | |
| 250 | 427 | AT | TCP | AQLPPWNPADTLRQHAAAAANAKPRKTKTLISPPPPQPEETEHHRIGEE | 284 | 1.083 |
| | | G53 | | EDNESSFLPASMDSDSIADTIKSFFPVASTQQSYHHQPPSRGNTQNQDL | | 05605 |
| | | 230 | | LRLSLQSFQNGPPFPNQTEPALFSGQSNNQLAFDSSTASWEQSHQSPEF | | 1 |
| | | | | GKIQRLVSWNNVGAAESAGSTGGFVFASPSSLHPVYSQSQLLSQRGPLQ | | |
| | | | | SINTPMIRAWFDPHHHHHHHQQSMTTDDLHHHHPYHIPPGIHQSAIPGI | | |
| | | | | AFASSGEFSGFRIPARFQGEQEEHGGDNKPSSASSDSRH | | |
| 251 | 47 | AT5 | AP2- | RSDASEVTSTSSQSEVCTVETPGCVHVKTEDPDCESKPFSGGVEPMYCL | 200 | 1.083 |
| | | G05 | EREBP | ENGAEEMKRGVKADKHWLSEFEHNYWSDILKEKEKQKEQGIVETCQQQQ | | 10917 |
| | | 410 | | QDSLSVADYGWPNDVDQSHLDSSDMFDVDELLRDLNGDDVFAGLNQDRY | | 9 |
| | | | | PGNSVANGSYRPESQQSGFDPLQSLNYGIPPFQLEGKDGNGFFDDLSYL | | |
| | | | | DLEN | | |
| 252 | 470 | AT1 | WRKY | LTSSTRNGPKPKPEPKPEPEPEVEPEAEEEDNKFMVLGRGIETTPSCVD | 125 | 1.085 |
| | | G29 | | EFAWFTEMETTSSTILESPIFSSEKKTAVSGADDVAVFFPMGEEDESLF | | 79017 |
| | | 280 | | ADLGELPECSVVFRHRSSVVGSQVEIF | | 5 |
| 253 | 495 | AT2 | bHLH | MLEGLVSQESLSLNSMDMSVLERLKWVQQQQQQLQQVVSHSSNNSPELL | 241 | 1.100 |
| | | G18 | | QILQFHGSNNDELLESSFSQFQMLGSGFGPNYNMGFGPPHESISRTSSC | | 55657 |
| | | 300 | | HMEPVDTMEVLLKTGEETRAVALKNKRKPEVKTREEQKTEKKIKVEAET | | 7 |
| | | | | ESSMKGKSNMGNTEASSDTSKETSKGASENQKLDYIHVRARRGQATDRH | | |
| | | | | SLAERARREKISKKMKYLQDIVPGCNKVTGKAGMLDEIINYVQCL | | |
| 254 | 289 | AT1 | MYB | IKKGIDPVTHKGITSGTDKSENLPEKQNVNLTTSDHDLDNDKAKKNNKN | 235 | 1.110 |
| | | G18 | | FGLSSASFLNKVANRFGKRINQSVLSEIIGSGGPLASTSHTTNTTTTSV | | 45093 |
| | | 570 | | SVDSESVKSTSSSFAPTSNLLCHGTVATTPVSSNFDVDGNVNLTCSSST | | 7 |
| | | | | FSDSSVNNPLMYCDNFVGNNNVDDEDTIGFSTFLNDEDFMMLEESCVEN | | |
| | | | | TAFMKELTRFLHEDENDVVDVTPVYERQDLFDEIDNYFG | | |
| 255 | 384 | AT4 | NAC | TQPRQCGGSVAAAATAKDRPYLHGLGGGGGRHLHYHLHHNNGNGKSNGS | 86 | 1.129 |
| | | G28 | | GGTAGAGEYYHNIPAIISFNQTGIQNHLVHDSQPFIP | | 73433 |
| | | 500 | | | | |
| 256 | 389 | AT1 | NAC | RLAAVRRMGDYDSSPSHWYDDQLSFMASELETNGQRRILPNHHQQQQHE | 239 | 1.139 |
| | | G12 | | HQQHMPYGLNASAYALNNPNLQCKQELELHYNHLVQRNHLLDESHLSFL | | 56011 |
| | | 260 | | QLPQLESPKIQQDNSNCNSLPYGTSNIDNNSSHNANLQQSNIAHEEQLN | | |
| | | | | QGNQNFSSLYMNSGNEQVMDQVTDWRVLDKFVASQLSNEEAATASASIQ | | |
| | | | | NNAKDTSNAEYQVDEEKDPKRASDMGEEYTASTSSSCQIDLWK | | |
| 257 | 349 | AT2 | NAC | TEATKKYISTSSSSTSHHHNNHTRASILSTNNNNPNYSSDLLQLPPHLQ | 151 | 1.140 |
| | | G24 | | PHPSLNINQSLMANAVHLAELSRVFRASTSTTMDSSHQQLMNYTHMPVS | | 98705 |
| | | 430 | | GLNLNLGGALVQPPPVVSLEDVAAVSASYNGENGFGNVEMSQCMDLDGY | | 5 |
| | | | | WPSY | | |
| 258 | 10 | AT1 | AP2- | EEIEDLPRPSTCTPRDIQVAAAKAANAVKIIKMGDDDVAGIDDGDDFWE | 91 | 1.148 |
| | | G01 | EREBP | GIELPELMMSGGGWSPEPFVAGDDATWLVDGDLYQYQFMACL | | 34264 |
| | | 250 | | | | 7 |
| 259 | 367 | AT5 | NAC | TNAVSSQRSIPQSWVYPTIPDNNQQSHNNTATLLASSDVLSHISTRQNF | 146 | 1.148 |
| | | G39 | | IPSPVNEPASFTESAASYFASQMLGVTYNTARNNGTGDALFLRNNGTGD | | 64989 |
| | | 820 | | ALVLSNNENNYENNLTGGLTHEVPNVRSMVMEETTGSEMSATSYSTNN | | 7 |
| 260 | 489 | AT2 | bHLH | MDSNNHLYDPNPTGSGLLRFRSAPSSVLAAFVDDDKIGFDSDRLLSRFV | 27 | 1.151 |
| | | G42 | | TSNGVNGDLGSPKFEDKSPVSLTNTSVSYAATLPPPPQLEPSSFLGLPP | | 58320 |
| | | 280 | | HYPRQSKGIMNSVGLDQFLGINNHHTKPVESNLLRQSSSPAGMFTNLSD | | 2 |
| | | | | QNGYGSMRNLMNYEEDEESPSNSNGLRRHCSLSSRPPSSLGMLSQIPEI | | |
| | | | | APETNFPYSHWNDPSSFIDNLSSLKREAEDDGKLFLGAQNGESGNRMQL | | |
| | | | | LSHHLSLPKSSSTASDMVSVDKYLQLQDSVPCKI | | |
| 261 | 210 | AT4 | HB | RLEEEYNKLKNSHDNVVVDKCRLESEVIQLKEQLYDAEREIQRLAERVE | 106 | 1.206 |
| | | G36 | | GGSSNSPISSSVSVEANETPFFGDYKVGDDGDDYDHLFYPVPENSYIDE | | 22627 |
| | | 740 | | AEWMSLYI | | 6 |
| 262 | 18 | AT3 | AP2- | ELSGLLPRPVSCSPKDIQAAATKAAEATTWHKPVIDKKLADELSHSELL | 129 | 1.207 |
| | | G60 | EREBP | STAQSSTSSSFVFSSDTSETSSTDKESNEETVFDLPDLFTDGLMNPNDA | | 48542 |
| | | 490 | | FCLCNGTFTWQLYGEEDVGFRFEEPENWQND | | 6 |
| 263 | 49 | AT3 | AP2-P | AERVQESLSEIKYTYEDGCSPVVALKRKHSMRRRMTNKKTKDSDEDHRS | 79 | 1.213 |
| | | G23 | EREB | VKLDNVVVFEDLGEQYLEELLGSSENSGTW | | 76046 |
| | | 240 | | | | 6 |
| 264 | 503 | AT2 | bZIP | MGNSSEEPKPPTKSDKPSSPPVDQTNVHVYPDWAAMQAYYGPRVAMPPY | 258 | 1.215 |
| | | G46 | | YNSAMAASGHPPPPYMWNPQHMMSPYGAPYAAVYPHGGGVYAHPGIPMG | | 19700 |
| | | 270 | | SLPQGQKDPPLTTPGTLLSIDTPTKSTGNTDNGLMKKLKEFDGLAMSLG | | 6 |
| | | | | NGNPENGADEHKRSRNSSETDGSTDGSDGNTTGADEPKLKRSREGTPTK | | |
| | | | | DGKQLVQASSFHSVSPSSGDTGVKLIQGSGAILSPGVSANSNPFMSQSL | | |
| | | | | AMVPPETWLQNER | | |
| 265 | 28 | AT4 | AP2- | MDFDEELNLCITKGKNVDHSFGGEASSTSPRSMKKMKSPSRPKPYFQSS | 141 | 1.222 |
| | | G28 | EREBP | SSPYSLEAFPFSLDPTLQNQQQQLGSYVPVLEQRQDPTMQGQKQMISES | | 01246 |
| | | 140 | | PQQQQQQQQYMAQYWSDTLNLSPRGRMMMMMSQEAVQPYIATK | | 1 |
| 266 | 457 | AT5 | WRKY | CSQAANVGTTMPIQNLEPNQTQEHGNLDMVKESVDNYNHQAHLHHNLHY | 132 | 1.241 |
| | | G24 | | PLSSTPNLENNNAYMLQMRDQNIEYFGSTSFSSDLGTSINYNFPASGSA | | 33233 |
| | | 110 | | SHSASNSPSTVPLESPFESYDPNHPYGGFGGFYS | | 6 |
| 267 | 372 | AT1 | NAC | KGATERRGPPPPVVYGDEIMEEKPKVTEMVMPPPPQQTSEFAYEDTSDS | 131 | 1.274 |
| | | G01 | | VPKLHTTDSSCSEQVVSPEFTSEVQSEPKWKDWSAVSNDNNNTLDFGEN | | 43378 |
| | | 720 | | YIDATVDNAFGGGGSSNQMFPLQDMFMYMQKPY | | 4 |
| 268 | 104 | AT2 | C2C2- | GKSGNSKSSSSSQNKQSTSMVNATSPTNTSNVQLQTNSQFPFLPTLQNL | 192 | 1.283 |
| | | G28 | DOF | TQLGGIGLNLAAINGNNGGNGNTSSSFLNDLGFFHGGNTSGPVMGNNNE | | 54370 |
| | | 810 | | NNLMTSLGSSSHFALFDRTMGLYNFPNEVNMGLSSIGATRVSQTAQVKM | | 7 |
| | | | | EDNHLGNISRPVSGLTSPGNQSNQYWTGQGLPGSSSNDHHHQHLM | | |
| 269 | 275 | AT5 | MYB | GLGDHSTAVKAACGVESPPSMALITTTSSSHQEISGGKNSTLRFDTLVD | 103 | 1.294 |
| | | G40 | | ESKLKPKSKLVHATPTDVEVAATVPNLFDTFWVLEDDFELSSLTMMDET | | 39435 |
| | | 330 | | NGYCL | | |
| 270 | 518 | AT5 | bZIP | MQPNYDSSSLNNMQQQDYFNLNNYYNNLNPSTNNNNLNILQYPQIQELN | 71 | 1.305 |
| | | G15 | | LQSPVSNNSTTSDDATEEIFVI | | 94914 |
| | | 830 | | | | 6 |
| 271 | 75 | AT5 | AP2- | NHFPNNSQLSLKIRNLLHQKQSMKQQQQQQHKPVSSLTDCNINYISTAT | 175 | 1.322 |
| | | G19 | EREBP | SLTTTTTTTTTTAIPLNNVYRPDSSVIGQPETEGLQLPYSWPLVSGENH | | 24508 |
| | | 790 | | QIPLAQAGGETHGHLNDHYSTDQHLGLAEIERQISASLYAMNGANSYYD | | 8 |
| | | | | NMNAEYAIFDPTDPIWDLPSLSQLFCPT | | |
| 272 | 167 | AT5 | C3H | ELRPLYPSTGSGVPSPRSSFSSCNSSTAFDMGPISPLPIGATTTPPLSP | 299 | 1.325 |
| | | G58 | | NGVSSPIGGGKTWMNWPNITPPALQLPGSRLKSALNAREIDFSEEMQSL | | 60745 |
| | | 620 | | TSPTTWNNTPMSSPFSGKGMNRLAGGAMSPVNSLSDMFGTEDNTSGLQI | | 4 |
| | | | | RRSVINPQLHSNSLSSSPVGANSLFSMDSSAVLASRAAEFAKQRSQSFI | | |
| | | | | ERNNGLNHHPAISSMTTTCLNDWGSLDGKLDWSVQGDELQKLRKSTSFR | | |
| | | | | LRAGGMESRLPNEGTGLEEPDVSWVEPLVKEPQETRLAPVWMEQSYMET | | |
| | | | | EQTVA | | |
| 273 | 357 | AT3 | NAC | NGICSELESERQLQTGQCSFTTASMEEINSNNNNNYNNDYETMSPEVGV | 90 | 1.346 |
| | | G17 | | SSACVEEVVDDKDDSWMQFITDDAWDTSSNGAAMGHGQGVY | | 02704 |
| | | 730 | | | | |
| 274 | 417 | AT1 | TCP | TGTGTIPANFSTLNASLRSGGGSTLFSQASKSSSSPLSFHSTGMSLYED | 258 | 1.352 |
| | | G72 | | NNGTNGSSVDPSRKLLNSAANAAVFGFHHQMYPPIMSTERNPNTLVKPY | | 66906 |
| | | 010 | | REDYFKEPSSAAEPSESSQKASQFQEQELAQGRGTANVVPQPMWAVAPG | | 4 |
| | | | | TTNGGSAFWMLPMSGSGGREQMQQQPGHQMWAFNPGNYPVGTGRVVTAP | | |
| | | | | MGSMMLGGQQLGLGVAEGNMAAAMRGSRGDGLAMTLDQHQHQLQHQEPN | | |
| | | | | QSQASENGGDDKK | | |
| 275 | 362 | AT4 | NAC | TQPRQCNWSSSTSSLNAIGGGGGEASSGGGGGEYHMRRDSGTTSGGSCS | 283 | 1.368 |
| | | G29 | | SSREIINVNPPNRSDEIGGVGGGVMAVAAAAAAVAAGLPSYAMDQLSFV | | 22000 |
| | | 230 | | PFMKSFDEVARRETPQTGHATCEDVMAEQHRHRHQPSSSTSHHMAHDHH | | 3 |
| | | | | HHHHQQQQQRHHAFNISQPTHPISTIISPSTSLHHASINILDDNPYHVH | | |
| | | | | RILLPNENYQTQQQLRQEGEEEHNDGKMGGRSASGLEELIMGCTSSTTH | | |
| | | | | HDVKDGSSSMGNQQEAEWLKYSTFWPAPDSSDNQDHHG | | |
| 276 | 478 | AT5 | ZF-HD | QPPPPPPGFYRLPAPVSYRPPPSQAPPLQLALPPPQRERSEDPMETSSA | 144 | 1.385 |
| | | G65 | | EAGGGIRKRHRTKFTAEQKERMLALAERIGWRIQRQDDEVIQRFCQETG | | 27983 |
| | | 410 | | VPRQVLKVWLHNNKHTLGKSPSPLHHHQAPPPPPPQSSFHHEQDQP | | 2 |
| 277 | 449 | AT4 | WRKY | MADDWDLHAVVRGCSAVSSSATTTVYSPGVSSHTNPIFTVGRQSNAVSF | 127 | 1.385 |
| | | G01 | | GEIRDLYTPFTQESVVSSFSCINYPEEPRKPQNQKRPLSLSASSGSVTS | | 53073 |
| | | 250 | | KPSGSNTSRSKRRKIQHKKVCHVAAEALN | | 2 |
| 278 | 356 | AT3 | NAC | QTSAQKQAYNNLMTSGREYSNNGSSTSSSSHQYDDVLESLHEIDNRSLG | 155 | 1.389 |
| | | G15 | | FAAGSSNALPHSHRPVLTNHKTGFQGLAREPSFDWANLIGQNSVPELGL | | 34523 |
| | | 500 | | SHNVPSIRYGDGGTQQQTEGIPRENNNSDVSANQGFSVDPVNGFGYSGQ | | 3 |
| | | | | QSSGFGFI | | |
| 279 | 529 | AT3 | zf- | MKKITIPVESLDEEDDELLQLAAIEAEAAAKRPRVSSIPEGPYMAALKG | 238 | 1.390 |
| | | G42 | GRF | SKSDQWQQSPLNPASKSRSVAVTTGGFQRSDGGGGVAGEQDFPEKSCPC | | 97923 |
| | | 860 | | GVGICLILTSNTPKNPGRKFYKCPNREENGGCGFFQWCDAVQSSGTSTT | | 5 |
| | | | | TSNSYGNGNDTKFPDHQCPCGAGLCRVLTAKTGENVGRQFYRCPVFEGS | | |
| | | | | CGFFKWCNDNVVSSPTSYSVTKNSNFGDSDTRGYQNAKTGTP | | |
| 280 | 57 | AT5 | AP2- | MYGQCNIESDYALLESITRHLLGGGGENELRLNESTPSSCFTESWGGLP | 115 | 1.391 |
| | | G47 | EREBP | LKENDSEDMLVYGLLKDAFHFDTSSSDLSCLFDFPAVKVEPTENFTAME | | 44746 |
| | | 220 | | EKPKKAIPVTETAVKAK | | 6 |
| 281 | 509 | AT1 | bZIP | VRARQQGLCVRNSSDTSYLGPAGNMNSGIAAFEMEYTHWLEEQNRRVSE | 246 | 1.399 |
| | | G22 | | IRTALQAHIGDIELKMLVDSCLNHYANLFRMKADAAKADVFFLMSGMWR | | 05821 |
| | | 070 | | TSTERFFQWIGGFRPSELLNVVMPYVEPLTDQQLLEVRNLQQSSQQAEE | | 6 |
| | | | | ALSQGLDKLQQGLVESIAIQIKVVESVNHGAPMASAMENLQALESFVNQ | | |
| | | | | ADHLRQQTLQQMSKILTTRQAARGLLALGEYFHRLRALSSLWAARPREH | | |
| | | | | T | | |
| 282 | 265 | AT3 | MYB | VKRSISSSSSDVTNHSVSSTSSSSSSISSVLQDVIIKSERPNQEEEFGE | 121 | 1.402 |
| | | G12 | | ILVEQMACGFEVDAPQSLECLFDDSQVPPPISKPDSLQTHGKSSDHEFW | | 57391 |
| | | 820 | | SRLIEPGEDDYNEWLIFLDNQTC | | 7 |
| 283 | 425 | AT3 | TCP | TGSGTIPASALASSAATSNHHQGGSLTAGLMISHDLDGGSSSSGRPLNW | 182 | 1.436 |
| | | G27 | | GIGGGEGVSRSSLPTGLWPNVAGFGSGVPTTGLMSEGAGYRIGFPGFDF | | 79445 |
| | | 010 | | PGVGHMSFASILGGNHNQMPGLELGLSQEGNVGVLNPQSFTQIYQQMGQ | | 3 |
| | | | | AQAQAQGRVLHHMHHNHEEHQQESGEKDDSQGSGR | | |
| 284 | 506 | AT5 | bZIP | DRARQQGFYVGNGIDTNSLGFSETMNPGIAAFEMEYGHWVEEQNRQICE | 244 | 1.438 |
| | | G65 | | LRTVLHGHINDIELRSLVENAMKHYFELFRMKSSAAKADVFFVMSGMWR | | 51669 |
| | | 210 | | TSAERFFLWIGGFRPSDLLKVLLPHFDVLTDQQLLDVCNLKQSCQQAED | | 3 |
| | | | | ALTQGMEKLQHTLADCVAAGQLGEGSYIPQVNSAMDRLEALVSFVNQAD | | |
| | | | | HLRHETLQQMYRILTTRQAARGLLALGEYFQRLRALSSSWATRHREPT | | |
| 285 | 366 | AT5 | NAC | RADGTKVPMSMLDPHINRMEPAGLPSLMDCSQRDSFTGSSSHVTCFSDQ | 115 | 1.452 |
| | | G39 | | ETEDKRLVHESKDGFGSLFYSDPLFLQDNYSLMKLLLDGQETQFSGKPF | | 57915 |
| | | 610 | | DGRDSSGTEELDCVWNF | | 4 |
| 286 | 493 | AT1 | bHLH | MDPSGMMNEGGPFNLAEIWQFPLNGVSTAGDSSRRSFVGPNQFGDADLT | 147 | 1.460 |
| | | G59 | | TAANGDPARMSHALSQAVIEGISGAWKRREDESKSAKIVSTIGASEGEN | | 98317 |
| | | 640 | | KRQKIDEVCDGKAEAESLGTETEQKKQQMEPTKDYIHVRARRGQATDSH | | |
| 287 | 77 | AT1 | AP2- | ENVGTQTIQRNSHFLQNSMQPSLTYIDQCPTLLSYSRCMEQQQPLVGML | 75 | 1.461 |
| | | G43 | EREBP | QPTEEENHFFEKPWTEYDQYNYSSFG | | 45341 |
| | | 160 | | | | 9 |
| 288 | 463 | AT3 | WRKY | MEDRRCDVLFPCSSSVDPRLTEFHGVDNSAQPTTSSEEKPRSKKKKKER | 147 | 1.474 |
| | | G01 | | EARYAFQTRSQVDILDDGYRWRKYGQKAVKNNPFPRSYYKCTEEGCRVK | | 83771 |
| | | 970 | | KQVQRQWGDEGVVVTTYQGVHTHAVDKPSDNFHHILTQMHIFPPFCLKE | | 6 |
| 289 | 467 | AT2 | WRKY | MYSYKKISYQMEEVMSMIFHGMKLVKSLESSLPEKPPESLLTSLDEIVK | 172 | 1.492 |
| | | G40 | | TFSDANERLKMLLEIKNSETALNKTKPVIVSVANQMLMQMEPGLMQEYW | | 82154 |
| | | 740 | | LRYGGSTSSQGTEAMFQTQLMAVDGGGERNLTAAVERSGASGSSTPRQR | | 7 |
| | | | | RRKDEGEEQTVLVAALRTGNTDLPP | | |
| 290 | 501 | AT2 | bZIP | MVTRETKLTSEREVESSMAQARHNGGGGGENHPFTSLGRQSSIYSLTLD | 354 | 1.503 |
| | | G36 | | EFQHALCENGKNFGSMNMDEFLVSIWNAEENNNNQQQAAAAAGSHSVPA | | 76783 |
| | | 270 | | NHNGFNNNNNNGGEGGVGVFSGGSRGNEDANNKRGIANESSLPRQGSLT | | 6 |
| | | | | LPAPLCRKTVDEVWSEIHRGGGSGNGGDSNGRSSSSNGQNNAQNGGETA | | |
| | | | | ARQPTFGEMTLEDFLVKAGVVREHPTNPKPNPNPNQNQNPSSVIPAAAQ | | |
| | | | | QQLYGVFQGTGDPSFPGQAMGVGDPSGYAKRTGGGGYQQAPPVQAGVCY | | |
| | | | | GGGVGFGAGGQQMGMVGPLSPVSSDGLGHGQVDNIGGQYGVDMGGLRGR | | |
| | | | | KRVVDGPVEKV | | |
| 291 | 42 | AT1 | AP2- | DSAWRLPVPASTDPDTIRRTAAEAAEMFRPPEFSTGITVLPSASEFDTS | 95 | 1.518 |
| | | G63 | EREBP | DEGVAGMMMRLAEEPLMSPPRSYIDMNTSVYVDEEMCYEDLSLWSY | | 14925 |
| | | 030 | | | | 9 |
| 292 | 276 | AT3 | MYB | EAQNYGKLFEWRGNTGEELLHKYKETEITRTKTTSQEHGFVEVVSMESG | 125 | 1.523 |
| | | G53 | | KEANGGVGGRESFGVMKSPYENRISDWISEISTDQSEANLSEDHSSNSC | | 95718 |
| | | 200 | | SENNINIGTWWFQETRDFEEFSCSLWS | | 5 |
| 293 | 8 | AT5 | AP2- | MCVLKVANQEDNVGKKAESIRDDDHRTLSEIDQWLYLFAAEDDHHRHSF | 183 | 1.527 |
| | | G64 | EREBP | PTQQPPPSSSSSSLISGFSREMEMSAIVSALTHVVAGNVPQHQQGGGEG | | 90621 |
| | | 750 | | SGEGTSNSSSSSGQKRRREVEEGGAKAVKAANTLTVDQYFSGGSSTSKV | | 1 |
| | | | | REASSNMSGPGPTYEYTTTATASSETSSFSGDQPRR | | |
| 294 | 414 | AT2 | SBP | QPASLSVLASRYGRIAPSLYENGDAGMNGSFLGNQEIGWPSSRTLDTRV | 227 | 1.536 |
| | | G42 | | MRRPVSSPSWQINPMNVFSQGSVGGGGTSFSSPEIMDTKLESYKGIGDS | | 75012 |
| | | 200 | | NCALSLLSNPHQPHDNNNNNNNNNNNNNNTWRASSGFGPMTVTMAQPPP | | 7 |
| | | | | APSQHQYLNPPWVFKDNDNDMSPVLNLGRYTEPDNCQISSGTAMGEFEL | | |
| | | | | SDHHHQSRRQYMEDENTRAYDSSSHHTNWSL | | |
| 295 | 118 | AT5 | C2C2- | SKTKQVPSSSSADKPTTTQDDHHVEEKSSTGSHSSSESSSLTASNSTTV | 202 | 1.543 |
| | | G60 | DOF | AAVSVTAAAEVASSVIPGFDMPNMKIYGNGIEWSTLLGQGSSAGGVFSE | | 10757 |
| | | 850 | | IGGFPAVSAIETTPFGFGGKFVNQDDHLKLEGETVQQQQFGDRTAQVEF | | 3 |
| | | | | QGRSSDPNMGFEPLDWGSGGGDQTLFDLTSTVDHAYWSQSQWTSSDQDQ | | |
| | | | | SGLYLP | | |
| 296 | 419 | AT5 | TCP | TGTGTTPASFSTASLSTSSPFTLGKRVVRAEEGESGGGGGGGLTVGHTM | 154 | 1.556 |
| | | G08 | | GTSLMGGGGSGGFWAVPARPDFGQVWSFATGAPPEMVFAQQQQPATLFV | | 37792 |
| | | 330 | | RHQQQQQASAAAAAAMGEASAARVGNYLPGHHLNLLASLSGGANGSGRR | | 7 |
| | | | | EDDHEPR | | |
| 297 | 526 | AT1 | bZIP | MGSSEMEKSGKEKEPKTTPPSTSSSAPATVVSQEPSSAVSAGVAVTQDW | 294 | 1.583 |
| | | G32 | | SGFQAYSPMPPHGYVASSPQPHPYMWGVQHMMPPYGTPPHPYVTMYPPG | | 21464 |
| | | 150 | | GMYAHPSLPPGSYPYSPYAMPSPNGMAEASGNTGSVIEGDGKPSDGKEK | | 4 |
| | | | | LPIKRSKGSLGSLNMIIGKNNEAGKNSGASANGACSKSAESGSDGSSDG | | |
| | | | | SDANSQNDSGSRHNGKDGETASESGGSAHGPPRNGSNLPVNQTVAIMPV | | |
| | | | | SATGVPGPPTNLNIGMDYWSGHGNVSGAVPGVVVDGSQSQPWLQVSDER | | |
| 298 | 313 | AT5 | MYB | IRMGIDPNTHRRFDQQKVNEEETILVNDPKPLSETEVSVALKNDTSAVL | 121 | 1.589 |
| | | G62 | | SGNLNQLADVDGDDQPWSFLMENDEGGGGDAAGELTMLLSGDITSSCSS | | 74749 |
| | | 320 | | SSSLWMKYGEFGYEDLELGCFDV | | 2 |
| 299 | 274 | AT1 | MYB | HHSQDQNNKEDFVSTTAAEMPTSPQQQSSSSADISAITTLGNNNDISNS | 130 | 1.598 |
| | | G06 | | NKDSATSSEDVLAIIDESFWSEVVLMDCDISGNEKNEKKIENWEGSLDR | | 53736 |
| | | 180 | | NDKGYNHDMEFWFDHLTSSSCIIGEMSDISEF | | 2 |
| 300 | 245 | AT2 | LOBAS2 | ASLELPQPQTRPQPMPQPQPLFFTPPPPLAITDLPASVSPLPSTYDLAS | 124 | 1.606 |
| | | G45 | | IFDQTTSSSAWATQQRRFIDPRHQYGVSSSSSSVAVGLGGENSHDLQAL | | 67546 |
| | | 420 | | AHELLHRQGSPPPAATDHSPSRTMSR | | 6 |
| 301 | 421 | AT1 | TCP | VQAKNLNNDDEDFGNIGGDVEQEEEKEEDDNGDKSFVYGLSPGYGEEEV | 214 | 1.617 |
| | | G67 | | VCEATKAGIRKKKSELRNISSKGLGAKARGKAKERTKEMMAYDNPETAS | | 18713 |
| | | 260 | | DITQSEIMDPFKRSIVFNEGEDMTHLFYKEPIEEFDNQESILTNMTLPT | | |
| | | | | KMGQSYNQNNGILMLVDQSSSSNYNTFLPQNLDYSYDQNPFHDQTLYVV | | |
| | | | | TDKNFPKGKVWIQDSFVN | | |
| 302 | 279 | AT4 | MYB | LQMGIDPVTHEPRTNDLSPILDVSQMLAAAINNGQFGNNNLLNNNTALE | 243 | 1.636 |
| | | G17 | | DILKLQLIHKMLQIITPKAIPNISSFKTNLLNPKPEPVVNSENTNSVNP | | 61173 |
| | | 785 | | KPDPPAGLFINQSGITPEAASDFIPSYENVWDGFEDNQLPGLVTVSQES | | 9 |
| | | | | LNTAKPGTSTTTKVNDHIRTGMMPCYYGDQLLETPSTGSVSVSPETTSL | | |
| | | | | NHPSTAQHSSGSDFLEDWEKFLDDETSDSCWKSFELDLTSPTSSPVPW | | |
| 303 | 78 | AT4 | AP2- | MHYPNNRTEFVGAPAPTRYQKEQLSPEQELSVIVSALQHVISGENETAP | 135 | 1.644 |
| | | G34 | EREBP | CQGFSSDSTVISAGMPRLDSDTCQVCRIEGCLGCNYFFAPNQRIEKNHQ | | 95452 |
| | | 410 | | QEEEITSSSNRRRESSPVAKKAEGGGKIRKRKNKKNG | | 2 |
| 304 | 452 | AT5 | WRKY | MGSFDRQRAVPKFKTATPSPLPLSPSPYFTMPPGLTPADFLDSPLLFTS | 110 | 1.646 |
| | | G07 | | SNILPSPTTGTFPAQSLNYNNNGLLIDKNEIKYEDTTPPLFLPSMVTQP | | 78435 |
| | | 100 | | LPQLDLFKSEIM | | |
| 305 | 324 | AT2 | MYB- | MNRGIEVMSPATYLETSNWLFQENRGTKWTAEENKKFENALAFYDKDTP | 134 | 1.649 |
| | | G38 | related | DRWSRVAAMLPGKTVGDVIKQYRELEEDVSDIEAGLIPIPGYASDSFTL | | 71479 |
| | | 090 | | DWGGYDGASGNNGFNMNGYYFSAAGGKRGSAARTAE | | 9 |
| 306 | 486 | AT2 | bHLH | MGCFDPNTSAEVTVESSFSQSEQPPPPPQVLVAGSTSNSNCSVEVEELS | 236 | 1.651 |
| | | G31 | | EFHLSPQDCPQASSTPLQFHINPPPPPPPPCDQFHNNLIHQMASHQQHS | | 92631 |
| | | 220 | | SWENGYQDFVNLGPNSATTPDLLSLLHLPRWSLPPNHHPSSMLPNSSIS | | 5 |
| | | | | FSDIMSSSSAAAVMYDPLFHLNFPMQPRDQNQLRNGSCLLGVEDQIQMD | | |
| | | | | ANGGVNVMYFEGANNNNNNGGFENEILEFNNGVTRKGRGS | | |
| 307 | 224 | AT3 | HSF | NPDRWEFANEGFLRGQKHLLKNIRRRKTSNNSNQMQQPQSSEQQSLDNF | 281 | 1.654 |
| | | G22 | | CIEVGRYGLDGEMDSLRRDKQVLMMELVRLRQQQQSTKMYLTLIEEKLK | | 24817 |
| | | 830 | | KTESKQKQMMSFLARAMQNPDFIQQLVEQKEKRKEIEEAISKKRQRPID | | |
| | | | | QGKRNVEDYGDESGYGNDVAASSSALIGMSQEYTYGNMSEFEMSELDKL | | |
| | | | | AMHIQGLGDNSSAREEVLNVEKGNDEEEVEDQQQGYHKENNEIYGEGFW | | |
| | | | | EDLLNEGQNFDFEGDQENVDVLIQQLGYLGSSSHTN | | |
| 308 | 56 | AT2 | AP2- | VEVVRESLKKMENVNLHDGGSPVMALKRKHSLRNRPRGKKRSSSSSSSS | 100 | 1.660 |
| | | G31 | EREBP | SNSSSCSSSSSTSSTSRSSSKQSVVKQESGTLVVFEDLGAEYLEQLLMS | | 93493 |
| | | 230 | | SC | | 7 |
| 309 | 199 | AT3 | G2- | MYIKAIMNRHRLLSAATDECNKKLGQACSSSLSPVHNFLNVQPEHRKTP | 237 | 1.667 |
| | | G13 | like | FIRSQSPDSPGQLWPKNSSQSTFSRSSTFCTNLYLSSSSTSETQKHLGN | | 08732 |
| | | 040 | | SLPFLPDPSSYTHSASGVESARSPSIFTEDLGNQCDGGNSGSLLKDELN | | 7 |
| | | | | LSGDACSDGDFHDFGCSNDSYCLSDQMELQFLSDELELAITDRAETPRL | | |
| | | | | DEIYETPLASNPVTRLSPSQSCVPGAMSVDVVSSHPSPGSA | | |
| 310 | 258 | AT5 | MADS | PYDTNPEVWPSNSGVQRVVSEFRTLPEMDQHKKMVDQEGELKQRIAKAT | 272 | 1.690 |
| | | G48 | | ETLRRQRKDSRELEMTEVMFQCLIGNMEMFHLNIVDLNDLGYMIEQYLK | | 46018 |
| | | 670 | | DVNRRIEILRNSGTEIGESSSVAVAASEGNIPMPNLVATTAPTTTIYEV | | 8 |
| | | | | GSSSSFAAVANFVNPIDLQQQFRHPAAQHVGLNEQPQNLNLNLNQNYNQN | | |
| | | | | QEWFMEMMNHPEQMRYQTEQMGYQFMDDNHHNHIHHQPQEHQHQIHDES | | |
| | | | | SNALDAANSSSIIPVTSSSITNKTWFH | | |
| 311 | 30 | AT4 | AP2- | ELSKLLPRPVSLSPRDVRAAATKAALMDFDTTAFRSDTETSETTTSNKM | 146 | 1.693 |
| | | G32 | EREBP | SESSESNETVSFSSSSWSSVTSIEESTVSDDLDEIVKLPSLGTSLNESN | | 23603 |
| | | 800 | | EFVIFDSLEDLVYMPRWLSGTEEEVFTYNNNDSSLNYSSVFESWKHFP | | 1 |
| 312 | 81 | AT5 | AP2- | DLAGSFPRPSSLSPRDIQVAALKAAHMETSQSFSSSSSLTFSSSQSSSS | 126 | 1.704 |
| | | G25 | EREBP | LESLVSSSATGSEELGEIVELPSLGSSYDGLTQLGNEFIFSDSADLWPY | | 38598 |
| | | 810 | | PPQWSEGDYQMIPASLSQDWDLQGLYNY | | |
| 313 | 332 | AT5 | MYB- | SGGKDKRRASIHDITTVNLEEEASLETNKSSIVVGDQRSRLTAFPWNQT | 97 | 1.714 |
| | | G58 | related | DNNGTQADAFNITIGNAISGVHSYGQVMIGGYNNADSCYDAQNTMFQL | | 32600 |
| | | 900 | | | | 6 |
| 314 | 237 | AT3 | Homeo | QLERDYDLLKSTYDQLLSNYDSIVMDNDKLRSEVTSLTEKLQGKQETAN | 149 | 1.725 |
| | | G01 | box | EPPGQVPEPNQLDPVYINAAAIKTEDRLSSGSVGSAVLDDDAPQLLDSC | | 28485 |
| | | 470 | | DSYFPSIVPIQDNSNASDHDNDRSCFADVFVPTTSPSHDHHGESLAFWG | | |
| | | | | WP | | |
| 315 | 424 | AT5 | TCP | PPLQFPPGFHQLNPNLTGLGESFPGVFDLGRTQREALDLEKRKWVNLDH | 151 | 1.725 |
| | | G08 | | VFDHIDHHNHFSNSIQSNKLYFPTITSSSSSYHYNLGHLQQSLLDQSGN | | 89670 |
| | | 070 | | VTVAFSNNYNNNNLNPPAAETMSSLFPTRYPSFLGGGQLQLFSSTSSQP | | 3 |
| | | | | DHIE | | |
| 316 | 500 | AT1 | bZIP | MDGSMNLGNEPPGDGGGGGGLTRQGSIYSLTFDEFQSSVGKDFGSMNMD | 335 | 1.733 |
| | | G45 | | ELLKNIWSAEETQAMASGVVPVLGGGQEGLQLQRQGSLTLPRTLSQKTV | | 45299 |
| | | 249 | | DQVWKDLSKVGSSGVGGSNLSQVAQAQSQSQSQRQQTLGEVTLEEFLVR | | 4 |
| | | | | AGVVREEAQVAARAQIAENNKGGYFGNDANTGFSVEFQQPSPRVVAAGV | | |
| | | | | MGNLGAETANSLQVQGSSLPLNVNGARTTYQQSQQQQPIMPKQPGFGYG | | |
| | | | | TQMGQLNSPGIRGGGLVGLGDQSLTNNVGFVQGASAAIPGALGVGAVSP | | |
| | | | | VTPLSSEGIGKSNGDSSSLSPSPYMENGGVRGRKSGTVEKV | | |
| 317 | 260 | AT1 | MYB | VMMKFQNGIINENKTNLATDISSCNNNNNGCNHNKRTTNKGQWEKKLQT | 214 | 1.736 |
| | | G74 | | DINMAKQALFQALSLDQPSSLIPPDPDSPKPHHHSTTTYASSTDNISKL | | 24156 |
| | | 650 | | LQNWTSSSSSKPNTSSVSNNRSSSPGEGGLFDHHSLFSSNSESGSVDEK | | 2 |
| | | | | LNLMSETSMFKGESKPDIDMEATPTTTTTDDQGSLSLIEKWLEDDQGLV | | |
| | | | | QCDDSQEDLIDVSLEELK | | |
| 318 | 407 | AT2 | SBP | NPEPGANGNPSDDHSSNYLLITLLKILSNMHNHTGDQDLMSHLLKSLVS | 701 | 1.758 |
| | | G47 | | HAGEQLGKNLVELLLQGGGSQGSLNIGNSALLGIEQAPQEELKQFSARQ | | 32929 |
| | | 070 | | DGTATENRSEKQVKMNDFDLNDIYIDSDDTDVERSPPPTNPATSSLDYP | | 5 |
| | | | | SWIHQSSPPQTSRNSDSASDQSPSSSSEDAQMRTGRIVFKLFGKEPNEF | | |
| | | | | PIVLRGQILDWLSHSPTDMESYIRPGCIVLTIYLRQAETAWEELSDDLG | | |
| | | | | FSLGKLLDLSDDPLWTTGWIYVRVQNQLAFVYNGQVVVDTSLSLKSRDY | | |
| | | | | SHIISVKPLAIAATEKAQFTVKGMNLRQRGTRLLCSVEGKYLIQETTHD | | |
| | | | | STTREDDDFKDNSEIVECVNFSCDMPILSGRGFMEIEDQGLSSSFFPFL | | |
| | | | | VVEDDDVCSEIRILETTLEFTGTDSAKQAMDFIHEIGWLLHRSKLGESD | | |
| | | | | PNPGVFPLIRFQWLIEFSMDREWCAVIRKLLNMFFDGAVGEFSSSSNAT | | |
| | | | | LSELCLLHRAVRKNSKPMVEMLLRYIPKQQRNSLFRPDAAGPAGLTPLH | | |
| | | | | IAAGKDGSEDVLDALTEDPAMVGIEAWKTCRDSTGFTPEDYARLRGHES | | |
| | | | | YIHLIQRKINKKSTTEDHVVVNIPVSFSDREQKEPKSGPMASALEITQI | | |
| | | | | PCKLCDHKLVYGTTRRSVAYRPAMLSMVAIAAVCVCVALLFKSCPEVLY | | |
| | | | | VFQPFRWELLDYGTS | | |
| 319 | 288 | AT5 | MYB | LRMGIDPVTHCPRINLLQLSSFLTSSLFKSMSQPMNTPFDLTTSNINPD | 203 | 1.766 |
| | | G54 | | ILNHLTASLNNVQTESYQPNQQLQNDLNTDQTTFTGLLNSTPPVQWQNN | | 31761 |
| | | 230 | | GEYLGDYHSYTGTGDPSNNKVPQAGNYSSAAFVSDHINDGENFKAGWNF | | |
| | | | | SSSMLAGTSSSSSTPLNSSSTFYVNGGSEDDRESFGSDMLMFHHHHDHN | | |
| | | | | NNALNLS | | |
| 320 | 484 | AT5 | bHLH | EKVHMYEDSHQMWYQSPTKLIPWRNSHGSVAEENDHPQIVKSFSSNDKV | 212 | 1.768 |
| | | G38 | | AASSGFLLDTYNSVNPDIDSAVSTKIPEHSPVSAVSSYLRTEPSLQFVQ | | 02006 |
| | | 860 | | HDFWQPKTSCGTINCFTNELLTSDEKTSASLSTVCSQRVLNTLTEALKS | | |
| | | | | SGVNMSETMISVQLSLRKREDREYSVAAFASEDNGNSIADEEGDSPTET | | |
| | | | | RSFCNDIDHSQKRIRR | | |
| 321 | 409 | AT5 | SBP | QPEHIGRPANFFTGFQGSKLLEFSGGSHVFPTTSVLNPSWGNSLVSVAV | 184 | 1.785 |
| | | G50 | | AANGSSYGQSQSYVVGSSPAKTGIMFPISSSPNSTRSIAKQFPFLQEEE | | 72584 |
| | | 670 | | SSRTASLCERMTSCIHDSDCALSLLSSSSSSVPHLLQPPLSLSQEAVET | | 3 |
| | | | | VFYGSGLFENASAVSDGSVISGNEAVRLPQTFPFHWE | | |
| 322 | 22 | AT1 | AP2- | MADLFGGGHGGELMEALQPFYKSASTSASNPAFASSNDAFASAPNDLFS | 143 | 1.794 |
| | | G36 | EREBP | SSSYYNPHASLFPSHSTTSYPDIYSGSMTYPSSFGSDLQQPENYQSQFH | | 12293 |
| | | 060 | | YQNTITYTHQDNNTCMLNFIEPSQPGFMTQPGPSSGSVSKPAKLY | | 9 |
| 323 | 17 | AT3 | AP2- | HLQRNTRPSLSNSQRFKWVPSRKFISMFPSCGMLNVNAQPSVHIIQQRL | 193 | 1.805 |
| | | G57 | EREBP | EELKKTGLLSQSYSSSSSSTESKTNTSFLDEKTSKGETDNMFEGGDQKK | | 36951 |
| | | 600 | | PEIDLTEFLQQLGILKDENEAEPSEVAECHSPPPWNEQEETGSPERTEN | | 2 |
| | | | | FSWDTLIEMPRSETTTMQFDSSNFGSYDFEDDVSFPSIWDYYGSLD | | |
| 324 | 322 | AT1 | MYB- | MNRDRRRSSIHDITTVNNQAPAVTGGGQQPQVVKHRPAQPQPQPQPQPQ | 129 | 1.888 |
| | | G49 | related | QHHPPTMAGLGMYGGAPVGQPIIAPPDHMGSAVGTPVMLPPPMGTHHHH | | 65258 |
| | | 010 | | HHHHLGVAPYAVPAYPVPPLPQQHPAPSTMH | | |
| 325 | 453 | AT5 | WRKY | MSSEDWDLFAVVRSCSSSVSTTNSCAGHEDDIGNCKQQQDPPPPPLFQA | 158 | 1.894 |
| | | G52 | | SSSCNELQDSCKPFLPVTTTTTTTWSPPPLLPPPKASSPSPNILLKQEQ | | 24181 |
| | | 830 | | VLLESQDQKPPLSVRVFPPSTSSSVFVFRGQRDQLLQQQSQPPLRSRKR | | 2 |
| | | | | KNQQKRTICHV | | |
| 326 | 308 | AT5 | MYB | IQMGFDPMTHRPRTDIFSGLSQLMSLSSNLRGFVDLQQQFPIDQEHTIL | 218 | 1.914 |
| | | G10 | | KLQTEMAKLQLFQYLLQPSSMSNNVNPNDEDTLSLLNSIASFKETSNNT | | 45863 |
| | | 280 | | TSNNLDLGFLGSYLQDFHSLPSLKTLNSNMEPSSVFPQNLDDNHFKEST | | 3 |
| | | | | QRENLPVSPIWLSDPSSTTPAHVNDDLIFNQYGIEDVNSNITSSSGQES | | |
| | | | | GASASAAWPDHLLDDSIFSDIP | | |
| 327 | 386 | AT2 | NAC | RATGQAKNTETWSSSYFYDEVAPNGVNSVMDPIDYISKQQHNIFGKGLM | 207 | 1.924 |
| | | G18 | | CKQELEGMVDGINYIQSNQFIQLPQLQSPSLPLMKRPSSSMSITSMDNN | | 10691 |
| | | 060 | | YNYKLPLADEESFESFIRGEDRRKKKKQVMMTGNWRELDKFVASQLMSQ | | 2 |
| | | | | EDNGTSSFAGHHIVNEDKNNNDVEMDSSMFLSEREEENRFVSEFLSTNS | | |
| | | | | DYDIGICVEDN | | |
| 328 | 520 | AT5 | bZIP | MQPSTNIFSLHGCPPSYLSHIPTSSPFCGQNPNPFFSFETGVNTSQFMS | 69 | 1.929 |
| | | G38 | | LISSNNSTSDEAEENHKEII | | 35931 |
| | | 800 | | | | |
| 329 | 180 | AT2 | E2F- | CPGDEDADVSVLQLQAEIENLALEEQALDNQIRWLFVTEEDIKSLPGFQ | 233 | 1.931 |
| | | G36 | DP | NQTLIAVKAPHGTTLEVPDPDEAADHPQRRYRIILRSTMGPIDVYLVSE | | 75862 |
| | | 010 | | FEGKFEDTNGSGAAPPACLPIASSSGSTGHHDIEALTVDNPETAIVSHD | | 9 |
| | | | | HPHPQPGDTSDLNYLQEQVGGMLKITPSDVENDESDYWLLSNAEISMTD | | |
| | | | | IWKTDSGIDWDYGIADVSTPPPGMGEIAPTAVDSTPR | | |
| 330 | 222 | AT3 | HSF | DPDRWEFANEGFLRGQKQILKSIVRRKPAQVQPPQQPQVQHSSVGACVE | 381 | 1.940 |
| | | G02 | | VGKFGLEEEVERLQRDKNVLMQELVRLRQQQQVTEHHLQNVGQKVHVME | | 67659 |
| | | 990 | | QRQQQMMSFLAKAVQSPGFLNQFSQQSNEANQHISESNKKRRLPVEDQM | | 1 |
| | | | | NSGSHGVNGLSRQIVRYQSSMNDATNTMLQQIQQMSNAPSHESLSSNNG | | |
| | | | | SFLLGDVPNSNISDNGSSSNGSPEVTLADVSSIPAGFYPAMKYHEPCET | | |
| | | | | NQVMETNLPFSQGDLLPPTQGAAASGSSSSDLVGCETDNGECLDPIMAV | | |
| | | | | LDGALELEADTLNELLPEVQDSFWEQFIGESPVIGETDELISGSVENEL | | |
| | | | | ILEQLELQSTLSNVWSKNQQMNHLTEQMGLLTSDALRK | | |
| 331 | 36 | AT4 | AP2- | DSAWRLRIPESTCAKDIQKAAAEAALAFQDEMCDATTDHGEDMEETLVE | 109 | 1.945 |
| | | G25 | EREBP | AIYTAEQSENAFYMHDEAMFEMPSLLANMAEGMLLPLPSVQWNHNHEVD | | 05367 |
| | | 480 | | GDDDDVSLWSY | | 6 |
| 332 | 112 | AT5 | C2C2- | PSSSNSSSSTSSGKKPSNIVTANTSDLMALAHSHQNYQHSPLGFSHFGG | 245 | 1.963 |
| | | G62 | DOF | MMGSYSTPEHGNVGFLESKYGGLLSQSPRPIDFLDSKFDLMGVNNDNLV | | 16117 |
| | | 940 | | MVNHGSNGDHHHHHNHHMGLNHGVGLNNNNNNGGFNGISTGGNGNGGGL | | 1 |
| | | | | MDISTCQRLMLSNYDHHHYNHQEDHQRVATIMDVKPNPKLLSLDWQQDQ | | |
| | | | | CYSNGGGSGGAGKSDGGGYGNGGYINGLGSSWNGLMNGYGTSTKTNSLV | | |
| 333 | 497 | AT1 | bHLH | MGGESNEGGEMGFKHGDDESGGISRVGITSMPLYAKADPFFSSADWDPV | 214 | 1.963 |
| | | G10 | | VNAAAAGFSSSHYHPSMAMDNPGMSCFSHYQPGSVSGFAADMPASLLPF | | 51007 |
| | | 120 | | GDCGGGQIGHFLGSDKKGERLIRAGESSHEDHHQVSDDAVLGASPVGKR | | 1 |
| | | | | RLPEAESQWNKKAVEEFQEDPQRGNDQSQKKHKNDQSKETVNKESSQSE | | |
| | | | | EAPKENYIHMRARRGQAT | | |
| 334 | 15 | AT1 | AP2- | ELATYLPRPASSSPRDVQAAAAVAAAMDESPSSSSLVVSDPTTVIAPAE | 145 | 1.974 |
| | | G77 | EREBP | TQLSSSSYSTCTSSSLSPSSEEAASTAEELSEIVELPSLETSYDESLSE | | 35322 |
| | | 200 | | FVYVDSAYPPSSPWYINNCYSFYYHSDENGISMAEPFDSSNFGPLFP | | 6 |
| 335 | 468 | AT2 | WRKY | MNYPSNPNPSSTDFTEFFKFDDEDDTFEKIMEEIGREDHSSSPTLSWSS | 102 | 1.983 |
| | | G21 | | SEKLVAAEITSPLQTSLATSPMSFEIGDKDEIKKRKRHKEDPIIHVEKT | | 90167 |
| | | 900 | | KSSI | | 4 |
| 336 | 309 | AT1 | MYB | IQMGIDPVTHQPRTDLFASLPQLIALANLKDLIEQTSQFSSMQGEAAQL | 249 | 2.016 |
| | | G34 | | ANLQYLQRMENSSASLTNNNGNNFSPSSILDIDQHHAMNLLNSMVSWNK | | 87073 |
| | | 670 | | DQNPAFDPVLELEANDQNQDLFPLGFIIDQPTQPLQQQKYHLNNSPSEL | | 3 |
| | | | | PSQGDPLLDHVPFSLQTPLNSEDHFIDNLVKHPTDHEHEHDDNPSSWVL | | |
| | | | | PSLIDNNPKTVTSSLPHNNPADASSSSSYGGCEAASFYWPDICEDESLM | | |
| | | | | NVIS | | |
| 337 | 504 | AT2 | bZIP | TAQMEELSTRLQSLNEIVDLVQSNGAGFGVDQIDGCGFDDRTVGIDGYY | 79 | 2.022 |
| | | G18 | | DDMNMMSNVNHWGGSVYTNQPIMANDINMY | | 27544 |
| | | 160 | | | | 1 |
| 338 | 365 | AT5 | NAC | NNPSTTTQPMTRIPVEDFTRMDSLENIDHLLDFSSLPPLIDPSFMSQTE | 163 | 2.037 |
| | | G18 | | QPNFKPINPPTYDISSPIQPHHENSYQSIFNHQVFGSASGSTYNNNNEM | | 93247 |
| | | 270 | | IKMEQSLVSVSQETCLSSDVNANMTTTTEVSSGPVMKQEMGMMGMVNGS | | 9 |
| | | | | KSYEDLCDLRGDLWDF | | |
| 339 | 353 | AT3 | NAC | SHASLSSPDVALVTSNQEHEENDNEPFVDRGTFLPNLQNDQPLKRQKSS | 173 | 2.044 |
| | | G04 | | CSFSNLLDATDLTFLANFLNETPENRSESDFSFMIGNFSNPDIYGNHYL | | 55451 |
| | | 070 | | DQKLPQLSSPTSETSGIGSKRERVDFAEETINASKKMMNTYSYNNSIDQ | | 5 |
| | | | | MDHSMMQQPSFLNQELMMSSHLQYQG | | |
| 340 | 390 | AT5 | NAC | KLTTMNYNNPRTMMGSSSGQESNWFTQQMDVGNGNYYHLPDLESPRMFQ | 192 | 2.049 |
| | | G62 | | GSSSSSLSSLHQNDQDPYGVVLSTINATPTTIMQRDDGHVITNDDDHMI | | 78393 |
| | | 380 | | MMNTSTGDHHQSGLLVNDDHNDQVMDWQTLDKFVASQLIMSQEEEEVNK | | 4 |
| | | | | DPSDNSSNETFHHLSEEQAATMVSMNASSSSSPCSFYSWAQNTHT | | |
| 341 | 524 | AT1 | bZIP | MEKSDPPPVPKPGATIIPSSDPIPNADPIPSSSFHRRSRSDDMSMEMFM | 147 | 2.061 |
| | | G06 | | DPLSSAAPPSSDDLPSDDDLESSFIDVDSLTSNPNPFQNPSLSSNSVSG | | 07363 |
| | | 850 | | AANPPPPPSSRPRHRHSNSVDAGCAMYAGDIMDAKKAMPPEKLSELWNI | | |
| 342 | 376 | AT5 | NAC | SGTGPKNGEQYGAPYLEEEWEEDGMTYVPAQDAFSEGLALNDDVYVDID | 408 | 2.083 |
| | | G04 | | DIDEKPENLVVYDAVPILPNYCHGESSNNVESGNYSDSGNYIQPGNNVV | | 67959 |
| | | 410 | | DSGGYFEQPIETFEEDRKPIIREGSIQPCSLFPEEQIGCGVQDENVVNL | | 3 |
| | | | | ESSNNNVFVADTCYSDIPIDHNYLPDEPFMDPNNNLPLNDGLYLETNDL | | |
| | | | | SCAQQDDFNFEDYLSFFDDEGLTFDDSLLMGPEDFLPNQEALDQKPAPK | | |
| | | | | ELEKEVAGGKEAVEEKESGEGSSSKQDTDFKDFDSAPKYPFLKKTSHML | | |
| | | | | GAIPTPSSFASQFQTKDAMRLHAAQSSGSVHVTAGMMRISNMTLAADSG | | |
| | | | | MGWSYDKNGNLNVVLSFGVVQQDDAMTASGSKTGITATRAMLVFMCLWV | | |
| | | | | LLLSVSFKIVTMVSAR | | |
| 343 | 490 | AT1 | bHLH | MGSEYKHILKSLCLSHGWSYAVFWRYDPINSMILRFEEAYNDEQSVALV | 353 | 2.085 |
| | | G64 | | DDMVLQAPILGQGIVGEVASSGNHQWLFSDTLFQWEHEFQNQFLCGFKI | | 89457 |
| | | 625 | | LIRQFTYTQTIAIIPLGSSGVVQLGSTQKILESTEILEQTTRALQETCL | | 1 |
| | | | | KPHDSGDLDTLFESLGDCEIFPAESFQGFSFDDIFAEDNPPSLLSPEMI | | |
| | | | | SSEAASSNQDLTNGDDYGFDILQSYSLDDLYQLLADPPEQNCSSMVIQG | | |
| | | | | VDKDLFDILGMNSQTPTMALPPKGLFSELISSSLSNNTCSSSLTNVQEY | | |
| | | | | SGVNQSKRRKLDTSSAHSSSLFPQEETVTSRSLWIDDDERSSIGGNWKK | | |
| | | | | PHEEGVKKKR | | |
| 344 | 23 | AT1 | AP2- | ESLRSYPETASSQASHTTPSSNTGGKSSDSESPCSSNEMSSCGRVTDEI | 107 | 2.106 |
| | | G75 | EREBP | SWEHINVDLPVMDDSSIWEEATMSLGFPWVHEGDNNISRFDTCISGGFS | | 63066 |
| | | 490 | | NWDSFHSPL | | 2 |
| 345 | 80 | AT5 | AP2- | VVKSEEGSDHVKDVNSPLMSPKSLSELLNAKLRKSCKDLTPSLTCLRLD | 126 | 2.123 |
| | | G11 | EREBP | TDSSHIGVWQKRAGSKTSPTWVMRLELGNVVNESAVDLGLTTMNKQNVE | | 38147 |
| | | 190 | | KEEEEEEAIISDEDQLAMEMIEELLNWS | | 5 |
| 346 | 110 | AT3 | C2C2- | SSSATKSLRTTPEPTMTHDGKSFPTASFGYNNNNISNEQMELGLAYALL | 151 | 2.128 |
| | | G45 | DOF | NKQPLGVSSHLGFGSSQSPMAMDGVYGTTSHQMENTGYAFGNGGGGMEQ | | 36132 |
| | | 610 | | MATSDPNRVLWGFPWQMNMGGGSGHGHGHVDQIDSGREIWSSTVNYINT | | 3 |
| | | | | GALL | | |
| 347 | 371 | AT3 | NAC | TVSSRKYTPDWRELANGKRVKQQQSNYQEAYINFGDNESSSSTNVMNVR | 118 | 2.142 |
| | | G12 | | EGKGNYERSVFQLQQTPYQHQNQPILMDTTHVDSFQHFSNDNIHHETYE | | 59515 |
| | | 910 | | TWPDELRSVVEFAFPPSFLS | | 7 |
| 348 | 212 | AT5 | HB | KLEEEYAKLKNHHDNVVLGQCQLESQILKLTEQLSEAQSEIRKLSERLE | 102 | 2.147 |
| | | G66 | | EMPTNSSSSSLSVEANNAPTDFELAPETNYNIPFYMLDNNYLQSMEYWD | | 22285 |
| | | 700 | | GLYV | | 5 |
| 349 | 24 | AT1 | AP2- | NITTTSPFLMNIDEKTLLSPKSIQKVAAQAANSSSDHFTPPSDENDHDH | 145 | 2.193 |
| | | G77 | EREBP | DDGLDHHPSASSSAASSPPDDDHHNDDDGDLVSLMESFVDYNEHVSLMD | | 39193 |
| | | 640 | | PSLYEFGHNEIFFTNGDPFDYSPQLHSSEATMDDFYDDVDIPLWSFS | | 6 |
| 350 | 65 | AT1 | AP2- | YKGIRRRPWGRWAAEIRDPIKGVRVWLGTFNTAEEAARAYDLEAKRIRG | 187 | 2.206 |
| | | G72 | EREBP | AKAKLNFPNESSGKRKAKAKTVQQVEENHEADLDVAVVSSAPSSSCLDF | | 86638 |
| | | 360 | | LWEENNPDTLLIDTQWLEDIIMGDANKKHEPNDSEEANNVDASLLSEEL | | 6 |
| | | | | LAFENQTEYFSQMPFTEGNCDSSTSLSSLFDGGNDMGLWS | | |
| 351 | 29 | AT4 | AP2- | TDKKPQLPEGSVRPLSKLDIQTIATNYASSVVHVPSHATTLPATTQVPS | 104 | 2.226 |
| | | G31 | EREB | EVPASSDVSASTEITEMVDEYYLPTDATAESIFSVEDLQLDSFLMMDID | | 48569 |
| | | 060 | P | WINNLI | | 6 |
| 352 | 76 | AT1 | AP2- | EENMKANSQKRSVKANLQKPVAKPNPNPSPALVQNSNISFENMCFMEEK | 177 | 2.243 |
| | | G53 | EREBP | HQVSNNNNNQFGMTNSVDAGCNGYQYFSSDQGSNSFDCSEFGWSDQAPI | | 21548 |
| | | 910 | | TPDISSAVINNNNSALFFEEANPAKKLKSMDFETPYNNTEWDASLDELN | | 9 |
| | | | | EDAVTTQDNGANPMDLWSIDEIHSMIGGVF | | |
| 353 | 295 | AT1 | MYB | NKSDSDERSRSENIALQTSSTRNTINHRSTYASSTENISRLLEGWMRAS | 164 | 2.252 |
| | | G08 | | PKSSTSTTFLEHKMQNRTNNFIDHHSDQFPYEQLQGSWEEGHSKGINGD | | 09986 |
| | | 810 | | DDQGIKNSENNNGDDVHHEDGDHEDDDDHNATPPLTFIEKWLLEETSTT | | 2 |
| | | | | GGQMEEMSHLMELSNML | | |
| 354 | 396 | AT1 | NLP | MEDSFLQSENVVMDADEMDGLLLDGCWLETTDGSEFLNIAPSTSSVSPF | 539 | 2.282 |
| | | G20 | | DPTSFMWSPTQDTSALCTSGVVSQMYGQDCVERSSLDEFQWNKRWWIGP | | 62329 |
| | | 640 | | GGGGSSVTERLVQAVEHIKDYTTARGSLIQLWVPVNRGGKRVLTTKEQP | | 1 |
| | | | | FSHDPLCQRLANYREISVNYHFSAEQDDSKALAGLPGRVELGKLPEWTP | | |
| | | | | DVRFFKSEEYPRVHHAQDCDVRGTLAIPVFEQGSKICLGVIEVVMTTEM | | |
| | | | | VKLRPELESICRALQAVDLRSTELPIPPSLKGCDLSYKAALPEIRNLLR | | |
| | | | | CACETHKLPLAQTWVSCQQQNKSGCRHNDENYIHCVSTIDDACYVGDPT | | |
| | | | | VREFHEACSEHHLLKGQGVAGQAFLINGPCFSSDVSNYKKSEYPLSHHA | | |
| | | | | NMYGLHGAVAIRLRCIHTGSADFVLEFFLPKDCDDLEEQRKMLNALSTI | | |
| | | | | MAHVPRSLRTVTDKELEEESEVIEREEIVTPKIENASELHGNSPWNASL | | |
| | | | | EEIQRSNNTSNPQNLGLVFDGGDKPNDGFGLKRGFDYTMDSNVNESSTF | | |
| 355 | 505 | AT4 | bZIP | RAQLDELNHRLQSLNDIIEFLDSSNNNNNNNMGMCSNPLVGLECDDFFV | 71 | 2.288 |
| | | G34 | | NQMNMSYIMNQPLMASSDALMY | | 89811 |
| | | 590 | | | | 2 |
| 356 | 70 | AT5 | AP2- | YSDMPPSSSVTSIVSPDDPPPPPPPPAPPSNDPVDYMMMENQYSSTDSP | 135 | 2.309 |
| | | G13 | EREBP | MLQPHCDQVDSYMFGGSQSSNSYCYSNDSSNELPPLPSDLSNSCYSQPQ | | 92334 |
| | | 910 | | WTWTGDDYSSEYVHSPMFSRMPPVSDSFPQGENYFGS | | 2 |
| 357 | 346 | AT1 | NAC | NIQIPKRKGEEEEAEEESTSVGKEEEEEKEKKWRKCDGNYIEDESLKRA | 148 | 2.319 |
| | | G54 | | SAETSSSELTQGVLLDEANSSSIFALHFSSSLLDDHDHLFSNYSHQLPY | | 85069 |
| | | 330 | | HPPLQLQDFPQLSMNEAEIMSIQQDFQCRDSMNGTLDEIFSSSATFPAS | | 1 |
| | | | | L | | |
| 358 | 270 | AT1 | MYB | RQLNIDSNSHKFIEVVRSFWFPRLINEIKDNSYTNNIKANAPDLLGPIL | 161 | 2.329 |
| | | G25 | | RDSKDLGENNMDCSTSMSEDLKKTSQFMDFSDLETTMSLEGSRGGSSQC | | 70097 |
| | | 340 | | VSEVYSSFPCLEEEYMVAVMGSSDISALHDCHVADSKYEDDVTQDLMWN | | 6 |
| | | | | MDDIWQFNEYAHEN | | |
| 359 | 111 | AT4 | C2C2- | PCSLQVISSPPLFSNGTSSASRELVRNHPSTAMMMMSSGGFSGYMFPLD | 151 | 2.333 |
| | | G38 | DOF | PNFNLASSSIESLSSENQDLHQKLQQQRLVTSMFLQDSLPVNEKTVMFQ | | 25157 |
| | | 000 | | NVELIPPSTVTTDWVFDRFATGGGATSGNHEDNDDGEGNLGNWFHNANN | | 5 |
| | | | | NALL | | |
| 360 | 378 | AT1 | NAC | RGASKLLNEQEGFMDEVLMEDETKVVVNEAERRTEEEIMMMTSMKLPRT | 107 | 2.340 |
| | | G69 | | CSLAHLLEMDYMGPVSHIDNESQFDHLHQPDSESSWFGDLQFNQDEILN | | 01759 |
| | | 490 | | HHRQAMFKF | | 8 |
| 361 | 388 | AT5 | NAC | RTTIPTKRRQLWDPNCLFYDDATLLEPLDKRARHNPDFTATPFKQELLS | 130 | 2.348 |
| | | G66 | | EASHVQDGDFGSMYLQCIDDDQFSQLPQLESPSLPSEITPHSTTFSENS | | 54222 |
| | | 300 | | SRKDDMSSEKRITDWRYLDKFVASQFLMSGED | | 4 |
| 362 | 297 | AT1 | MYB | RQLNIESNSDKFFDAVRSFWVPRLIEKMEQNSSTTTTYCCPQNNNNNSL | 163 | 2.353 |
| | | G68 | | LLPSQSHDSLSMQKDIDYSGFSNIDGSSSTSTCMSHLTTVPHFMDQSNT | | 10097 |
| | | 320 | | NIIDGSMCFHEGNVQEFGGYVPGMEDYMVNSDISMECHVADGYSAYEDV | | 5 |
| | | | | TQDPMWNVDDIWQFRE | | |
| 363 | 46 | AT2 | AP2- | EDLGGGRKKDEEAESSGGYWLETNKAGNGVIETEGGKDYVVYNEDAIEL | 110 | 2.355 |
| | | G38 | EREBP | GHDKTQNPMTDNEIVNPAVKSEEGYSYDRFKLDNGLLYNEPQSSSYHQG | | 43423 |
| | | 340 | | GGFDSYFEYFRF | | 2 |
| 364 | 183 | AT3 | EIL | PPLSLSGGSCSLLMNDCSQYDVEGFEKESHYEVEELKPEKVMNSSNFGM | 321 | 2.366 |
| | | G20 | | VAKMHDFPVKEEVPAGNSEFMRKRKPNRDLNTIMDRTVFTCENLGCAHS | | 85348 |
| | | 770 | | EISRGFLDRNSRDNHQLACPHRDSRLPYGAAPSRFHVNEVKPVVGFPQP | | 9 |
| | | | | RPVNSVAQPIDLTGIVPEDGQKMISELMSMYDRNVQSNQTSMVMENQSV | | |
| | | | | SLLQPTVHNHQEHLQFPGNMVEGSFFEDLNIPNRANNNNSSNNQTFFQG | | |
| | | | | NNNNNNVFKFDTADHNNFEAAHNNNNNSSGNRFQLVEDSTPFDMASFDY | | |
| | | | | RDDMSMPGVVGTMDGMQQKQQDVSIWF | | |
| 365 | 316 | AT3 | MYB- | QEADSRSEGSVKAIVIPPPRPKRKPAHPYPRKSPVPYTQSPPPNLSAME | 222 | 2.370 |
| | | G10 | related | KGTKSPTSVLSSFGSEDQNNYTTSKQPFKDDSDIGSTPISSITLFGKIV | | 44477 |
| | | 113 | | LVAEESHKPSSYNDDDLKQMTCQENHYSGMLVDTNLSLGVWETFCTGSN | | 2 |
| | | | | AFGSVTEASENLEKSAEPISSSWKRLSSLEKQGSCNPVNASGFRPYKRC | | |
| | | | | LSEREVTSSLTLVASDEKKSQRARIC | | |
| 366 | 377 | AT1 | NAC | NNSTASRHHHHLHHIHLDNDHHRHDMMIDDDRFRHVPPGLHFPAIFSDN | 143 | 2.386 |
| | | G52 | | NDPTAIYDGGGGGYGGGSYSMNHCFASGSKQEQLFPPVMMMTSLNQDSG | | 36389 |
| | | 880 | | IGSSSSPSKRFNGGGVGDCSTSMAATPLMQNQGGIYQLPGLNWYS | | 9 |
| 367 | 273 | AT3 | MYB | KSSSKQDKVKKSLSRKQQQVDLKPQPQAQSENHQSQLVSQDHMNIDNDH | 145 | 2.386 |
| | | G30 | | NIASSLYYPTSVFDDKLYMPQSVATTSSDHSMIDEGHLWGSLWNLDEDD | | 94502 |
| | | 210 | | PHSFGGGSGQGTAADIDEKFPDSGIEAPSCGSGDYSYTGVYMGGYIF | | 6 |
| 368 | 383 | AT1 | NAC | KNHFRGFHQEQEQDHHHHHQYISTNNDHDHHHHIDSNSNNHSPLILHPL | 205 | 2.393 |
| | | G79 | | DHHHHHHHIGRQIHMPLHEFANTLSHGSMHLPQLESPDSAAAAAAAAAS | | 04474 |
| | | 580 | | AQPFVSPINTTDIECSQNLLRLTSNNNYGGDWSFLDKLLTTGNMNQQQQ | | 3 |
| | | | | QQVQNHQAKCFGDLSNNDNNDQADHLGNNNGGSSSSPVNQRFPFHYLGN | | |
| | | | | DANLLKFPK | | |
| 369 | 348 | AT2 | NAC | PGVEDHPSVPRSLSTRHHNHNSSTSSRLALRQQQHHSSSSNHSDNNLNN | 216 | 2.399 |
| | | G02 | | NNNINNLEKLSTEYSGDGSTTTTTTNSNSDVTIALANQNIYRPMPYDTS | | 96640 |
| | | 450 | | NNTLIVSTRNHQDDDETAIVDDLQRLVNYQISDGGNINHQYFQIAQQFH | | 5 |
| | | | | HTQQQNANANALQLVAAATTATTLMPQTQAALAMNMIPAGTIPNNALWD | | |
| | | | | MWNPIVPDGNRDHYTNIPFK | | |
| 370 | 494 | AT3 | bHLH | MYPSIEDDDDLLAALCFDQSNGVEDPYGYMQTNEDNIFQDFGSCGVNLM | 153 | 2.478 |
| | | G23 | | QPQQEQFDSFNGNLEQVCSSFRGGNNGVVYSSSIGSAQLDLAASFSGVL | | 46466 |
| | | 210 | | QQETHQVCGFRGQNDDSAVPHLQQQQGQVFSGVVEINSSSSVGAVKEEF | | 8 |
| | | | | EEECSG | | |
| 371 | 11 | AT1 | AP2- | GSVGSYPVPESTSAADIRAAAAAAAAMKGCEEGEEEKKAKEKKSSSSKS | 118 | 2.545 |
| | | G12 | EREBP | RARECHVDNDVGSSSWCGTEFMDEEEVLNMPNLLANMAEGMMVAPPSWM | | 66376 |
| | | 630 | | GSRPSDDSPENSNDEDLWGY | | 3 |
| 372 | 79 | AT5 | AP2- | GLALTYVAPVSNSAADIRAAASRAAEMKQPDQGGDEKVLEPVQPGKEEE | 112 | 2.570 |
| | | G52 | EREBP | LEEVSCNSCSLEFMDEEAMLNMPTLLTEMAEGMLMSPPRMMIHPTMEDD | | 84775 |
| | | 020 | | SPENHEGDNLWSYK | | 4 |
| 373 | 21 | AT1 | AP2- | HLLNPSLVSRTSPRSIQQAASNAGMAIDAGIVHSTSVNSGCGDTTTYYE | 72 | 2.587 |
| | | G22 | EREBP | NGADQVEPLNISVYDYLGGHDHV | | 42183 |
| | | 810 | | | | 8 |
| 374 | 316 | AT4 | NAC | NELKKNSKSLKNKNEQDIGSCYSSLATSPCRDEASQIQSFKPSSTTNDS | 102 | 2.613 |
| | | G17 | | SSIWISPDFILDSSKDYPQIKEVASECFPNYHFPVTTANHHVEFPLQEM | | 11805 |
| | | 980 | | LVRS | | 1 |
| 375 | 387 | AT4 | NAC | KPMTGQAKNTETWSSSYFYDELPSGVRSVTEPLNYVSKQKQNVFAQDLM | 218 | 2.618 |
| | | G36 | | FKQELEGSDIGLNFIHCDQFIQLPQLESPSLPLTKRPVSLTSITSLEKN | | 05379 |
| | | 160 | | KNIYKRHLIEEDVSFNALISSGNKDKKKKKTSVMTTDWRALDKFVASQL | | |
| | | | | MSQEDGVSGFGGHHEEDNNKIGHYNNEESNNKGSVETASSTLLSDREEE | | |
| | | | | NRFISGLLCSNLDYDLYRDLHV | | |
| 376 | 16 | AT3 | AP2- | ELASLFPRPASSSPHDIQTAAAEAAAMVVEEKLLEKDEAPEAPPSSESS | 119 | 2.625 |
| | | G16 | EREBP | YVAAESEDEERLEKIVELPNIEEGSYDESVTSRADLAYSEPFDCWVYPP | | 96943 |
| | | 280 | | VMDFYEEISEFNFVELWSFNH | | 2 |
| 377 | 352 | AT3 | NAC | NAPSTTITTTKQLSRIDSLDNIDHLLDFSSLPPLIDPGFLGQPGPSFSG | 167 | 2.662 |
| | | G04 | | ARQQHDLKPVLHHPTTAPVDNTYLPTQALNFPYHSVHNSGSDFGYGAGS | | 98037 |
| | | 060 | | GNNNKGMIKLEHSLVSVSQETGLSSDVNTTATPEISSYPMMMNPAMMDG | | 4 |
| | | | | SKSACDGLDDLIFWEDLYTS | | |
| 378 | 514 | AT1 | bZIP | MEGGGRGPNQTILSEIEHMPEAPRQRISHHRRARSETFFSGESIDDLLL | 193 | 2.696 |
| | | G43 | | FDPSDIDESSLDELNAPPPPQQSQQQPQASPMSVDSEETSSNGVVPPNS | | 65723 |
| | | 700 | | LPPKPEARFGRHVRSFSVDSDFFDDLGVTEEKFIATSSGEKKKGNHHHS | | 6 |
| | | | | RSNSMDGEMSSASFNIESILASVSGKDSGKKNMGMGGDRLAELALL | | |
| 379 | 61 | AT2 | AP2- | EITNRSSSTAATATVSGSVTAFSDESEVCAREDTNASSGFGQVKLEDCS | 213 | 2.701 |
| | | G40 | EREBP | DEYVLLDSSQCIKEELKGKEEVREEHNLAVGFGIGQDSKRETLDAWLMG | | 72159 |
| | | 340 | | NGNEQEPLEFGVDETFDINELLGILNDNNVSGQETMQYQVDRHPNFSYQ | | 6 |
| | | | | TQFPNSNLLGSLNPMEIAQPGVDYGCPYVQPSDMENYGIDLDHRRENDL | | |
| | | | | DIQDLDFGGDKDVHGST | | |
| 380 | 294 | AT1 | MYB | SSETNLNADEAGSKGSLNEEENSQESSPNASMSFAGSNISSKDDDAQIS | 156 | 2.704 |
| | | G16 | | QMFEHILTYSEFTGMLQEVDKPELLEMPFDLDPDIWSFIDGSDSFQQPE | | 28312 |
| | | 490 | | NRALQESEEDEVDKWFKHLESELGLEENDNQQQQQQHKQGTEDEHSSSL | | 6 |
| | | | | LESYELLIH | | |
| 381 | 12 | AT1 | AP2- | YSDMPRGSSVTSFVSPDESQRFISELFNPPSQLEATNSNNNNNNNLYSS | 150 | 2.853 |
| | | G28 | EREBP | TNNQNQNSIEFSYNGWPQEAECGYQSITSNAEHCDHELPPLPPSTCFGA | | 83935 |
| | | 160 | | ELRIPETDSYWNVAHASIDTFAFELDGFVDQNSLGQSGTEGENSLPSTF | | 3 |
| | | | | FYQ | | |
| 382 | 13 | AT1 | AP2- | EISTSLYHIINNGDNNNDMSPKSIQRVAAAAAAANTDPSSSSVSTSSPL | 119 | 2.877 |
| | | G44 | EREBP | LSSPSEDLYDVVSMSQYDQQVSLSESSSWYNCFDGDDQFMFINGVSAPY | | 77055 |
| | | 830 | | LTTSLSDDFFEEGDIRLWNFC | | 3 |
| 383 | 201 | AT5 | G2- | MTLANDEGYSTAMSSSYSALHTSVEDRYHKLPNSFWVSSGQELMNNPVP | 227 | 2.878 |
| | | G29 | like | CQSVSGGNSGGYLFPSSSGYCNVSAVLPHGRNLQNQPPVSTVPRDRLAM | | 06777 |
| | | 000 | | QDCPLIAQSSLINHHPQEFIDPLHEFFDFSDHVPVQNLQAESSGVRVDS | | 9 |
| | | | | SVELHKKSEWQDWADQLISVDDGSEPNWSELLGDSSSHNPNSEIPTPFL | | |
| | | | | DVPRLDITANQQQQMVSSEDQLSGRNSSSSV | | |
| 384 | 69 | AT5 | AP2- | YNPNAIPTSSSKLLSATLTAKLHKCYMASLQMTKQTQTQTQTQTARSQS | 117 | 2.878 |
| | | G25 | EREBP | ADSDGVTANESHLNRGVTETTEIKWEDGNANMQQNFRPLEEDHIEQMIE | | 22256 |
| | | 190 | (ESE3) | ELLHYGSIELCSVLPTQTL | | |
| 385 | 19 | AT4 | AP2- | LETVIKAMEMDCNPNYYRMNNSNTSDPLRSSRKIGLRTGKEAVKAYDEV | 135 | 2.921 |
| | | G18 | EREBP | VDGMVENHCALSYCSTKEHSETRGLRGSEETWFDLRKRRRSNEDSMCQE | | 91324 |
| | | 450 | | VEMQKTVTGEETVCDVFGLFEFEDLGSDYLETLLSSF | | 8 |
| 386 | 4 | AT3 | ABI3- | EEEEVDVINLEEDDVYTNLTRIENTVVNDLLLQDENHHNNNNNNNSNSN | 119 | 2.924 |
| | | G26 | VP1 | SNKCSYYYPVIDDVTTNTESFVYDTTALTSNDTPLDFLGGHTTTTNNYY | | 92207 |
| | | 790 | | SKFGTFDGLGSVENISLDDFY | | |
| | | (FUS3) | | | | |
| 387 | 59 | AT2 | AP2- | ELAYHLPRPASADPKDIQAAAAAAAAAVAIDMDVETSSPSPSPTVTETS | 93 | 2.938 |
| | | G35 | EREBP | SPAMIALSDDAFSDLPDLLLNVNHNIDGFWDSFPYEEPFLSQSY | | 38664 |
| | | 700 | | | | 9 |
| | | (ERF38) | | | | |
| 388 | 39 | AT1 | AP2- | KRDVSSSETSQCSRSSPVVPVEQDDTSASALTCVNNPDDVSTVAPTAPT | 154 | 2.964 |
| | | G68 | EREBP | PNVPAGGNKETLFDFDFTNLQIPDFGFLAEEQQDLDEDCFLADDQFDDE | | 63832 |
| | | 550 | | GLLDDIQGFEDNGPSALPDFDFADVEDLQLADSSFGFLDQLAPINISCP | | 8 |
| | | (CRF10) | | LKSFAAS | | |
| 389 | 41 | AT1 | AP2- | DSAWRLPVPESNDPDVIRRVAAEAAEMFRPVDLESGITVLPCAGDDVDL | 123 | 2.977 |
| | | G12 | EREBP | GFGSGSGSGSGSEERNSSSYGFGDYEEVSTTMMRLAEGPLMSPPRSYME | | 80166 |
| | | 610 | | DMTPTNVYTEEEMCYEDMSLWSYRY | | 7 |
| | | (DDF1) | | | | |
| 390 | 464 | AT2 | WRKY | TCNNITSPKTTTNFSVSLTNTNIFEGNRVHVTEQSEDMKPTKSEEVMIS | 134 | 2.978 |
| | | G46 | | LEDLENKKNIFRTFSFSNHEIENGVWKSNLFLGNFVEDLSPATSGSAIT | | 14662 |
| | | 400 | | SEVLSAPAAVENSETADSYFSSLDNIIDFGQDWLWS | | 5 |
| | | (WRKY46) | | | | |
| 391 | 34 | AT4 | AP2- | DSAWRLRIPESTCAKDIQKAAAEAALAFQDETCDTTTTNHGLDMEETMV | 109 | 3.009 |
| | | G25 | EREBP | EAIYTPEQSEGAFYMDEETMFGMPTLLDNMAEGMLLPPPSVQWNHNYDG | | 66593 |
| | | 490 | | EGDGDVSLWSY | | 1 |
| | | (CBF1) | | | | |
| 392 | 35 | AT4 | AP2- | DSAWRLRIPESTCAKEIQKAAAEAALNFQDEMCHMTTDAHGLDMEETLV | 109 | 3.047 |
| | | G25 | EREBP | EAIYTPEQSQDAFYMDEEAMLGMSSLLDNMAEGMLLPSPSVQWNYNFDV | | 16588 |
| | | 470 | | EGDDDVSLWSY | | 7 |
| | | (CBF2) | | | | |
| 393 | 499 | AT1 | bHLH | MQSTHISGGSSGGGGGGGGEVSRSGLSRIRSAPATWIETLLEEDEEEGL | 181 | 3.173 |
| | | G35 | | KPNLCLTELLTGNNNSGGVITSRDDSFEFLSSVEQGLYNHHQGGGFHRQ | | 52894 |
| | | 460 | | NSSPADFLSGSGSGTDGYESNFGIPANYDYLSTNVDISPTKRSRDMETQ | | 5 |
| | | (FBH1) | | FSSQLKEEQMSGGISGMMDMNMDKIFEDSVPCRV | | |
| 394 | 48 | AT1 | AP2- | TSSSSHHLLDNLLDENTLLSPKSIQRVAAQAANSENHFAPTSSAVSSPS | 124 | 3.225 |
| | | G21 | EREBP | DHDHHHDDGMQSLMGSFVDNHVSLMDSTSSWYDDHNGMFLEDNGAPFNY | | 37532 |
| | | 910 | | SPQLNSTTMLDEYFYEDADIPLWSEN | | 4 |
| | | (DREB26) | | | | |
| 395 | 369 | AT5 | NAC | NGLGPRHGSQYGAPFKEEDWSDKEEEYTQNHLVAGPSKETSLAAKASHS | 200 | 3.250 |
| | | G64 | | YAPKDGLTGVISESCVSDVPPLTATVLPPLTSDVIAYNPFSSSPLLEVP | | 64405 |
| | | 060 | | QVSLDGGELNSMLDLFSVDNDDCLLFDDFDYHNEVRHPDGFVNKEAPVF | | 7 |
| | | (NAC103) | | LGDGNFSGMFDLSNDQVVELQDLIQSPTPHPPSPPAQASIPDDSRSNGQ | | |
| | | | | TKDD | | |
| 396 | 368 | AT5 | NAC | NEIKTNTKIRKIPSEQTIGSGESSGLSSRVTSPSRDETMPFHSFANPVS | 134 | 3.321 |
| | | G46 | | TETDSSNIWISPEFILDSSKDYPQIQDVASQCFQQDFDFPIIGNQNMEF | | 52637 |
| | | 590 | | PASTSLDQNMDEFMQNGYWTNYGYDQTGLFGYSDES | | 5 |
| | | (NAC096) | | | | |
| 397 | 73 | AT5 | AP2- | YTPTDVHTILTNPNLHSLIVSPYNNNQSFLPNSSPQFVIDHHPHYQNYH | 237 | 3.334 |
| | | G18 | EREBP | QPQQPKHTLPQTVLPAASFKTPVRHQSVDIQAFGNSPQNSSSNGSLSSS | | 73788 |
| | | 560 | | LDEENNFFFSLTSEEHNKSNNNSGYLDCIVPNHCLKPPPEATTTQNQAG | | 4 |
| | | (PUCHI) | | ASFTTPVASKASEPYGGFSNSYFEDGEMMMMNHHEFGSCDLSAMITNYG | | |
| | | | | AAAASMSMEDYGMMEPQDLSSSSIAAFGDVVADTTGFYSVF | | |
| 398 | 20 | AT1 | AP2- | DNPPVISGGRNLSRSEIREAAARFANSAEDDSSGGAGYEIRQESASTSM | 117 | 3.722 |
| | | G19 | EREBP | DVDSEFLSMLPTVGSGNFASEFGLFPGEDDESDEYSGDRFREQLSPTQD | | 76030 |
| | | 210 | | YYQLGEETYADGSMFLWNF | | 8 |
| | | (ERF017) | | | | |
| 399 | 14 | AT1 | AP2- | ELASSLPRPADSSSDSIRMAVHEATLCRTTEGTESAMQVDSSSSSNVAP | 103 | 3.799 |
| | | G71 | EREBP | TMVRLSPREIQAINESTLGSPTTMMHSTYDPMEFANDVEMNAWETYQSD | | 33650 |
| | | 450 | | FLWDP | | 1 |
| | | (FUF1) | | | | |
| 400 | 37 | AT5 | AP2- | DSAWRLRIPETTCPKEIQKAASEAAMAFQNETTTEGSKTAAEAEEAAGE | 114 | 3.863 |
| | | G51 | EREBP | GVREGERRAEEQNGGVFYMDDEALLGMPNFFENMAEGMLLPPPEVGWNH | | 08175 |
| | | 990 | | NDFDGVGDVSLWSFDE | | 2 |
| | | (CBF4) | | | | |
| 401 | 105 | AT3 | C2C2- | PKSSSGNNTKTSLTANSGNPGGGSPSIDLALVYANFLNPKPDESILQEN | 168 | 3.867 |
| | | G52 | DOF | CDLATTDFLVDNPTGTSMDPSWSMDINDGHHDHYINPVEHIVEECGYNG | | 64889 |
| | | 440 | | LPPFPGEELLSLDTNGVWSDALLIGHNHVDVGVTPVQAVHEPVVHFADE | | 4 |
| | | (DOF3.5) | | SNDSTNLLFGSWSPFDFTADG | | |
| 402 | 68 | AT3 | AP2- | HEYQMMKDGPNGSHENAVASSSSGYRGGGGGDDGREVIEFEYLDDSLLE | 68 | 3.963 |
| | | G23 | EREBP | ELLDYGERSNQDNCNDANR | | 17332 |
| | | 220 | | | | 2 |
| | | (ESE1) | | | | |
| 403 | 187 | AT2 | G2- | MIPNDDDDANSMKNYPLNDDDANSMKNYPLNDDDANSMENYPLRSIPTE | 227 | 3.968 |
| | | G20 | like | LSHTCSLIPPSLPNPSEAAADMSENSELNQIMARPCDMLPANGGAVGHN | | 80251 |
| | | 400 | | PFLEPGFNCPETTDWIPSPLPHIYFPSGSPNLIMEDGVIDEIHKQSDLP | | 4 |
| | | (PHL4) | | LWYDDLITTDEDPLMSSILGDLLLDTNFNSASKVQQPSMQSQIQQPQAV | | |
| | | | | LQQPSSCVELRPLDRTVSSNSNNNSNSNNAA | | |

A synthetic transcription factor (TF) comprising (a) a DNA-binding domain of a transcription factor linked to (b) an activator domain or repressor domain, and (c) a nuclear localization sequence (NLS).

In some embodiments, the DNA-binding domain is a DNA-binding domain of a eukaryotic TF or a prokaryotic TF.

In some embodiments, the DNA-binding domain is a DNA-binding domain of a eukaryotic TF.

In some embodiments, the eukaryotic TF is a yeast TF. In some embodiments, the yeast TF is a Saccharomyces TF. In some embodiments, the Saccharomyces TF is a Saccharomyces cerevisiae TF. In some embodiments, the S. cerevisiae TF is Ga14, YAP1, GAT1, MATAL1, MATAL2, MCM1, Abf1, Adr1, Ash1, Gcn4, Gcr1, Hap4, Hsf1, Ime1, Ino2/Ino4, Leu3, Lys14, Mata2, Mga2, Met4, Mig1, Rap1, Rgt1, Rlm1, Smp1, Rme1, Rox1, Rtg3, Spt23, Teal, Ume6, or Zap1. In some embodiments, the S. cerevisiae TF is Ga14, YAP1, GAT1, MATAL1, MATAL2, MCM1, or Rap1.

In some embodiments, the synthetic TF comprises the activator domain which is a herpes simplex virus VP16, maize C1, or a yeast activator domain.

In some embodiments, the activator domain is the yeast activator domain. In some embodiments, the yeast activator domain is a Saccharomyces activator domain. In some embodiments, the Saccharomyces activator domain is a Saccharomyces cerevisiae activator domain.

In some embodiments, the S. cerevisiae activator domain is a Ga14, YAP1, GAT1, MATAL1, MATAL2, MCM1, Abf1, Adr1, Ash1, Gcn4, Gcr1, Hap4, Hsf1, Ime1, Ino2/Ino4, Leu3, Lys14, Mga2, Met4, Rap1, Rlm1, Smp1, Rtg3, Spt23, Tea1, Ume6, or Zap1 activator domain.

In some embodiments, the synthetic TF comprises the repressor domain. In some embodiments, the repressor domain comprises an EAR motif, TLLLFR motif, R/KLFGV motif, LxLxPP motif, or a yeast repressor domain.

In some embodiments, the yeast repressor domain is a Saccharomyces repressor domain. In some embodiments, the Saccharomyces repressor domain is a Saccharomyces cerevisiae repressor domain. In some embodiments, the S. cerevisiae repressor domain is an Ash1, Mata2, Mig1, Rap1, Rgt1, Rme1, Rox1, or Ume6 repressor domain.

In some embodiments, the NLS is monopartite or bipartite. In some embodiments, the NLS comprises a M9 domain or PY-NLS motif. In some embodiments, the NLS comprises the amino acid sequence KIPIK (yeast Mata2).

In some embodiments, any two, or all, of the DNA-binding domain, the activator domain, the repressor domain, and the NLS are heterologous to each other.

In some embodiments, the dCas9 comprises the following amino acid sequence:

(SEQ ID NO: 439)

```
                10 20 30 40
MDKKYSIGLA IGTNSVGWAV ITDEYKVPSK KFKVLGNTDR
                50 60 70 80
HSIKKNLIGA LLFDSGETAE ATRLKRTARR RYTRRKNRIC
                90 100 110 120
YLQEIFSNEM AKVDDSFFHR LEESFLVEED KKHERHPIFG
              130 140 150 160
NIVDEVAYHE KYPTIYHLRK KLVDSTDKAD LRLIYLALAH
              170 180 190 200
MIKFRGHFLI EGDLNPDNSD VDKLFIQLVQ TYNQLFEENP
              210 220 230 240
INASGVDAKA ILSARLSKSR RLENLIAQLP GEKKNGLFGN
              250 260 270 280
LIALSLGLTP NFKSNFDLAE DAKLQLSKDT YDDDLDNLLA
              290 300 310 320
QIGDQYADLF LAAKNLSDAI LLSDILRVNT EITKAPLSAS
              330 340 350 360
MIKRYDEHHQ DLTLLKALVR QQLPEKYKEI FFDQSKNGYA
              370 380 390 400
GYIDGGASQE EFYKFIKPIL EKMDGTEELL VKLNREDLLR
              410 420 430 440
KQRTFDNGSI PHQIHLGELH AILRRQEDFY PFLKDNREKI
              450 460 470 480
EKILTFRIPY YVGPLARGNS RFAWMTRKSE ETITPWNFEE
              490 500 510 520
VVDKGASAQS FIERMTNEDK NLPNEKVLPK HSLLYEYFTV
              530 540 550 560
YNELTKVKYV TEGMRKPAFL SGEQKKAIVD LLFKTNRKVT
              570 580 590 600
VKQLKEDYFK KIECFDSVEI SGVEDRENAS LGTYHDLLKI
              610 620 630 640
IKDKDFLDNE ENEDILEDIV LTLTLFEDRE MIEERLKTYA
              650 660 670 680
HLEDDKVMKQ LKRRRYTGWG RLSRKLINGI RDKQSGKTIL
              690 700 710 720
DFLKSDGFAN RNFMQLIHDD SLTFKEDIQK AQVSGQGDSL
              730 740 750 760
HEHIANLAGS PAIKKGILQT VKVVDELVKV MGRHKPENIV
              770 780 790 800
IEMARENQTT QKGQKNSRER MKRIEEGIKE LGSQILKEHP
              810 820 830 840
VENTQLQNEK LYLYYLQNGR DMYVDQELDI NRLSDYDVDA
              850 860 870 880
IVPQSFLKDD SIDNKVLTRS DKNRGKSDNV PSEEVVKKMK
              890 900 910 920
NYWRQLLNAK LITQRKEDNL TKAERGGLSE LDKAGFIKRQ
              930 940 950 960
LVETRQITKH VAQILDSRMN TKYDENDKLI REVKVITLKS
              970 980 990 1000
KLVSDERKDF QFYKVREINN YHHAHDAYLN AVVGTALIKK
            1010 1020 1030 1040
YPKLESEFVY GDYKVYDVRK MIAKSEQEIG KATAKYFFYS
            1050 1060 1070 1080
NIMNFFKTEI TLANGEIRKR PLIETNGETG EIVWDKGRDF
            1090 1100 1110 1120
ATVRKVLSMP QVNIVKKTEV QTGGFSKESI LPKRNSDKLI
            1130 1140 1150 1160
ARKKDWDPKK YGGFDSPTVA YSVLVVAKVE KGKSKKLKSV
            1170 1180 1190 1200
KELLGITIME RSSFEKNPID FLEAKGYKEV KKDLIIKLPK
            1210 1220 1230 1240
YSLFELENGR KRMLASAGEL QKGNELALPS KYVNFLYLAS
            1250 1260 1270 1280
HYEKLKGSPE DNEQKQLFVE QHKHYLDEII EQISEFSKRV
            1290 1300 1310 1320
ILADANLDKV LSAYNKHRDK PIREQAENII HLFTLTNLGA
            1330 1340 1350 1360
PAAFKYFDTT IDRKRYTSTK EVLDATLIHQ SITGLYETRI
DLSQLGGD
```

In some embodiments, one or more, or all, of the DNA-binding domain, the activator domain, the repressor domain, and the NLS are obtained or derived from a non-viral organism.

In some embodiments, the DNA-binding domain, the NLS, and the activator domain or repressor domain are linked in this order from N- to C-terminus.

A nucleic acid encoding the synthetic TF of any one of claims 1-54 operatively linked to a promoter capable of expressing the synthetic TF in vitro or in vivo.

A vector comprising the nucleic acid of the present invention.

In some embodiments, the vector is capable of stably integrating into a chromosome of a host cell or stably residing in a host cell.

In some embodiments, the vector is an expression vector.

A host cell comprising the vector of the present invention, wherein the host cell is capable of expressing the synthetic TF.

A system comprising a nucleic acid of the present invention and a second nucleic acid, or the nucleic acid, encodes a gene of interest (GOI) operatively linked to a promoter and one or more activator/repressor binding domains, or combination thereof, wherein the synthetic TF binds at least one of the one or more activator/repressor binding domain such that the synthetic TF modulates the expression of the GOI.

A genetically modified eukaryotic cell or organism, such as a plant cell or plant, comprising: (a) (i) one or more nucleic acids each encoding one or more transcription activators operatively linked to a first promoter, (ii) one or more nucleic acids each encoding one or more transcription repressors each operatively linked to a second promoter, or (iii) combinations thereof; and (b) one or more nucleic acids each encoding one or more independent genes of interest (GOI) each operatively linked to a promoter that is activated by the one or more transcription activators, repressed by the one or more transcription repressors, or a combination of both; wherein at least one transcription activator or transcription repressor is a synthetic transcription factor (TF) of the present invention.

In some embodiments, the first promoter, the second promoter, or both, is a tissue-specific or inducible promoter.

In some embodiments, the transcription activator is the synthetic TF.

In some embodiments, the transcription repressor is the synthetic TF.

In some embodiments, any domain of the synthetic TF is heterologous to the eukaryotic cell or organism, such as a plant cell or plant, one or more of the GOI, any other transcription activator or transcription repressor, and/or any of the promoters.

In some embodiments, the transcription activator is heterologous to the eukaryotic cell or organism, such as a plant cell or plant, one or more of the GOI, any other or transcription activator, transcription repressor, and/or any of the promoters.

In some embodiments, the transcription repressor is heterologous to the eukaryotic cell or organism, such as a plant cell or plant, one or more of the GOI, any other transcription activator, and/or any of the promoters.

In some embodiments, the genetically modified plant cell or plant comprises: (a) a first nucleic acid encoding a transcription activator operatively linked to a first tissue-specific or inducible promoter, (b) optionally a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription activators, repressed by the transcription repressors, or a combination of both.

In some embodiments, the genetically modified plant cell or plant comprises: (a) optionally a first nucleic acid encoding a transcription activator operatively linked to a first tissue-specific or inducible promoter, (b) a second nucleic acid encoding a transcription repressor operatively linked to a second tissue-specific or inducible promoter; and (c) one or more nucleic acids each encoding one or more independent genes of interest (GOI) each operatively linked to a promoter that is activated by the transcription activators, repressed by the transcription repressors, or a combination of both.

In some embodiments, each GOI is operatively linked to a promoter that is activated by the transcription activator, repressed by the transcription repressors, or a combination of both.

In some embodiments, the promoter comprises one or more DNA-binding sites specific for the transcription activator, one or more DNA-binding sites specific for the transcription repressor, or a combination of both.

In some embodiments, the promoter comprises 1, 2, 3, 4, 5, 6, 7, 8, 9, or 10 DNA-binding sites specific for the transcription activator), 1, 2, 3, 4, 5, 6, 7, 8, 9, or 10 DNA-binding sites specific for the transcription repressor, or a combination of both.

In some embodiments, the eukaryotic cell or organism is a plant cell or plant. In some embodiments, the eukaryotic cell or organism is a yeast. In some embodiments, the yeast is Saccharomyces species, such as a Saccharomyces cerevisiae.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Determination of Genome Wide Transcriptional Effector Activity Elucidates Functional Dynamics of Plant Gene Regulatory Networks

The effector domains of transcription factors play a key role in controlling gene expression, however, their regulatory and functional nature are poorly understood, hampering our ability to understand a fundamental dimension of gene regulatory networks. To explore the trans-regulatory landscape in plants, the putative effector domains of over 400 Arabidopsis thaliana transcription factors are systematically characterized for their capacity to modulate transcription, providing insight into both the biochemical basis of plant transcriptional regulation and the convergence of broader network motifs. By integrating effector activity into transcriptional networks the missing functional interactions needed to elucidate the underlying wiring of biological systems are provided. Finally, plant activators to enhance Cas9-based genome engineering tools are utilized and reveal how plant activators utilize a general eukaryotic mechanism for activation.

Modulating the expression of plant genes has been a key area of focus for precision crop engineering, as many agronomically important traits are the result of altered gene expression (7, 8). The intrinsic trans-regulatory elements embedded in plant TF proteins offer a unique resource to mine for novel effector domains that may advance plant engineering efforts. To expand the understanding of plant transcriptional regulation, the activation and/or repression activity of putative effector domains from over 400 A. thaliana TFs are systematically measured, providing unique insights into the underlying biochemical properties of plant effectors and their functional role in network motifs. The resulting library of effector domains established in this Example demonstrate how genome-wide functional characterization of TF regulatory domains can enhance the understanding of the transcriptional regulation of biological systems, both on a biochemical and systems level.

### RESULTS

### Genome-Wide Identification of Effector Domains Elucidates Biochemical Trends Underlying Plant Gene Regulation

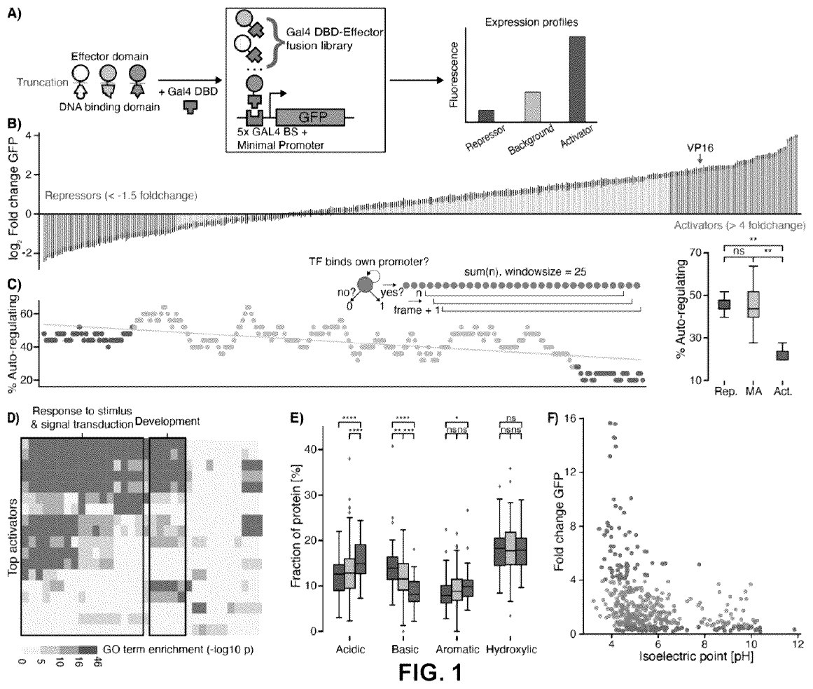

The DNA binding activity of 529 A. thaliana TFs has been previously studied but the lack of a large scale characterization of effector activity, hampered the understanding of plant gene regulation and circuitry. The effector domains of a large set of A. thaliana TFs whose DNA binding motifs and downstream targets had previously been mapped (1) is experimentally characterized. Putative effector domains are selected by identifying sequences in the Arabidopsis TF domains adjacent to conserved DNA binding domains, and fused the resulting sequences to the yeast Gal4 DBD (Supplementary Table 1). The Gal4 DBD localizes the effector candidate to a minimal promoter with 5 concatenated Gal4 binding sites driving the fluorescent reporter GFP, a system that was established previously (Belcher et al. 2020). By reading out modulation of GFP one can individually characterize the effector domain independent of its regular genomic context. Using this approach 403 synthetic TFs are individually characterized using a transient expression system in Nicotiana benthamiana. (FIG. 1, Panel A). 69 activator domains are identified that increased GFP expression by at least 400% and 72 repressor domains are identified which reduced GFP expression by at least 65% in comparison to basal expression of the reporter (Supplementary Table 2). 53 activators are found displaying stronger trans-activation than the benchmark viral activator VP16, with the strongest activator derived from PHL4 (PHL4-Eff), achieving 236% higher activation than VP16, a 16-fold increase of GFP expression (FIG. 1, Panel B). These findings demonstrate the potential of well-characterized endogenous parts (e.g., effector domains) for the development of enhanced genetic engineering tools, providing alternatives to broadly used effector domains like VP16, and the development of stronger effector domains in various biological systems.

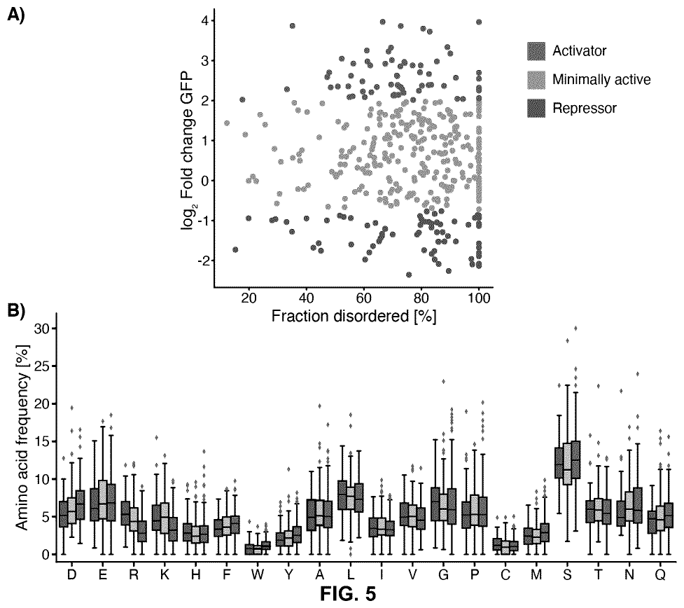

TFs lack significant sequence conservation outside their DBDs both within and between TF families. As a result, most effectors lack known sequence motifs explaining their activity (11, 12). Analysis of these putative effector domains with VSL2, a predictor of intrinsic disorder in proteins (Peng et al. 2006), predicted on average 75% of residues to be intrinsically disordered (FIG. 5, Panel A), in agreement with analyses of eukaryotic effector domains (13). It has been previously demonstrated that acidic residues in combination with hydrophobic clusters are essential for activator activity, promoting transcription by forming a protein interface with the Mediator complex (6, 14-16). With an effector screen, one sought to investigate the biochemical properties underlying effector activity. It is found that there are biases in amino acid composition both in the repressor and activator populations (FIG. 1, Panel E). Notably, among activators acidic and hydrophobic residues are significantly overrepresented and basic residues (e.g arginine, lysine and histidine) were significantly depleted. Hydrophobic, aromatic residues are also overrepresented in the activator population supporting the necessity of these residues for activator activity (FIG. 5, Panel C). For repressors, only arginine is significantly overrepresented, indicating its role as an important residue for plant repressor activity (17)).

Given the importance of charged residues on effector activity (18), the isoelectric point of each effector is compared to its performance in our screen. It is observed that effectors in the activator population tend to show lower isoelectric points than both repressor and the minimally active populations, suggesting that the overall charge of a sequence may play a role for activator activity (FIG. 1, Panel F). In comparison, it is found that repressors with a wide range of isoelectric points perhaps reflecting the underlying complexity of transcriptional repression which can be mediated through several disparate mechanisms (e.g., chromatin modification, recruitment of corepressors) (19, 20, 21). This functional characterization of over 400 plant effector domains provides the aggregate data required to begin to elucidate the biochemical trends underlying transcription and provides a basis for future studies of effector domains in gene regulation.

### Characterization of Effector Function Reveals Emergence of Genome-Wide Transcriptional Network Motifs

Biological systems do not organize their transcriptional networks randomly, but rather have converged recurring network motifs to enable disparate forms of regulation (22). Large scale TF-DNA binding studies have been used to identify network motifs (23), and effector activity integration has the potential to complete the information encoded in these motifs.

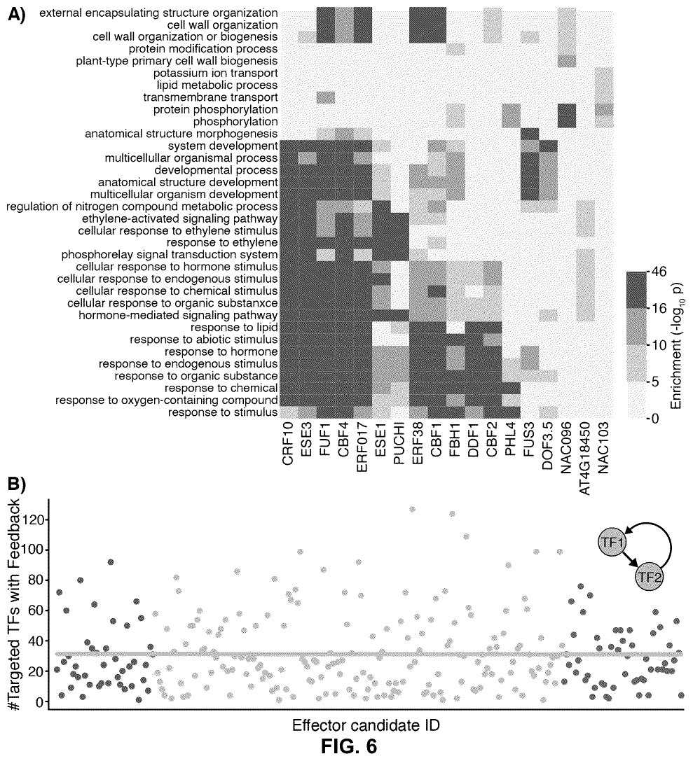

A widely observed network motif is the phenomenon of negative autoregulation (NAR), where a repressor downregulates its own expression (24). NAR enables the acceleration of response times and reduces cell-to-cell variation in protein concentration thus enabling robust regulation of their targets (22, 25). To investigate usage of NAR in plant TFs, effector activity is combined with published DNA binding data (1). A binary value is assigned to each TF based on whether the TF binds its own promoter region (1=Binding, 0=No binding). The binary values for all TFs screened are arranged based on the effector activity measured and summarized the values for each sliding-window of 25 TFs from repression to activation (FIG. 1, Panel C). We found autoregulation to be more prominent in repressors than in activators, consistent with observations in prokaryotes (24), demonstrating NAR as a genome-wide logic for transcriptional control in plants (p=0.008, Mann-Whitney-U test). Feedback loops, i.e., two TFs regulating each other, also searched for, but any differences between activators and repressors is not observed (FIG. 6, Panel B).

The wide range of effector activity raises the question where strong effectors reside within GRNs, as strong TF effector activity can lead to developmental decision making and could destabilize the transcriptome. To study the position of strong activators inside the GRN the gene ontology (GO) terms of genes targeted by these TFs is analyzed. Interestingly, it is found that the GO terms of these direct target genes are enriched for terms linked to signal transduction and response to hormones, stresses, external stimuli, and development and depleted in GO terms linked to primary or secondary metabolism (FIG. 1D, fully annotated figure, FIG. 6, Panel A). This suggests that strong plant activators are more likely to be situated inside signaling cascades than activating metabolic pathway genes, highlighting a requirement for strong gene activation to enact the rapid changes to transcriptional programming needed for a concerted response to stimuli.

### Mapping the Functional Dynamics of Plant Transcriptional Networks

Unraveling the functional dynamics of GRNs is a key challenge of systems biology with the promise to decode the concerted, genome-wide responses of biological systems to environmental cues. Novel approaches have utilized time-series experiments to understand the dynamics of TFs and their targets in temporal GRNs. Still, these updated GRNs try to infer TF activity based on the RNA level of genes targeted by said TF, due to the missing knowledge on how TF effector activity translates into the modulation of gene expression. Thus, it is sought to bridge this gap by incorporating this effector characterization data into previously established GRNs, adding causality to gene expression patterns after TF interaction.

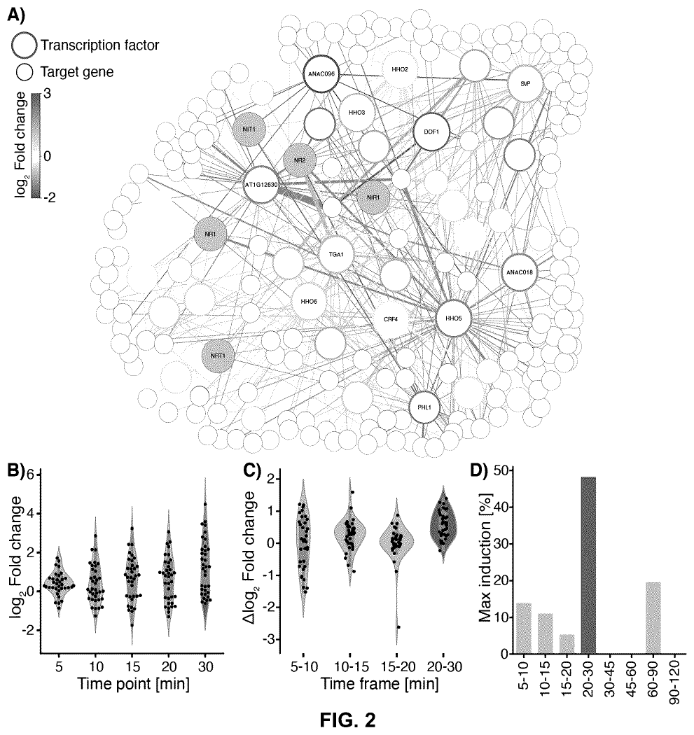

The transcriptional response to nitrate has been thoroughly studied in A. thaliana (5), providing an ideal case study for incorporating our effector data. The functional dynamics in a published GRN describing the temporal transcriptional responses to nitrate availability in A. thaliana is investigated (4). The links between TFs and their targets as activating or repressing are annotated, thereby generating the first GRN integrating effector activity data with published DNA binding data and temporal RNA-seq co-expression analysis for 37 TFs and 171 direct genomic targets, all responsive to the presence of nitrate (FIG. 2A, Table 1). The temporal aspect of this GRN allows one to study how the expression of TFs at specific time points influences target genes during the response.

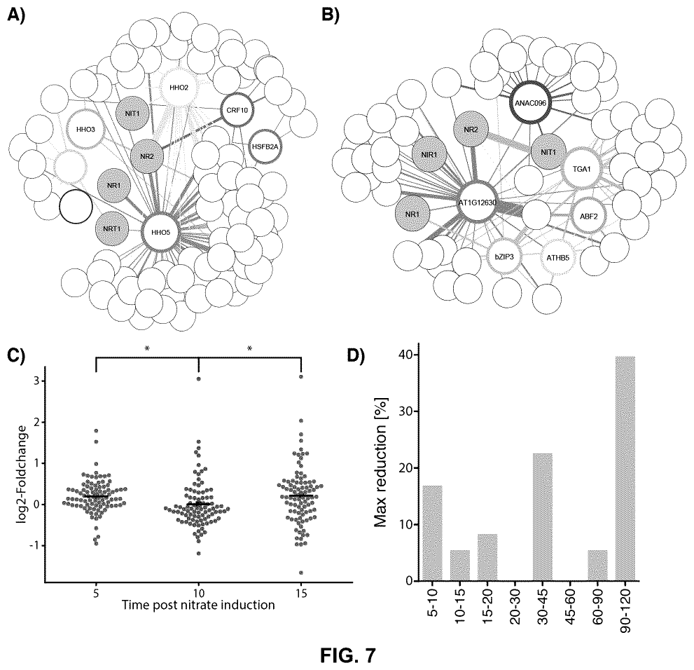

The response to nitrate alters gene expression within the first 20 minutes of the response (26) and more than 100 TFs are active over the course of 120 min which could make the analysis over the entire time frame difficult as more and more TFs can interfere with the observations. Therefore the early nitrogen response between 0-30 min is focused on. Subnetworks of induced TFs relative to baseline at 0 mins and their respective targets 10 and 15 minutes post nitrate induction are extracted. Most TFs expressed at 10 mins have repressor activity according to the screen and members from the HRSI/HHO repressor family (namely HHO2/5/6), which are known to control the nitrogen utilization by repression (27, 28), are overrepresented. This suggests that the network initiates its response with a burst of repression. To support this claim, the expression of all genes in the GRN is compared and a significant reduction of gene expression at 10 min compared to both at 5 min and 15 min post induction (p <0.005, two-sided Mann-Whitney U test, FIG. 7, Panel C) is found, demonstrating how effector activity can translate into biological observation.

At 15 minutes post nitrate induction, a set of six activators which target primary nitrate response genes (nitrate reductase 1 and 2 (NR1/2), and nitrite reductase 1 (NIT1)) (FIG. 7, Panel B) is identified and annotated. If the annotated effector activity for these TFs indeed overlays with in vivo function, one should be able to observe a spike of expression in genes targeted by this group. The expression profiles for all target genes at every time point (FIG. 2, Panel B) is visualized and calculated the rate of expression change in between every time point (FIG. 2, Panel C). Indeed, it is found that in between 20 and 30 min the majority of genes in the 15 min sub network shows their largest rate of expression increase (FIG. 2, Panel D), and no gene shows its strongest deceleration of expression (FIG. 7, Panel D). This suggests that effector activity observed in the assay can predict their in vivo transcriptional output, priming these TFs for further study (FIG. 2, Panel C). Importantly, NR1 shows its highest rate of induction between 20 and 30 min (FIG. 7, Panel E), implying the importance of the interacting activators bZIP3 and AT1G12630. Only bZIP3 has been linked to nitrogen signaling (29), marking the unnamed and unstudied TF AT1G12630 as a target for future studies in nitrate response.

Network motifs can simplify GRNs and display gene circuits that describe the functional dynamics underlying the network as a whole. One such motif is the single-input module, describing one TF targeting multiple genes downstream. This behavior for genes targeted by TFs from the 10 and 15 min subnetwork is studied by only observing genes targeted by a single activator or single repressors characterized by the screen. It is found that genes targeted by single activators are more likely to show increased expression at later time points than genes targeted by single repressors (FIG. 7, Panel C). This demonstrates the causal link between effector activity and transcriptional output, highlighting the potential mechanistic insights one can achieve with this analysis and marking these links as potential targets for bioengineering efforts.

This GRN represents an important step in systems biology, where integrated effector activity can help elucidate both the dynamics of GRN response as well as the location of TFs with strong regulatory activity inside a signaling cascade hierarchy. These observations suggest that nitrogen signaling is initiated through coordinated gene repression before a burst of activation of genes inside the pathway. Hence, effector characterization provides an important means to fill in major gaps in the knowledge of GRNs that top-down observations have been unable to resolve and a full genome coverage characterization of effector domains will be critical to providing a holistic understanding of global transcriptional regulation.

### Novel Plant Activators Boost Performance of Gene Expression Systems

Having shown that effector activity can be effectively incorporated into GRNs, it is aimed to explore the potential of our effector set in synthetic biology, which aims to control gene expression robustly and with a dynamic range of expression profiles. Previously developed plant synthetic biology tools have relied on a small subset of characterized effectors, especially the herpes simplex virus-based VP16 domain, which has been the state-of-the-art activator since its discovery over 30 years ago (30-32). Moreover, prior studies have demonstrated that different classes of activators may provide different levels of activity when working in conjunction with other co-activators or specific promoters (33). Consequently, these characterized effectors provide the opportunity to mine for plant-specific activator domains that can increase expression strength beyond the state-of-the-art VP16 domains that are commonly used in genome engineering approaches (e.g., dCas9-based CRISPR activation, synthetic transcription factors, etc).

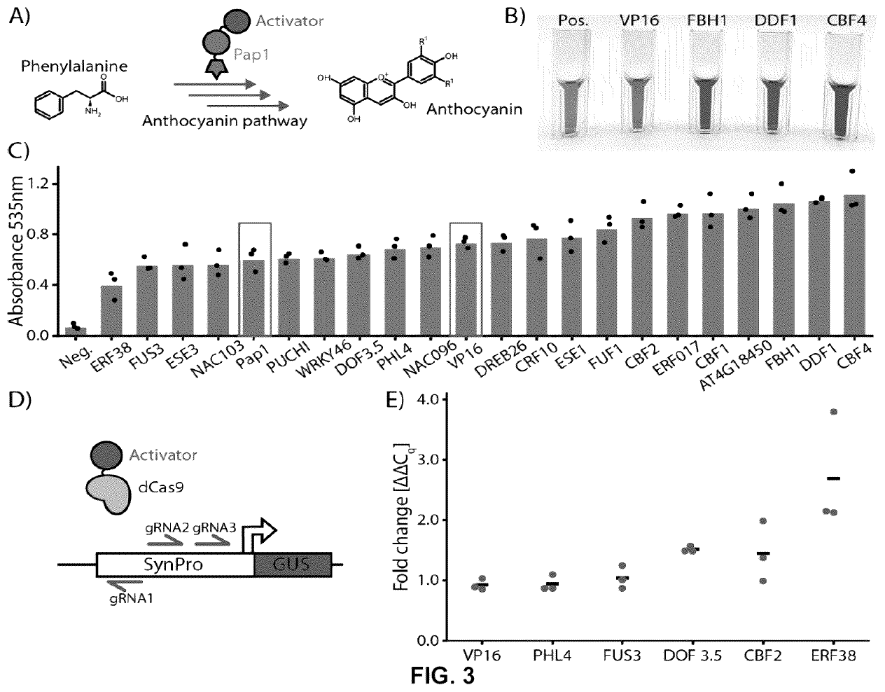

To explore the transferability of the qualitative biological activity of effectors, the activator domains are fused to other TFs to test their means to enhance the transcriptional output. The anthocyanin master regulator PAP1 is targeted as it activates the expression of multiple anthocyanin pathway genes resulting in a quantitative readout via elevated levels of anthocyanins in plant tissue ((34), FIG. 3, Panel A). PAP1-effector fusions are expressed in N. benthamiana for 3 days and quantified the anthocyanin content by absorbance measurements. Multiple activators show increased expression of anthocyanins in comparison to PAP1 and a PAP1-VP16 fusion (FIG. 3, Panels B and C). Of 20 activator candidates, 8 display significantly higher absorbance values than PAP1 and 7 higher than PAP1-VP16 (two-sided Student's t-test, p<0.05, Supplementary Table 4). It is demonstrate that the panel of top activator domains may be broadly applicable as a means to screen and optimize the transcriptional output of target TFs by directly fusing and engineering TFs with various strong activator domains.

Fusions of activators to a deactivated RNA-guided nuclease variant of Cas9 (dCas9) can alter gene expression in a modular manner when selectively defined by engineered guide RNAs (35, 36). The versatility of the DNA binding capability of dCas9-effector constructs has been leveraged to enable genome wide CRISPR activation screens, but again have mostly relied on VP16-based viral activators ((32), (36)). Hence it is sought to benchmark the top activator candidates against VP16. We fused the five strongest activators found in our screen to dCas9 and compared these novel dCas9-effector fusions to dCas9-VP16 by targeting them to a synthetic promoter (FIG. 3D). Transcript abundance is quantified by qRT-PCR with RNA extracted from N. benthamiana leaf tissue 3 days post Agrobacterium transformation. It is observed that dCas9-VP16 display extremely low activity in comparison to two activator domains from ERF38 (p=0.0336) and DOF3.5 (p=0.0006, FIG. 3E, SI Table 5). The larger genome engineering field has embraced the use of VP16 based activators, and has largely coped with its low activation activity by recruiting large numbers of VP16 via various strategies (i.e., suntag, MS2, refs). As an alternative, this effector screen demonstrates how identification of entirely novel, host-specific effector domains can result in an increased dynamic range of gene expression, and decrease reliance on effectors that are not optimized to work in plants like VP16. Ultimately, this genome-wide screen enable one to identify strong activator domains that can be used to tunably enhance transcription in a genome-specific manner, thereby providing a foundation for rapid generation of functional genomics toolsets.

### Conserved Mechanisms in Transcriptional Activation Across Eukaryotes

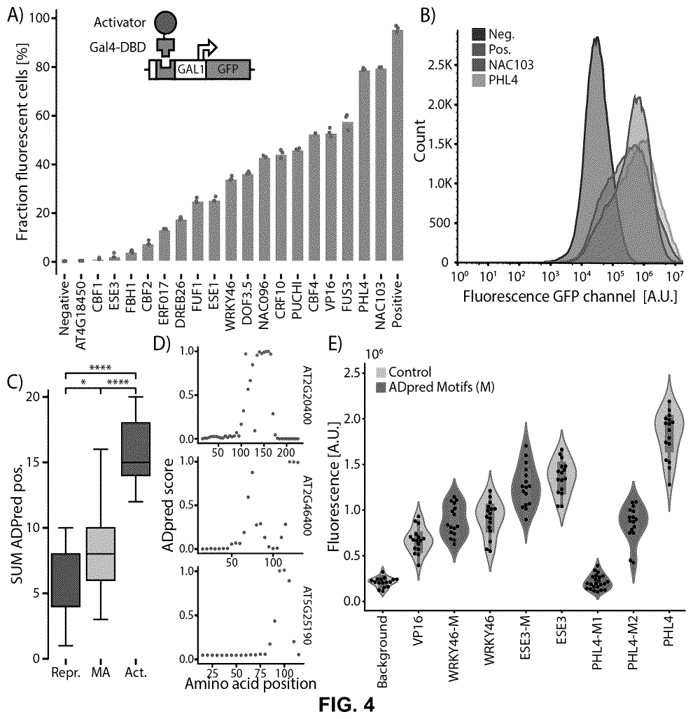

Just as the function of VP16 can cross eukaryotic super families, transcriptional activation may utilize molecular machinery and mechanisms broadly conserved between distantly related species. In order to investigate the potential in translating our newly identified plant activator domains into other eukaryotes, we tested the ability of our twenty strongest activators to promote constitutive gene expression in the model fungal system, Saccharomyces cerevisiae. An expression cassette is designed utilizing the well-characterized yeast inducible GAL1 promoter, which is induced in presence of galactose, repressed by glucose and contains Gal4 binding sites (37), driving the fluorescent reporter GFP. It is then observed the ability of Ga14-DBD-effector fusions to induce gene expression using flow cytometry (FIG. 4, Panel A). TF activity is quantified by measuring the fractions of cells overlapping with the gate of GAL1-GFP induced by galactose, while excluding observations that fall into the gate of GAL1-GFP in glucose. When the Gal4-DBD-effector fusions are expressed constitutively, GFP expression is observed in 80% to <1% of the cell populations (FIG. 4, Panel A, Supplementary Table 6). Notably, NAC103-Eff and PHL4-Eff are able to outperform VP16, making them strong candidates for further optimization in fungi (FIG. 4, Panel B). The Gal4-DBD-activator fusions are tested in presence of glucose, in the repressed state of the GALI promoter. Still, multiple activators are able to enhance GFP expression, highlighting their potential for developing novel activation tools. Surprisingly, although some TF families like the AP2-EREBP TF family are plant-specific (38), activators from this family function in yeast, suggesting that while evolved uniquely in plants, disparate TF families may have converged on similar mechanisms of activation.

Recently, trans elements have been extensively studied in unicellular systems in high throughput enabling the training of machine learning models that can localize activation domains within an effector (16) . Technical challenges have hampered similar approaches to be translated into plant systems, therefore limiting our capability to build similar models. Because there is a mechanism of activation conserved between eukaryotes (Fischer et al. 1988; Ma et al. 1998), the effector candidates are analyzed using ADpred, a machine learning algorithm trained on a large set of putative activation domains in 30 amino acid long protein sequences in S. cerevisiae (FIG. 4, Panel C). It is calculated the ADpred score for 30 amino segments of all effectors in this example as described (Erijman et al. 2020), and assigned a binary value to every effector depending on whether it contained an amino acid section with an ADpred score>=0.9. It is found that activators are more likely to contain consecutive amino acid residues predicted to be activation domains than the repressor and minimally active populations (FIG. 4, Panel C, two-sided Fisher's exact test, p=0.00012). To further validate the predictability of activation domains in plant the predicted activation domains for three TFs are extracted (FIG. 4, Panel D), and benchmarked them against their full length effector domains and VP16. The ADpred predicted motifs of ESE3 and WRKY46 induce the expression of GFP similar to their full length effectors and outperform VP16, showcasing the potential to mine plant TFs using a fungal predictor. The two motifs of PHL4 are not able to induce GFP in the same manner as their parent effector, suggesting that either the two motifs need to function as a bipartite motif or the parent effector uses a mechanism that the model cannot predict. Taken together these results demonstrate that a universal mechanism for activation is likely present in all eukaryotes and the study of this mechanism could enable reliable gene activation in all eukaryotes.

### DISCUSSION

Recent technological advances have focused on the cis regulatory landscape of entire organisms (1, 23, 39), linking TFs to their respective genomic targets. Still, the map for the trans regulatory landscape remains incomplete due to a lack of characterization of the underlying biochemical potential of TFs to modulate target gene expression. Such a dearth in knowledge represents a large blind spot in genome scale transcriptional networks. By annotating effector activity into a temporal GRN with mapped cis-elements, there is a causal explanation for downstream gene expression patterns rectifying this blindspot. This is a novel approach for observing GRNs, where only a combination of DNA binding, gene effector activity and quantified transcripts of each TF with temporal resolution are utilized to judge target gene expression. This ‘full picture’ approach not only links gene expression patterns to interacting TFs but can also help illustrate synergistic activity of multiple TFs targeting the same gene or ambivalence of TFs acting both as activators and repressors (29, 40). Furthermore, this work suggests novel TF targets for further study which could increase throughput of otherwise time ineffective gene perturbations in plants. In an ideal approach one would first measure the activity of all TFs of a given organism to then unravel how a deviation from this behavior comes into being in vivo, generating a middle ground between bottom up, single TF characterization, and top down, systems level approaches.

Activator activity is transferable between eukaryotic families suggesting a conserved activation mechanism common to all eukaryotes (41-42). Here it is shown that predictive machine learning models trained from fungal datasets can correctly predict activation domains inside plant TF sequences, implying that plants rely on a similar mechanism for activation as distant eukaryotes. Importantly the model is not able to localize activation domains in all effectors marked as activators in this study, implying the presence of plant specific features of activation which are either divergent from fungi or have yet to be discovered in fungi. Due to this divergence, it is necessary to generate adjusted machine learning models based on plant data, such as through transfer-learning, to fully exhaust the potential of predictive extraction of plant activation domains from entire plant genomes. Such an achievement would unlock a vast amount of novel synthetic biology tools, either species-specific or universally active, for engineering enhanced traits in different eukaryotic systems.

The targeted control of gene expression using modified site-specific nucleases (32), (32, 36) has been utilized in genome engineering efforts, with the potential to enhance crop yields and promote flux through metabolic pathways (7). However, the vast majority of studies utilize a small repertoire of effector domains to manipulate transcription (e.g., VP16, (35-36)) instead of exploring novel effector domains that are derived from the host system. Analogously, the vast majority of functional genomics screens rely on only a handful of effector Cas9 fusions to probe systems-level regulation. Here, it is demonstrated that reliable tuning of Cas9 based tools, widening the dynamic range of expression for genome editing and functional genomics tool sets, thus opening avenues for improved bioengineering efforts in plants and higher-resolution functional genomic screens.

This study is a landmark towards understanding plant effector activity, transcriptional logic, and ‘full-picture’ GRN architecture. In the future it is believed a concerted effort to map both the cis and trans regulatory landscape of biological organisms can fullfill the promise of systems biologys to link phenotypic observation to genetic cause.

### MATERIALS AND METHODS

### Design of Regulatory-Motifs

The 529 candidate TF sequences are obtained from the work by O'Malley (1). The DBDs of each candidate are identified using ScanProsite (43). In case of C- or N-terminal localization of the DNA binding domain the DBD was removed from the TF sequence leaving a putative TF effector candidate. In case of DBD localization in the center of the protein the longest remaining TF effector candidate after truncation is chosen.

### Construct Design and Assembly

All TFs are synthesized by the core facility of the joint genome institute and cloned into vector pms7997 using Golden Gate cloning and construct specific primers (Supplementary Table 7). Plasmid assemblies are transformed into E. coli strain DH5a and purified plasmids verified with sanger sequencing using primers pms7997_insertseq_fwd & pms7997_insertseq_rev. The PAP1-effector fusion constructs are assembled using golden gate cloning into vector pms057 with PAP1 amplified from A. thaliana genomic DNA. Fusions of effectors with dCas are generated by replacing VP64 in vector pYPQ152 using restriction sites SpeI and AatI and otherwise assembled as described (44). All vectors used for yeast experiments are generated using Gibson assembly of backbone pAI9, native yeast GAL4-DBD amplified from yeast strain W303a gDNA, and amplified effectors with necessary overhangs. All primers used in this study are summarized in Supplementary Table 7.

### Utilization of N. Benthamiana for Characterization of Regulatory Domains

In this study N. benthamiana is used for characterization of A. thaliana regulatory domains. N. benthamiana has the major advantage that no stable line transformations are necessary to prove the activity of a given regulatory domain and expression systems like anthocyanin production can be handled within one week from infection to extraction. The synchronized Agrobacterium mediated transformation using leaf infiltration allows one to observe the behavior of our candidate regulatory domains in parallel.

### Screening of A. Thaliana TFs Agrobacterium Mediated Transient Transformation in N. Benthamiana

Generated binary vectors are transformed into A. tumefaciens strain GV3101. Selected transformants are inoculated in liquid media with appropriate selection and for experiments diluted to an OD600=0.5 and mixed with the assay reporter construct to a final OD600=1.0. N. benthamiana plants grown for four weeks were infiltrated as described by Sparkes et al. (45). Post infiltration N. benthamiana plants are maintained in Percival-Scientific growth chambers at 25° C. in 16/8-hour light/dark cycles and 60% humidity. Leaves are harvested three days post infiltration and eight biological replicates (eight leaf disks) per construct were collected. The leaf disks are floated on 200 μL of water in 96 well microtiter plates and GFP and RFP fluorescence measured using a Synergy 4 microplate reader (Bio-tek). The reporter construct for the screen is pms6370. GFP expression is driven by a fusion of a previously characterized GAL4 binding site and the core MAS promoter (46).

### Quantification of Anthocyanin Content

Anthocyanin production experiments in N. benthamiana plants are performed as described above with the divergence that the entire infiltrated leaf tissue was collected from 2 infiltrated leaves per replicate. Collected tissue is flash frozen in liquid nitrogen and freeze dried at −50° C. in vacuum for 24 h. The dried tissue is ground using bead beating for 5 min at 30 hz and 50 mg tissue is used for extraction. Anthocyanin is extracted three times using 1% hydrochloric acid in methanol and chlorophyll removed with aqueous chloroform. Anthocyanin content is quantified by measuring absorbance at 535 nm on a Spectronic™ 200 spectrophotometer (Thermo Fisher Scientific).

### Quantitative Real-Time PCR (qrtPCR) Experiments

Primers targeting the GUS and Kan genes are designed using the PrimerQuest software (IDT) (Supplementary Table 7) and pre-screened for target specificity via Primer-Blast against the N. benthamiana and A. thaliana genomes. qPCR experiments are conducted on a BioRad CFX 96-well instrument using SYBR Green (BioRad). Reaction conditions were 1× ssoAdvance SYBR Green Supermix (BioRad) and 500 nM primers in 20 μL reactions, qPCR cycling parameters were 95° C. for 3 min, followed by 40 cycles of 30 s at 95° C. and 45 s at 56° C. The linear dynamic range and efficiency of every primer set is verified over 1×10² to 10⁹ copies per μl plasmid template, with values listed in Supplementary Table 6. Target specificity is experimentally validated via melting temperature analysis.

For total RNA isolation, ˜75 mg of leaf tissue is harvested from three plant 5 days post-transformation, where one half of the leaf is treated with reporter alone as reference and the other half with reporter and dCas9-effector candidate as the sample. Leaf tissue is flash frozen in liquid nitrogen and RNA extracted using the EZNA Plant RNA Kit I (Omega Biotek). DNA contamination is removed by treating total RNA with Turbo DNase with inactivation reagent (Invitrogen). cDNA is generated from 1.0 μg total RNA using SuperScript IV Vilo reverse transcriptase (Thermo Fisher Scientific). RT-qPCR is carried out using 1 μl of the reverse transcription reaction as a template. For all experiments, a no template-, a no reverse transcription control is run. All primers are tested with wild type cDNA from plant tissue treated with Agrobacterium containing an empty vector control with Cq>36 as the threshold for no off-target activity. The ΔΔCq method is used to determine normalized expression with GUS as the sample- and KAN as the reference gene quantified.

### Flow Cytometry

For experiments in S. cerevisiae lab strain W303a (MATa/MATα{leu2-3,112 trp1-1 can1-100 ura3-1 ade2-1 his3-11,15 } [phi+]) is used (47). The GAL1-GFP reporter cassette is integrated into the URA3 locus. The Native Gal4-effector fusions are expressed using the TEF1 promoter off a 2μ-plasmid in the reporter strain. For flow cytometry experiments all strains are grown in CSM-URA (Sunrise Science Products) media prepared following the suppliers manual with 2% w/v Glucose, except for the positive control which is grown in 2% w/v Galactose. Experiments are performed on the BD Accuri™ C6 flow cytometer (BD Biosciences), samples are washed with cold 1×PBS (137 mmol NaCl, 2.7 mM KCl, 1.8 mM KH₂PO₄, 10 mM Na₂HPO₄) once before measurement in 1×PBS. Per sample 100.000 events are recorded and samples are analyzed using the FlowJo™ software.

### Negative Autoregulation

DNA binding targets of TFs in this study are obtained from the Arabidopsis Dap seq database (website for: neomorph.salk.edu/PlantCistromeDB) (1). To TFs with available DNA binding information a boolean is assigned based on verified binding of its own promoter region. The boolean value 1 is assigned to TFs binding and 0 to TFs with no binding. Then the booleans are sorted based on the performance of the respective TF in the effector screen. A sliding window analysis is performed, calculating the sum of all booleans within a window of size 25 starting with the repressor population. The window is then moved with step size one along all booleans until all booleans are incorporated into at least one window. Windows describing repressor and activator populations are analyzed for significant differences in their means using a student's t-test.

### Gene Ontology Enrichment

DNA binding targets of TFs in this study are obtained from the Arabidopsis Dap seq database (website for: neomorph.salk.edu/PlantCistromeDB) (1). GO term enrichment of the target genes of TFs screened in this study is performed using the g:Profiler web service accessed via the Python API (48) with the datasource limited to GO:biological process and the significance threshold method set to default g_SCS. The top 3 enriched GO terms for the top 20 activators are visualized in a heatmap using the seaborn python package.

### Generating an Enhanced Nitrogen Response GRN

The extended nitrogen response GRN is built on a version including DNA binding information and a co-expression machine learning model based on temporal RNA-seq data (4). The effector activity is added as a weight metric to the directed edges of TFs targeting downstream genes and extracted subnetworks at time points 10 min and 15 min post induction. RNA-seq analysis is based on the same study and performed using the limma package and DESeq2 in R (49, 50). Illustrations and subnetworks are generated using Cytoscape v3.9.0 (51).

### Analysis of Effector Domains Using ADpred

Effector domains are analyzed using the ADpred model (16). The model can analyze sequence stretches of 30 amino acids maximum and needs secondary structure information. Therefore, the secondary structure of full length effector domains is predicted using the PsiPred workbench (52). The effector domain protein sequence is then fragmented into 30 amino acid sections along its sequence with a frame size of 5 amino acids. If one section of the effector domain scored at >=0.9 in the ADpred model the effector potentially contained an AD. A Boolean is assigned to every effector candidate based on the scoring, 0 for no AD and 1 for containing a potential AD. The booleans are sorted by the performance of the effectors in the initial screen and 20 booleans summed with a sliding window of size 1.

References cited herein:
1. R. C. O'Malley, S. -S. C. Huang, L. Song, M. G. Lewsey, A. Bartlett, J. R. Nery, M. Galli, A. Gallavotti, J. R. Ecker, Cistrome and epicistrome features shape the regulatory DNA landscape. Cell. 165, 1280-1292 (2016).
2. ENCODE Project Consortium, An integrated encyclopedia of DNA elements in the human genome. Nature. 489, 57-74 (2012).
3. A. P. Marand, Z. Chen, A. Gallavotti, R. J. Schmitz, A cis-regulatory atlas in maize at single-cell resolution. Cell. 184, 3041-3055.e21 (2021).
4. K. Varala, A. Marshall-Colon, J. Cirrone, M. D. Brooks, A. V. Pasquino, S. Leran, S. Mittal, T. M. Rock, M. B. Edwards, G. J. Kim, S. Ruffel, W. R. McCombie, D. Shasha, G. M. Coruzzi, Data from: Temporal transcriptional logic of dynamic regulatory networks underlying nitrogen signaling and use in plants. Dryad (2019), doi:10.5061/dryad.248g184.
5. A. Gaudinier, J. Rodriguez-Medina, L. Zhang, A. Olson, C. Liseron-Monfils, A. -M. Bagman, J. Foret, S. Abbitt, M. Tang, B. Li, D. E. Runcie, D. J. Kliebenstein, B. Shen, M. J. Frank, D. Ware, S. M. Brady, Transcriptional regulation of nitrogen-associated metabolism and growth. Nature. 563, 259-264 (2018).
6. P. S. Brzovic, C. C. Heikaus, L. Kisselev, R. Vernon, E. Herbig, D. Pacheco, L. Warfield, P. Littlefield, D. Baker, R. E. Klevit, S. Hahn, The acidic transcription activator Gcn4 binds the mediator subunit Gall 1/Med15 using a simple protein interface forming a fuzzy complex. Mol. Cell. 44, 942-953 (2011).
7. S. Soyk, Z. H. Lemmon, F. J. Sedlazeck, J. M. Jimenez-Gomez, M. Alonge, S. F. Hutton, J. Van Eck, M. C. Schatz, Z. B. Lippman, Duplication of a domestication locus neutralized a cryptic variant that caused a breeding barrier in tomato. Nat. Plants. 5,471-479 (2019).
8. M. B. Hufford, X. Xu, J. van Heerwaarden, T. Pyhajarvi, J. -M. Chia, R. A. Cartwright, R. J. Elshire, J. C. Glaubitz, K. E. Guill, S. M. Kaeppler, J. Lai, P. L. Morrell, L. M. Shannon, C. Song, N. M. Springer, R. A. Swanson-Wagner, P. Tiffin, J. Wang, G. Zhang, J. Doebley, J. Ross-Ibarra, Comparative population genomics of maize domestication and improvement. Nat. Genet. 44, 808-811 (2012).
9. Z. Wang, Z. Zheng, L. Song, D. Liu, Functional characterization of arabidopsis PHL4 in plant response to phosphate starvation. Front. Plant Sci. 9, 1432 (2018).
10. Y. Shi, J. Huang, T. Sun, X. Wang, C. Zhu, Y. Ai, H. Gu, The precise regulation of different COR genes by individual CBF transcription factors in Arabidopsis thaliana. J. Integr. Plant Biol. 59, 118-133 (2017).
11. M. Martchenko, A. Levitin, M. Whiteway, Transcriptional activation domains of the Candida albicans Gcn4p and Gal4p homologs. Eukaryotic Cell. 6, 291-301 (2007).
12. Eukaryotic Transcription Factors-5th Edition, (available at website for: elsevier.com/books/eukaryotic-transcription-factors/latchman/978-0-12-373983-4).
13. J. Liu, N. B. Perumal, C. J. Oldfield, E. W. Su, V. N. Uversky, A. K. Dunker, Intrinsic disorder in transcription factors. Biochemistry. 45, 6873-6888 (2006).
14. I. A. Hope, S. Mahadevan, K. Struhl, Structural and functional characterization of the short acidic transcriptional activation region of yeast GCN4 protein. Nature. 333, 635-640 (1988).
15. B. M. Jackson, C. M. Drysdale, K. Natarajan, A. G. Hinnebusch, Identification of seven hydrophobic clusters in GCN4 making redundant contributions to transcriptional activation. Mol. Cell. Biol. 16, 5557-5571 (1996).
16. A. Erijman, L. Kozlowski, S. Sohrabi-Jahromi, J. Fishburn, L. Warfield, J. Schreiber, W. S. Noble, J. Soding, S. Hahn, A High-Throughput Screen for Transcription Activation Domains Reveals Their Sequence Features and Permits Prediction by Deep Learning. Mol. Cell. 78, 890-902.e6 (2020).
17. A. L. Sanborn, B. T. Yeh, J. T. Feigerle, C. V. Hao, R. J. Townshend, E. Lieberman Aiden, R. O. Dror, R. D. Kornberg, Simple biochemical features underlie transcriptional activation domain diversity and dynamic, fuzzy binding to Mediator. eLife. 10 (2021), doi:10.7554/eLife.68068.
18. M. V. Staller, E. Ramirez, S. R. Kotha, A. S. Holehouse, R. V. Pappu, B. A. Cohen, Directed mutational scanning reveals a balance between acidic and hydrophobic residues in strong human activation domains. Cell Syst. (2022), doi:10.1016/j.cels.2022.01.002.
19. K. Hill, H. Wang, S. E. Perry, A transcriptional repression motif in the MADS factor AGL15 is involved in recruitment of histone deacetylase complex components. Plant J. 53, 172-185 (2008).
20. F. Baile, W. Merini, I. Hidalgo, M. Calonje, EAR domain-containing transcription factors trigger PRC2-mediated chromatin marking in Arabidopsis. Plant Cell. 33, 2701-2715 (2021).
21. H. Szemenyei, M. Hannon, J. A. Long, TOPLESS mediates auxin-dependent transcriptional repression during Arabidopsis embryogenesis. Science. 319, 1384-1386 (2008).
22. U. Alon, Network motifs: theory and experimental approaches. Nat. Rev. Genet. 8, 450-461 (2007).
23. D. Chen, W. Yan, L. -Y. Fu, K. Kaufmann, Architecture of gene regulatory networks controlling flower development in Arabidopsis thaliana. Nat. Commun. 9, 4534 (2018).
24. D. Thieffry, A. M. Huerta, E. Perez-Rueda, J. Collado-Vides, From specific gene regulation to genomic networks: a global analysis of transcriptional regulation in Escherichia coli. Bioessays. 20, 433-440 (1998).
25. N. Rosenfeld, M. B. Elowitz, U. Alon, Negative autoregulation speeds the response times of transcription networks. J. Mol. Biol. 323, 785-793 (2002).
26. G. Krouk, P. Mirowski, Y. LeCun, D. E. Shasha, G. M. Coruzzi, Predictive network modeling of the high-resolution dynamic plant transcriptome in response to nitrate. Genome Biol. 11, R123 (2010).
27. A. Safi, A. Medici, W. Szponarski, F. Martin, A. Clement-Vidal, A. Marshall-Colon, S. Ruffel, F. Gaymard, H. Rouached, J. Leclercq, G. Coruzzi, B. Lacombe, G. Krouk, GARP transcription factors repress Arabidopsis nitrogen starvation response via ROS-dependent and -independent pathways. J. Exp. Bot. 72, 3881-3901 (2021).
28. T. Kiba, J. Inaba, T. Kudo, N. Ueda, M. Konishi, N. Mitsuda, Y. Takiguchi, Y. Kondou, T. Yoshizumi, M. Ohme-Takagi, M. Matsui, K. Yano, S. Yanagisawa, H. Sakakibara, Repression of Nitrogen Starvation Responses by Members of the Arabidopsis GARP-Type Transcription Factor NIGT1/HRS1 Subfamily. Plant Cell. 30, 925-945 (2018).
29. M. D. Brooks, J. Cirrone, A. V. Pasquino, J. M. Alvarez, J. Swift, S. Mittal, C. -L. Juang, K. Varala, R. A. Gutierrez, G. Krouk, D. Shasha, G. M. Coruzzi, Network Walking charts transcriptional dynamics of nitrogen signaling by integrating validated and predicted genome-wide interactions. Nat. Commun. 10, 1569 (2019).
30. M. E. Campbell, J. W. Palfreyman, C. M. Preston, Identification of herpes simplex virus DNA sequences which encode a trans-acting polypeptide responsible for stimulation of immediate early transcription. J. Mol. Biol. 180, 1-19 (1984).
31. W. D. Cress, S. J. Triezenberg, Critical structural elements of the VP16 transcriptional activation domain. Science. 251, 87-90 (1991).
32. L. G. Lowder, J. Zhou, Y. Zhang, A. Malzahn, Z. Zhong, T. -F. Hsieh, D. F. Voytas, Y. Zhang, Y. Qi, Robust Transcriptional Activation in Plants Using Multiplexed CRISPR-Act2.0 and mTALE-Act Systems. Mol. Plant. 11, 245-256 (2018).
33. G. Stampfel, T. Kazmar, O. Frank, S. Wienerroither, F. Reiter, A. Stark, Transcriptional regulators form diverse groups with context-dependent regulatory functions. Nature. 528, 147-151 (2015).
34. H. Yan, X. Pei, H. Zhang, X. Li, X. Zhang, M. Zhao, V. L. Chiang, R. R. Sederoff, X. Zhao, MYB-Mediated Regulation of Anthocyanin Biosynthesis. Int. J. Mol. Sci. 22 (2021), doi:10.3390/ijms22063103.
35. C. Pan, X. Wu, K. Markel, A. A. Malzahn, N. Kundagrami, S. Sretenovic, Y. Zhang, Y. Cheng, P. M. Shih, Y. Qi, CRISPR-Act3.0 for highly efficient multiplexed gene activation in plants. Nat. Plants. 7, 942-953 (2021).
36. A. Chavez, M. Tuttle, B. W. Pruitt, B. Ewen-Campen, R. Chari, D. Ter-Ovanesyan, S. J. Hague, R. J. Cecchi, E. J. K. Kowal, J. Buchthal, B. E. Housden, N. Perrimon, J. J. Collins, G. Church, Comparison of Cas9 activators in multiple species. Nat. Methods. 13, 563-567 (2016).
37. C. Ricci-Tam, I. Ben-Zion, J. Wang, J. Palme, A. Li, Y. Savir, M. Springer, Decoupling transcription factor expression and activity enables dimmer switch gene regulation. Science. 372, 292-295 (2021).
38. J. K. Okamuro, B. Caster, R. Villarroel, M. Van Montagu, K. D. Jofuku, The AP2 domain of APETALA2 defines a large new family of DNA binding proteins in Arabidopsis. Proc Natl Acad Sci USA. 94, 7076-7081 (1997).
39. X. Tu, M. K. Mejia-Guerra, J. A. Valdes Franco, D. Tzeng, P.-Y. Chu, W. Shen, Y. Wei, X. Dai, P. Li, E. S. Buckler, S. Zhong, Reconstructing the maize leaf regulatory network using ChIP-seq data of 104 transcription factors. Nat. Commun. 11, 5089 (2020).
40. P. Perez-Pinera, D. G. Ousterout, J. M. Brunger, A. M. Farin, K. A. Glass, F. Guilak, G. E. Crawford, A. J. Hartemink, C. A. Gersbach, Synergistic and tunable human gene activation by combinations of synthetic transcription factors. Nat. Methods. 10, 239-242 (2013).
41. J. Ma, E. Przibilla, J. Hu, L. Bogorad, M. Ptashne, Yeast activators stimulate plant gene expression. Nature. 334, 631-633 (1988).
42. J. A. Fischer, E. Giniger, T. Maniatis, M. Ptashne, GAL4 activates transcription in Drosophila. Nature. 332, 853-856 (1988).
43. C. J. A. Sigrist, E. de Castro, L. Cerutti, B. A. Cuche, N. Hulo, A. Bridge, L. Bougueleret, I. Xenarios, New and continuing developments at PROSITE. Nucleic Acids Res. 41, D344-7 (2013).
44. L. G. Lowder, D. Zhang, N. J. Baltes, J. W. Paul, X. Tang, X. Zheng, D. F. Voytas, T. -F. Hsieh, Y. Zhang, Y. Qi, A crispricas9 toolbox for multiplexed plant genome editing and transcriptional regulation. Plant Physiol. 169, 971-985 (2015).
45. I. A. Sparkes, J. Runions, A. Kearns, C. Hawes, Rapid, transient expression of fluorescent fusion proteins in tobacco plants and generation of stably transformed plants. Nat. Protoc. 1, 2019-2025 (2006).
46. M. S. Belcher, K. M. Vuu, A. Zhou, N. Mansoori, A. Agosto Ramos, M. G. Thompson, H. V. Scheller, D. Logue, P. M. Shih, Design of orthogonal regulatory systems for modulating gene expression in plants. Nat. Chem. Biol. 16, 857-865 (2020).
47. M. Ralser, H. Kuhl, M. Ralser, M. Werber, H. Lehrach, M. Breitenbach, B. Timmermann, The Saccharomyces cerevisiae W303-K6001 cross-platform genome sequence: insights into ancestry and physiology of a laboratory mutt. Open Biol. 2, 120093 (2012).
48. U. Raudvere, L. Kolberg, I. Kuzmin, T. Arak, P. Adler, H. Peterson, J. Vilo, g:Profiler: a web server for functional enrichment analysis and conversions of gene lists (2019 update). Nucleic Acids Res. 47, W191—W198 (2019).
49. M. E. Ritchie, B. Phipson, D. Wu, Y. Hu, C. W. Law, W. Shi, G. K. Smyth, limma powers differential expression analyses for RNA-sequencing and microarray studies. Nucleic Acids Res. 43, e47 (2015).
50. M. I. Love, W. Huber, S. Anders, Moderated estimation of fold change and dispersion for RNA-seq data with DESeq2. Genome Biol. 15, 550 (2014).
51. P. Shannon, A. Markiel, O. Ozier, N. S. Baliga, J. T. Wang, D. Ramage, N. Amin, B. Schwikowski, T. Ideker, Cytoscape: a software environment for integrated models of biomolecular interaction networks. Genome Res. 13, 2498-2504 (2003).
52. D. W. A. Buchan, D. T. Jones, The PSIPRED Protein Analysis Workbench: 20 years on. Nucleic Acids Res. 47, W402—W407 (2019).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A synthetic transcription factor (IF) comprising (a) a DNA-binding domain of a transcription factor linked to (b) an effector domain comprising an amino acid sequence of any one of SEQ ID NO:1-403.

2. The synthetic TF of claim 1, wherein the synthetic TF further comprises (c) a nuclear localization sequence (NLS).

3. The synthetic TF of claim 1, wherein the DNA-binding domain is a deactivated RNA-guided nuclease variant of Cas9 (dCas9).

4. A nucleic acid encoding the synthetic TF of claim 1.

5. A nucleic acid encoding an effector domain comprising an amino acid sequence of any one of SEQ ID NO:1-403.

6. A vector comprising the nucleic acid of claim 4.

7. A host cell comprising the vector of claim 6, wherein he host cell is capable of expressing the synthetic TF or effector domain.

8. A system comprising a nucleic acid of claim 4 and a second nucleic acid, or the nucleic acid, encodes a gene of interest (GOI) operatively linked to a promoter and one or more activatorlrepressor binding domains, or combination thereof, wherein the synthetic TF binds at least one of the one or more activatorlrepressor binding domain such that the synthetic TF modulates the expression of the GOI.

9. A genetically modified eukaryotic cell or organism comprising: (a) (i) one or more nucleic acids each encoding one or more transcription activators operatively linked to a first promoter, (ii) one or more nucleic acids each encoding one or more transcription repressors each operatively linked to a second promoter, or (iii) combinations thereof; and (b) one or more nucleic acids each encoding one or more independent genes of interest (GOI) each operatively linked to a promoter that is activated by the one or more transcription activators, repressed by the one or more transcription repressors, or a combination of both; wherein at least one transcription activator or transcription repressor is a synthetic transcription factor (TF) of claim 1.


SEQUENCE LISTING (403 sequences)

SEQ ID NO: 1 - protein, 271 aa, Arabidopsis thaliana
DEPNEGDQGFSFEHGAGYTYNLSQLPMLQSFDQRPSSSLGYGGGSWTDHRRQIYRSPWRGLTTRENTRTRQTMFSSQPGERYHGVSNSILNDKNKTISFRINSHEGVHDNNGVAGAVPRIHRSFLEGMKTFNKSWGQSLSSNLKSSTATIPQDHIATTLNSYQWENAGVAEGSENVLKRKRLLFSDDCNKSDQDLDLSLSLKVPRTHDNLGECLLEDEVKEHDDHQDIKSLSLSLSSSGSSKLDRTIRKEDQTDHKKRKISVLASPLDLTL

SEQ ID NO: 2 - protein, 76 aa, Arabidopsis thaliana
KKRRTLISNRSEDKKKKSHNRNPKFGDSLKQRLMELGREVMMQRSTAENQRRNKLGEEEQAAVLLMALSYASSVYA

SEQ ID NO: 3 - protein, 83 aa, Arabidopsis thaliana
MALDTLNSPTSTTTTTAPPPFLRCLDETEPENLESWTKRKRTKRHRIDQPNPPPSEEEYLALCLLMLARGSSDHHSPPSDHHS

SEQ ID NO: 4 - protein, 87 aa, Arabidopsis thaliana
MMGYQTNSNFSMFFSSENDDQNHHNYDPYNNFSSSTSVDCTLSLGTPSTRLDDHHRFSSANSNNISGDFYIHGGNAKTSSYKKGGVA

SEQ ID NO: 5 - protein, 107 aa, Arabidopsis thaliana
RSGSSPSSNLKNQTVAEKPDHHGSGSEEKEERVSGQEMNPTRMLYGLPVGDPNGASFSSLLASNMQMGGLVYESGSRWLPGMDLGLGSVRRSDDTWTDLAMNRMEKN

SEQ ID NO: 6 - protein, 129 aa, Arabidopsis thaliana
MMMGKEDLGLSLSLGFAQNHPLQLNLKPTSSPMSNLQMFPWNQTLVSSSDQQKQQFLRKIDVNSLPTTVDLEEETGVSSPNSTISSTVSGKRRSTEREGTSGGGCGDDLDITLDRSSSRGTSDEEEDYG

SEQ ID NO: 7 - protein, 292 aa, Arabidopsis thaliana
MVSHKCVEEFGYASYLVPSNARAPRSARKRRSIEKRISKEDDNMCAIDLLATVAGHLSFESGSSLMSIDKLIEDHRVKEEFPEEEKPLMPVALSPYRGSLSPCGFSSVINGKVENEVDGFSYSGGSDACQVGNFSQDVKPDIDGDAVVLDARPNVVVSLGSSSRTEVPSIGNCVSHGVRDDVNLFSRDDDENFSKYIHPRVTKHSPRTVPRIGDRRIRKILASRHWKGGSRHSDTKPWRNYYLHQQRSYPIKKRKNFDHISDSVTDDYRMRTKMHRGSRKGQGASFVASDSH

SEQ ID NO: 8 - protein, 414 aa, Arabidopsis thaliana
NLPWKLKQRTSKEVRKRVYVCPEKSCVHHHPTRALGDLTGIKKHFCRKHGEKKWKCEKCAKRYAVQSDWKAHSKTCGTREYRCDCGTIFSRRDSFITHRAFCDALAEETARLNAASHLKSFAATAGSNLNYHYLMGTLIPSPSLPQPPSFPFGPPQPQHHHHHQFPITTNNFDHQDVMKPASTLSLWSGGNINHHQQVTIEDRMAPQPHSPQEDYNWVFGNANNHGELITTSDSLITHDNNINIVQSKENANGATSLSVPSLFSSVDQITQDANAASVAVANMSATALLQKAAQMGATSSTSPTTTITTDQSAYLQSFASKSNQIVEDGGSDRFFASFGSNSVELMSNNNNGLHEIGNPRNGVTVVSGMGELQNYPWKRRRVDIGNAGGGGQTRDFLGVGVQTICHSSSINGWI

SEQ ID NO: 9 - protein, 152 aa, Arabidopsis thaliana
MEAFEEATKEQSLILKGKRTKRQRPQSPIPFSISPPIVSTPENNMEEEYTDLDSKDNALGNDEGNHKKDGVITSSSSSASWSSQNNHTLKAAEDEEDQDIANCLILLAQGHSLPHNNHHLPNSNNNNTYRFTSRRFLETSSSNSGGKAGYYV

SEQ ID NO: 10 - protein, 124 aa, Arabidopsis thaliana
MMMGKEDLGLSLSLGFSQNHNPLQMNLNPNSSLSNNLQRLPWNQTFDPTSDLRKIDVNSFPSTVNCEEDTGVSSPNSTISSTISGKRSEREGISGTGVGSGDDHDEITPDRGYSRGTSDEEEDG

SEQ ID NO: 11 - protein, 207 aa, Arabidopsis thaliana
MFGRHSIIPNNQIGTASASAGEDHVSASATSGHIPYDDMEEIPHPDSIYGAASDLIPDGSQLVAHRSDGSELLVSRPPEGANQLTISFRGQVYVFDAVGADKVDAVLSLLGGSTELAPGPQVMELAQQQNHMPVVEYQSRCSLPQRAQSLDRFRKKRNARCFEKKVRYGVRQEVALRMARNKGQFTSSKMTDGAYNSGTDQDSAQDD

SEQ ID NO: 12 - protein, 141 aa, Arabidopsis thaliana
EEEQRPSQLSYETESDVSSSDPKFAFTSSVLLEDGESESESSRNVINLTRKRSKRTRKLDSFVTKKVKTSQLGYKPESDQEPPHSSASDTTTEEDLAFCLMMLSRDKWKKNKSNKEVVEEIETEEESEGYNKINRATTKGR

SEQ ID NO: 13 - protein, 194 aa, Arabidopsis thaliana
KRFNGTNMTTPSSSPNSVMMAANDHYHPLLHHHHGVPMQRPANSVNVKLNQDHHLYHHNKPYPSFNNGNLNHASSGTECGVVNASNGYMSSHVYGSMEQDCSMNYNNVGGGWANMDHHYSSAPYNFFDRAKPLFGLEGHQEEEECGGDAYLEHRRTLPLFPMHGEDHINGGSGAIWKYGQSEVRPCASLELRLN

SEQ ID NO: 14 - protein, 71 aa, Arabidopsis thaliana
KKRRGGTEDNKKLKKSSSGGGNRKFGESLKQSLMDLGIRKRSTVEKQRQKLGEEEQAAVLLMALSYGSVYA

SEQ ID NO: 15 - protein, 242 aa, Arabidopsis thaliana
ARQQGVFISGTGDQAHSTGGNGALAFDAEHSRWLEEKNKQMNELRSALNAHAGDSELRIIVDGVMAHYEELFRIKSNAAKNDVFHLLSGMWKTPAERCFLWLGGFRSSELLKLLANQLEPMTERQLMGINNLQQTSQQAEDALSQGMESLQQSLADTLSSGTLGSSSSGNVASYMGQMAMAMGKLGTLEGFIRQADNLRLQTLQQMIRVLTTRQSARALLAIHDYFSRLRALSSLWLARPRE

SEQ ID NO: 16 - protein, 341 aa, Arabidopsis thaliana
NLPWKLKQRSNKDVVRKKVYVCPEPGCVHHHPSRALGDLTGIKKHFFRKHGEKKWKCEKCSKKYAVQSDWKAHAKTCGTKEYKCDCGTLFSRRDSFITHRAFCDALAEESARAMPNPIMIQASNSPHHHHHQTQQNIGFSSSSQNIISNSNLHGPMKQEESQHHYQNIPPWLISSNPNPNGNNGNLFPPVASSVNTGRSSFPHPSPAMSATALLQKAAQMGSTKSTTPEEEERSSRSSYNNLITTTMAAMMTSPPEPGFGFQDYYMMNHQHHGGGEAFNGGFVPGEEKNDVVDDGGGETRDFLGLRSLMSHNEILSFANNLGNCLNTSATEQQQQQHSHQD

SEQ ID NO: 17 - protein, 458 aa, Arabidopsis thaliana
SVNGWGRRPAALRALSQRLSRGFNEAVNGFTDEGWSVIGDSMDDVTITVNSSPDKLMGLNLTFANGFAPVSNVVLCAKASMLLQNVPPAILLRFLREHRSEWADNNIDAYLAAAVKVGPCSARVGGFGGQVILPLAHTIEHEEFMEVIKLEGLGHSPEDAIVPRDIFLLQLCSGMDENAVGTCAELIFAPIDASFADDAPLLPSGFRIIPLDSAKQEVSSPNRTLDLASALEIGSAGTKASTDQSGNSTCARSVMTIAFEFGIESHMQEHVASMARQYVRGIISSVQRVALALSPSHISSQVGLRTPLGTPEAQTLARWICQSYRGYMGVELLKSNSDGNESILKNLWHHTDAIICCSMKALPVFTFANQAGLDMLETTLVALQDISLEKIFDDNGRKTLCSEFPQIMQQGFACLQGGICLSSMGRPVSYERAVAWKVLNEEENAHCICFVFINWSFV

SEQ ID NO: 18 - protein, 92 aa, Arabidopsis thaliana
QKEKRKTVNGDDLLWAMATLGFEDYLEPLKIYLARYREVFETNSVLFIPWDWLLTHHLLMQLEGDNKGSGKSGDGSNRDAGGGVSGEEMPSW

SEQ ID NO: 19 - protein, 100 aa, Arabidopsis thaliana
MSKPEETSDPNPTGPDPSRSSSDEVTVTVADRAPSDLNHVSEELSDQLRNVGLDDSAKELSVPISVPQGNVETDSRALFGSDQKEEEEGSEKRMMMVYPV

SEQ ID NO: 20 - protein, 57 aa, Arabidopsis thaliana
REKASNVLVTHSFMPETTTVTTLKKSSSGKRVACLDFDLTSVESFVNTELELGRTMY

SEQ ID NO: 21 - protein, 378 aa, Arabidopsis thaliana
NLPWKLKQRTSKEVRKRVYVCPEKTCVHHHSSRALGDLTGIKKHFCRKHGEKKWTCEKCAKRYAVQSDWKAHSKTCGTREYRCDCGTIFSRRDSFITHRAFCDALAEETAKINAVSHLNGLAAAGAPGSVNLNYQYLMGTFIPPLQPFVPQPQTNPNHHHQHFQPPTSSSLSLWMGQDIAPPQPQPDYDWVFGNAKAASACIDNNNTHDEQITQNANASLTTTTTLSAPSLFSSDQPQNANANSNVNMSATALLQKAAEIGATSTTTAATNDPSTFLQSFPLKSTDQTTSYDSGEKFFALFGSNNNIGLMSRSHDHQEIENARNDVTVASALDELQNYPWKRRRVDGGGEVGGGGQTRDFLGVGVQTLCHPSSINGWI

SEQ ID NO: 22 - protein, 108 aa, Arabidopsis thaliana
ISRELRRKLFGRYRRSYRRGSRSRSRSISPRRKREHSRERERGDVRDRDRHGNGKRSSDRSERHDRDGGGRRRHGSPKRSRSPRNVREGSEERRARIEQWNRERDEGV

SEQ ID NO: 23 - protein, 113 aa, Arabidopsis thaliana
MSEIEELVCIEASVTRKSTSNTVEIRESRRNKVTLGSSDSPAFPTPHLFLKNIVSFDEQSMYNLLYPRLQDPNLCSILSFKIAFEAKRVPGPLYISYDVTLTPQIFEEPDMET

SEQ ID NO: 24 - protein, 207 aa, Arabidopsis thaliana
LKMGIDPVTHTPRLDLLDISSILSSSIYNSSHHHHHHHQQHMNMSRLMMSDGNHQPLVNPEILKLATSLFSNQNHPNNTHENNTVNQTEVNQYQTGYNMPGNEELQSWFPIMDQFTNFQDLMPMKTTVQNSLSYDDDCSKSNFVLEPYYSDFASVLTTPSSSPTPLNSSSSTYINSSTCSTEDEKESYYSDNITNYSFDVNGFLQFQ

SEQ ID NO: 25 - protein, 233 aa, Arabidopsis thaliana
MAVELMTRNYISGVGADSFAVQEAAASGLKSIENFIGLMSRDSFNSDQPSSSSASASASAAADLESARNTTADAAVSKFKRVISLLDRTRTGHARFRRAPVHVISPVLLQEEPKTTPFQSPLPPPPQMIRKGSFSSSMKTIDFSSLSSVTTESDNQKKIHHHQRPSETAPFASQTQSLSTTVSSFSKSTKRKCNSENLLTGKCASASSSGRCHCSKKRKIKQRRIIRVPAISA

SEQ ID NO: 26 - protein, 367 aa, Arabidopsis thaliana
NLPWKLRQKSNKEVKKKVYVCPEVSCVHHDPSRALGDLTGIKKHFCRKHGEKKWKCDKCSKKYAVQSDWKAHSKICGTKEYKCDCGTLFSRRDSFITHRAFCDALAEENARSHHSQSKKQNPEILTRKNPVPNPVPAPVDTESAKIKSSSTLTIKQSESPKTPPEIVQEAPKPTSLNVVTSNGVFAGLFESSSASPSIYTTSSSSKSLFASSSSIEPISLGLSTSHGSSFLGSNRFHAQPAMSATALLQKAAQMGAASSGGSLLHGLGIVSSTSTSIDAIVPHGLGLGLPCGGESSSGLKELMMGNSSVFGPKQTTLDFLGLGRAVGNGNGPSNGLSTLVGGGTGIDMATTFGSGEFSGKDISRRKS

SEQ ID NO: 27 - protein, 212 aa, Arabidopsis thaliana
VPDRWEFSNDFFKRGEKRLLREIQRRKITTTHQTVVAPSSEQRNQTMVVSPSNSGEDNNNNQVMSSSPSSWYCHQTKTTGNGGLSVELLEENEKLRSQNIQLNRELTQMKSICDNIYSLMSNYVGSQPTDRSYSPGGSSSQPMEFLPAKRFSEMEIEEEEEASPRLFGVPIGLKRTRSEGVQVKTTAVVGENSDEETPWLRHYNRTNQRVCN

SEQ ID NO: 28 - protein, 305 aa, Arabidopsis thaliana
EELVLGEEDSKSDEVEEPAVSSPTVEVTKSEVSEVIKTEDVKRHDIAESSLVISGDSHSDACDEATTAELVDFKWYPELESLDFTLFSPLHSQVQSELGSSYNTFQPGSSNFSGNNNNSFQIQTQYGTNEVDTYISDFLDSILKSPDEDPEKHKYVLQSGFDVVAPDQIAQVCQQGSAVDMSNDVSVTGIQIKSRQAQPSGYTNDYIAQGNGPRRLRLQSNFNGINTKNPELQAIKREAEDTVGESIKKRCGKLMRSKNVTGFVFKKITSVKCSYGGLFRAAVVAVVFLMSVCSLTVDFRASAVS

SEQ ID NO: 29 - protein, 213 aa, Arabidopsis thaliana
MVQTETDQRMGLNLNLSIYSLPKPLSQFLDEVSRIKDNHSKLSEIDGYVGKLEEERNKIDVFKRELPLCMLLLNEEIVFLCVAIGALKDEARKGLSLMASNGKFDDVERAKPETDKKSWMSSAQLWISNPNSQFRSTNEEEEDRCVSQNPFQTCNYPNQGGVFMPFNRPPPPPPPAPLSLMTPTSEMMMDYSRIEQSHHHHQFNKPSSQSHHI

SEQ ID NO: 30 - protein, 353 aa, Arabidopsis thaliana
KHEAMAKENRIACCVNSDNKRLLFPDGISTPLKAESESPLTKKMRRSHIPNLTEIKSYGDRSHIKVESTMNQQRRHPFSVVAHNATSSDGTEEQKQIGNVKESDGEDKSNQEVFLKKDDSKVTALMQQAELLSSLAQKVNADNTDQSMENAWKVLQDFLNKSKENDLFRYGIPDIDFQLDEFKDLVEDLRSSNEDSQSSWRQPDLHDSPASSEYSSGSGSGSTIMTHPSGDKTQQLMSDTQTTSHQQNGGELLQDNGIVSDATVEQVGLLSTGHDVLKNSNETVPIPGEEEFNSPVQVTPLFRSLAAGIPSPQFSESERNFLLKTLGVESPSPYPSANPSQPPPCKRVLLDSL

SEQ ID NO: 31 - protein, 163 aa, Arabidopsis thaliana
TGDRKNVGLIHNQISYLHNHSLSTTHHHHHEALPLLIEPSNKTLTNFPSLLYDDPHQNYNNNNFLHGSSGHNIDELKALINPVVSQLNGIIFPSGNNNNDEDDFDFNLGVKTEQSSNGNEIDVRDYLENPLFQEASYGLLGFSSSPGPLHMLLDSPCPLGFQL

SEQ ID NO: 32 - protein, 79 aa, Arabidopsis thaliana
MALEALTSPRLASPIPPLFEDSSVFHGVEHWTKGKRSKRSRSDFHHQNLTEEEYLAFCLMLLARDNRQPPPPPAVEKLS

SEQ ID NO: 33 - protein, 110 aa, Arabidopsis thaliana
MSGREDEEEDLGTAMQKIPIPVNVFDKEPMDLDTVFGFADGVREIIEDSNLLLEESREFDTNDSKPSRNFSNLPTATRGRLHAPKRSGNKRGRQKRLSFKSPSDLFDSKF

SEQ ID NO: 34 - protein, 92 aa, Arabidopsis thaliana
ELLAGLTVSNGGGRGGDLSAAYIRRKAAEVGAQVDALGATVVVNTGGENRGDYEKIENCRKSGNGSLERVDLNKLPDPENSDGDDDECVKRR

SEQ ID NO: 35 - protein, 177 aa, Arabidopsis thaliana
MSNPACSNLFNNGCDHNSFNYSTSLSYIYNSHGSYYYSNTTNPNYINHTHTTSTSPNSPPLREALPLLSLSPIRHQEQQDQHYFMDTHQISSSNFLDDPLVTVDLHLGLPNYGVGESIRSNIAPDATTDEQDQDHDRGVEVTVESHLDDDDDHHGDLHRGHHYWIPTPSQILIGPTQ

SEQ ID NO: 36 - protein, 274 aa, Arabidopsis thaliana
MTVELMMSSYSGGGGGGDGFPAIAAAAKMEDTALREAASAGIHGVEEFLKLIGQSQQPTEKSQTEITAVTDVAVNSFKKVISLLGRSRTGHARFRRAPASTQTPFKQTPVVEEEVEVEEKKPETSSVLTKQKTEQYHGGGSAFRVYCPTPIHRRPPLSHNNNNNQNQTKNGSSSSSPPMLANGAPSTINFAPSPPVSATNSFMSSHRCDTDSTHMSSGFEFTNPSQLSGSRGKPPLSSASLKRRCNSSPSSRCHCSKKRKSRVKRVIRVPAVSS

SEQ ID NO: 37 - protein, 206 aa, Arabidopsis thaliana
TTLASTGAVSEGGGGGGATVSVSSGTGPSKKTKVPSTISRNYQEQPSSPSSVSLPPLLDPTTTLGYTDSSCSYDSRSTNTTVTASAITEHVSCFSTVPTTTTALGLDVNSFSRLPPPLGFDFDPFPRFVSRNVSTQSNFRSFQENFNQFPYFGSSSASTMTSAVNLPSFQGGGGVSGMNYWLPATAEENESKVGVLHAGLDCIWNY

SEQ ID NO: 38 - protein, 143 aa, Arabidopsis thaliana
RERSKLRPRGLGHDGTVAATGMIGNYKDCDKERRLATTTAINFPYQFSHINHFQVLKEFLTGKIGFRNSTTPIQEGAIDQTKRPMEFYNFLQVNTDSKIHELIDNSRKDEEEDVDQNNRIPNENCVPFFDFLSVGNSASQGLC

SEQ ID NO: 39 - protein, 92 aa, Arabidopsis thaliana
TTLHHKRRRTSLFDMVSAGNVEENSTTKRICNDHIGSSSKVVWKQGLLNPRLGYPDPKVSVSGSGNSGGLDLELKLASIQSPESNIRPISVT

SEQ ID NO: 40 - protein, 245 aa, Arabidopsis thaliana
MTSIPNGLNSYVDDTVNICGFTPIEMSSNLRNHESKMVHSMFNTSDHTNHHGLFSSSRVFNFYQDSHVSSSSFGFNNSHMAYHMRKNMVSTFGMPCITQNSNNPHLSQISITQTITNSYSAIVPTYNLITSQNEYQRAKEPNIFNPPFYPPNFVDKNVGNQCQILNPTPLNTIFPHQASIFPRNVDKFSFSPKQNPHQYVSYRQPLKRHCRPTKKFENTFSDFDSGKDIEYDGRTHSLPYEKYGP

SEQ ID NO: 41 - protein, 194 aa, Arabidopsis thaliana
SGGVAVTTVTETEEDQDRPKKRRSVSFDSAFAPVDTGLYMSPESPNGIDVSDSSTIPSPSSPVAQLFKPMPISGGFTVVPQPLPVEMSSSSEDPPTSLSLSLPGAENTSSSHNNNNNALMFPRFESQMKINVEERGEGRRGEFMTVVQEMIKAEVRSYMAEMQKTSGGFVVGGLYESGGNGGFRDCGVITPKVE

SEQ ID NO: 42 - protein, 208 aa, Arabidopsis thaliana
MELFPAQPDLSLQISPPNSKPSSTWQRRRSTTDQEDHEELDLGFWRRALDSRTSSLVSNSTSKTINHPFQDLSLSNISHHQQQQQHHHPQLLPNCNSSNILTSFQFPTQQQQQHLQGFLAHDLNTHLRPIRGIPLYHNPPPHHHPHRPPPPCFPFDPSSLIPSSSTSSPALTGNNNSFNTSSVSNPNYHNHHHQTLNRARFMPRFPAK

SEQ ID NO: 43 - protein, 170 aa, Arabidopsis thaliana
RVNQPSVARMVSVETQRGNNQPFSNVQENVHLVGSFGASSSSSVGAVGNLFGSLYDIHGGMVTNLHPTRTVRPNHRLAFHDGSFEQDYYDVGSDNLLVNQQVGGYGYHMNPVDQFKWNQSFNNTMNMNYNNDSTSGSSRGSDMNVNHDNKKIRYRNSVIMHPCHLEKDGP

SEQ ID NO: 44 - protein, 166 aa, Arabidopsis thaliana
INRGIDPTSHRPIQESSASQDSKPTQLEPVTSNTINISFTSAPKVETFHESISFPGKSEKISMLTFKEEKDECPVQEKFPDLNLELRISLPDDVDRLQGHGKSTTPRCFKCSLGMINGMECRCGRMRCDVVGGSSKGSDMSNGFDFLGLAKKETTSLLGFRSLEMK

SEQ ID NO: 45 - protein, 216 aa, Arabidopsis thaliana
MESVELTLKNSNMKDKTLTGGAQNGDDFSVDDLLDFSKEEEDDDVLVEDEAELKVQRKRGVSDENTLHRSNDFSTADFHTSGLSVPMDDIAELEWLSNFVDDSSFTPYSAPTNKPVWLTGNRRHLVQPVKEETCFKSQHPAVKTRPKRARTGVRVWSHGSQSLTDSSSSSTTSSSSSPRPSSPLWLASGQFLDEPMTKTQKKKKVWKNAGQTQTQT

SEQ ID NO: 46 - protein, 219 aa, Arabidopsis thaliana
NVSRRKRRSSLFDMVPDEVGDIPMDLQEPEEDNIPVETEMQGADSIHQTLAPSSLHAPSILEIEECESMDSTNSTTGEPTATAAAASSSSRLEETTQLQSQLQPQPQLPGSFPILYPTYFSPYYPFPFPIWPAGYVPEPPKKEETHEILRPTAVHSKAPINVDELLGMSKLSLAESNKHGESDQSLSLKLGGGSSSRQSAFHPNPSSDSSDIKSVIHAL

SEQ ID NO: 47 - protein, 82 aa, Arabidopsis thaliana
HTEMECEYLKRWFGSLKEQNRRLQIEVEELRALKPSSTSALTMCPRCERVTDAVDNDSNAVQEGAVLSSRSRMTISSSSSLC

SEQ ID NO: 48 - protein, 116 aa, Arabidopsis thaliana
LRHKYQEATTITSLQNNFNSTTTTSSVSCDQHALASAILLPPPPPPPPTPRPPRLLSSQPAPPPTPPVSLPSPSMVVSSSSSSNSSATNSMYNPPPSSTAGYSNSLSSDNNVHYFD

SEQ ID NO: 49 - protein, 207 aa, Arabidopsis thaliana
MKQTLSRYGNHQSSSASKAEEDCAEVDILKDQLSKLQEKHLQLQGKGLNPLTFKELQSLEQQLYHALITVRERKERLLTNQLEESRLKEQRAELENETLRRQVQELRSFLPSFTHYVPSYIKCFAIDPKNALINHDSKCSLQNTDSDTTLQLGLPGEAHDRRTNEGERESPSSDSVTTNTSSETAERGDQSSLANSPPEAKRQRFSV

SEQ ID NO: 50 - protein, 125 aa, Arabidopsis thaliana
FTARGPLLHPIATFHANPSTSKEMALVEYTPPSIRYHNTHPPSQGSSSFTAIGTIEGKFDCGYLVKVKLGSEILNGVLYHSAQPGPSSSPTAVLNNAVVPYVETGRRRRRLGKRRRSRRREDPNY

SEQ ID NO: 51 - protein, 417 aa, Arabidopsis thaliana
NLPWKLRQRSTKEVRKKVYVCPVSGCVHHDPSRALGDLTGIKKHFCRKHGEKKWKCEKCSKKYAVQSDWKAHSKICGTKEYKCDCGTLFSRRDSFITHRAFCDALAEESAKNHTQSKKLYPETVTRKNPEIEQKSPAAVESSPSLPPSSPPSVAIAPAPAISVETESVKIISSSVLPIQNSPESQENNNHPEVIIEEASRTIGFNVSSSDLSNDHSNNNGGYAGLFVSSTASPSLYASSTASPSLFAPSSSMEPISLCLSTNPSLFGPTIRDPPHFLTPLPPQPAMSATALLQKAAQMGSTGSGGSLLRGLGIVSTTSSSMELSNHDALSLAPGLGLGLPCSSGGSGSGLKELMMGNSSVFGPKQTTLDFLGLGRAVGNGGNTGGGLSALLTSIGGGGGIDLFGSGEFSGKDIGRSS

SEQ ID NO: 52 - protein, 257 aa, Arabidopsis thaliana
ARSQGVFFGGSLIGGDQQQGGLPIGPGNISSEAAVFDMEYARWLEEQQRLLNELRVATQEHLSENELRMFVDTCLAHYDHLINLKAMVAKTDVFHLISGAWKTPAERCFLWMGGFRPSEIIKVIVNQIEPLTEQQIVGICGLQQSTQEAEEALSQGLEALNQSLSDSIVSDSLPPASAPLPPHLSNFMSHMSLALNKLSALEGFVLQADNLRHQTIHRLNQLLTTRQEARCLLAVAEYFHRLQALSSLWLARPRQDG

SEQ ID NO: 53 - protein, 572 aa, Arabidopsis thaliana
KEAEVKGIPVCQALDIEIPPPRPKRKPNTPYPRKPGNNGTSSSQVSSAKDAKLVSSASSSQLNQAFLDLEKMPFSEKTSTGKENQDENCSGVSTVNKYPLPTKQVSGDIETSKTSTVDNAVQDVPKKNKDKDGNDGTTVHSMQNYPWHFHADIVNGNIAKCPQNHPSGMVSQDFMFHPMREETHGHANLQATTASATTTASHQAFPACHSQDDYRSFLQISSTFSNLIMSTLLQNPAAHAAATFAASVWPYASVGNSGDSSTPMSSSPPSITAIAAATVAAATAWWASHGLLPVCAPAPITCVPFSTVAVPTPAMTEMDTVENTQPFEKQNTALQDQNLASKSPASSSDDSDETGVTKLNADSKTNDDKIEEVVVTAAVHDSNTAQKKNLVDRSSCGSNTPSGSDAETDALDKMEKDKEDVKETDENQPDVIELNNRKIKMRDNNSNNNATTDSWKEVSEEGRIAFQALFARERLPQSFSPPQVAENVNRKQSDTSMPLAPNFKSQDSCAADQEGVVMIGVGTCKSLKTRQTGFKPYKRCSMEVKESQVGNINNQSDEKVCKRLRLEGEAST

SEQ ID NO: 54 - protein, 212 aa, Arabidopsis thaliana
MDDLHGRNGRMHIGVAQNPMHVQYEDHGLHHIDNENSMMDDHADGGMDEGVETDIPSHPGNSADNRGEVVDRGIENGDQLTLSFQGQVYVFDRVSPEKVQAVLLLLGGREVPHTLPTTLGSPHQNNRVLGLSGTPQRLSVPQRLASLLRFREKRKGRNFDKTIRYTVRKEVALRMQRKKGQFTSAKSSNDDSGSTGSDWGSNQSWAVEGTET

SEQ ID NO: 55 - protein, 141 aa, Arabidopsis thaliana
IDSSSPPPPNLRFNQIRNQNQNQVDPFMDHRLFTDHQQQFPIVNRPTSSSMSSTVESFSGPRPTTMKPATTKRYPRTPPVVPEDCHSDCDSSSSVIDDDDDIASSSRRRNPPFQFDLNFPPLDCVDLFNGADDLHCTDLRL

SEQ ID NO: 56 - protein, 242 aa, Arabidopsis thaliana
ARQQGVFISSSGDQAHSTAGDGAMAFDVEYRRWQEDKNRQMKELSSAIDSHATDSELRIIVDGVIAHYEELYRIKGNAAKSDVFHLLSGMWKTPAERCFLWLGGFRSSELLKLIASQLEPLTEQQSLDINNLQQSSQQAEDALSQGMDNLQQSLADTLSSGTLGSSSSGNVASYMGQMAMAMGKLGTLEGFIRQADNLRLQTYQQMVRLLTTRQSARALLAVHNYTLRLRALSSLWLARPRE

SEQ ID NO: 57 - protein, 157 aa, Arabidopsis thaliana
MVSPFSMPFIAQTSGFVNYSQVFITQTIAKRYHALIPTSNMVIVQNDNDRVNRFMTSYPPILKSTVNPPNDFDKQYETFTPKPIDFFCSQQDYACRQHLDIFSSSPKHYHEQYVHKNGRSVKYICKPTEVLEEIHDEIDYEKDGGWIYSLPFEKDSS

SEQ ID NO: 58 - protein, 120 aa, Arabidopsis thaliana
MGLDDSCNTGLVLGLGLSPTPNNYNHAIKKSSSTVDHRFIRLDPSLTLSLSGESYKIKTGAGAGDQICRQTSSHSGISSFSSGRVKREREISGGDGEEEAEETTERVVCSRVSDDHDDEE

SEQ ID NO: 59 - protein, 267 aa, Arabidopsis thaliana
LMSSTVSTSTNPSPINCNGRKSMLKLAKRMTDNFCGGVCASSLQKWSKLNVGNVDEDVRIMTRKSVNNPGEPPGIILNAATSVWMPVSPRRLFDFLGNERLRSEWDILSNGGPMKEMAHIAKGHDRSNSVSLLRASAINANQSSMLILQETSIDAAGAVVVYAPVDIPAMQAVMNGGDSAYVALLPSGFAILPNGQAGTQRCAAEERNSIGNGGCMEEGGSLLTVAFQILVNSLPTAKLTVESVETVNNLISCTVQKIKAALHCDST

SEQ ID NO: 60 - protein, 200 aa, Arabidopsis thaliana
RTVDFNKVVIALKDYAALRERTADGDPNPVVVNNNTSSSGIDPDAVAAIRRQRLSEISLWFGPHCSTNNNNSSNSAAAGTASSQVTSEQPVGIVNFDILPMESRATKWAVEGTGILLATGLLTVTLAWLIAPRVGKRTAKSGLHILLGGLCALTVVIFFRFVVLTRIRYGPARYWAILFVFWFLVFGIWASRSHASHSST

SEQ ID NO: 61 - protein, 453 aa, Arabidopsis thaliana
YSGGRQPAVLRTFSQRLCRGFNDAVNGFVDDGWSPMSSDGGEDITIMINSSSAKFAGSQYGSSFLPSFGSGVLCAKASMLLQNVPPLVLIRFLREHRAEWADYGVDAYSAASLRATPYAVPCVRTGGFPSNQVILPLAQTLEHEEFLEVVRLGGHAYSPEDMGLSRDMYLLQLCSGVDENVVGGCAQLVFAPIDESFADDAPLLPSGFRVIPLDQKTNPNDHQSASRTRDLASSLDGSTKTDSETNSRLVLTIAFQFTFDNHSRDNVATMARQYVRNVVGSIQRVALAITPRPGSMQLPTSPEALTLVRWITRSYSIHTGADLFGADSQSCGGDTLLKQLWDHSDAILCCSLKTNASPVFTFANQAGLDMLETTLVALQDIMLDKTLDDSGRRALCSEFAKIMQQGYANLPAGICVSSMGRPVSYEQATVWKVVDDNESNHCLAFTLVSWSFV

SEQ ID NO: 62 - protein, 83 aa, Arabidopsis thaliana
GWDNNQRVFNNNSNNKNGLAMTNSSGSGGFSVNNNGVGVNREIVNGGYASRNVQGGWENLKHDQRQQCYAVINNGFKQHYLPL

SEQ ID NO: 63 - protein, 33 aa, Arabidopsis thaliana
KGSYNHLIKSASIKRATAAATAAAAAVAAVPES

SEQ ID NO: 64 - protein, 140 aa, Arabidopsis thaliana
TTIRWEFSNEMFRKGQRELMSNIRRRKSQHWSHNKSNHQVVPTTTMVNQEGHQRIGIDHHHEDQQSSATSSSFVYTALLDENKCLKNENELLSCELGKTKKKCKQLMELVERYRGEDEDATDESDDEEDEGLKLFGVKLE

SEQ ID NO: 65 - protein, 151 aa, Arabidopsis thaliana
SPGTQIAILATPLSSHSHASFYSFGHSSVDHVVSSLLHNQHPSLPTNQDNRSGLGFWWEDQAFDRLENVDELKEAVDAVSRMLNNVRLRLDDAVKSNQRDGSLVIHQEDEEVLQLGYKDTNQITKLEGETSASASLLKNVVDNLHIDDRYY

SEQ ID NO: 66 - protein, 239 aa, Arabidopsis thaliana
MAVDLMRFPKIDDQTAIQEAASQGLQSMEHLIRVLSNRPEQQHNVDCSEITDFTVSKFKTVISLLNRTGHARFRRGPVHSTSSAASQKLQSQIVKNTQPEAPIVRTTTNHPQIVPPPSSVTLDFSKPSIFGTKAKSAELEFSKENFSVSLNSSFMSSAITGDGSVSNGKIFLASAPLQPVNSSGKPPLAGHPYRKRCLEHEHSESFSGKVSGSAYGKCHCKKSRKNRMKRTVRVPAISA

SEQ ID NO: 67 - protein, 179 aa, Arabidopsis thaliana
MKEVLERHNLQSKNLEKLDQPSLELQLVENSDHARMSKEIADKSHRLRQMRGEELQGLDIEELQQLEKALETGLTRVIETKSDKIMSEISELQKKGMQLMDENKRLRQQGTQLTEENERLGMQICNNVHAHGGAESENAAVYEEGQSSESITNAGNSTGAPVDSESSDTSLRLGLPYGG

SEQ ID NO: 68 - protein, 188 aa, Arabidopsis thaliana
AEINFVHNINNHNFVFGSPTYPTARFYPVTPEYSMPYRSFPPFYQNQFQEREYLGYGYGRVVNGNGVRYYAGSPLDQHHQWNLGRSEPLVYDSVPVFPAGRVPPSAPPQPSTTKKLRLFGVDVEESSSSGDTRGEMGVAGYSSSSPVVIRDDDQSFWRSPRGEMASSSSAMQLSDDEEYKRKGKSLEL

SEQ ID NO: 69 - protein, 205 aa, Arabidopsis thaliana
MMMFKSGDMDYTQKMKRCHEYVEALEEEQKKIQVFQRELPLCLELVTQAIESCRKELSESSEHVGGQSECSERTTSECGGAVFEEFMPIKWSSASSDETDKDEEAEKTEMMTNENNDGDKKKSDWLRSVQLWNQSPDPQPNNKKPMVIEVKRSAGAFQPFQKEKPKAADSQPLIKAITPTSTTTTSSTAETVGGGKEFEEQKQSH

SEQ ID NO: 70 - protein, 117 aa, Arabidopsis thaliana
NNGELNLPGSTLILSSSVEKEPSSHQGSGSGRARRDSAARAMQGWHAQRLVGSGEVTAPAVKDKGLISTPKHKKLKSIGLQKHKQQTSMDHVVTNEADKQLAAEVVDVGPVADWVKI

SEQ ID NO: 71 - protein, 237 aa, Arabidopsis thaliana
IDFEKAKNIGTGSLVVDDSGFDRTTTVASSEETLSSGGGCHVTTPIVSPEGKEATTSMEMSEEQCVEKTNGEGISRQDDKDPPTLFRPVPRLSSFNACNHMEGSPSPHIQDQNQLQSSKQDAAMLRLLEGAYSERFVPQTCGGGCCSNNPDGSFQQESLLGPEFVDYLDSPTFPSSELAAIATEIGSLAWLRSGLESSSVRVMEDAVGRLRPQGSRGHRDHYLVSEQGTNITNVLST

SEQ ID NO: 72 - protein, 199 aa, Arabidopsis thaliana
DTERWEFANEHFLKGERHLLKNIKRRKTSSQTQTQSLEGEIHELRRDRMALEVELVRLRRKQESVKTYLHLMEEKLKVTEVKQEMMMNFLLKKIKKPSFLQSLRKRNLQGIKNREQKQEVISSHGVEDNGKFVKAEPEEYGDDIDDQCGGVFDYGDELHIASMEHQGQGEDEIEMDSEGIWKGFVLSEEEMCDLVEHFI

SEQ ID NO: 73 - protein, 128 aa, Arabidopsis thaliana
MQMDSAQNQFNKRARLFEDPELKDAKVIYPSNPESTEPVNKGYGGSTAIQSFFKESKAEETPKVLKKRGRKKKNPNPEEVNSSTPGGDDSENRSKFYESASARKRTVTAEERERAVNAAKTFEPTNPY

SEQ ID NO: 74 - protein, 208 aa, Arabidopsis thaliana
GEETEISSSSTGSEIEQIHSLIPLVNSSGGSEGSSFHSQELQNSSQSGVFANVQGESQIDDATTPIEEEWKTWLNNDGDEQRNIMFMQDHRSDYTPLKSLTGVFSDDSSDDNDSDLISPKTNSIGTSSTCASFASSNHQIDQTQHSPDSTVQLVSLTQEVSQGPGQVTVIREHKLGEESVKKKRASFVYRMIHRLVKKIHQCYSISRT

SEQ ID NO: 75 - protein, 169 aa, Arabidopsis thaliana
EDYQPAKPKTSNKKKGTKPKSESVITSSNSTRSESELADSSNPSGESLFSTSPSTSEVSSMTLISHDGYSNEINMDNKPGDISTIDQECVSFETFGADIDESFWKETLYSQDEHNYVSNDLEVAGLVEIQQEFQNLGSANNEMIFDSEMDFWFDVLARTGGEQDLLAGL

SEQ ID NO: 76 - protein, 120 aa, Arabidopsis thaliana
METLHPFSHLPISDHRFVVQEMVSLHSSSSGSWTKEENKMFERALAIYAEDSPDRWFKVASMIPGKTVFDVMKQYSKLEEDVFDIEAGRVPIPGYPAASSPLGFDTDMCRKRPSGARGSD

SEQ ID NO: 77 - protein, 85 aa, Arabidopsis thaliana
HRENRKTVNGDDIWWALSTLGLDNYADAVGRHLHKYREAERERTEHNKGSNDSGNEKETNTRSDVQNQSTKFIRVVEKGSSSSAR

SEQ ID NO: 78 - protein, 214 aa, Arabidopsis thaliana
MEDEAHEFFHTSDFAVDDLLVDFSNDDDEENDVVADSTTTTTITDSSNFSAADLPSFHGDVQDGTSFSGDLCIPSDDLADELEWLSNIVDESLSPEDVHKLELISGFKSRPDPKSDTGSPENPNSSSPIFTTDVSVPAKARSKRSRAAACNWASRGLLKETFYDSPFTGETILSSQQHLSPPTSPPLLMAPLGKKQAVDGGHRRKKDVSSPESG

SEQ ID NO: 79 - protein, 254 aa, Arabidopsis thaliana
VPDRWEFSNDCFKRGEKILLRDIQRRKISQPAMAAAAAAAAAAVAASAVTVAAVPVVAHIVSPSNSGEEQVISSNSSPAAAAAAIGGVVGGGSLQRTTSCTTAPELVEENERLRKDNERLRKEMTKLKGLYANIYTLMANFTPGQEDCAHLLPEGKPLDLLPERQEMSEAIMASEIETGIGLKLGEDLTPRLFGVSIGVKRARREEELGAAEEEDDDRREAAAQEGEQSSDVKAEPMEENNSGNHNGSWLELGK

SEQ ID NO: 80 - protein, 238 aa, Arabidopsis thaliana
WGSRKKAKLALKRTPPGTKQDDNNTALTIVALTNDDERAKPTSPGGSGGGSPRTCASKRSITSLDKIIFEAITNLRELRGSDRTSIFLYIEENFKTPPNMKRHVAVRLKHLSSNGTLVKIKHKYRFSSNFIPAGARQKAPQLFLEGNNKKDPTKPEENGANSLTKFRVDGELYMIKGMTAQEAAEAAARAVAEAEFAITEAEQAAKEAERAEAEAEAAQIFAKAAMKALKFRIRNHPW

SEQ ID NO: 81 - protein, 256 aa, Arabidopsis thaliana
LISSSVTSHDNTSITPGGRKSMLKLAQRMTFNFCSGISAPSVHNWSKLTVGNVDPDVRVMTRKSVDDPGEPPGIVLSAATSVWLPAAPQRLYDFLRNERMRCEWDILSNGGPMQEMAHITKGQDQGVSLLRSNAMNANQSSMLILQETCIDASGALVVYAPVDIPAMHVVMNGGDSSYVALLPSGFAVLPDGGIDGGGSGDGDQRPVGGGSLLTVAFQILVNNLPTAKLTVESVETVNNLISCTVQKIRAALQCES

SEQ ID NO: 82 - protein, 214 aa, Arabidopsis thaliana
LPDSSSEGKKTDKKESGDMLSGLDGSSGMQITEALKLQMEVQKRLHEQLEVQRQLQLRIEAQGKYLKKIIEEQQRLSGVLGEPSAPVTGDSDPATPAPTSESPLQDKSGKDCGPDKSLSVDESLSSYREPLTPDSGCNIGSPDESTGEERLSKKPRLVRGAAGYTPDIVVGHPILESGLNTSYHQSDHVLAFDQPSTSLLGAEEQLDKVSGDNL

SEQ ID NO: 83 - protein, 284 aa, Arabidopsis thaliana
MVSHKVLEFGDDGYKLPAQARAPRSLRKKRIYEKKIPGDDKMCAIDLLATVAGSLLLESKSPVNACLVVQNTVKNEYPADENPVKAVPYSESPSLFDNGKCGFSSVITNPNHLLVGDKVGKEVEGFSSLGVSGDVKPDVVASIGSNSSTEVGACGNGSPNESRDDVNLFSRNDDDENFSGYIRTRMTRPVPRIGDRRIRKILASRHWKGGSKNNTDAKPWYCSKRSYYLHHHQRSYPIKKRKYFDSVYDSNSDDYRLQGKTHKGSRTISSMKSRNASFVSRDHH

SEQ ID NO: 84 - protein, 188 aa, Arabidopsis thaliana
MGSLGDELSLGSIFGRGVSMNVVAVEKVDEHVKKLEEEKRKLESCQLELPLSLQILNDAILYLKDKRCSEMETQPLLKDFISVNKPIQGERGIELLKREELMREKKFQQWKANDDHTSKIKSKLEIKRNEEKSPMLLIPKVETGLGLGLSSSSIRRKGIVASCGFTSNSMPQPPTPAVPQQPAFLKQQ

SEQ ID NO: 85 - protein, 161 aa, Arabidopsis thaliana
IDCSPSSPLQPLTYLHNQNLCSPPVIQNQIDPFMDHRLYGGGNFQEQQQQQIISRPASSSMSSTVKSCSGPRPMEAAAASSSVAKPLHAIKRYPRTPPVAPEDCHSDCDSSSSVIDDGDDIASSSSRRKTPFQFDLNFPPLDGVDLFAGGIDDLHCTDLRL

SEQ ID NO: 86 - protein, 61 aa, Arabidopsis thaliana
MATQDSQGIKLFGKTITFNANITQTIKKEEQQQQQQPELQATTAVRSPSSDLTAEKRPDKI

SEQ ID NO: 87 - protein, 399 aa, Arabidopsis thaliana
NLPWKLKQRSKQEVIKKKVYICPIKTCVHHDASRALGDLTGIKKHYSRKHGEKKWKCEKCSKKYAVQSDWKAHAKTCGTREYKCDCGTLFSRKDSFITHRAFCDALTEEGARMSSLSNNNPVISTTNLNFGNESNVMNNPNLPHGFVHRGVHHPDINAAISQFGLGFGHDLSAMHAQGLSEMVQMASTGNHHLFPSSSSSLPDFSGHHQFQIPMTSTNPSLTLSSSSTSQQTSASLQHQTLKDSSFSPLFSSSSENKQNKPLSPMSATALLQKAAQMGSTRSNSSTAPSFFAGPTMTSSSATASPPPRSSSPMMIQQQLNNFNTNVLRENHNRAPPPLSGVSTSSVDNNPFQSNRSGLNPAQQMGLTRDFLGVSNEHHPHQTGRRPFLPQELARFAPLG

SEQ ID NO: 88 - protein, 139 aa, Arabidopsis thaliana
IFENRFIDGSASLCDRNVPKKRAFGTELTNVNAKRNKSGCSKEDSKRNGNQNTSIVIKQEQCDDVKPDVKNFASGSSTPAGTSESNDMGNNIRPRGRLGVIEALSTLYQPSYCNPELLGLFAHYNETFRSYQEEFGREK

SEQ ID NO: 89 - protein, 107 aa, Arabidopsis thaliana
ELLPGEKFSDEDMSAATIRKKATEVGAQVDALGTAVQNNRHRVFGQNRDSDVDNKNFHRNYQNGEREEEEEDEDDKRLRSGGRLLDRVDLNKLPDPESSDEEWESKH

SEQ ID NO: 90 - protein, 368 aa, Arabidopsis thaliana
RAGLPLYPHEIQHQGIDIDDEFEFDLTSFQFQNQDLDHNHQNMIQYTNSSNTSSSSSSFSSSSSQPSKRLRPDPLVSTNPGLNPIPDSSMDFQMFSLYNNSLENDNNQFGFSVPLSSSSSSNEVCNPNHILEYISENSDTRNTNKKDIDAMSYSSLLMGDLEIRSSSFPLGLDNSVLELPSNQRPTHSFSSSPIIDNGVHLEPPSGNSGLLDALLEESQALSRGGLFKDVRVSSSDLCEVQDKRVKMDFENLLIDHLNSSNHSSLGANPNIHNKYNEPTMVKVTVDDDDELLTSLLNNFPSTTTPLPDWYRVTEMQNEASYLAPPSGILMGNHQGNGRVEPPTVPPSSSVDPMASLGSCYWSNMPSIC

SEQ ID NO: 91 - protein, 229 aa, Arabidopsis thaliana
MASSSELSLDCKPQSYSMLLKSFGDNFQSDPTTHKLEDLLSRLEQERLKIDAFKRELPLCMQLLNNAVEVYKQQLEAYRANSNNNNQSVGTRPVLEEFIPLRNQPEKTNNKGSNWMTTAQLWSQSETKPKNIDSTTDQSLPKDEINSSPKLGHFDAKQRNGSGAFLPFSKEQSLPELALSTEVKRVSPTNEHTNGQDGNDESMINNDNNYNNNNNNNSNSNGVSSTTSQ

SEQ ID NO: 92 - protein, 135 aa, Arabidopsis thaliana
NLVKILDRYGKQHADDLKALDHQSKALNYGSHYELLELVDSKLVGSNVKNVSIDALVQLEEHLETALSVTRAKKTELMLKLVENLKEKEKMLKEENQVLASQMENNHHVGAEAEMEMSPAGQISDNLPVTLPLLN

SEQ ID NO: 93 - protein, 103 aa, Arabidopsis thaliana
QLEQLYDSLRQEYDVVSREKQMLHDEVKKLRALLRDQGLIKKQISAGTIKVSGEEDTVEISSVVVAHPRTENMNANQITGGNQVYGQYNNPMLVASSGWPSYP

SEQ ID NO: 94 - protein, 66 aa, Arabidopsis thaliana
DLLLQEEDHLSAATTADMPAALIREKAAEVGARVDALLASAAPSMAHSTPPVIKPDLNQIPESGDI

SEQ ID NO: 95 - protein, 90 aa, Arabidopsis thaliana
CYNINAHCLSLTQSLSQSSTVESSFPNLNLGSDSVSSRFPFPKIQVKAGMMVFDERSESDSSSVVMDVVRYEGRRVVLDLDLNFPPPPEN

SEQ ID NO: 96 - protein, 141 aa, Arabidopsis thaliana
TFLELSDQKVPTGFARSPSQSSTLDCASPPTLVVPSATAGNVPPQLELSLGGGGGGSCYQIPMSRPVYFLDLMGIGNVGRGQPPPVTSAFRSPVVHVATKMACGAQSDSDSSSVVDFEGGMEKRSQLLDLDLNLPPPSEQA

SEQ ID NO: 97 - protein, 235 aa, Arabidopsis thaliana
SSSSSSSNILQTIPSSLPDLNPPILFSNQIHNKSKGSSQDLNLLSFPVMQDQHHHHVHMSQFLQMPKMEGNGNITHQQQPSSSSSVYGSSSSPVSALELLRTGVNVSSRSGINSSFMPSGSMMDSNTVLYTSSGFPTMVDYKPSNLSFSTDHQGLGHNSNNRSEALHSDHHQQGRVLFPFGDQMKELSSSITQEVDHDDNQQQKSHGNNNNNNNSSPNNGYWSGMFSTTGGGSSW

SEQ ID NO: 98 - protein, 224 aa, Arabidopsis thaliana
SKRKHKRESNADNNDRDASPSAKRPCILQDYIKSIERNNINKDNDEKKNENTISVISTPNLDQIYSDGDSASSILGGPYDEELDYFQNIFANHPISLENLGLSQTSDEVTQSSSSGFMIKNPNPNLHDSVGIHHQEATITAPANTPHLASDIYLSYLLNGTTSSYSDTHFPSSSSSTSSTTVEHGGHNEFLEPQANSTSERREMDLIEMLSGSIQGSNICFPLV

SEQ ID NO: 99 - protein, 111 aa, Arabidopsis thaliana
LPGESTTVNDGGENDSYVNRTTVTTAREMTRQRFPFACHRERKVVGGYASAGFFFDPSRAASLRAELSRVCPVRFDPVNIELSIGIRETVKVEPRRELNLDLNLAPPVVDV

SEQ ID NO: 100 - protein, 280 aa, Arabidopsis thaliana
MELPQPRPFKTQEFRTGRKPTHDFLSLCSHSTVHPDPKPTPPPSSQGSHLKTHDFLQPLECVGAKEDVSRINSTTTASEKPPPPAPPPPLQHVLPGGIGTYTISPIPYFHHHHQRIPKPELSPPMMFNANERNVLDENSNSNCSSYAAASSGFTLWDESASGKKGQTRKENSVGERVNMRADVAATVGQWPVAERRSQSLTNNHMSGFSSLSSSQGSVLKSQSFMDMIRSAKGSSQEDDLDDEEDFIMKKESSSTSQSHRVDLRVKADVRGSPNDQKLNT

SEQ ID NO: 101 - protein, 133 aa, Arabidopsis thaliana
NLNRRRRRSSLFDITTETVTEMAMEQDPTQENSPLPETNISSGQQAMQVFTDVPTKTENAPETFHLNDPYLVPVTFQAKPTFNLNTDAAPLSLNLCLASSFNLNEQPNSRHSAFTMMPSFSDGDSNSSIIRVA

SEQ ID NO: 102 - protein, 210 aa, Arabidopsis thaliana
KSGTGEHLPPPRPKRKAAHPYPQKAHKNVQLQVPGSFKSTSEPNDPSFMFRPESSSMLMTSPTTAAAAPWTNNAQTISFTPLPKAGAGANNNCSSSSENTPRPRSNRDARDHGNVGHSLRVLPDFAQVYGFIGSVFDPYASNHLQKLKKMDPIDVETVLLLMRNLSINLSSPDFEDHRRLLSSYDIGSETATDHGGVNKTLNKDPPEIST

SEQ ID NO: 103 - protein, 153 aa, Arabidopsis thaliana
RINQPSVAQMVSVGIQPGSHKPFFNVQENNDFVGSFGASSSSFVAAVGNRFSSLSHIHGGMVTNVHPTQTFRPNHRLAFHNGSFEQDYYDVGSDNLLVNQQVGGYVDNHNGYHMNQVDQYNWNQSFNNAMNMNYNNASTSGRMHPSHLEKGGP

SEQ ID NO: 104 - protein, 315 aa, Arabidopsis thaliana
NNIGPPSGNRYAPFMEEEWADGGGALIPGIDVRVRVEALPQANGNNQMDQWADLLKLHNSIKFAITFCRTQLNLTALSNERCSTREIFIVFWLICKEMHSASKDLININELPRDATPMDIEPNQQNHHESAFKPQESNNHSGYEEDEDTLKREHAEEDERPPSLCILNKEAPLPLLQYKRRRQNESNNNSSRNTQDHCSSTITTVDNTTTLISSSAAAATNTAISALLEFSLMGISDKKENQQKEETSPPSPIASPEEKVNDLQKEVHQMSVERETFKLEMMSAEAMISILQSRIDALRQENEELKKKNASGQAS

SEQ ID NO: 105 - protein, 149 aa, Arabidopsis thaliana
MQKTIERYRKYTKDHETSNHDSQIHLQQLKQEASHMITKIELLEFHKRKLLGQGIASCSLEELQEIDSQLQRSLGKVRERKAQLFKEQLEKLKAKEKQLLEENVKLHQKNVINPWRGSSTDQQQEKYKVIDLNLEVETDLFIGLPNRNC

SEQ ID NO: 106 - protein, 210 aa, Arabidopsis thaliana
MCSVSELLDMENFQGDLTDVVRGIGGHVLSPETPPSNIWPLPLSHPTPSPSDLNINPFGDPFVSMDDPLLQELNSITNSGYFSTVGDNNNNIHNNNGFLVPKVFEEDHIKSQCSIFPRIRISHSNIIHDSSPCNSPAMSAHVVAAAAAASPRGIINVDTNSPRNCLLVDGTTFSSQIQISSPRNLGLKRRKSQAKKVVCIPAPAAMNSRS

SEQ ID NO: 107 - protein, 301 aa, Arabidopsis thaliana
IPGALKELQEEGVKDTFHRFYVNENVKGSDDEDDDEESSQPHSSSQTDSSKPGSLPQSSDPSKIDNRREKSLGLLTQNFIKLFICSEAIRIISLDDAAKLLLGDAHNTSIMRTKVRRLYDIANVLSSMNLIEKTHTLDSRKPAFKWLGYNGEPTFTLSSDLLQLESRKRAFGTDITNVNVKRSKSSSSSQENATERRLKMKKHSTPESSYNKSFDVHESRHGSRGGYHFGPFAPGTGTYPTAGLEDNSRRAFDVENLDSDYRPSYQNQVLKDLFSHYMDAWKTWFSEVTQENPLPNTSQHR

SEQ ID NO: 108 - protein, 181 aa, Arabidopsis thaliana
LSQGLDPSTHNLMPSHKRSSSSNNNNIPKPNKTTSIMKNPTDLDQSTTAFSITNINPPTSTKPNKLKSPNQTTIPSQTVIPINDNMSSTQTMIPINDPMSSLLDDENMIPHWSDVDGMAIHEAPMLPSDKAVVGVDDDDLNMDILFNTPSSSAFDPDFASIFSSAMSIDFNPMDDLGSWTF

SEQ ID NO: 109 - protein, 194 aa, Arabidopsis thaliana
QLEKDYGVLKTQYDSLRHNFDSLRRDNESLLQEISKLKTKLNGGGGEEEEEENNAAVTTESDISVKEEEVSLPEKITEAPSSPPQFLEHSDGLNYRSFTDLRDLLPLKAAASSFAAAAGSSDSSDSSALLNEESSSNVTVAAPVTVPGGNFFQFVKMEQTEDHEDFLSGEEACEFFSDEQPPSLHWYSTVDHWN

SEQ ID NO: 110 - protein, 191 aa, Arabidopsis thaliana
IESTIERYNRCYNCSLSNNKPEETTQSWCQEVTKLKSKYESLVRTNRNLLGEDLGEMGVKELQALERQLEAALTATRQRKTQVMMEEMEDLRKKERQLGDINKQLKIKFETEGHAFKTFQDLWANSAASVAGDPNNSEFPVEPSHPNVLDCNTEPFLQIGFQQHYYVQGEGSSVSKSNVAGETNFVQGWVL

SEQ ID NO: 111 - protein, 91 aa, Arabidopsis thaliana
MDREDINPMLSRLDVENNNTFSSFVDKTLMMMPPSTFSGEVEPSSSSSWYPESFHVHAPPLPPENDQIGEKGKELKEKRSRKVPRIAFHTR

SEQ ID NO: 112 - protein, 223 aa, Arabidopsis thaliana
PPLPISPENFSIFNHHQSFLNLGQRPGQDPTQLGFKINGCVQKSTTTSREENDREKGENDVVYTNNHHVGSYGTYHNLEHHHHHHQHLSLQADYHSHQLHSLVPFPSQILVCPMTTSPTTTTIQSLFPSSSSAGSGTMETLDPRQMVSHFQMPLMGNSSSSSSQNISTLYSLLHGSSSNNGGRDIDNRMSSVQFNRTNSTTTANMSRHLGSERCTSRGSDHHM

SEQ ID NO: 113 - protein, 213 aa, Arabidopsis thaliana
WPSSNHYLQVTSEDCDNNNSGTILSFGSSESSVTETGKHQSGDTAKISADSVSQENKSYQGFLPPQVMLPNNSSPWPYQWSPTGPNASFYPVPFYWGCTVPIYPTSETSSCLGKRSRDQTEGRINDTNTTITTTRARLVSESLRMNIEASKSAVWSKLPTKPEKKTQGFSLFNGFDTKGNSNRSSLVSETSHSLQANPAAMSRAMNFRESMQQ

SEQ ID NO: 114 - protein, 159 aa, Arabidopsis thaliana
VNDKRKRRASLFDISLEDQKEKERNSQDASTKTPPKQPITGIQQPVVQGHTQTEISNRFQNLSMEYMPIYQPIPPYYNFPPIMYHPNYPMYYANPQVPVRFVHPSGIPVPRHIPIGLPLSQPSEASNMTNKDGLDLHIGLPPQATGASDLTGHGVIHVK

SEQ ID NO: 115 - protein, 142 aa, Arabidopsis thaliana
FRSNGQIPPDSMQSPSARPCFIQGAIRPSQELQALTFTPQPKINTAEFLGGSLAGSNVVGVIDGKFESGYLVTVTIGSEQLKGVLYQLLPQNTVSYQTPQQSHGVLPNTLNISANPQGVAGGVTKRRRRRKKSEIKRRDPDH

SEQ ID NO: 116 - protein, 50 aa, Arabidopsis thaliana
MSMRRSKAEGKRSLRELSEEEEEEEETEDEDTFEEEEALEKKQKGKATSS

SEQ ID NO: 117 - protein, 261 aa, Arabidopsis thaliana
KEFKKAKQHEDKATSGGSTKMSYYNEIEDIFRERKKKVAFYKSPATTTPSSAKVDSFMQFTDKGFEDTGISFTSVEANGRPTLNLETELDHDGLPLPIAADPITANGVPPWNWRDTPGNGVDGQPFAGRIITVKFGDYTRRVGIDGTAEAIKEAIRSAFRLRTRRAFWLEDEEQVIRSLDRDMPLGNYILRIDEGIAVRVCHYDESDPLPVHQEEKIFYTEEDYRDFLARRGWTCLREFDAFQNIDNMDELQSGRLYRGMR

SEQ ID NO: 118 - protein, 27 aa, Arabidopsis thaliana
ESYMPWSHGFLNMLDLLFTRTVNGTTL

SEQ ID NO: 119 - protein, 363 aa, Arabidopsis thaliana
NLPWKLKQKSNKEVRRKVYLCPEPSCVHHDPARALGDLTGIKKHYYRKHGEKKWKCDKCSKRYAVQSDWKAHSKTCGTKEYRCDCGTIFSRRDSYITHRAFCDALIQESARNPTVSFTAMAAGGGGGARHGFYGGASSALSHNHFGNNPNSGFTPLAAAGYNLNRSSSDKFEDFVPQATNPNPGPTNFLMQCSPNQGLLAQNNQSLMNHHGLISLGDNNNNNHNFFNLAYFQDTKNSDQTGVPSLFTNGADNNGPSALLRGLTSSSSSSVVVNDFGDCDHGNLQGLMNSLAATTDQQGRSPSLFDLHFANNLSMGGSDRLTLDFLGVNGGIVSTVNGRGGRSGGPPLDAEMKFSHPNHPYGKA

SEQ ID NO: 120 - protein, 58 aa, Arabidopsis thaliana
EEVFKDGNGGEGLGGDMSPTLIRKKAAEVGARVDAELRLENRMVENLDMNKLPEAYGL

SEQ ID NO: 121 - protein, 67 aa, Arabidopsis thaliana
FLSSSTTRKEAKTTRPNKAHPSTSSSSSSSRWSNLLSSAEAGISRLGNDISQKLQFSSSKDNGIVEV

SEQ ID NO: 122 - protein, 139 aa, Arabidopsis thaliana
MDQYSSSLVDTSLDLTIGVTRMRVEEDPPTSALVEELNRVSAENKKLSEMLTLMCDNYNVLRKQLMEYVNKSNITERDQISPPKKRKSPAREDAFSCAVIGGVSESSSTDQDEYLCKKQREETVVKEKVSRVYYKTEAS

SEQ ID NO: 123 - protein, 223 aa, Arabidopsis thaliana
MDMRSHEMIERRREDNGNNNGGVVISNIISTNIDDNCNGNNNNTRVSCNSQTLDHHQSKSPSSFSISAAAKPTVRYRECLKNHAASVGGSVHDGCGEFMPSGEEGTIEALRCAACDCHRNFHRKEMDGVGSSDLISHHRHHHYHHNQYGGGGGRRPPPPNMMLNPLMLPPPPNYQPIHHHKYGMSPPGGGGMVTPMSVAYGGGGGGAESSSEDLNLYGQSSGE

SEQ ID NO: 124 - protein, 106 aa, Arabidopsis thaliana
TLCEKPTSYFVRKCGHAEKTKASHTGYEQEEHINSDIDTASAQLPVISPTSTVRVSEGKYPLSGFKKMRRELSNDNLDQKADVEMISAGSNKKALSLAKRAISPDG

SEQ ID NO: 125 - protein, 433 aa, Arabidopsis thaliana
MEQGGGGGGNEVVEEASPISSRPPANNLEELMRFSAAADDGGLGGGGGGGGGGSASSSSGNRWPREETLALLRIRSDMDSTFRDATLKAPLWEHVSRKLLELGYKRSSKKCKEKFENVQKYYKRTKETRGGRHDGKAYKFFSQLEALNTTPPSSSLDVTPLSVANPILMPSSSSSPFPVFSQPQPQTQTQPPQTHNVSFTPTPPPLPLPSMGPIFTGVTFSSHSSSTASGMGSDDDDDDMDVDQANIAGSSSRKRKRGNRGGGGKMMELFEGLVRQVMQKQAAMQRSFLEALEKREQERLDREEAWKRQEMARLAREHEVMSQERAASASRDAAIISLIQKITGHTIQLPPSLSSQPPPPYQPPPAVTKRVAEPPLSTAQSQSQQPIMAIPQQQILPPPPPSHPHAHQPEQKQQQQPQQEMVMSSEQSSLPSS

SEQ ID NO: 126 - protein, 216 aa, Arabidopsis thaliana
TMCKKIRRSSDQSEEIKVESDSDEQNQASDDVLSLDEDDDDSDYNCGEDNDSDDYADEAAVEKDDNDADDEDVDNVADDVPVEDDDYVEAFDSRDHAKADDDDEDERQYLDDRENPSFTLILNPKKKSQLLIPARVIKDYDLHFPESITLVDPLVKKFGTLEKQIKIQTNGSVFVKGFGSIIRRNKVKTTDKMIFEIKKTGDNNLVQTIKIHIISG

SEQ ID NO: 127 - protein, 142 aa, Arabidopsis thaliana
NKKGKRFSIHDMTLGDAENVTVPVSNLNSMGQQPHFDDQSPPDHYQDYFSQSNVTIPGCNMHFMGQQPRFGDQIPPGEYHPYSRDNVTVTGSNLNSIGQQPHFNDQISPDQYGRYLQENFGFFDDDGEDDGSLASFQQLYKA

SEQ ID NO: 128 - protein, 69 aa, Arabidopsis thaliana
GFQDLLLNPVLTAGCSTDFSLQSTHQNYISDCNLGYFLQIGFQQHYEQGEGSSVTKSNARSDAETNFVQ

SEQ ID NO: 129 - protein, 216 aa, Arabidopsis thaliana
MDDLHGSNARMHIREAQDPMHVQFEHHALHHIHNGSGMVDDQADDGNAGGMSEGVETDIPSHPGNVTDNRGEVVDRGSEQGDQLTLSFQGQVYVFDSVLPEKVQAVLLLLGGRELPQAAPPGLGSPHQNNRVSSLPGTPQRFSIPQRLASLVRFREKRKGRNFDKKIRYTVRKEVALRMQRNKGQFTSAKSNNDEAASAGSSWGSNQTWAIESSEA

SEQ ID NO: 130 - protein, 205 aa, Arabidopsis thaliana
IQMGIDPVTHRPRTDHLNVLAALPQLLAAANFNNLLNLNQNIQLDATSVAKAQLLHSMIQVLSNNNTSSSFDIHHTTNNLFGQSSFLENLPNIENPYDQTQGLSHIDDQPLDSFSSPIRVVAYQHDQNFIPPLISTSPDESKETQMMVKNKEIMKYNDHTSNPSSTSTFTQDHQPWCDIIDDEASDSYWKEIIEQTCSEPWPFRE

SEQ ID NO: 131 - protein, 290 aa, Arabidopsis thaliana
MFRFPVSLGGSRDEDRHDQITPLDDHRVVVDEVDFFSEKRDRVSRENINDDDDEGNKVLIKMEGSRVEENDRSRDVNIGLNLLTANTGSDESTVDDGLSMDMEDKRAKIENAQLQEELKKMKIENQRLRDMLSQATTNFNALQMQLVAVMRQQEQRNSSQDHLLAQESKAEGRKRQELQIMVPRQFMDLGPSSGAAEHGAEVSSEERTTVRSGSPPSLLESSNPRENGKRLLGREESSEESESNAWGNPNKVPKHNPSSSNSNGNRNGNVIDQSAAEATMRKARVSVRAR

SEQ ID NO: 132 - protein, 59 aa, Arabidopsis thaliana
MEGQRTQRRGYLKDKATVSNLVEEEMENGMDGEEEDGGDEDKRKKVMERVRGPSTDRVP

SEQ ID NO: 133 - protein, 253 aa, Arabidopsis thaliana
LLSSEDHTGLSHAGTKSILKLAQRMKLNFYSGITASCIHKWEKLLAENVGQDTRILTRKSLEPSGIVLSAATSLWLPVTQQRLFEFLCDGKCRNQWDILSNGASMENTLLVPKGQQEGSCVSLLRAAGNDQNESSMLILQETWNDVSGALVVYAPVDIPSMNTVMSGGDSAYVALLPSGFSILPDGSSSSSDQFDTDGGLVNQESKGCLLTVGFQILVNSLPTAKLNVESVETVNNLIACTIHKIRAALRIPA

SEQ ID NO: 134 - protein, 113 aa, Arabidopsis thaliana
MDTNKAKKLKVMNQLVEGHDLTTQLQQLLSQPGSGLEDLVAKILVCFNNTISVLDTFEPISSSSSLAAVEGSQNASCDNDGKFEDSGDSRKRLGPVKGKRGCYKRKKRSETCT

SEQ ID NO: 135 - protein, 153 aa, Arabidopsis thaliana
MDVYGMSSPDLLRIDDLLDFSNDEIFSSSSTVTSSAASSAASSENPFSFPSSTYTSPTLLTDFTHDLCVPSDDAAHLEWLSRFVDDSFSDFPANPLTMTVRPEISFTGKPRSRRSRAPAPSVAGTWAPMSESELCHSVAKPKPKKVYNAESVT

SEQ ID NO: 136 - protein, 161 aa, Arabidopsis thaliana
VHEQFMKTQRKHMDHVTDQLMVELHRGRRLDDLDLSEINALISFSRENIILLRKELEFVQHSPLGDPRVPPFEAQFEELTTIANDVFVRGGQVDERAWKNYEATKRVSIGNALRGNQSHYLVDKWLFASPKPREPTNQSRLTYQTIFYTKEAVATDALIWI

SEQ ID NO: 137 - protein, 94 aa, Arabidopsis thaliana
TGHGVTTTSNEDIQPNRNFPSYTFNGDNISNNVFPCTVVNTGHRQMVFPVSTMTDHAPSTNYSTISDNYNSTFNGNATASDTTSAATTTATTTV

SEQ ID NO: 138 - protein, 292 aa, Arabidopsis thaliana
LNGQANNSFNKIGIMTMMEEKTPDADEIQSENLSIGPQPNKNSPIGEALQMQIEVQRRLHEQLEVQRHLQLRIEAQGKYLQSVLEKAQETLGRQNLGAAGIEAAKVQLSELVSKVSAEYPNSSFLEPKELQNLCSQQMQTNYPPDCSLESCLTSSEGTQKNSKMLENNRLGLRTYIGDSTSEQKEIMEEPLFQRMELTWTEGLRGNPYLSTMVSEAEQRISYSERSPGRLSIGVGLHGHKSQHQQGNNEDHKLETRNRKGMDSTTELDLNTHVENYCTTRTKQFDLNGFSWN

SEQ ID NO: 139 - protein, 176 aa, Arabidopsis thaliana
MDGSSFLDISLDLNTNPFSAKLPKKEVSVLASTHLKRKWLEQDESASELREELNRVNSENKKLTEMLARVCESYNELHNHLEKLQSRQSPEIEQTDIPIKKRKQDPDEFLGFPIGLSSGKTENSSSNEDHHHHHQQHEQKNQLLSCKRPVTDSFNKAKVSTVYVPTETSDTSLTVK

SEQ ID NO: 140 - protein, 130 aa, Arabidopsis thaliana
SMNKDRRRSSIHDITSVGNADVSTPQGPITGQNNSNNNNNNNNNNSSPAVAGGGNKSAKQAVSQAPPGPPMYGTPAIGQPAVGTPVNLPAPPHMAYGVHAAPVPGSVVPGAAMNIGQMPYTMPRTPTAHR

SEQ ID NO: 141 - protein, 355 aa, Arabidopsis thaliana
MAASFLTMDNSRTRQNMNGSANWSQQSGRTSTSSLEDLEIPKFRSFAPSSISISPSLVSPSTCFSPSLFLDSPAFVSSSANVLASPTTGALITNVTNQKGINEGDKSNNNNFNLFDFSFHTQSSGVSAPTTTTTTTTTTTTTNSSIFQSQEQQKKNQSEQWSQTETRPNNQAVSYNGREQRKGEDGYNWRKYGQKQVKGSENPRSYYKCTFPNCPTKKKVERSLEGQITEIVYKGSHNHPKPQSTRRSSSSSSTFHSAVYNASLDHNRQASSDQPNSNNSFHQSDSFGMQQEDNTTSDSVGDDEFEQGSSIVSRDEEDCGSEPEAKRWKGDNETNGGNGGGSKTVREPRIVVQTT

SEQ ID NO: 142 - protein, 156 aa, Arabidopsis thaliana
KLRLQVMEQQAKLRDALNEQLKKEVERLKFATGEVSPADAYNLGMAHMQYQQQPQQSFFQHHHQQQTDAQNLQQMTHQFHLFQPNNNQNQSSRTNPPTAHQLMHHATSNAPAQSHSYSEAMHEDHLGRLQGLDISSCGRGSNFGRSDTVSESSSTM

SEQ ID NO: 143 - protein, 272 aa, Arabidopsis thaliana
MDKEKSPAPPPSGGLPPPSGRYSAFSPNGSSFAMKAESSFPPLTPSGSNSSDANRFSHDISRMPDNPPKNLGHRRAHSEILTLPDDLSFDSDLGVVGAADGPSFSDDTDEDLLYMYLDMEKFNSSATSTSQMGEPSEPTWRNELASTSNLQSTPGSSSERPRIRHQHSQSMDGSTTIKPEMLMSGNEDVSGVDSKKAISAAKLSELALIDPKRAKRIWANRQSAARSKERKMRYIAELERKVQTLQTEATSLSAQLTLLQRDTNGLGVENNE

SEQ ID NO: 144 - protein, 303 aa, Arabidopsis thaliana
RLGLPVYPDEVREHAMNAATHSGLNTDSLDGHHSQEYMEADTVEIPEVDFEHLPLNRSSSYYQSMLRHVPPTNVFVRQKPCFFQPPNVYNLIPPSPYMSTGKRPREPETAFPCPGGYTMNEQSPRLWNYPFVENVSEQLPDSHLLGNAAYSSPPGPLVHGVENFEFPSFQYHEEPGGWGADQPNPMPEHESDNTLVQSPLTAQTPSDCPSSSLYDGLLESVVYGSSGEKPATDTDSESSLFQSFTPANENITGKTCFLTLYALHALHCLCNQFKKSPLLHLHDKLNWCNKFRFNSFKSGTHIL

SEQ ID NO: 145 - protein, 207 aa, Arabidopsis thaliana
NKVNQDSHQELDRSSLSSSPSSSSANSNSNISRGQWERRLQTDIHLAKKALSEALSPAVAPIITSTVTTTSSSAESRRSTSSASGFLRTQETSTTYASSTENIAKLLKGWVKNSPKTQNSADQIASTEVKEVIKSDDGKECAGAFQSFSEFDHSYQQAGVSPDHETKPDITGCCSNQSQWSLFEKWLFEDSGGQIGDILLDENTNFF

SEQ ID NO: 146 - protein, 284 aa, Arabidopsis thaliana
SSSHYRHITISEALEAARLDPGLQANTRVLSFGLEAQQQHVAAPMTPVMKLQEDQKVSNGARNRFHGLADQRLVARVENGDDCSSGSSVTTSNNHSVDESRAQSGSVVEAQMNNNNNNNMNGYACIPGVPWPYTWNPAMPPPGFYPPPGYPMPFYPYWTIPMLPPHQSSSPISQKCSNTNSPTLGKHPRDEGSSKKDNETERKQKAGCVLVPKTLRIDDPNEAAKSSIWTTLGIKNEAMCKAGGMFKGFDHKTKMYNNDKAENSPVLSANPAALSRSHNFHEQI

SEQ ID NO: 147 - protein, 215 aa, Arabidopsis thaliana
TRYKACETTEPDAIRQQFPFYNEIQSIFEARMQRMLWSEATEPSTSSKRKHHQFSSDDEEEEVDEPNQDINEELLSLVETQKRETEVITTSTSTNPRKRAKKGKGVASGTKAETAGNTLKDILEEFMRQTVKMEKEWRDAWEMKEIEREKREKEWRRRMAELEEERAATERRWMEREEERRLREEARAQKRDSLIDALLNRLNRDHNDDHHNQGF

SEQ ID NO: 148 - protein, 190 aa, Arabidopsis thaliana
MNMDKETEQTLNYLPLGQSDPFGNGNEGTIGDFLGRYCNNPQEISPLTLQSFSLNSQISENFPISGGIRFPPYPGQFGSDREFGSQPTTQESNKSSLLDPDSVSDRVHTTKSNSRKRKSIPSGNGKESPASSSLTASNSKVSGENGGSKGGKRSKQDVAGSSKNGVEKCDSKGDNKDDAKPPEAPKDYIH

SEQ ID NO: 149 - protein, 306 aa, Arabidopsis thaliana
MEEIEGTNRAAVESCHRVLNLLHRSQQQDHVGFEKNLVSETREAVIRFKRVGSLLSSSVGHARFRRAKKLQSHVSQSLLLDPCQQRTTEVPSSSSQKTPVLRSGFQELSLRQPSDSLTLGTRSFSLNSNAKAPLLQLNQQTMPPSNYPTLFPVQQQQQQQQQQQQQEQQQQQQQQQQQFHERLQAHHLHQQQQLQKHQAELMLRKCNGGISLSFDNSSCTPTMSSTRSFVSSLSIDGSVANIEGKNSFHFGVPSSTDQNSLHSKRKCPLKGDEHGSLKCGSSSRCHCAKKRKHRVRRSIRVPAISN

SEQ ID NO: 150 - protein, 169 aa, Arabidopsis thaliana
RSRTCSNSSSSSVSGVVSNSNGVPLQTTPVLFPQSSISNGVTHTVTESDGKGSALSLCGSFTSTLLNHNAAATATHGSGSVIGIGGFGIGLGSGFDDVSFGLGRAMWPFSTVGTATTTNVGSNGGHHAVPMPATWQFEGLESNAGGGFVSGEYFAWPDLSITTPGNSLK

SEQ ID NO: 151 - protein, 244 aa, Arabidopsis thaliana
DRARQQGFYVGNGVDTNALSFSDNMSSGIVAFEMEYGHWVEEQNRQICELRTVLHGQVSDIELRSLVENAMKHYFQLFRMKSAAAKIDVFYVMSGMWKTSAERFFLWIGGFRPSELLKVLLPHFDPLTDQQLLDVCNLRQSCQQAEDALSQGMEKLQHTLAESVAAGKLGEGSYIPQMTCAMERLEALVSFVNQADHLRHETLQQMHRILTTRQAARGLLALGEYFQRLRALSSSWAARQREPT

SEQ ID NO: 152 - protein, 109 aa, Arabidopsis thaliana
MNGLVDSSRDKKMKNPRFSFRTKSDADILDDGYRWRKYGQKSVKNSLYPRSYYRCTQHMCNVKKQVQRLSKETSIVETTYEGIHNHPCEELMQTLTPLLHQLQFLSKFT

SEQ ID NO: 153 - protein, 129 aa, Arabidopsis thaliana
AGHQTSAAGDLRHSSESTNQFMTWQQTSVSPIGSAYSTPYNHHQPYYGHVNPNNPVSPQSSLEESFSNTSSDVTTTANVRETHHQTGGGVYGHDGIGFHEGYPNKKRSVSYCSSDLGELQALALRMMKN

SEQ ID NO: 154 - protein, 96 aa, Arabidopsis thaliana
SGAKDKRRPSIHDITTVNLLNANLSRPSSDHGCLVSKQAEPKLGFTDRDNAEEGVMFLGQNLSSVFSSYDPAIKFSGANVYGEGGYCISQDLETRK

SEQ ID NO: 155 - protein, 193 aa, Arabidopsis thaliana
SKSRSKSTVVVSTDNTTSTSSLTSRPSYSNPSKFHSYGQIPEFNSNLPILPPLQSLGDYNSSNTGLDFGGTQISNMISGMSSSGGILDAWRIPPSQQAQQFPFLINTTGLVQSSNALYPLLEGGVSATQTRNVKAEENDQDRGRDGDGVNNLSRNFLGNININSGRNEEYTSWGGNSSWTGFTSNNSTGHLSF

SEQ ID NO: 156 - protein, 92 aa, Arabidopsis thaliana
LEAGKHEDLGDNKKTISLKAKRKRQVTEDESQLISRKAVKREEAQVQADACPLTPSSWKGFWDGADSKDMGIFSVPLLSPCPSLGHSQLVVT

SEQ ID NO: 157 - protein, 76 aa, Arabidopsis thaliana
ELLPCTSAEDMSAATIRKKATEVGAQVDAIGATVVQNNKRRRVFSQKRDFGGGLLELVDLNKLPDPENLDDDLVGK

SEQ ID NO: 158 - protein, 384 aa, Arabidopsis thaliana
RAGLPLYPPEMHVEALEWSQEYAKSRVMGEDRRHQDFLQLGSCESNVFFDTLNFTDMVPGTFDLADMTAYKNMGNCASSPRYENFMTPTIPSSKRLWESELLYPGCSSTIKQEFSSPEQFRNTSPQTISKTCSFSVPCDVEHPLYGNRHSPVMIPDSHTPTDGIVPYSKPLYGAVKLELPSFQYSETTFDQWKKSSSPPHSDLLDPFDTYIQSPPPPTGGEESDLYSNFDTGLLDMLLLEAKIRNNSTKNNLYRSCASTIPSADLGQVTVSQTKSEEFDNSLKSFLVHSEMSTQNADETPPRQREKKRKPLLDITRPDVLLASSWLDHGLGIVKETGSMSDALAVLLGDDIGNDYMNMSVGASSGVGSCSWSNMPPVCQMTELP

SEQ ID NO: 159 - protein, 296 aa, Arabidopsis thaliana
KPVHSHSLPNLQAQLNPLTDSERVRMNNQIERLTKEKEGLLEELHKQDEEREVFEMQVKELKERLQHMEKRQKTMVSFVSQVLEKPGLALNLSPCVPETNERKRRFPRIEFFPDEPMLEENKTCVVVREEGSTSPSSHTREHQVEQLESSIAIWENLVSDSCESMLQSRSMMTLDVDESSTFPESPPLSCIQLSVDSRLKSPPSPRIIDMNCEPDGSKEQNTVAAPPPPPVAGANDGFWQQFFSENPGSTEQREVQLERKDDKDKAGVRTEKCWWNSRNVNAITEQLGHLTSSERS

SEQ ID NO: 160 - protein, 177 aa, Arabidopsis thaliana
MIKKFSNMDYNQKRERCGQYIEALEEERRKIHVFQRELPLCLDLVTQAIEACKRELPEMTTENMYGQPECSEQTTGECGPVLEQFLTIKDSSTSNEEEDEEFDDEHGNHDPDNDSEDKNTKSDWLKSVQLWNQPDHPLLPKEERLQQETMTRDESMRKDPMVNGGEGRKREAEKDGG

SEQ ID NO: 161 - protein, 139 aa, Arabidopsis thaliana
RSRTYSSAATTSVVGSRNFPLQATPVLFPQSSSNGGITTAKGSASSFYGGFSSLINYNAAVSRNGPGGGFNGPDAFGLGLGHGSYYEDVRYGQGITVWPFSSGATDAATTTSHIAQIPATWQFEGQESKVGFVSGDYVA

SEQ ID NO: 162 - protein, 162 aa, Arabidopsis thaliana
MSNYGVKELTWENGQLTVHGLGDEVEPTTSNNPIWTQSLNGCETLESVVHQAALQQPSKFQLQSPNGPNHNYESKDGSCSRKRGYPQEMDRWFAVQEESHRVGHSVTASASGTNMSWASFESGRSLKTARTGDRDYFRSGSETQDTEGDEQETRGEAGRSNG

SEQ ID NO: 163 - protein, 283 aa, Arabidopsis thaliana
REATGGDGSSVEPIVIPPPRPKRKPAHPYPRKFGNEADQTSRSVSPSERDTQSPTSVLSTVGSEALCSLDSSSPNRSLSPVSSASPPAALTTTANAPEELETLKLELFPSERLLNRESSIKEPTKQSLKLFGKTVLVSDSGMSSSLTTSTYCKSPIQPLPRKLSSSKTLPIIRNSQEELLSCWIQVPLKQEDVENRCLDSGKAVQNEGSSTGSNTGSVDDTGHTEKTTEPETMLCQWEFKPSERSAFSELRRTNSESNSRGFGPYKKRKMVTEEEEHEIHLHL

SEQ ID NO: 164 - protein, 217 aa, Arabidopsis thaliana
KKMNDSCDSTINNGLDNKDFSISNKNTTSHQSSNSSKGQWERRLQTDINMAKQALCDALSIDKPQNPTNFSIPDLGYGPSSSSSSTTTTTTTTRNTNPYPSGVYASSAENIARLLQNFMKDTPKTSVPLPVAATEMAITTAASSPSTTEGDGEGIDHSLFSFNSIDEAEEKPKLIDHDINGLITQGSLSLFEKWLFDEQSHDMIINNMSLEGQEVLF

SEQ ID NO: 165 - protein, 168 aa, Arabidopsis thaliana
GVEIIDVPLGVEPETEPFHPTPKKPHKETTPASSFASGSGCSANGGTNGRGKQRSSDVKNPERYLLNPENPYFVQAVTKRNDVLYVSRPVVQSYRLKFGPVKSTITYLLPGEKKEEGENRIYNGKPCFSGWSVLCRRHNLNIGDSVVCELERSGGVVTAVRVHFVKKD

SEQ ID NO: 166 - protein, 156 aa, Arabidopsis thaliana
TKQLEKDYDTLKRQFDTLKAENDLLQTHNQKLQAEIMGLKNREQTESINLNKETEGSCSNRSDNSSDNLRLDISTAPPSNDSTLTGGHPPPPQTVGRHFFPPSPATATTTTTTMQFFQNSSSGQSMVKEENSISNMFCAMDDHSGFWPWLDQQQYN

SEQ ID NO: 167 - protein, 165 aa, Arabidopsis thaliana
MSSTSFTDLLGSSGVDCYEDDEDLRVSGSSFGGYYPERTGSGLPKFKTAQPPPLPISQSSHNFTFSDYLDSPLLLSSSHSLISPTTGTFPLQGFNGTTNNHSDFPWQLQSQPSNASSALQETYGVQDHEKKQEMIPNEIATQNNNQSFGTERQIKIPAYMVSRNS

SEQ ID NO: 168 - protein, 210 aa, Arabidopsis thaliana
GDYKKIKEWESQIKEETESYWVMRNDVRREKKLPGFFDKEVYDIVDGGVIPPAVPVLSLGLAPASDEGLLSDLDRRESPEKLNSTPVAKSVTDVIDKEKQEACVADQGRVKEKQPEAANVEGGSTSQEERKRKRTSFGEKEEEEEEGETKKMQNQLIEILERNGQLLAAQLEVQNLNLKLDREQRKDHGDSLVAVLNKLADAVAKIADKM

SEQ ID NO: 169 - protein, 136 aa, Arabidopsis thaliana
LIGYYGISSATPVNNNLSETVSDGNANLPLVGDDGNALASPVNNTLSETARDGTLPSDCHDMLSPGVAEAVAGFFLDLPEVIALKEELDRVCPDQFESIDMGLTIGPQTAVEEPETSSAVDCKLRMEPDLDLNASP

SEQ ID NO: 170 - protein, 390 aa, Arabidopsis thaliana
SGSGPKNGEQYGAPFVEEEWEEEDDMTFVPDQEDLGSEDHVYVHMDDIDQKSENFVVYDAIPIPLNFIHGESSNNVETNYSDSINYIQQTGNYMDSGGYFEQPAESYEKDQKPIIRDRDGSLQNEGIGCGVQDKHSETLQSSDNIFGTDTSCYNDFPVESNYLIGEAFLDPNSNLLENDGLYLETNDLSSTQQDGFDFEDYLTFFDETFDPSQLMGNEDVFFDQEELFQEVETKELEKEETSRSKHVVEEKEKDEASCSKQVDADATEFEPDYKYPLLKKASHMLGAIPAPLANASEFPTKDAAIRLHAAQSSGSVHVTAGMITISDSNMGWSYGKNENLDLILSLGLVQGNTAPEKSGNSSAWAMLIFMCFWVLLLSVSFKVSILVSSR

SEQ ID NO: 171 - protein, 90 aa, Arabidopsis thaliana
MSSSDSVNNGVNSRMYFRNPSFSNVILNDNWSDLPLSVDDSQDMAIYNTLRDAVSSGWTPSVPPVTSPAEENKPPATKASGSHAPRQKGM

SEQ ID NO: 172 - protein, 61 aa, Arabidopsis thaliana
DKDNTGLGDGDKDNTCKGDDDKEKSGSGGCEKENEGNGGSGKDNNGNGDSQPAECSTGQKQ

SEQ ID NO: 173 - protein, 183 aa, Arabidopsis thaliana
MEFESVFKMHYPYLAAVIYDDSSTLKDFHPSLTDDFSCVHNVHHKPSMPHTYEIPSKETIRGITPSPCTEAFEACFHGTSNDHVFFGMAYTTPPTIEPNVSHVSHDNTMWENDQNQGFIFGTESTLNQAMADSNQFNMPKPLLSANEDTIMNRRQNNQVMIKTEQIKKKNKRFQMRRICKPTK

SEQ ID NO: 174 - protein, 138 aa, Arabidopsis thaliana
SGVVSRETNLISSSSSSAVTGEFSSAGSAIAPIINTFATEHVSCFSNNSAAHTDASFHTFLPAPPPSLPPRQPRHVGDGVAFGQFLDLGSSGQIDFDAAAAAFFPNLPSLPPTVLPPPPSFAMYGGGSPAVSVWPFTL

SEQ ID NO: 175 - protein, 408 aa, Arabidopsis thaliana
RAGLPLYPPEIYVDDLHWSEEYTKSNIIRVDRRRRHQDFLQLGNSKDNVLFDDLNFAASLLPAASDLSDLVACNMLGTGASSSRYESYMPPILPSPKQIWESGSRFPMCSSNIKHEFQSPEHFQNTAVQKNPRSCSISPCDVDHHPYENQHSSHMMMVPDSHTVTYGMHPTSKPLFGAVKLELPSFQYSETSAFDQWKTTPSPPHSDLLDSVDAYIQSPPPSQVEESDCFSSCDTGLLDMLLHEAKIKTSAKHSLLMSSPQKSFSSTTCTTNVTQNVPRGSENLIKSGEYEDSQKYLGRSEITSPSQLSAGGFSSAFAGNVVKTEELDQVWEPKRVDITRPDVLLASSWLDQGCYGIVSDTSSMSDALALLGGDDIGNSYVTVGSSSGQAPRGVGSYGWTNMPPVWSL

SEQ ID NO: 176 - protein, 204 aa, Arabidopsis thaliana
SGMGIDPVTHKPFSHLMAEITTTLNPPQVSHLAEAALGCFKDEMLHLLTKKRVDLNQINFSNHNPNPNNFHEIADNEAGKIKMDGLDHGNGIMKLWDMGNGFSYGSSSSSFGNEERNDGSASPAVAAWRGHGGIRTAVAETAAAEEEERRKLKGEVVDQEEIGSEGGRGDGMTMMRNHHHHQHVFNVDNVLWDLQADDLINHMV

SEQ ID NO: 177 - protein, 133 aa, Arabidopsis thaliana
ESVKEYEEQKKEKTMESVVLVKKSSLVLDENHQPSSSSSSDGNRNSSSSNLPEIEVRVSGKDVLIKILCEKQKGNVIKIMGEIEKLGLSITNSNVLPFGPTFDISIIAQKNNNFDMKIEDVVKNLSFGLSKLT

SEQ ID NO: 178 - protein, 475 aa, Arabidopsis thaliana
SALANKIEEQHHGTKKNKGTTNSEQSTSSTCLYSDGMYENLENSGYPVSPETGGLTQLGNNSSSDMETIENKWSQFMSHDTSFNFPPQSQYGTISYPPSKVDIALECARLQNRMLPPVPPLYVEGLTHNEYFGNNVANDTDEMLSKIIALAQASHEPRNSLDSWDGGSASGNFHGDFNYSGEKVSCLEANVEAVDMQEHHVNFKEERLVENLRWVGVSSKELEKSFVEEHSTVIPIEDIWRYHNDNQEQEHHDQDGMDVNNNNGDVDDAFTLEFSENEHNENLLDKNDHETTSSSCFEVVKKVEVSHGLFVTTRQVTNTFFQQIVPSQTVIVYINPTDGNECCHSMTSKEEVHVRKKINPRINGVSSTVLGQWRKFAHVIGFIPMLLLMRCVHRGNSNKNRGSEGYSRQPTRGDCNNRGTILMMENAVVRRKIWKKKKEKNMVDEQGFRFQDSFVLKKLGLSLAIILAVSTISLI

SEQ ID NO: 179 - protein, 71 aa, Arabidopsis thaliana
MIPAEINGYFQYLSPEYNVINMPSSPTSSLNYLNDLIINNNNYSSSSNSQDLMISNNSTSDEDHHQSIMVL

SEQ ID NO: 180 - protein, 161 aa, Arabidopsis thaliana
VQPEPEPVQEQEQEPESNMSVSISESMDDSQHLSSPTSVLNYQTYVSEEPIDSLIKPVKQEFLEPEQEPISWHLGEGNTNTNDDSFPLDITFLDNYFNESLPDISIFDQPMSPIQPTENDFFNDLMLFDSNAEEYYSSEIKEIGSSFNDLDDSLISDLLLV

SEQ ID NO: 181 - protein, 115 aa, Arabidopsis thaliana
ERAQLASNTSTTTGPPNYYSSNNQIYYSNPQTNPQTIPYFNQYYYNQYLHQGGNSNDALSYSLAGGETGGSMYNHQTLSTTNSSSSGGSSRQQDDEQDYARYLRFGDSSPPNSGF

SEQ ID NO: 182 - protein, 245 aa, Arabidopsis thaliana
METEDDLCNTNWGSSSSKSREPGSSDCGNSTFAGFTSQQKWEDASILDYEMGVEPGLQESIQANVDFLQGVRAQAWDPRTMLSNLSFMEQKIHQLQDLVHLLVGRGGQLQGRQDELAAQQQQLITTDLTSIIIQLISTAGSLLPSVKHNMSTAPGPFTGQPGSAVFPYVREANNVASQSQNNNNCGAREFDLPKPVLVDEREGHVVEEHEMKDEDDVEEGENLPPGSYEILQLEKEEILAPHTHF

SEQ ID NO: 183 - protein, 187 aa, Arabidopsis thaliana
TKSNSNNNNNSTATSNNTSFSSGNASTISTILSSHYGGNQESILSQILSPARLMNPTYNHLGDLTSNTKTDNNMSLLNYGGLSQDLRSIHMGASGGSLMSCVDEWRSASYHQQSSMGGGNLEDSSNPNPSANGFYSFESPRITSASISSALASQFSSVKVEDNPYKWVNVNGNCSSWNDLSAFGSSR

SEQ ID NO: 184 - protein, 238 aa, Arabidopsis thaliana
ITISYIETAGSTLTRQKSLKEQYLFHCQCARCSNFGKPHDIEESAILEGYRCANEKCTGFLLRDPEEKGFVCQKCLLLRSKEEVKKLASDLKTVSEKAPTSPSAEDKQAAIELYKTIEKLQVKLYHSFSIPLMRTREKLLKMLMDVEIWREALNYCRLIVPVYQRVYPATHPLIGLQFYTQGKLEWLLGETKEAVSSLIKAFDILRISHGISTPFMKELSAKLEEARAEASYKQLALH

SEQ ID NO: 185 - protein, 230 aa, Arabidopsis thaliana
MAPPMTNCLTFSLSPMEMLKSTDQSHFSSSYDDSSTPYLIDNFYAFKEEAEIEAAAASMADSTTLSTFFDHSQTQIPKLEDFLGDSFVRYSDNQTETQDSSSLTPFYDPRHRTVAEGVTGFFSDHHQPDFKTINSGPEIFDDSTTSNIGGTHLSSHVVESSTTAKLGFNGDCTTTGGVLSLGVNNTSDQPLSCNNGERGGNSNKKKTVSKKETSDDSKKKIVETLGQRTS

SEQ ID NO: 186 - protein, 135 aa, Arabidopsis thaliana
MATPNEVSALFLIKKYLLDELSPLPTTATTNRWMNDFTSFDQTGFEFSEFETKPEIIDLVTPKPEIFDFDVKSEIPSESNDSFTFQSNPPRVTVQSNRKPPLKIAPPNRTKWIQFATGNPKPELPVPVVAAEEKR

SEQ ID NO: 187 - protein, 179 aa, Arabidopsis thaliana
ADFRASSTQKMEDGVVQDDGYVGQRGGLEKEDKSYYESEHQIPNGDIAESSNVVEDQADTDDDCYAEILNDDIIKLDEEALKASQAFRPTNPTHQETISSESSSKRSKCGIKKESTETMNCYALFRIKNVAGTDSSWRFPNPFKIKKDDSQRLMKNVLATTVFLAILFSFFWTVLIARN

SEQ ID NO: 188 - protein, 122 aa, Arabidopsis thaliana
REQSSSYRRRKTMVSLKPLINPNPHIFNDFDPTRLALTHLASSDHKQLMLPVPCFPGYDHENESPLMVDMFETQMMVGDYIAWTQEATTFDFLNQTGKSEIFERINEEKKPPFFDFLGLGTV

SEQ ID NO: 189 - protein, 302 aa, Arabidopsis thaliana
MELLAGDCRKRVGDDFEEDINPFDGSDGGCGWMYGTRQMGSNGNDDALATLADLASPPQKLKPIRCGVKLPSSSEDRHPLDILAGTLDRLPEMGFGCFEAPLGSKIADVEESGQLTRGFSKEEDDSLPPLQMEFQARNRISWDGLSLSSSVDSSDSDSSPDVRKTVTGKRKRETRVKLEHFLEKLVGSMMKRQEKMHNQLINVMEKMEVERIRREEAWRQQETERMTQNEEARKQEMARNLSLISFIRSVTGDEIEIPKQCEFPQPLQQILPEQCKDEKCESAQREREIKFRYSSGSGSSGR

SEQ ID NO: 190 - protein, 108 aa, Arabidopsis thaliana
VTSQRNPTILPPNRKPVITLTDTCSKTSSLDSDHTSHRTVDSMSHEPPLPQPQNPYWNQHIVGFNQPTYTGNDNNLLMSFWNGNGGDFIGDSASWDELRSVIDGNTKP

SEQ ID NO: 191 - protein, 233 aa, Arabidopsis thaliana
INRYDVKAILESSTLPIGGGAAKRLKEAQALESSRKREAEMIALGSSFQYGGGSSTGSGSTSSRLQLQPYPLSIQQPLEPFLSLQNNDISHYNNNNAHDSSSFNHHSYIQTQLHLHQQTNNYLQQQSSQNSQQLYNAYLHSNPALLHGLVSTSIVDNNNNNGGSSGSYNTAAFLGNHGIGIGSSSTVGSTEEFPTVKTDYDMPSSDGTGGYSGWTSESVQGSNPGGVFTMWNE

SEQ ID NO: 192 - protein, 285 aa, Arabidopsis thaliana
MFRFPVSLGGGPRENLKPSDEQHQRAVVNEVDFFRSAEKRDRVSREEQNIIADETHRVHVKRENSRVDDHDDRSTDHINIGLNLLTANTGSDESMVDDGLSVDMEEKRTKCENAQLREELKKASEDNQRLKQMLSQTTNNFNSLQMQLVAVMRQQEDHHHLATTENNDNVKNRHEVPEMVPRQFIDLGPHSDEVSSEERTTVRSGSPPSLLEKSSSRQNGKRVLVREESPETESNGWRNPNKVPKHHASSSICGGNGSENASSKVIEQAAAEATMRKARVSVRAR

SEQ ID NO: 193 - protein, 110 aa, Arabidopsis thaliana
DIVRQGHYKQILSPSINAKIESICNSSDLPLPQIEKQNKTEEVLSGFSKPEKEPEFGEIYGCGYSGSSPESDITLLDFSSDCVKEDESFLMGLHKYPSLEIDWDAIEKLF

SEQ ID NO: 194 - protein, 273 aa, Arabidopsis thaliana
NSNVTETHRRGNNADRRKPVVNSDSDYDVDGTEEASGSVSSKDSRRNQIQKEQPTAISHSVRDQDKAEKHRRRKRPRIRSGTVNRQEEEQPEAQQRNILPDMNHVDAPLLEYNINGTHQEDDVVDPNIALGPEDNGLELVVPEFNNNYTYLPLVNEQTMMPVDERPMLYGPNPNQELQFGSGYNFYNPSAVFVHNQEDDILHTQIEMNTQAPPHNSGFEEAPGGVLQPLGLLGNEDGVTGSELPQYQSGILSPLTDLDFDYGGFGDDFSWFGA

SEQ ID NO: 195 - protein, 779 aa, Arabidopsis thaliana
DSYMSSGLLDQYQAMPLAPYERSSTLQSTFMQSNIDGNGCLNGQAENEIDSRQNSSMVGCSLSARDFQNGTINIGHDFHPCGNSQENEQTAYHSEQFYYPELEDISVSISEVSYDMEDCSQFPDHNVSTSPSQDYQFDFQELSDISLEMRHNMSEIPMPYTKESKESTLGAPNSTLNIDVATYTNSANVLTPETECCRVLFPDQESEGHSVSRSLTQEPNEFNQVDRRDPILYSSASDRQISEATKSPTQSSSSRFTATAASGKGTLRPAPLIISPDKYSKKSSGLICHPFEVEPKCTTNGNGSFICIGDPSSSTCVDEGTNNSSEEDQSYHVNDPKKLVPVNDFASLAEDRPHSLPKHEPNMTNEQHHEDMGASSSLGFPSFDLPVFNCDLLQSKNDPLHDYSPLGIRKLLMSTMTCMSPLRLWESPTGKKTLVGAQSILRKRTRDLLTPLSEKRSDKKLEIDIAASLAKDFSRLDVMFDETENRQSNFGNSTGVIHGDRENHFHILNGDGEEWSGKPSSLFSHRMPEETMHIRKSLEKVDQICMEANVREKDDSEQDVENVEFFSGILSEHNTGKPVLSTPGQSVTKAEKAQVSTPRNQLQRTLMATSNKEHHSPSSVCLVINSPSRARNKEGHLVDNGTSNENFSIFCGTPFRRGLESPSAWKSPFYINSLLPSPRFDTDLTIEDMGYIFSPGERSYESIGVMTQINEHTSAFAAFADAMEVSISPTNDDARQKKELDKENNDPLLAERRVLDFNDCESPIKATEEVSSYLLKGCR

SEQ ID NO: 196 - protein, 71 aa, Arabidopsis thaliana
RAQVLELNHRLQSLNEIVDFVESSSSGFGMETGQGLFDGGLFDGVMNPMNLGFYNQPIMASASTAGDVFNC

SEQ ID NO: 197 - protein, 206 aa, Arabidopsis thaliana
KNGTLAHVPPPRPKRKAAHPYPQKASKNAQMPLQVSTSFTTTRNGDMPGYASWDDASMLLNRVISPQHELATLRGAEADIGSKGLLNVSSPSTSGMGSSSRTVSGSEIVRKAKQPPVLHGVPDFAEVYNFIGSVFDPETRGHVEKLKEMDPINFETVLLLMRNLTVNLSNPDLESTRKVLLSYDNVTTELPSVVSLVKNSTSDKSA

SEQ ID NO: 198 - protein, 211 aa, Arabidopsis thaliana
MMVEMDYAKKMQKCHEYVEALEEEQKKIQVFQRELPLCLELVTQAIEACRKELSGTTTTTSEQCSEQTTSVCGGPVFEEFIPIKKISSLCEEVQEEEEEDGEHESSPELVNNKKSDWLRSVQLWNHSPDLNPKEERVAKKAKVVEVKPKSGAFQPFQKRVLETDLQPAVKVASSMPATTTSSTTETCGGKSDLIKAGDEERRIEQQQSQSH

SEQ ID NO: 199 - protein, 78 aa, Arabidopsis thaliana
TQPRQCGSMEPKPKNLVNLNRFSYENIQAGFGYEHGGKSEETTQVIRELVVREGDGSCSFLSFTCDASKGKESFMKNQ

SEQ ID NO: 200 - protein, 322 aa, Arabidopsis thaliana
IVIEAKPRDQHRSYVHAMSNVSGNCSSSFDTCSDLEISSTTHQVQNTFQPRFGNERFNSNAISNEDWSQYYGSSYRPFPTPYKVNTEIECSMLQHNIYLPPLRVENSAFSDSDFFTSMTHNNDHGVFDDFTFAASNSNHNNSVGDQVIHVGNYDEQLITSNRHMNQTGYIKEQKIRSSLDNTDEDPGFHGNNTNDNIDIDDFLSFDIYNEDNVNQIEDNEDVNTNETLDSSGFEVVEEETRFNNQMLISTYQTTKILYHQVVPCHTLKVHVNPISHNVEERTLFIEEDKDSWLQRAEKITKTKLTLFSLMAQQYYKCLAIFF

SEQ ID NO: 201 - protein, 106 aa, Arabidopsis thaliana
LEKPVSSLQSTDEALKSLANESPNPEEGIDEPQVGYEVQGFIDGKFDSGYLVTMKLGSQELKGVLYHIPQTPSQSQQTMETPSAIVQSSQRRHRKKSKLAVVDTQK

SEQ ID NO: 202 - protein, 232 aa, Arabidopsis thaliana
KNLWNSCLKKKLRLRGIDPVTHKLLTEIETGTDDKTKPVEKSQQTYLVETDGSSSTTTCSTNQNNNTDHLYTGNFGFQRLSLENGSRIAAGSDLGIWIPQTGRNHHHHVDETIPSAVVLPGSMFSSGLTGYRSSNLGLIELENSFSTGPMMTEHQQIQESNYNNSTFFGNGNLNWGLTMEENQNPFTISNHSNSSLYSDIKSETNFFGTEATNVGMWPCNQLQPQQHAYGHI

SEQ ID NO: 203 - protein, 368 aa, Arabidopsis thaliana
SGSGPKNGEQYGAPFIEEEWAEDDDDDVDEPANQLVVSASVDNSLWGKGLNQSELDDNDIEELMSQVRDQSGPTLQQNGVSGLNSHVDTYNLENLEEDMYLEINDLMEPEPEPTSVEVMENNWNEDGSGLLNDDDFVGADSYFLDLGVTNPQLDFVSGDLKNGFAQSLQVNTSLMTYQANNNQFQQQSGKNQASNWPLRNSYTRQINNGSSWVQELNNDGLTVTRFGEAPGTGDSSEFLNPVPSGISTTNEDDPSKDESSKFASSVWTFLESIPAKPAYASENPFVKLNLVRMSTSGGRFRFTSKSTGNNVVVMDSDSAVKRNKSGGNNDKKKKKNKGFFCLSIIGALCALFWVIIGTMGGSGRPLLW

SEQ ID NO: 204 - protein, 238 aa, Arabidopsis thaliana
LSPPRPLGTSTQRNPSSSLAGSRLKAMALDCEMVGGGADGTIDQCASVCLVDDDENVIFSTHVQPLLPVTDYRHEITGLTKEDLKDGMPLEHVRERVFSFLCGGQNDGAGRLLLVGHDLRHDMSCLKLEYPSHLLRDTAKYVPLMKTNLVSQSLKYLTKSYLGYKIQCGKHEVYEDCVSAMRLYKRMRDQEHVCSGKAEGNGLNSRKQSDLEKMNAEELYQKSTSEYRCWCLDRLSNP

SEQ ID NO: 205 - protein, 60 aa, Arabidopsis thaliana
RAQASELTDRLRSLNSVLEMVEEISGQALDIPEIPESMQNPWQMPCPMQPIRASADMFDC

SEQ ID NO: 206 - protein, 124 aa, Arabidopsis thaliana
LQVKVLSMSRLGGAASASSQISEDAGGSHENTSSSGEAKMTEHQVAKLMEEDMGSAMQYLQGKGLCLMPISLATTISTATCPSRSPFVKDTGVPLSPNLSTTIVANGNGSSLVTVKDAPSVSKP

SEQ ID NO: 207 - protein, 318 aa, Arabidopsis thaliana
TGTGTIPANFTSLNISLRSSGSSMSLPSHFRSAASTFSPNNIFSPAMLQQQQQQQRGGGVGFHHPHLQGRAPTSSLFPGIDNFTPTTSFLNFHNPTKQEGDQDSEELNSEKKRRIQTTSDLHQQQQQHQHDQIGGYTLQSSNSGSTATAAAAQQIPGNFWMVAAAAAAGGGGGNNNQTGGLMTASIGTGGGGGEPVWTFPSINTAAAALYRSGVSGVPSGAVSSGLHFMNFAAPMAFLTGQQQLATTSNHEINEDSNNNEGGRSDGGGDHHNTQRHHHHQQQHHHNILSGLNQYGRQVSGDSQASGSLGGGDEEDQQD

SEQ ID NO: 208 - protein, 104 aa, Arabidopsis thaliana
KEERRASTARNSTSGGGSTAAGVPTLDHQASANYYYNNNNQYASSSPWHHQHNTQRVPYYSPANNEYSYVDDVRVVDHDVTTDPFLSWRLNVADRTGLVHDFTM

SEQ ID NO: 209 - protein, 233 aa, Arabidopsis thaliana
MEEHIQDRREIAFLHSGEFLHGDSDSKDHQPNESPVERHHESSIKEVDFFAAKSQPFDLGHVRTTTIVGSSGFNDGLGLVNSCHGTSSNDGDDKTKTQISRLKLELERLHEENHKLKHLLDEVSESYNDLQRRVLLARQTQVEGLHHKQHEDVPQAGSSQALENRRPKDMNHETPATTLKRRSPDDVDGRDMHRGSPKTPRIDQNKSTNHEEQQNPHDQLPYRKARVSVRARS

SEQ ID NO: 210 - protein, 145 aa, Arabidopsis thaliana
HSEINYHSTGLMDSAHNHFKRARLFEDLEDEDAEVIFPSSVYPSPLPESTVPANKGYASSAIQTLFTGPVKAEEPTPTPKIPKKRGRKKKNADPEEINSSAPRDDDPENRSKFYESASARKRTVTAEERERAINAAKTFEPTNPF

SEQ ID NO: 211 - protein, 185 aa, Arabidopsis thaliana
QLERDYGVLKSNFDALKRNRDSLQRDNDSLLGQIKELKAKLNVEGVKGIEENGALKAVEANQSVMANNEVLELSHRSPSPPPHIPTDAPTSELAFEMFSIFPRTENFRDDPADSSDSSAVLNEEYSPNTVEAAGAVAATTVEMSTMGCFSQFVKMEEHEDLFSGEEACKLFADNEQWYCSDQWNS

SEQ ID NO: 212 - protein, 98 aa, Arabidopsis thaliana
VIVGSSPTQSSTVVDSPTAARFITPPHLELSLGGGGACRRKIPLVHPVYYYNMATYPKMTTCGVQSESETSSVVDFEGGAGKISPPLDLDLNLAPPAE

SEQ ID NO: 213 - protein, 262 aa, Arabidopsis thaliana
LSVPASSSRDLGGVILSPEGKRSMMRLAQRMISNYCLSVSRSNNTRSTVVSELNEVGIRVTAHKSPEPNGTVLCAATTFWLPNSPQNVFNFLKDERTRPQWDVLSNGNAVQEVAHISNGSHPGNCISVLRGSNATHSNNMLILQESSTDSSGAFVVYSPVDLAALNIAMSGEDPSYIPLLSSGFTISPDGNGSNSEQGGASTSSGRASASGSLITVGFQIMVSNLPTAKLNMESVETVNNLIGTTVHQIKTALSGPTASTTA

SEQ ID NO: 214 - protein, 390 aa, Arabidopsis thaliana
DPDRWEFANEGFLRGRKQLLKSIVRRKPSHVQQNQQQTQVQSSSVGACVEVGKFGIEEEVERLKRDKNVLMQELVRLRQQQQATENQLQNVGQKVQVMEQRQQQMMSFLAKAVQSPGFLNQLVQQNNNDGNRQIPGSNKKRRLPVDEQENRGDNVANGLNRQIVRYQPSINEAAQNMLRQFLNTSTSPRYESVSNNPDSFLLGDVPSSTSVDNGNPSSRVSGVTLAEFSPNTVQSATNQVPEASLAHHPQAGLVQPNIGQSPAQGAAPADSWSPEFDLVGCETDSGECFDPIMAVLDESEGDAISPEGEGKMNELLEGVPKLPGIQDPFWEQFFSVELPAIADTDDILSGSVENNDLVLEQEPNEWTRNEQQMKYLTEQMGLLSSEAQRK

SEQ ID NO: 215 - protein, 263 aa, Arabidopsis thaliana
KEFKKAKHHDRGNGSAKMSYYKEIEDILRERSKKVTPPQYNKSPNTPPTSAKVDSFMQFTDKGFDDTSISFGSVEANGRPALNLERRLDHDGHPLAITTAVDAVAANGVTPWNWRETPGNGDDSHGQPFGGRVITVKFGDYTRRIGVDGSAEAIKEVIRSAFGLRTRRAFWLEDEDQIIRCLDRDMPLGNYLLRLDDGLAIRVCHYDESNQLPVHSEEKIFYTEEDYREFLARQGWSSLQVDGFRNIENMDDLQPGAVYRGVR

SEQ ID NO: 216 - protein, 196 aa, Arabidopsis thaliana
KNGTLAHVPPPRPKRKAAHPYPQKASKNAQMSLHVSMSFPTQINNLPGYTPWDDDTSALLNIAVSGVIPPEDELDTLCGAEVDVGSNDMISETSPSASGIGSSSRTLSDSKGLRLAKQAPSMHGLPDFAEVYNFIGSVFDPDSKGRMKKLKEMDPINFETVLLLMRNLTVNLSNPDFEPTSEYVDAAEEGHEHLSS

SEQ ID NO: 217 - protein, 216 aa, Arabidopsis thaliana
PLLNTNFDHLDQNQNQTKSACSSGTSESSLLSLSRTEIRGKARERARERTAKDRDKDLQNAHSSFTQLLTGGFDQQPSNRNWTGGSDCFNPVQLQIPNSSSQEPMNHPFSFVPDYNFGISSSSSAINGGYSSRGTLQSNSQSLFLNNNNNITQRSSISSSSSSSSPMDSQSISFFMATPPPLDHHNHQLPETFDGRLYLYYGEGNRSSDDKAKERR

SEQ ID NO: 218 - protein, 130 aa, Arabidopsis thaliana
TESLNKARELVLRNDSFPPHQGPPSFSYHQGDVHIGDLTQFKPMMYPPRHFSLPGSSSILQLQPPYLYPPLSSPFPQHNTNIGNNGTRHQTLTNSVCGGRALPDSSYTFIGAPVANGSRVAPHLPPHHGL

SEQ ID NO: 219 - protein, 104 aa, Arabidopsis thaliana
RVEDEYTKLKNAYETTVVEKCRLDSEVIHLKEQLYEAEREIQRLAKRVEGTLSNSPISSSVTIEANHTTPFFGDYDIGFDGEADENLLYSPDYIDGLDWMSQFM

SEQ ID NO: 220 - protein, 219 aa, Arabidopsis thaliana
TGTGTIPANFTSLNISLRSSRSSLSAAHLRTTPSSYYFHSPHQSMTHHLQHQHQVRPKNESHSSSSSSSQLLDHNQMGNYLVQSTAGSLPTSQSPATAPFWSSGDNTQNLWAFNINPHHSGVVAGDVYNPNSGGSGGGSGVHLMNFAAPIALFSGQPLASGYGGGGGGGGEHSHYGVLAALNAAYRPVAETGNHNNNQQNRDGDHHHNHQEDGSTSHHS

SEQ ID NO: 221 - protein, 282 aa, Arabidopsis thaliana
KYHKRTKEGRTGKSEGKTYRFFDQLEALESQSTTSLHHHQQQTPLRPQQNNNNNNNNNNNSSIFSTPPPVTTVMPTLPSSSIPPYTQQINVPSFPNISGDFLSDNSTSSSSSYSTSSDMEMGGGTATTRKKRKRKWKVFFERLMKQVVDKQEELQRKFLEAVEKREHERLVREESWRVQEIARINREHEILAQERSMSAAKDAAVMAFLQKLSEKQPNQPQPQPQPQQVRPSMQLNNNNQQQPPQRSPPPQPPAPLPQPIQAVVSTLDTTKTDNGGDQNMTP

SEQ ID NO: 222 - protein, 106 aa, Arabidopsis thaliana
MNDADTNLGSSFSDDTHSVFEFPELDLSDEWMDDDLVSAVSGMNQSYGYQTSDVAGALFSGSSSCFSHPESPSTKTYVAATATASADNQNKKEKKKIKGRVAFKTR

SEQ ID NO: 223 - protein, 181 aa, Arabidopsis thaliana
KNLFKVVNEGSSSINSLDQHNHDASNNNHALQARSFMHRDSPYQLVRNHGAMTFELNKPDLALHQYPPIFHKPPSLGFDYSSGLARDSESAASEGLQYQQACEPGLDVGTCETVASHNHQQGLGEWAMMDRLVTCHMGNEDSSRGITYEDGNNNSSSVVQPVPATNQLTLRSEMDFWGYSK

SEQ ID NO: 224 - protein, 155 aa, Arabidopsis thaliana
PHKEHSQNHSICIRDTNRASMLDLRRNAVFTTSPLIIGRNMNEMQMEVQRRIEEEVVIERQVNQRIAAQGKYMESMLEKACETQEASLTKDYSTLFFDRTNICNNTSSIPIPWFEDHFPSSSSMDSTLILPDINSNFSLQDSRSSITKGRTVCLG

SEQ ID NO: 225 - protein, 203 aa, Arabidopsis thaliana
MFSNFLESLYDGIGDDDAADDDEDNNNDEKTPKASTERHDFSRNAVRLSPEEEAQARGVKDDLTELGHTLTRQFRGVANFLAPLPDGSSSSSSDLSNHPRFNQSRSSDPGLNQSRSSDRDESCVGSDTPETGIRFRSWDLEEKLAEGNDPEDEEEEEEETDEEEEEEEEIAAVALTDEVLAFARNIAMHPETWLDFPLDPDED

SEQ ID NO: 226 - protein, 119 aa, Arabidopsis thaliana
PKIDQSSVSQMILAEIQQGNHQPFKKFQENISVSVSSSSDVSIVGNHFDDLSELHGITNSTPIRSFTMDRLDFGEESFQQDLYDVGSNDLIGNPLINQSIGGYVDNHKDEHKLQFEYES

SEQ ID NO: 227 - protein, 291 aa, Arabidopsis thaliana
KYHKRTKEGRTGKSEGKTYRFFEELEAFETLSSYQPEPESQPAKSSAVITNAPATSSLIPWISSSNPSTEKSSSPLKHHHQVSVQPITTNPTFLAKQPSSTTPFPFYSSNNTTTVSQPPISNDLMNNVSSLNLFSSSTSSSTASDEEEDHHQVKSSRKKRKYWKGLFTKLTKELMEKQEKMQKRFLETLEYREKERISREEAWRVQEIGRINREHETLIHERSNAAAKDAAIISFLHKISGGQPQQPQQHNHKPSQRKQYQSDHSITFESKEPRAVLLDTTIKMGNYDNNH

SEQ ID NO: 228 - protein, 162 aa, Arabidopsis thaliana
TAGGKKIPISTLIRIGSYGTGSSLPPLTDSSPYNDKTKTEPVYVPCFSNQAETRGTILNCFSNPSLSSIQPDFLQMIPLYQPQSLNISESSNPVLTQEQSVLQAMMENNRRQNFKTLSISQETGVSNTDNSSVFEFGRKRFDHQEVPSPSSGPVDLEPFWNY

SEQ ID NO: 229 - protein, 139 aa, Arabidopsis thaliana
KLAGELPRPVTNSPKDIQAAASLAAVNWQDSVNDVSNSEVAEIVEAEPSRAVVAQLFSSDTSTTTTTQSQEYSEASCASTSACTDKDSEEEKLFDLPDLFTDENEMMIRNDAFCYYSSTWQLCGADAGFRLEEPFFLSE

SEQ ID NO: 230 - protein, 78 aa, Arabidopsis thaliana
MQPQTDVFSLHNYLNSSILQSPYPSNFPISTPFPTNGQNPYLLYGFQSPTNNPQSMSLSSNNSTSDEAEEQQTNNNII

SEQ ID NO: 231 - protein, 120 aa, Arabidopsis thaliana
MADHTTKEQKSFSFLAHSPSFDHSSLSYPLFDWEEDLLALQENSGSQAFPFTTTSLPLPDLEPLSEDVLNSYSSASWNETEQNRGDGASSEKKRENGTVKETTKKRKINERHREHSVRII

SEQ ID NO: 232 - protein, 132 aa, Arabidopsis thaliana
MNPQANDRKEFQGDCSATGDLTAKHDSAGGNGGGGARYKLMSPAKLPISRSTDITIPPGLSPTSFLESPVFISNIKPEPSPTTGSLFKPRPVHISASSSSYTGRGFHQNTFTEQKSSEFEFRPPASNMVYAE

SEQ ID NO: 233 - protein, 319 aa, Arabidopsis thaliana
GEAAEISYEPSPSLVSDSHTVIAITGEPEPELQVEQPGKENLLGMSVDDLIEPMNQQEEPQGPHLAPNDDEFIRGLRHVDRGTVEYLFANEENMDGLSMNDLRIPMIVQQEDLSEWEGFNADTFFSDNNNNYNLNVHHQLTPYGDGYLNAFSGYNEGNPPDHELVMQENRNDHMPRKPVTGTIDYSSDSGSDAGSISTTSYQGTSSPNISVGSSSRHLSSCSSTDSCKDLQTCTDPSIISREIRELTQEVKQEIPRAVDAPMNNESSLVKTEKKGLFIVEDAMERNRKKPRFIYLMKMIIGNIISVLLPVKRLIPVKKL

SEQ ID NO: 234 - protein, 154 aa, Arabidopsis thaliana
MATPNEVSALWFIEKHLLDEASPVATDPWMKHESSSATESSSDSSSIIFGSSSSSFAPIDFSESVCKPEIIDLDTPRSMEFLSIPFEFDSEVSVSDFDFKPSNQNQNQFEPELKSQIRKPPLKISLPAKTEWIQFAAENTKPEVTKPVSEEEKK

SEQ ID NO: 235 - protein, 120 aa, Arabidopsis thaliana
MYPSLDDDFVSDLFCFDQSNGAELDDYTQFGVNLQTDQEDTFPDFVSYGVNLQQEPDEVFSIGASQLDLSSYNGVLSLEPEQVGQQDCEVVQEEEVEINSGSSGGAVKEEQEHLDDDCSR

SEQ ID NO: 236 - protein, 185 aa, Arabidopsis thaliana
KNLHKTLNSPVGGASLSGGGDTPKTTSSQIFNEDTLDQFLELMGRSCKEELNLDPFMKLPNLESPNSQAINNCHVSSPDTNHNIHVSNVVDTSFVTSWAALDRLVASQLNGPTSYSITAVNESHVGHDHLALPSVRSPYPSLNRSASYHAGLTQEYTPEMELWNTTTSSLSSSPGPFCHVSNGSG

SEQ ID NO: 237 - protein, 243 aa, Arabidopsis thaliana
MAEKEEKEPSKLKSSTGVSRPTISLPPRPFGEMFFSGGVGFSPGPMTLVSNLFSDPDEFKSFSQLLAGAMASPAAAAVAAAAVVATAHHQTPVSSVGDGGGSGGDVDPRFKQSRPTGLMITQPPGMFTVPPGLSPATLLDSPSFFGLFSPLQGTFGMTHQQALAQVTAQAVQGNNVHMQQSQQSEYPSSTQQQQQQQQQASLTEIPSFSSAPRSQIRASVQETSQGQRETSEISVFEHRSQPQ

SEQ ID NO: 238 - protein, 96 aa, Arabidopsis thaliana
LDVRVTSETCSGEGVIGLGKRKRDKGSPPEEEKAARVKVEEEESNTSETTEAEVEPVVPLTPSSWMGFWDVGAGDGIFSIPPLSPTSPNFSVISVT

SEQ ID NO: 239 - protein, 71 aa, Arabidopsis thaliana
DVKMDEDEVDFLNSHSKSEIVDMLRKHTYNEELEQSKRRRNGNGNMTRTLLTSGLSNDGVSTTGFRSAEAL

SEQ ID NO: 240 - protein, 215 aa, Arabidopsis thaliana
EKVQKYEGSYPGWSQEPTKLTPWRNNHWRVQSLGNHPVAINNGSGPGIPFPGKFEDNTVTSTPAIIAEPQIPIESDKARAITGISIESQPELDDKGLPPLQPILPMVQGEQANECPATSDGLGQSNDLVIEGGTISISSAYSHELLSSLTQALQNAGIDLSQAKLSVQIDLGKRANQGLTHEEPSSKNPLSYDTQGRDSSVEEESEHSHKRMKTL

SEQ ID NO: 241 - protein, 221 aa, Arabidopsis thaliana
QPTTALFTSHYSRIAPSLYGNPNAAMIKSVLGDPTAWSTARSVMQRPGPWQINPVRETHPHMNVLSHGSSSFTTCPEMINNNSTDSSCALSLLSNSYPIHQQQLQTPTNTWRPSSGFDSMISFSDKVTMAQPPPISTHQPPISTHQQYLSQTWEVIAGEKSNSHYMSPVSQISEPADFQISNGTTMGGFELYLHQQVLKQYMEPENTRAYDSSPQHFNWSL

SEQ ID NO: 242 - protein, 217 aa, Arabidopsis thaliana
HPQQQQQVVVNRNLSFSGHGSGSWAYNKKLDMVHGLDLGLGQASCSRGSCSERSSFLQEDDDHSHNRCSSSSGSNLCWLLPKQSDSQDQETVNATTSYGGEGGGGSTLTFSTNLKPKNLMSQNYGLYNGAWSRFLVGQEKKTEHDVSSSCGSSDNKESMLVPSCGGERMHRPELEERTGYLEMDDLLEIDDLGLLIGKNGDFKNWCCEEFQHPWNWF

SEQ ID NO: 243 - protein, 250 aa, Arabidopsis thaliana
TKNSSGGGGGSTSSGNSKSQDSATSNDQYHHRAMANNQMGPPSSSSSLSSLLSSYNAGLIPGHDHNSNNNNILGLGSSLPPLKLMPPLDFTDNFTLQYGAVSAPSYHIGGGSSGGAAALLNGFDQWRFPATNQLPLGGLDPFDQQHQMEQQNPGYGLVTGSGQYRPKNIFHNLISSSSSASSAMVTATASQLASVKMEDSNNQLNLSRQLFGDEQQLWNIHGAAAASTAAATSSWSEVSNNFSSSSTSNI

SEQ ID NO: 244 - protein, 211 aa, Arabidopsis thaliana
GERREFSVATGSGIKHTHSLIPPTNNSGVLSVETEGSLFHSQESQNPSQFSGFLDVDALDRDFCNILSDDFKGFFNDDDEQSKIVSMQDDRNNHTPQKPLTGVFSDHSTDGSDSDPISATTISIQTLSTCPSFGSSNPLYQITDLQESPNSIKLVSLAQEVSKTPGTGIDNDAQGTEIGEHKLGQETIKNKRAGFFHRMIQKFVKKIHLRT

SEQ ID NO: 245 - protein, 171 aa, Arabidopsis thaliana
QLETEYNILRQNYDNLASQFESLKKEKQALVSELQRLKEATQKKTQEEERQCSGDQAVVALSSTHHESENEENRRRKPEEVRPEMEMKDDKGHHGVMCDHHDYEDDDNGYSNNIKREYFGGFEEEPDHLMNIVEPADSCLTSSDDWRGFKSDTTTLLDQSSNNYPWRDFWS

SEQ ID NO: 246 - protein, 77 aa, Arabidopsis thaliana
KVSSENMMNHQHHCSGNSQSSGMTTQGSSGKAIDTAESFSQAKTTTFNVVEQQSNENYWNVEDLWPVHLLNGDHHVI

SEQ ID NO: 247 - protein, 87 aa, Arabidopsis thaliana
STLRGTVAAEHLLVHRGGGGSLLHSFPRHHQDFLMMKHSPANYQSVGSLSYEHGHGTSSYNFNNNQPVVDYGLLQDIVPSMFSKNES

SEQ ID NO: 248 - protein, 126 aa, Arabidopsis thaliana
KRHRSFSTTATSSSSSSSVITTTTQEPATTEASQTKVTNLISGHGSFASLLGLGSGNGGLDYGFGYGYGLEEMSIGYLGDSSVGEIPVVDGCGGDTWQIGEIEGKSGGDSLIWPGLEISMQTNDVK

SEQ ID NO: 249 - protein, 236 aa, Arabidopsis thaliana
KKINESGEEDNDGVSSSNTSSQKNHQSTNKGQWERRLQTDINMAKQALCEALSLDKPSSTLSSSSSLPTPVITQQNIRNFSSALLDRCYDPSSSSSSTTTTTTSNTTNPYPSGVYASSAENIARLLQDFMKDTPKALTLSSSSPVSETGPLTAAVSEEGGEGFEQSFFSFNSMDETQNLTQETSFFHDQVIKPEITMDQDHGLISQGSLSLFEKWLFDEQSHEMVGMALAGQEGMF

SEQ ID NO: 250 - protein, 284 aa, Arabidopsis thaliana
AQLPPWNPADTLRQHAAAAANAKPRKTKTLISPPPPQPEETEHHRIGEEEDNESSFLPASMDSDSIADTIKSFFPVASTQQSYHHQPPSRGNTQNQDLLRLSLQSFQNGPPFPNQTEPALFSGQSNNQLAFDSSTASWEQSHQSPEFGKIQRLVSWNNVGAAESAGSTGGFVFASPSSLHPVYSQSQLLSQRGPLQSINTPMIRAWFDPHHHHHHHQQSMTTDDLHHHHPYHIPPGIHQSAIPGIAFASSGEFSGFRIPARFQGEQEEHGGDNKPSSASSDSRH

SEQ ID NO: 251 - protein, 200 aa, Arabidopsis thaliana
RSDASEVTSTSSQSEVCTVETPGCVHVKTEDPDCESKPFSGGVEPMYCLENGAEEMKRGVKADKHWLSEFEHNYWSDILKEKEKQKEQGIVETCQQQQQDSLSVADYGWPNDVDQSHLDSSDMFDVDELLRDLNGDDVFAGLNQDRYPGNSVANGSYRPESQQSGFDPLQSLNYGIPPFQLEGKDGNGFFDDLSYLDLEN

SEQ ID NO: 252 - protein, 125 aa, Arabidopsis thaliana
LTSSTRNGPKPKPEPKPEPEPEVEPEAEEEDNKFMVLGRGIETTPSCVDEFAWFTEMETTSSTILESPIFSSEKKTAVSGADDVAVFFPMGEEDESLFADLGELPECSVVFRHRSSVVGSQVEIF

SEQ ID NO: 253 - protein, 241 aa, Arabidopsis thaliana
MLEGLVSQESLSLNSMDMSVLERLKWVQQQQQQLQQVVSHSSNNSPELLQILQFHGSNNDELLESSFSQFQMLGSGFGPNYNMGFGPPHESISRTSSCHMEPVDTMEVLLKTGEETRAVALKNKRKPEVKTREEQKTEKKIKVEAETESSMKGKSNMGNTEASSDTSKETSKGASENQKLDYIHVRARRGQATDRHSLAERARREKISKKMKYLQDIVPGCNKVTGKAGMLDEIINYVQCL

SEQ ID NO: 254 - protein, 235 aa, Arabidopsis thaliana
IKKGIDPVTHKGITSGTDKSENLPEKQNVNLTTSDHDLDNDKAKKNNKNFGLSSASFLNKVANRFGKRINQSVLSEIIGSGGPLASTSHTTNTTTTSVSVDSESVKSTSSSFAPTSNLLCHGTVATTPVSSNFDVDGNVNLTCSSSTFSDSSVNNPLMYCDNFVGNNNVDDEDTIGFSTFLNDEDFMMLEESCVENTAFMKELTRFLHEDENDVVDVTPVYERQDLFDEIDNYFG

SEQ ID NO: 255 - protein, 86 aa, Arabidopsis thaliana
TQPRQCGGSVAAAATAKDRPYLHGLGGGGGRHLHYHLHHNNGNGKSNGSGGTAGAGEYYHNIPAIISFNQTGIQNHLVHDSQPFIP

SEQ ID NO: 256 - protein, 239 aa, Arabidopsis thaliana
RLAAVRRMGDYDSSPSHWYDDQLSFMASELETNGQRRILPNHHQQQQHEHQQHMPYGLNASAYALNNPNLQCKQELELHYNHLVQRNHLLDESHLSFLQLPQLESPKIQQDNSNCNSLPYGTSNIDNNSSHNANLQQSNIAHEEQLNQGNQNFSSLYMNSGNEQVMDQVTDWRVLDKFVASQLSNEEAATASASIQNNAKDTSNAEYQVDEEKDPKRASDMGEEYTASTSSSCQIDLWK

SEQ ID NO: 257 - protein, 151 aa, Arabidopsis thaliana
TEATKKYISTSSSSTSHHHNNHTRASILSTNNNNPNYSSDLLQLPPHLQPHPSLNINQSLMANAVHLAELSRVFRASTSTTMDSSHQQLMNYTHMPVSGLNLNLGGALVQPPPVVSLEDVAAVSASYNGENGFGNVEMSQCMDLDGYWPSY

SEQ ID NO: 258 - protein, 91 aa, Arabidopsis thaliana
EEIEDLPRPSTCTPRDIQVAAAKAANAVKIIKMGDDDVAGIDDGDDFWEGIELPELMMSGGGWSPEPFVAGDDATWLVDGDLYQYQFMACL

SEQ ID NO: 259 - protein, 146 aa, Arabidopsis thaliana
TNAVSSQRSIPQSWVYPTIPDNNQQSHNNTATLLASSDVLSHISTRQNFIPSPVNEPASFTESAASYFASQMLGVTYNTARNNGTGDALFLRNNGTGDALVLSNNENNYFNNLTGGLTHEVPNVRSMVMEETTGSEMSATSYSTNN

SEQ ID NO: 260 - protein, 279 aa, Arabidopsis thaliana
MDSNNHLYDPNPTGSGLLRFRSAPSSVLAAFVDDDKIGFDSDRLLSRFVTSNGVNGDLGSPKFEDKSPVSLTNTSVSYAATLPPPPQLEPSSFLGLPPHYPRQSKGIMNSVGLDQFLGINNHHTKPVESNLLRQSSSPAGMFTNLSDQNGYGSMRNLMNYEEDEESPSNSNGLRRHCSLSSRPPSSLGMLSQIPEIAPETNFPYSHWNDPSSFIDNLSSLKREAEDDGKLFLGAQNGESGNRMQLLSHHLSLPKSSSTASDMVSVDKYLQLQDSVPCKI

SEQ ID NO: 261 - protein, 106 aa, Arabidopsis thaliana
RLEEEYNKLKNSHDNVVVDKCRLESEVIQLKEQLYDAEREIQRLAERVEGGSSNSPISSSVSVEANETPFFGDYKVGDDGDDYDHLFYPVPENSYIDEAEWMSLYI

SEQ ID NO: 262 - protein, 129 aa, Arabidopsis thaliana
ELSGLLPRPVSCSPKDIQAAATKAAEATTWHKPVIDKKLADELSHSELLSTAQSSTSSSFVFSSDTSETSSTDKESNEETVFDLPDLFTDGLMNPNDAFCLCNGTFTWQLYGEEDVGFRFEEPFNWQND

SEQ ID NO: 263 - protein, 79 aa, Arabidopsis thaliana
AERVQESLSEIKYTYEDGCSPVVALKRKHSMRRRMTNKKTKDSDFDHRSVKLDNVVVFEDLGEQYLEELLGSSENSGTW

SEQ ID NO: 264 - protein, 258 aa, Arabidopsis thaliana
MGNSSEEPKPPTKSDKPSSPPVDQTNVHVYPDWAAMQAYYGPRVAMPPYYNSAMAASGHPPPPYMWNPQHMMSPYGAPYAAVYPHGGGVYAHPGIPMGSLPQGQKDPPLTTPGTLLSIDTPTKSTGNTDNGLMKKLKEFDGLAMSLGNGNPENGADEHKRSRNSSETDGSTDGSDGNTTGADEPKLKRSREGTPTKDGKQLVQASSFHSVSPSSGDTGVKLIQGSGAILSPGVSANSNPFMSQSLAMVPPETWLQNER

SEQ ID NO: 265 - protein, 141 aa, Arabidopsis thaliana
MDFDEELNLCITKGKNVDHSFGGEASSTSPRSMKKMKSPSRPKPYFQSSSSPYSLEAFPFSLDPTLQNQQQQLGSYVPVLEQRQDPTMQGQKQMISFSPQQQQQQQQYMAQYWSDTLNLSPRGRMMMMMSQEAVQPYIATK

SEQ ID NO: 266 - protein, 132 aa, Arabidopsis thaliana
CSQAANVGTTMPIQNLEPNQTQEHGNLDMVKESVDNYNHQAHLHHNLHYPLSSTPNLENNNAYMLQMRDQNIEYFGSTSFSSDLGTSINYNFPASGSASHSASNSPSTVPLESPFESYDPNHPYGGFGGFYS

SEQ ID NO: 267 - protein, 131 aa, Arabidopsis thaliana
KGATERRGPPPPVVYGDEIMEEKPKVTEMVMPPPPQQTSEFAYFDTSDSVPKLHTTDSSCSEQVVSPEFTSEVQSEPKWKDWSAVSNDNNNTLDFGFNYIDATVDNAFGGGGSSNQMFPLQDMFMYMQKPY

SEQ ID NO: 268 - protein, 192 aa, Arabidopsis thaliana
GKSGNSKSSSSSQNKQSTSMVNATSPTNTSNVQLQTNSQFPFLPTLQNLTQLGGIGLNLAAINGNNGGNGNTSSSFLNDLGFFHGGNTSGPVMGNNNENNLMTSLGSSSHFALFDRTMGLYNFPNEVNMGLSSIGATRVSQTAQVKMEDNHLGNISRPVSGLTSPGNQSNQYWTGQGLPGSSSNDHHHQHLM

SEQ ID NO: 269 - protein, 103 aa, Arabidopsis thaliana
GLGDHSTAVKAACGVESPPSMALITTTSSSHQEISGGKNSTLRFDTLVDESKLKPKSKLVHATPTDVEVAATVPNLFDTFWVLEDDFELSSLTMMDFTNGYCL

SEQ ID NO: 270 - protein, 71 aa, Arabidopsis thaliana
MQPNYDSSSLNNMQQQDYFNLNNYYNNLNPSTNNNNLNILQYPQIQELNLQSPVSNNSTTSDDATEEIFVI

SEQ ID NO: 271 - protein, 175 aa, Arabidopsis thaliana
NHFPNNSQLSLKIRNLLHQKQSMKQQQQQQHKPVSSLTDCNINYISTATSLTTTTTTTTTTAIPLNNVYRPDSSVIGQPETEGLQLPYSWPLVSGFNHQIPLAQAGGETHGHLNDHYSTDQHLGLAEIERQISASLYAMNGANSYYDNMNAEYAIFDPTDPIWDLPSLSQLFCPT

SEQ ID NO: 272 - protein, 299 aa, Arabidopsis thaliana
ELRPLYPSTGSGVPSPRSSFSSCNSSTAFDMGPISPLPIGATTTPPLSPNGVSSPIGGGKTWMNWPNITPPALQLPGSRLKSALNAREIDFSEEMQSLTSPTTWNNTPMSSPFSGKGMNRLAGGAMSPVNSLSDMFGTEDNTSGLQIRRSVINPQLHSNSLSSSPVGANSLFSMDSSAVLASRAAEFAKQRSQSFIERNNGLNHHPAISSMTTTCLNDWGSLDGKLDWSVQGDELQKLRKSTSFRLRAGGMESRLPNEGTGLEEPDVSWVEPLVKEPQETRLAPVWMEQSYMETEQTVA

SEQ ID NO: 273 - protein, 90 aa, Arabidopsis thaliana
NGICSELESERQLQTGQCSFTTASMEEINSNNNNNYNNDYETMSPEVGVSSACVEEVVDDKDDSWMQFITDDAWDTSSNGAAMGHGQGVY

SEQ ID NO: 274 - protein, 258 aa, Arabidopsis thaliana
TGTGTIPANFSTLNASLRSGGGSTLFSQASKSSSSPLSFHSTGMSLYEDNNGTNGSSVDPSRKLLNSAANAAVFGFHHQMYPPIMSTERNPNTLVKPYREDYFKEPSSAAEPSESSQKASQFQEQELAQGRGTANVVPQPMWAVAPGTTNGGSAFWMLPMSGSGGREQMQQQPGHQMWAFNPGNYPVGTGRVVTAPMGSMMLGGQQLGLGVAEGNMAAAMRGSRGDGLAMTLDQHQHQLQHQEPNQSQASENGGDDKK

SEQ ID NO: 275 - protein, 283 aa, Arabidopsis thaliana
TQPRQCNWSSSTSSLNAIGGGGGEASSGGGGGEYHMRRDSGTTSGGSCSSSREIINVNPPNRSDEIGGVGGGVMAVAAAAAAVAAGLPSYAMDQLSFVPFMKSFDEVARRETPQTGHATCEDVMAEQHRHRHQPSSSTSHHMAHDHHHHHHQQQQQRHHAFNISQPTHPISTIISPSTSLHHASINILDDNPYHVHRILLPNENYQTQQQLRQEGEEEHNDGKMGGRSASGLEELIMGCTSSTTHHDVKDGSSSMGNQQEAEWLKYSTFWPAPDSSDNQDHHG

SEQ ID NO: 276 - protein, 144 aa, Arabidopsis thaliana
QPPPPPPGFYRLPAPVSYRPPPSQAPPLQLALPPPQRERSEDPMETSSAEAGGGIRKRHRTKFTAEQKERMLALAERIGWRIQRQDDEVIQRFCQETGVPRQVLKVWLHNNKHTLGKSPSPLHHHQAPPPPPPQSSFHHEQDQP

SEQ ID NO: 277 - protein, 127 aa, Arabidopsis thaliana
MADDWDLHAVVRGCSAVSSSATTTVYSPGVSSHTNPIFTVGRQSNAVSFGEIRDLYTPFTQESVVSSFSCINYPEEPRKPQNQKRPLSLSASSGSVTSKPSGSNTSRSKRRKIQHKKVCHVAAEALN

SEQ ID NO: 278 - protein, 155 aa, Arabidopsis thaliana
QTSAQKQAYNNLMTSGREYSNNGSSTSSSSHQYDDVLESLHEIDNRSLGFAAGSSNALPHSHRPVLTNHKTGFQGLAREPSFDWANLIGQNSVPELGLSHNVPSIRYGDGGTQQQTEGIPRFNNNSDVSANQGFSVDPVNGFGYSGQQSSGFGFI

SEQ ID NO: 279 - protein, 238 aa, Arabidopsis thaliana
MKKITIPVESLDEEDDFLLQLAAIEAEAAAKRPRVSSIPEGPYMAALKGSKSDQWQQSPLNPASKSRSVAVTTGGFQRSDGGGGVAGEQDFPEKSCPCGVGICLILTSNTPKNPGRKFYKCPNREENGGCGFFQWCDAVQSSGTSTTTSNSYGNGNDTKFPDHQCPCGAGLCRVLTAKTGENVGRQFYRCPVFEGSCGFFKWCNDNVVSSPTSYSVTKNSNFGDSDTRGYQNAKTGTP

SEQ ID NO: 280 - protein, 115 aa, Arabidopsis thaliana
MYGQCNIESDYALLESITRHLLGGGGENELRLNESTPSSCFTESWGGLPLKENDSEDMLVYGLLKDAFHFDTSSSDLSCLFDFPAVKVEPTENFTAMEEKPKKAIPVTETAVKAK

SEQ ID NO: 281 - protein, 246 aa, Arabidopsis thaliana
VRARQQGLCVRNSSDTSYLGPAGNMNSGIAAFEMEYTHWLEEQNRRVSEIRTALQAHIGDIELKMLVDSCLNHYANLFRMKADAAKADVFFLMSGMWRTSTERFFQWIGGFRPSELLNVVMPYVEPLTDQQLLEVRNLQQSSQQAEEALSQGLDKLQQGLVESIAIQIKVVESVNHGAPMASAMENLQALESFVNQADHLRQQTLQQMSKILTTRQAARGLLALGEYFHRLRALSSLWAARPREHT

SEQ ID NO: 282 - protein, 121 aa, Arabidopsis thaliana
VKRSISSSSSDVTNHSVSSTSSSSSSISSVLQDVIIKSERPNQEEEFGEILVEQMACGFEVDAPQSLECLFDDSQVPPPISKPDSLQTHGKSSDHEFWSRLIEPGFDDYNEWLIFLDNQTC

SEQ ID NO: 283 - protein, 182 aa, Arabidopsis thaliana
TGSGTIPASALASSAATSNHHQGGSLTAGLMISHDLDGGSSSSGRPLNWGIGGGEGVSRSSLPTGLWPNVAGFGSGVPTTGLMSEGAGYRIGFPGFDFPGVGHMSFASILGGNHNQMPGLELGLSQEGNVGVLNPQSFTQIYQQMGQAQAQAQGRVLHHMHHNHEEHQQESGEKDDSQGSGR

SEQ ID NO: 284 - protein, 244 aa, Arabidopsis thaliana
DRARQQGFYVGNGIDTNSLGFSETMNPGIAAFEMEYGHWVEEQNRQICELRTVLHGHINDIELRSLVENAMKHYFELFRMKSSAAKADVFFVMSGMWRTSAERFFLWIGGFRPSDLLKVLLPHFDVLTDQQLLDVCNLKQSCQQAEDALTQGMEKLQHTLADCVAAGQLGEGSYIPQVNSAMDRLEALVSFVNQADHLRHETLQQMYRILTTRQAARGLLALGEYFQRLRALSSSWATRHREPT

SEQ ID NO: 285 - protein, 115 aa, Arabidopsis thaliana
RADGTKVPMSMLDPHINRMEPAGLPSLMDCSQRDSFTGSSSHVTCFSDQETEDKRLVHESKDGFGSLFYSDPLFLQDNYSLMKLLLDGQETQFSGKPFDGRDSSGTEELDCVWNF

SEQ ID NO: 286 - protein, 147 aa, Arabidopsis thaliana
MDPSGMMNEGGPFNLAEIWQFPLNGVSTAGDSSRRSFVGPNQFGDADLTTAANGDPARMSHALSQAVIEGISGAWKRREDESKSAKIVSTIGASEGENKRQKIDEVCDGKAEAESLGTETEQKKQQMEPTKDYIHVRARRGQATDSH

SEQ ID NO: 287 - protein, 75 aa, Arabidopsis thaliana
ENVGTQTIQRNSHFLQNSMQPSLTYIDQCPTLLSYSRCMEQQQPLVGMLQPTEEENHFFEKPWTEYDQYNYSSFG

SEQ ID NO: 288 - protein, 147 aa, Arabidopsis thaliana
MEDRRCDVLFPCSSSVDPRLTEFHGVDNSAQPTTSSEEKPRSKKKKKEREARYAFQTRSQVDILDDGYRWRKYGQKAVKNNPFPRSYYKCTEEGCRVKKQVQRQWGDEGVVVTTYQGVHTHAVDKPSDNFHHILTQMHIFPPFCLKE

SEQ ID NO: 289 - protein, 172 aa, Arabidopsis thaliana
MYSYKKISYQMEEVMSMIFHGMKLVKSLESSLPEKPPESLLTSLDEIVKTFSDANERLKMLLEIKNSETALNKTKPVIVSVANQMLMQMEPGLMQEYWLRYGGSTSSQGTEAMFQTQLMAVDGGGERNLTAAVERSGASGSSTPRQRRRKDEGEEQTVLVAALRTGNTDLPP

SEQ ID NO: 290 - protein, 354 aa, Arabidopsis thaliana
MVTRETKLTSEREVESSMAQARHNGGGGGENHPFTSLGRQSSIYSLTLDEFQHALCENGKNFGSMNMDEFLVSIWNAEENNNNQQQAAAAAGSHSVPANHNGFNNNNNNGGEGGVGVFSGGSRGNEDANNKRGIANESSLPRQGSLTLPAPLCRKTVDEVWSEIHRGGGSGNGGDSNGRSSSSNGQNNAQNGGETAARQPTFGEMTLEDFLVKAGVVREHPTNPKPNPNPNQNQNPSSVIPAAAQQQLYGVFQGTGDPSFPGQAMGVGDPSGYAKRTGGGGYQQAPPVQAGVCYGGGVGFGAGGQQMGMVGPLSPVSSDGLGHGQVDNIGGQYGVDMGGLRGRKRVVDGPVEKV

SEQ ID NO: 291 - protein, 95 aa, Arabidopsis thaliana
DSAWRLPVPASTDPDTIRRTAAEAAEMFRPPEFSTGITVLPSASEFDTSDEGVAGMMMRLAEEPLMSPPRSYIDMNTSVYVDEEMCYEDLSLWSY

SEQ ID NO: 292 - protein, 125 aa, Arabidopsis thaliana
EAQNYGKLFEWRGNTGEELLHKYKETEITRTKTTSQEHGFVEVVSMESGKEANGGVGGRESFGVMKSPYENRISDWISEISTDQSEANLSEDHSSNSCSENNINIGTWWFQETRDFEEFSCSLWS

SEQ ID NO: 293 - protein, 183 aa, Arabidopsis thaliana
MCVLKVANQEDNVGKKAESIRDDDHRTLSEIDQWLYLFAAEDDHHRHSFPTQQPPPSSSSSSLISGFSREMEMSAIVSALTHVVAGNVPQHQQGGGEGSGEGTSNSSSSSGQKRRREVEEGGAKAVKAANTLTVDQYFSGGSSTSKVREASSNMSGPGPTYEYTTTATASSETSSFSGDQPRR

SEQ ID NO: 294 - protein, 227 aa, Arabidopsis thaliana
QPASLSVLASRYGRIAPSLYENGDAGMNGSFLGNQEIGWPSSRTLDTRVMRRPVSSPSWQINPMNVFSQGSVGGGGTSFSSPEIMDTKLESYKGIGDSNCALSLLSNPHQPHDNNNNNNNNNNNNNNTWRASSGFGPMTVTMAQPPPAPSQHQYLNPPWVFKDNDNDMSPVLNLGRYTEPDNCQISSGTAMGEFELSDHHHQSRRQYMEDENTRAYDSSSHHTNWSL

SEQ ID NO: 295 - protein, 202 aa, Arabidopsis thaliana
SKTKQVPSSSSADKPTTTQDDHHVEEKSSTGSHSSSESSSLTASNSTTVAAVSVTAAAEVASSVIPGFDMPNMKIYGNGIEWSTLLGQGSSAGGVFSEIGGFPAVSAIETTPFGFGGKFVNQDDHLKLEGETVQQQQFGDRTAQVEFQGRSSDPNMGFEPLDWGSGGGDQTLFDLTSTVDHAYWSQSQWTSSDQDQSGLYLP

SEQ ID NO: 296 - protein, 154 aa, Arabidopsis thaliana
TGTGTTPASFSTASLSTSSPFTLGKRVVRAEEGESGGGGGGGLTVGHTMGTSLMGGGGSGGFWAVPARPDFGQVWSFATGAPPEMVFAQQQQPATLFVRHQQQQQASAAAAAAMGEASAARVGNYLPGHHLNLLASLSGGANGSGRREDDHEPR

SEQ ID NO: 297 - protein, 294 aa, Arabidopsis thaliana
MGSSEMEKSGKEKEPKTTPPSTSSSAPATVVSQEPSSAVSAGVAVTQDWSGFQAYSPMPPHGYVASSPQPHPYMWGVQHMMPPYGTPPHPYVTMYPPGGMYAHPSLPPGSYPYSPYAMPSPNGMAEASGNTGSVIEGDGKPSDGKEKLPIKRSKGSLGSLNMIIGKNNEAGKNSGASANGACSKSAESGSDGSSDGSDANSQNDSGSRHNGKDGETASESGGSAHGPPRNGSNLPVNQTVAIMPVSATGVPGPPTNLNIGMDYWSGHGNVSGAVPGVVVDGSQSQPWLQVSDER

SEQ ID NO: 298 - protein, 121 aa, Arabidopsis thaliana
IRMGIDPNTHRRFDQQKVNEEETILVNDPKPLSETEVSVALKNDTSAVLSGNLNQLADVDGDDQPWSFLMENDEGGGGDAAGELTMLLSGDITSSCSSSSSLWMKYGEFGYEDLELGCFDV

SEQ ID NO: 299 - protein, 130 aa, Arabidopsis thaliana
HHSQDQNNKEDFVSTTAAEMPTSPQQQSSSSADISAITTLGNNNDISNSNKDSATSSEDVLAIIDESFWSEVVLMDCDISGNEKNEKKIENWEGSLDRNDKGYNHDMEFWFDHLTSSSCIIGEMSDISEF

SEQ ID NO: 300 - protein, 124 aa, Arabidopsis thaliana
ASLELPQPQTRPQPMPQPQPLFFTPPPPLAITDLPASVSPLPSTYDLASIFDQTTSSSAWATQQRRFIDPRHQYGVSSSSSSVAVGLGGENSHDLQALAHELLHRQGSPPPAATDHSPSRTMSR

SEQ ID NO: 301 - protein, 214 aa, Arabidopsis thaliana
VQAKNLNNDDEDFGNIGGDVEQEEEKEEDDNGDKSFVYGLSPGYGEEEVVCEATKAGIRKKKSELRNISSKGLGAKARGKAKERTKEMMAYDNPETASDITQSEIMDPFKRSIVFNEGEDMTHLFYKEPIEEFDNQESILTNMTLPTKMGQSYNQNNGILMLVDQSSSSNYNTFLPQNLDYSYDQNPFHDQTLYVVTDKNFPKGKVWIQDSFVN

SEQ ID NO: 302 - protein, 243 aa, Arabidopsis thaliana
LQMGIDPVTHEPRTNDLSPILDVSQMLAAAINNGQFGNNNLLNNNTALEDILKLQLIHKMLQIITPKAIPNISSFKTNLLNPKPEPVVNSFNTNSVNPKPDPPAGLFINQSGITPEAASDFIPSYENVWDGFEDNQLPGLVTVSQESLNTAKPGTSTTTKVNDHIRTGMMPCYYGDQLLETPSTGSVSVSPETTSLNHPSTAQHSSGSDFLEDWEKFLDDETSDSCWKSFLDLTSPTSSPVPW

SEQ ID NO: 303 - protein, 135 aa, Arabidopsis thaliana
MHYPNNRTEFVGAPAPTRYQKEQLSPEQELSVIVSALQHVISGENETAPCQGFSSDSTVISAGMPRLDSDTCQVCRIEGCLGCNYFFAPNQRIEKNHQQEEEITSSSNRRRESSPVAKKAEGGGKIRKRKNKKNG

SEQ ID NO: 304 - protein, 110 aa, Arabidopsis thaliana
MGSFDRQRAVPKFKTATPSPLPLSPSPYFTMPPGLTPADFLDSPLLFTSSNILPSPTTGTFPAQSLNYNNNGLLIDKNEIKYEDTTPPLFLPSMVTQPLPQLDLFKSEIM

SEQ ID NO: 305 - protein, 134 aa, Arabidopsis thaliana
MNRGIEVMSPATYLETSNWLFQENRGTKWTAEENKKFENALAFYDKDTPDRWSRVAAMLPGKTVGDVIKQYRELEEDVSDIEAGLIPIPGYASDSFTLDWGGYDGASGNNGFNMNGYYFSAAGGKRGSAARTAE

SEQ ID NO: 306 - protein, 236 aa, Arabidopsis thaliana
MGCFDPNTSAEVTVESSFSQSEQPPPPPQVLVAGSTSNSNCSVEVEELSEFHLSPQDCPQASSTPLQFHINPPPPPPPPCDQFHNNLIHQMASHQQHSSWENGYQDFVNLGPNSATTPDLLSLLHLPRWSLPPNHHPSSMLPNSSISFSDIMSSSSAAAVMYDPLFHLNFPMQPRDQNQLRNGSCLLGVEDQIQMDANGGVNVMYFEGANNNNNNGGFENEILEFNNGVTRKGRGS

SEQ ID NO: 307 - protein, 281 aa, Arabidopsis thaliana
NPDRWEFANEGFLRGQKHLLKNIRRRKTSNNSNQMQQPQSSEQQSLDNFCIEVGRYGLDGEMDSLRRDKQVLMMELVRLRQQQQSTKMYLTLIEEKLKKTESKQKQMMSFLARAMQNPDFIQQLVEQKEKRKEIEEAISKKRQRPIDQGKRNVEDYGDESGYGNDVAASSSALIGMSQEYTYGNMSEFEMSELDKLAMHIQGLGDNSSAREEVLNVEKGNDEEEVEDQQQGYHKENNEIYGEGFWEDLLNEGQNFDFEGDQENVDVLIQQLGYLGSSSHTN

SEQ ID NO: 308 - protein, 100 aa, Arabidopsis thaliana
VEVVRESLKKMENVNLHDGGSPVMALKRKHSLRNRPRGKKRSSSSSSSSSNSSSCSSSSSTSSTSRSSSKQSVVKQESGTLVVFEDLGAEYLEQLLMSSC

SEQ ID NO: 309 - protein, 237 aa, Arabidopsis thaliana
MYIKAIMNRHRLLSAATDECNKKLGQACSSSLSPVHNFLNVQPEHRKTPFIRSQSPDSPGQLWPKNSSQSTFSRSSTFCTNLYLSSSSTSETQKHLGNSLPFLPDPSSYTHSASGVESARSPSIFTEDLGNQCDGGNSGSLLKDFLNLSGDACSDGDFHDFGCSNDSYCLSDQMELQFLSDELELAITDRAETPRLDEIYETPLASNPVTRLSPSQSCVPGAMSVDVVSSHPSPGSA

SEQ ID NO: 310 - protein, 272 aa, Arabidopsis thaliana
PYDTNPEVWPSNSGVQRVVSEFRTLPEMDQHKKMVDQEGFLKQRIAKATETLRRQRKDSRELEMTEVMFQCLIGNMEMFHLNIVDLNDLGYMIEQYLKDVNRRIEILRNSGTEIGESSSVAVAASEGNIPMPNLVATTAPTTTIYEVGSSSSFAAVANFVNPIDLQQFRHPAAQHVGLNEQPQNLNLNLNQNYNQNQEWFMEMMNHPEQMRYQTEQMGYQFMDDNHHNHIHHQPQEHQHQIHDESSNALDAANSSSIIPVTSSSITNKTWFH

SEQ ID NO: 311 - protein, 146 aa, Arabidopsis thaliana
ELSKLLPRPVSLSPRDVRAAATKAALMDFDTTAFRSDTETSETTTSNKMSESSESNETVSFSSSSWSSVTSIEESTVSDDLDEIVKLPSLGTSLNESNEFVIFDSLEDLVYMPRWLSGTEEEVFTYNNNDSSLNYSSVFESWKHFP

SEQ ID NO: 312 - protein, 126 aa, Arabidopsis thaliana
DLAGSFPRPSSLSPRDIQVAALKAAHMETSQSFSSSSSLTFSSSQSSSSLESLVSSSATGSEELGEIVELPSLGSSYDGLTQLGNEFIFSDSADLWPYPPQWSEGDYQMIPASLSQDWDLQGLYNY

SEQ ID NO: 313 - protein, 97 aa, Arabidopsis thaliana
SGGKDKRRASIHDITTVNLEEEASLETNKSSIVVGDQRSRLTAFPWNQTDNNGTQADAFNITIGNAISGVHSYGQVMIGGYNNADSCYDAQNTMFQL

SEQ ID NO: 314 - protein, 149 aa, Arabidopsis thaliana
QLERDYDLLKSTYDQLLSNYDSIVMDNDKLRSEVTSLTEKLQGKQETANEPPGQVPEPNQLDPVYINAAAIKTEDRLSSGSVGSAVLDDDAPQLLDSCDSYFPSIVPIQDNSNASDHDNDRSCFADVFVPTTSPSHDHHGESLAFWGWP

SEQ ID NO: 315 - protein, 151 aa, Arabidopsis thaliana
PPLQFPPGFHQLNPNLTGLGESFPGVFDLGRTQREALDLEKRKWVNLDHVFDHIDHHNHFSNSIQSNKLYFPTITSSSSSYHYNLGHLQQSLLDQSGNVTVAFSNNYNNNNLNPPAAETMSSLFPTRYPSFLGGGQLQLFSSTSSQPDHIE

SEQ ID NO: 316 - protein, 335 aa, Arabidopsis thaliana
MDGSMNLGNEPPGDGGGGGGLTRQGSIYSLTFDEFQSSVGKDFGSMNMDELLKNIWSAEETQAMASGVVPVLGGGQEGLQLQRQGSLTLPRTLSQKTVDQVWKDLSKVGSSGVGGSNLSQVAQAQSQSQSQRQQTLGEVTLEEFLVRAGVVREEAQVAARAQIAENNKGGYFGNDANTGFSVEFQQPSPRVVAAGVMGNLGAETANSLQVQGSSLPLNVNGARTTYQQSQQQQPIMPKQPGFGYGTQMGQLNSPGIRGGGLVGLGDQSLTNNVGFVQGASAAIPGALGVGAVSPVTPLSSEGIGKSNGDSSSLSPSPYMFNGGVRGRKSGTVEKV

SEQ ID NO: 317 - protein, 214 aa, Arabidopsis thaliana
VMMKFQNGIINENKTNLATDISSCNNNNNGCNHNKRTTNKGQWEKKLQTDINMAKQALFQALSLDQPSSLIPPDPDSPKPHHHSTTTYASSTDNISKLLQNWTSSSSSKPNTSSVSNNRSSSPGEGGLFDHHSLFSSNSESGSVDEKLNLMSETSMFKGESKPDIDMEATPTTTTTDDQGSLSLIEKWLFDDQGLVQCDDSQEDLIDVSLEELK

SEQ ID NO: 318 - protein, 701 aa, Arabidopsis thaliana
NPEPGANGNPSDDHSSNYLLITLLKILSNMHNHTGDQDLMSHLLKSLVSHAGEQLGKNLVELLLQGGGSQGSLNIGNSALLGIEQAPQEELKQFSARQDGTATENRSEKQVKMNDFDLNDIYIDSDDTDVERSPPPTNPATSSLDYPSWIHQSSPPQTSRNSDSASDQSPSSSSEDAQMRTGRIVFKLFGKEPNEFPIVLRGQILDWLSHSPTDMESYIRPGCIVLTIYLRQAETAWEELSDDLGFSLGKLLDLSDDPLWTTGWIYVRVQNQLAFVYNGQVVVDTSLSLKSRDYSHIISVKPLAIAATEKAQFTVKGMNLRQRGTRLLCSVEGKYLIQETTHDSTTREDDDFKDNSEIVECVNFSCDMPILSGRGFMEIEDQGLSSSFFPFLVVEDDDVCSEIRILETTLEFTGTDSAKQAMDFIHEIGWLLHRSKLGESDPNPGVFPLIRFQWLIEFSMDREWCAVIRKLLNMFFDGAVGEFSSSSNATLSELCLLHRAVRKNSKPMVEMLLRYIPKQQRNSLFRPDAAGPAGLTPLHIAAGKDGSEDVLDALTEDPAMVGIEAWKTCRDSTGFTPEDYARLRGHFSYIHLIQRKINKKSTTEDHVVVNIPVSFSDREQKEPKSGPMASALEITQIPCKLCDHKLVYGTTRRSVAYRPAMLSMVAIAAVCVCVALLFKSCPEVLYVFQPFRWELLDYGTS

SEQ ID NO: 319 - protein, 203 aa, Arabidopsis thaliana
LRMGIDPVTHCPRINLLQLSSFLTSSLFKSMSQPMNTPFDLTTSNINPDILNHLTASLNNVQTESYQPNQQLQNDLNTDQTTFTGLLNSTPPVQWQNNGEYLGDYHSYTGTGDPSNNKVPQAGNYSSAAFVSDHINDGENFKAGWNFSSSMLAGTSSSSSTPLNSSSTFYVNGGSEDDRESFGSDMLMFHHHHDHNNNALNLS

SEQ ID NO: 320 - protein, 212 aa, Arabidopsis thaliana
EKVHMYEDSHQMWYQSPTKLIPWRNSHGSVAEENDHPQIVKSFSSNDKVAASSGFLLDTYNSVNPDIDSAVSTKIPEHSPVSAVSSYLRTEPSLQFVQHDFWQPKTSCGTINCFTNELLTSDEKTSASLSTVCSQRVLNTLTEALKSSGVNMSETMISVQLSLRKREDREYSVAAFASEDNGNSIADEEGDSPTETRSFCNDIDHSQKRIRR

SEQ ID NO: 321 - protein, 184 aa, Arabidopsis thaliana
QPEHIGRPANFFTGFQGSKLLEFSGGSHVFPTTSVLNPSWGNSLVSVAVAANGSSYGQSQSYVVGSSPAKTGIMFPISSSPNSTRSIAKQFPFLQEEESSRTASLCERMTSCIHDSDCALSLLSSSSSSVPHLLQPPLSLSQEAVETVFYGSGLFENASAVSDGSVISGNEAVRLPQTFPFHWE

SEQ ID NO: 322 - protein, 143 aa, Arabidopsis thaliana
MADLFGGGHGGELMEALQPFYKSASTSASNPAFASSNDAFASAPNDLFSSSSYYNPHASLFPSHSTTSYPDIYSGSMTYPSSFGSDLQQPENYQSQFHYQNTITYTHQDNNTCMLNFIEPSQPGFMTQPGPSSGSVSKPAKLY

SEQ ID NO: 323 - protein, 193 aa, Arabidopsis thaliana
HLQRNTRPSLSNSQRFKWVPSRKFISMFPSCGMLNVNAQPSVHIIQQRLEELKKTGLLSQSYSSSSSSTESKTNTSFLDEKTSKGETDNMFEGGDQKKPEIDLTEFLQQLGILKDENEAEPSEVAECHSPPPWNEQEETGSPFRTENFSWDTLIEMPRSETTTMQFDSSNFGSYDFEDDVSFPSIWDYYGSLD

SEQ ID NO: 324 - protein, 129 aa, Arabidopsis thaliana
MNRDRRRSSIHDITTVNNQAPAVTGGGQQPQVVKHRPAQPQPQPQPQPQQHHPPTMAGLGMYGGAPVGQPIIAPPDHMGSAVGTPVMLPPPMGTHHHHHHHHLGVAPYAVPAYPVPPLPQQHPAPSTMH

SEQ ID NO: 325 - protein, 158 aa, Arabidopsis thaliana
MSSEDWDLFAVVRSCSSSVSTTNSCAGHEDDIGNCKQQQDPPPPPLFQASSSCNELQDSCKPFLPVTTTTTTTWSPPPLLPPPKASSPSPNILLKQEQVLLESQDQKPPLSVRVFPPSTSSSVFVFRGQRDQLLQQQSQPPLRSRKRKNQQKRTICHV

SEQ ID NO: 326 - protein, 218 aa, Arabidopsis thaliana
IQMGFDPMTHRPRTDIFSGLSQLMSLSSNLRGFVDLQQQFPIDQEHTILKLQTEMAKLQLFQYLLQPSSMSNNVNPNDFDTLSLLNSIASFKETSNNTTSNNLDLGFLGSYLQDFHSLPSLKTLNSNMEPSSVFPQNLDDNHFKFSTQRENLPVSPIWLSDPSSTTPAHVNDDLIFNQYGIEDVNSNITSSSGQESGASASAAWPDHLLDDSIFSDIP

SEQ ID NO: 327 - protein, 207 aa, Arabidopsis thaliana
RATGQAKNTETWSSSYFYDEVAPNGVNSVMDPIDYISKQQHNIFGKGLMCKQELEGMVDGINYIQSNQFIQLPQLQSPSLPLMKRPSSSMSITSMDNNYNYKLPLADEESFESFIRGEDRRKKKKQVMMTGNWRELDKFVASQLMSQEDNGTSSFAGHHIVNEDKNNNDVEMDSSMFLSEREEENRFVSEFLSTNSDYDIGICVFDN

SEQ ID NO: 328 - protein, 69 aa, Arabidopsis thaliana
MQPSTNIFSLHGCPPSYLSHIPTSSPFCGQNPNPFFSFETGVNTSQFMSLISSNNSTSDEAEENHKEII

SEQ ID NO: 329 - protein, 233 aa, Arabidopsis thaliana
CPGDEDADVSVLQLQAEIENLALEEQALDNQIRWLFVTEEDIKSLPGFQNQTLIAVKAPHGTTLEVPDPDEAADHPQRRYRIILRSTMGPIDVYLVSEFEGKFEDTNGSGAAPPACLPIASSSGSTGHHDIEALTVDNPETAIVSHDHPHPQPGDTSDLNYLQEQVGGMLKITPSDVENDESDYWLLSNAEISMTDIWKTDSGIDWDYGIADVSTPPPGMGEIAPTAVDSTPR

SEQ ID NO: 330 - protein, 381 aa, Arabidopsis thaliana
DPDRWEFANEGFLRGQKQILKSIVRRKPAQVQPPQQPQVQHSSVGACVEVGKFGLEEEVERLQRDKNVLMQELVRLRQQQQVTEHHLQNVGQKVHVMEQRQQQMMSFLAKAVQSPGFLNQFSQQSNEANQHISESNKKRRLPVEDQMNSGSHGVNGLSRQIVRYQSSMNDATNTMLQQIQQMSNAPSHESLSSNNGSFLLGDVPNSNISDNGSSSNGSPEVTLADVSSIPAGFYPAMKYHEPCETNQVMETNLPFSQGDLLPPTQGAAASGSSSSDLVGCETDNGECLDPIMAVLDGALELEADTLNELLPEVQDSFWEQFIGESPVIGETDELISGSVENELILEQLELQSTLSNVWSKNQQMNHLTEQMGLLTSDALRK

SEQ ID NO: 331 - protein, 109 aa, Arabidopsis thaliana
DSAWRLRIPESTCAKDIQKAAAEAALAFQDEMCDATTDHGFDMEETLVEAIYTAEQSENAFYMHDEAMFEMPSLLANMAEGMLLPLPSVQWNHNHEVDGDDDDVSLWSY

SEQ ID NO: 332 - protein, 245 aa, Arabidopsis thaliana
PSSSNSSSSTSSGKKPSNIVTANTSDLMALAHSHQNYQHSPLGFSHFGGMMGSYSTPEHGNVGFLESKYGGLLSQSPRPIDFLDSKFDLMGVNNDNLVMVNHGSNGDHHHHHNHHMGLNHGVGLNNNNNNGGFNGISTGGNGNGGGLMDISTCQRLMLSNYDHHHYNHQEDHQRVATIMDVKPNPKLLSLDWQQDQCYSNGGGSGGAGKSDGGGYGNGGYINGLGSSWNGLMNGYGTSTKTNSLV

SEQ ID NO: 333 - protein, 214 aa, Arabidopsis thaliana
MGGESNEGGEMGFKHGDDESGGISRVGITSMPLYAKADPFFSSADWDPVVNAAAAGFSSSHYHPSMAMDNPGMSCFSHYQPGSVSGFAADMPASLLPFGDCGGGQIGHFLGSDKKGERLIRAGESSHEDHHQVSDDAVLGASPVGKRRLPEAESQWNKKAVEEFQEDPQRGNDQSQKKHKNDQSKETVNKESSQSEEAPKENYIHMRARRGQAT

SEQ ID NO: 334 - protein, 145 aa, Arabidopsis thaliana
ELATYLPRPASSSPRDVQAAAAVAAAMDFSPSSSSLVVSDPTTVIAPAETQLSSSSYSTCTSSSLSPSSEEAASTAEELSEIVELPSLETSYDESLSEFVYVDSAYPPSSPWYINNCYSFYYHSDENGISMAEPFDSSNFGPLFP

SEQ ID NO: 335 - protein, 102 aa, Arabidopsis thaliana
MNYPSNPNPSSTDFTEFFKFDDFDDTFEKIMEEIGREDHSSSPTLSWSSSEKLVAAEITSPLQTSLATSPMSFEIGDKDEIKKRKRHKEDPIIHVFKTKSSI

SEQ ID NO: 336 - protein, 249 aa, Arabidopsis thaliana
IQMGIDPVTHQPRTDLFASLPQLIALANLKDLIEQTSQFSSMQGEAAQLANLQYLQRMFNSSASLTNNNGNNFSPSSILDIDQHHAMNLLNSMVSWNKDQNPAFDPVLELEANDQNQDLFPLGFIIDQPTQPLQQQKYHLNNSPSELPSQGDPLLDHVPFSLQTPLNSEDHFIDNLVKHPTDHEHEHDDNPSSWVLPSLIDNNPKTVTSSLPHNNPADASSSSSYGGCEAASFYWPDICFDESLMNVIS

SEQ ID NO: 337 - protein, 79 aa, Arabidopsis thaliana
TAQMEELSTRLQSLNEIVDLVQSNGAGFGVDQIDGCGFDDRTVGIDGYYDDMNMMSNVNHWGGSVYTNQPIMANDINMY

SEQ ID NO: 338 - protein, 163 aa, Arabidopsis thaliana
NNPSTTTQPMTRIPVEDFTRMDSLENIDHLLDFSSLPPLIDPSFMSQTEQPNFKPINPPTYDISSPIQPHHFNSYQSIFNHQVFGSASGSTYNNNNEMIKMEQSLVSVSQETCLSSDVNANMTTTTEVSSGPVMKQEMGMMGMVNGSKSYEDLCDLRGDLWDF

SEQ ID NO: 339 - protein, 173 aa, Arabidopsis thaliana
SHASLSSPDVALVTSNQEHEENDNEPFVDRGTFLPNLQNDQPLKRQKSSCSFSNLLDATDLTFLANFLNETPENRSESDFSFMIGNFSNPDIYGNHYLDQKLPQLSSPTSETSGIGSKRERVDFAEETINASKKMMNTYSYNNSIDQMDHSMMQQPSFLNQELMMSSHLQYQG

SEQ ID NO: 340 - protein, 192 aa, Arabidopsis thaliana
KLTTMNYNNPRTMMGSSSGQESNWFTQQMDVGNGNYYHLPDLESPRMFQGSSSSSLSSLHQNDQDPYGVVLSTINATPTTIMQRDDGHVITNDDDHMIMMNTSTGDHHQSGLLVNDDHNDQVMDWQTLDKFVASQLIMSQEEEEVNKDPSDNSSNETFHHLSEEQAATMVSMNASSSSSPCSFYSWAQNTHT

SEQ ID NO: 341 - protein, 147 aa, Arabidopsis thaliana
MEKSDPPPVPKPGATIIPSSDPIPNADPIPSSSFHRRSRSDDMSMFMFMDPLSSAAPPSSDDLPSDDDLFSSFIDVDSLTSNPNPFQNPSLSSNSVSGAANPPPPPSSRPRHRHSNSVDAGCAMYAGDIMDAKKAMPPEKLSELWNI

SEQ ID NO: 342 - protein, 408 aa, Arabidopsis thaliana
SGTGPKNGEQYGAPYLEEEWEEDGMTYVPAQDAFSEGLALNDDVYVDIDDIDEKPENLVVYDAVPILPNYCHGESSNNVESGNYSDSGNYIQPGNNVVDSGGYFEQPIETFEEDRKPIIREGSIQPCSLFPEEQIGCGVQDENVVNLESSNNNVFVADTCYSDIPIDHNYLPDEPFMDPNNNLPLNDGLYLETNDLSCAQQDDFNFEDYLSFFDDEGLTFDDSLLMGPEDFLPNQEALDQKPAPKELEKEVAGGKEAVEEKESGEGSSSKQDTDFKDFDSAPKYPFLKKTSHMLGAIPTPSSFASQFQTKDAMRLHAAQSSGSVHVTAGMMRISNMTLAADSGMGWSYDKNGNLNVVLSFGVVQQDDAMTASGSKTGITATRAMLVFMCLWVLLLSVSFKIVTMVSAR

SEQ ID NO: 343 - protein, 353 aa, Arabidopsis thaliana
MGSEYKHILKSLCLSHGWSYAVFWRYDPINSMILRFEEAYNDEQSVALVDDMVLQAPILGQGIVGEVASSGNHQWLFSDTLFQWEHEFQNQFLCGFKILIRQFTYTQTIAIIPLGSSGVVQLGSTQKILESTEILEQTTRALQETCLKPHDSGDLDTLFESLGDCEIFPAESFQGFSFDDIFAEDNPPSLLSPEMISSEAASSNQDLTNGDDYGFDILQSYSLDDLYQLLADPPEQNCSSMVIQGVDKDLFDILGMNSQTPTMALPPKGLFSELISSSLSNNTCSSSLTNVQEYSGVNQSKRRKLDTSSAHSSSLFPQEETVTSRSLWIDDDERSSIGGNWKKPHEEGVKKKR

SEQ ID NO: 344 - protein, 107 aa, Arabidopsis thaliana
ESLRSYPETASSQASHTTPSSNTGGKSSDSESPCSSNEMSSCGRVTDEISWEHINVDLPVMDDSSIWEEATMSLGFPWVHEGDNNISRFDTCISGGFSNWDSFHSPL

SEQ ID NO: 345 - protein, 126 aa, Arabidopsis thaliana
VVKSEEGSDHVKDVNSPLMSPKSLSELLNAKLRKSCKDLTPSLTCLRLDTDSSHIGVWQKRAGSKTSPTWVMRLELGNVVNESAVDLGLTTMNKQNVEKEEEEEEAIISDEDQLAMEMIEELLNWS

SEQ ID NO: 346 - protein, 151 aa, Arabidopsis thaliana
SSSATKSLRTTPEPTMTHDGKSFPTASFGYNNNNISNEQMELGLAYALLNKQPLGVSSHLGFGSSQSPMAMDGVYGTTSHQMENTGYAFGNGGGGMEQMATSDPNRVLWGFPWQMNMGGGSGHGHGHVDQIDSGREIWSSTVNYINTGALL

SEQ ID NO: 347 - protein, 118 aa, Arabidopsis thaliana
TVSSRKYTPDWRELANGKRVKQQQSNYQEAYINFGDNESSSSTNVMNVREGKGNYERSVFQLQQTPYQHQNQPILMDTTHVDSFQHFSNDNIHHETYETWPDELRSVVEFAFPPSFLS

SEQ ID NO: 348 - protein, 102 aa, Arabidopsis thaliana
KLEEEYAKLKNHHDNVVLGQCQLESQILKLTEQLSEAQSEIRKLSERLEEMPTNSSSSSLSVEANNAPTDFELAPETNYNIPFYMLDNNYLQSMEYWDGLYV

SEQ ID NO: 349 - protein, 145 aa, Arabidopsis thaliana
NITTTSPFLMNIDEKTLLSPKSIQKVAAQAANSSSDHFTPPSDENDHDHDDGLDHHPSASSSAASSPPDDDHHNDDDGDLVSLMESFVDYNEHVSLMDPSLYEFGHNEIFFTNGDPFDYSPQLHSSEATMDDFYDDVDIPLWSFS

SEQ ID NO: 350 - protein, 187 aa, Arabidopsis thaliana
YKGIRRRPWGRWAAEIRDPIKGVRVWLGTFNTAEEAARAYDLEAKRIRGAKAKLNFPNESSGKRKAKAKTVQQVEENHEADLDVAVVSSAPSSSCLDFLWEENNPDTLLIDTQWLEDIIMGDANKKHEPNDSEEANNVDASLLSEELLAFENQTEYFSQMPFTEGNCDSSTSLSSLFDGGNDMGLWS

SEQ ID NO: 351 - protein, 104 aa, Arabidopsis thaliana
TDKKPQLPEGSVRPLSKLDIQTIATNYASSVVHVPSHATTLPATTQVPSEVPASSDVSASTEITEMVDEYYLPTDATAESIFSVEDLQLDSFLMMDIDWINNLI

SEQ ID NO: 352 - protein, 177 aa, Arabidopsis thaliana
EENMKANSQKRSVKANLQKPVAKPNPNPSPALVQNSNISFENMCFMEEKHQVSNNNNNQFGMTNSVDAGCNGYQYFSSDQGSNSFDCSEFGWSDQAPITPDISSAVINNNNSALFFEEANPAKKLKSMDFETPYNNTEWDASLDFLNEDAVTTQDNGANPMDLWSIDEIHSMIGGVF

SEQ ID NO: 353 - protein, 164 aa, Arabidopsis thaliana
NKSDSDERSRSENIALQTSSTRNTINHRSTYASSTENISRLLEGWMRASPKSSTSTTFLEHKMQNRTNNFIDHHSDQFPYEQLQGSWEEGHSKGINGDDDQGIKNSENNNGDDVHHEDGDHEDDDDHNATPPLTFIEKWLLEETSTTGGQMEEMSHLMELSNML

SEQ ID NO: 354 - protein, 539 aa, Arabidopsis thaliana
MEDSFLQSENVVMDADFMDGLLLDGCWLETTDGSEFLNIAPSTSSVSPFDPTSFMWSPTQDTSALCTSGVVSQMYGQDCVERSSLDEFQWNKRWWIGPGGGGSSVTERLVQAVEHIKDYTTARGSLIQLWVPVNRGGKRVLTTKEQPFSHDPLCQRLANYREISVNYHFSAEQDDSKALAGLPGRVFLGKLPEWTPDVRFFKSEEYPRVHHAQDCDVRGTLAIPVFEQGSKICLGVIEVVMTTEMVKLRPELESICRALQAVDLRSTELPIPPSLKGCDLSYKAALPEIRNLLRCACETHKLPLAQTWVSCQQQNKSGCRHNDENYIHCVSTIDDACYVGDPTVREFHEACSEHHLLKGQGVAGQAFLTNGPCFSSDVSNYKKSEYPLSHHANMYGLHGAVAIRLRCIHTGSADFVLEFFLPKDCDDLEEQRKMLNALSTIMAHVPRSLRTVTDKELEEESEVIEREEIVTPKIENASELHGNSPWNASLEEIQRSNNTSNPQNLGLVFDGGDKPNDGFGLKRGFDYTMDSNVNESSTF

SEQ ID NO: 355 - protein, 71 aa, Arabidopsis thaliana
RAQLDELNHRLQSLNDIIEFLDSSNNNNNNNMGMCSNPLVGLECDDFFVNQMNMSYIMNQPLMASSDALMY

SEQ ID NO: 356 - protein, 135 aa, Arabidopsis thaliana
YSDMPPSSSVTSIVSPDDPPPPPPPPAPPSNDPVDYMMMFNQYSSTDSPMLQPHCDQVDSYMFGGSQSSNSYCYSNDSSNELPPLPSDLSNSCYSQPQWTWTGDDYSSEYVHSPMFSRMPPVSDSFPQGFNYFGS

SEQ ID NO: 357 - protein, 148 aa, Arabidopsis thaliana
NIQIPKRKGEEEEAEEESTSVGKEEEEEKEKKWRKCDGNYIEDESLKRASAETSSSELTQGVLLDEANSSSIFALHFSSSLLDDHDHLFSNYSHQLPYHPPLQLQDFPQLSMNEAEIMSIQQDFQCRDSMNGTLDEIFSSSATFPASL

SEQ ID NO: 358 - protein, 161 aa, Arabidopsis thaliana
RQLNIDSNSHKFIEVVRSFWFPRLINEIKDNSYTNNIKANAPDLLGPILRDSKDLGFNNMDCSTSMSEDLKKTSQFMDFSDLETTMSLEGSRGGSSQCVSEVYSSFPCLEEEYMVAVMGSSDISALHDCHVADSKYEDDVTQDLMWNMDDIWQFNEYAHFN

SEQ ID NO: 359 - protein, 151 aa, Arabidopsis thaliana
PCSLQVISSPPLFSNGTSSASRELVRNHPSTAMMMMSSGGFSGYMFPLDPNFNLASSSIESLSSFNQDLHQKLQQQRLVTSMFLQDSLPVNEKTVMFQNVELIPPSTVTTDWVFDRFATGGGATSGNHEDNDDGEGNLGNWFHNANNNALL

SEQ ID NO: 360 - protein, 107 aa, Arabidopsis thaliana
RGASKLLNEQEGFMDEVLMEDETKVVVNEAERRTEEEIMMMTSMKLPRTCSLAHLLEMDYMGPVSHIDNFSQFDHLHQPDSESSWFGDLQFNQDEILNHHRQAMFKF

SEQ ID NO: 361 - protein, 130 aa, Arabidopsis thaliana
RTTIPTKRRQLWDPNCLFYDDATLLEPLDKRARHNPDFTATPFKQELLSEASHVQDGDFGSMYLQCIDDDQFSQLPQLESPSLPSEITPHSTTFSENSSRKDDMSSEKRITDWRYLDKFVASQFLMSGED

SEQ ID NO: 362 - protein, 163 aa, Arabidopsis thaliana
RQLNIESNSDKFFDAVRSFWVPRLIEKMEQNSSTTTTYCCPQNNNNNSLLLPSQSHDSLSMQKDIDYSGFSNIDGSSSTSTCMSHLTTVPHFMDQSNTNIIDGSMCFHEGNVQEFGGYVPGMEDYMVNSDISMECHVADGYSAYEDVTQDPMWNVDDIWQFRE

SEQ ID NO: 363 - protein, 110 aa, Arabidopsis thaliana
EDLGGGRKKDEEAESSGGYWLETNKAGNGVIETEGGKDYVVYNEDAIELGHDKTQNPMTDNEIVNPAVKSEEGYSYDRFKLDNGLLYNEPQSSSYHQGGGFDSYFEYFRF

SEQ ID NO: 364 - protein, 321 aa, Arabidopsis thaliana
PPLSLSGGSCSLLMNDCSQYDVEGFEKESHYEVEELKPEKVMNSSNFGMVAKMHDFPVKEEVPAGNSEFMRKRKPNRDLNTIMDRTVFTCENLGCAHSEISRGFLDRNSRDNHQLACPHRDSRLPYGAAPSRFHVNEVKPVVGFPQPRPVNSVAQPIDLTGIVPEDGQKMISELMSMYDRNVQSNQTSMVMENQSVSLLQPTVHNHQEHLQFPGNMVEGSFFEDLNIPNRANNNNSSNNQTFFQGNNNNNNVFKFDTADHNNFEAAHNNNNNSSGNRFQLVFDSTPFDMASFDYRDDMSMPGVVGTMDGMQQKQQDVSIWF

SEQ ID NO: 365 - protein, 222 aa, Arabidopsis thaliana
QEADSRSEGSVKAIVIPPPRPKRKPAHPYPRKSPVPYTQSPPPNLSAMEKGTKSPTSVLSSFGSEDQNNYTTSKQPFKDDSDIGSTPISSITLFGKIVLVAEESHKPSSYNDDDLKQMTCQENHYSGMLVDTNLSLGVWETFCTGSNAFGSVTEASENLEKSAEPISSSWKRLSSLEKQGSCNPVNASGFRPYKRCLSEREVTSSLTLVASDEKKSQRARIC

SEQ ID NO: 366 - protein, 143 aa, Arabidopsis thaliana
NNSTASRHHHHLHHIHLDNDHHRHDMMIDDDRFRHVPPGLHFPAIFSDNNDPTAIYDGGGGGYGGGSYSMNHCFASGSKQEQLFPPVMMMTSLNQDSGIGSSSSPSKRFNGGGVGDCSTSMAATPLMQNQGGIYQLPGLNWYS

SEQ ID NO: 367 - protein, 145 aa, Arabidopsis thaliana
KSSSKQDKVKKSLSRKQQQVDLKPQPQAQSENHQSQLVSQDHMNIDNDHNIASSLYYPTSVFDDKLYMPQSVATTSSDHSMIDEGHLWGSLWNLDEDDPHSFGGGSGQGTAADIDEKFPDSGIEAPSCGSGDYSYTGVYMGGYIF

SEQ ID NO: 368 - protein, 205 aa, Arabidopsis thaliana
KNHFRGFHQEQEQDHHHHHQYISTNNDHDHHHHIDSNSNNHSPLILHPLDHHHHHHHIGRQIHMPLHEFANTLSHGSMHLPQLFSPDSAAAAAAAAASAQPFVSPINTTDIECSQNLLRLTSNNNYGGDWSFLDKLLTTGNMNQQQQQQVQNHQAKCFGDLSNNDNNDQADHLGNNNGGSSSSPVNQRFPFHYLGNDANLLKFPK

SEQ ID NO: 369 - protein, 216 aa, Arabidopsis thaliana
PGVEDHPSVPRSLSTRHHNHNSSTSSRLALRQQQHHSSSSNHSDNNLNNNNNINNLEKLSTEYSGDGSTTTTTTNSNSDVTIALANQNIYRPMPYDTSNNTLIVSTRNHQDDDETAIVDDLQRLVNYQISDGGNINHQYFQIAQQFHHTQQQNANANALQLVAAATTATTLMPQTQAALAMNMIPAGTIPNNALWDMWNPIVPDGNRDHYTNIPFK

SEQ ID NO: 370 - protein, 153 aa, Arabidopsis thaliana
MYPSIEDDDDLLAALCFDQSNGVEDPYGYMQTNEDNIFQDFGSCGVNLMQPQQEQFDSFNGNLEQVCSSFRGGNNGVVYSSSIGSAQLDLAASFSGVLQQETHQVCGFRGQNDDSAVPHLQQQQGQVFSGVVEINSSSSVGAVKEEFEEECSG

SEQ ID NO: 371 - protein, 118 aa, Arabidopsis thaliana
GSVGSYPVPESTSAADIRAAAAAAAAMKGCEEGEEEKKAKEKKSSSSKSRARECHVDNDVGSSSWCGTEFMDEEEVLNMPNLLANMAEGMMVAPPSWMGSRPSDDSPENSNDEDLWGY

SEQ ID NO: 372 - protein, 112 aa, Arabidopsis thaliana
GLALTYVAPVSNSAADIRAAASRAAEMKQPDQGGDEKVLEPVQPGKEEELEEVSCNSCSLEFMDEEAMLNMPTLLTEMAEGMLMSPPRMMIHPTMEDDSPENHEGDNLWSYK

SEQ ID NO: 373 - protein, 72 aa, Arabidopsis thaliana
HLLNPSLVSRTSPRSIQQAASNAGMAIDAGIVHSTSVNSGCGDTTTYYENGADQVEPLNISVYDYLGGHDHV

SEQ ID NO: 374 - protein, 102 aa, Arabidopsis thaliana
NELKKNSKSLKNKNEQDIGSCYSSLATSPCRDEASQIQSFKPSSTTNDSSSIWISPDFILDSSKDYPQIKEVASECFPNYHFPVTTANHHVEFPLQEMLVRS

SEQ ID NO: 375 - protein, 218 aa, Arabidopsis thaliana
KPMTGQAKNTETWSSSYFYDELPSGVRSVTEPLNYVSKQKQNVFAQDLMFKQELEGSDIGLNFIHCDQFIQLPQLESPSLPLTKRPVSLTSITSLEKNKNIYKRHLIEEDVSFNALISSGNKDKKKKKTSVMTTDWRALDKFVASQLMSQEDGVSGFGGHHEEDNNKIGHYNNEESNNKGSVETASSTLLSDREEENRFISGLLCSNLDYDLYRDLHV

SEQ ID NO: 376 - protein, 119 aa, Arabidopsis thaliana
ELASLFPRPASSSPHDIQTAAAEAAAMVVEEKLLEKDEAPEAPPSSESSYVAAESEDEERLEKIVELPNIEEGSYDESVTSRADLAYSEPFDCWVYPPVMDFYEEISEFNFVELWSFNH

SEQ ID NO: 377 - protein, 167 aa, Arabidopsis thaliana
NAPSTTITTTKQLSRIDSLDNIDHLLDFSSLPPLIDPGFLGQPGPSFSGARQQHDLKPVLHHPTTAPVDNTYLPTQALNFPYHSVHNSGSDFGYGAGSGNNNKGMIKLEHSLVSVSQETGLSSDVNTTATPEISSYPMMMNPAMMDGSKSACDGLDDLIFWEDLYTS

SEQ ID NO: 378 - protein, 193 aa, Arabidopsis thaliana
MEGGGRGPNQTILSEIEHMPEAPRQRISHHRRARSETFFSGESIDDLLLFDPSDIDFSSLDFLNAPPPPQQSQQQPQASPMSVDSEETSSNGVVPPNSLPPKPEARFGRHVRSFSVDSDFFDDLGVTEEKFIATSSGEKKKGNHHHSRSNSMDGEMSSASFNIESILASVSGKDSGKKNMGMGGDRLAELALL

SEQ ID NO: 379 - protein, 213 aa, Arabidopsis thaliana
EITNRSSSTAATATVSGSVTAFSDESEVCAREDTNASSGFGQVKLEDCSDEYVLLDSSQCIKEELKGKEEVREEHNLAVGFGIGQDSKRETLDAWLMGNGNEQEPLEFGVDETFDINELLGILNDNNVSGQETMQYQVDRHPNFSYQTQFPNSNLLGSLNPMEIAQPGVDYGCPYVQPSDMENYGIDLDHRRFNDLDIQDLDFGGDKDVHGST

SEQ ID NO: 380 - protein, 156 aa, Arabidopsis thaliana
SSETNLNADEAGSKGSLNEEENSQESSPNASMSFAGSNISSKDDDAQISQMFEHILTYSEFTGMLQEVDKPELLEMPFDLDPDIWSFIDGSDSFQQPENRALQESEEDEVDKWFKHLESELGLEENDNQQQQQQHKQGTEDEHSSSLLESYELLIH

SEQ ID NO: 381 - protein, 150 aa, Arabidopsis thaliana
YSDMPRGSSVTSFVSPDESQRFISELFNPPSQLEATNSNNNNNNNLYSSTNNQNQNSIEFSYNGWPQEAECGYQSITSNAEHCDHELPPLPPSTCFGAELRIPETDSYWNVAHASIDTFAFELDGFVDQNSLGQSGTEGFNSLPSTFFYQ

SEQ ID NO: 382 - protein, 119 aa, Arabidopsis thaliana
EISTSLYHIINNGDNNNDMSPKSIQRVAAAAAAANTDPSSSSVSTSSPLLSSPSEDLYDVVSMSQYDQQVSLSESSSWYNCFDGDDQFMFINGVSAPYLTTSLSDDFFEEGDIRLWNFC

SEQ ID NO: 383 - protein, 227 aa, Arabidopsis thaliana
MTLANDFGYSTAMSSSYSALHTSVEDRYHKLPNSFWVSSGQELMNNPVPCQSVSGGNSGGYLFPSSSGYCNVSAVLPHGRNLQNQPPVSTVPRDRLAMQDCPLIAQSSLINHHPQEFIDPLHEFFDFSDHVPVQNLQAESSGVRVDSSVELHKKSEWQDWADQLISVDDGSEPNWSELLGDSSSHNPNSEIPTPFLDVPRLDITANQQQQMVSSEDQLSGRNSSSSV

SEQ ID NO: 384 - protein, 117 aa, Arabidopsis thaliana
YNPNAIPTSSSKLLSATLTAKLHKCYMASLQMTKQTQTQTQTQTARSQSADSDGVTANESHLNRGVTETTEIKWEDGNANMQQNFRPLEEDHIEQMIEELLHYGSIELCSVLPTQTL

SEQ ID NO: 385 - protein, 135 aa, Arabidopsis thaliana
LETVIKAMEMDCNPNYYRMNNSNTSDPLRSSRKIGLRTGKEAVKAYDEVVDGMVENHCALSYCSTKEHSETRGLRGSEETWFDLRKRRRSNEDSMCQEVEMQKTVTGEETVCDVFGLFEFEDLGSDYLETLLSSF

SEQ ID NO: 386 - protein, 119 aa, Arabidopsis thaliana
EEEEVDVINLEEDDVYTNLTRIENTVVNDLLLQDFNHHNNNNNNNSNSNSNKCSYYYPVIDDVTTNTESFVYDTTALTSNDTPLDFLGGHTTTTNNYYSKFGTFDGLGSVENISLDDFY

SEQ ID NO: 387 - protein, 93 aa, Arabidopsis thaliana
ELAYHLPRPASADPKDIQAAAAAAAAAVAIDMDVETSSPSPSPTVTETSSPAMIALSDDAFSDLPDLLLNVNHNIDGFWDSFPYEEPFLSQSY

SEQ ID NO: 388 - protein, 154 aa, Arabidopsis thaliana
KRDVSSSETSQCSRSSPVVPVEQDDTSASALTCVNNPDDVSTVAPTAPTPNVPAGGNKETLFDFDFTNLQIPDFGFLAEEQQDLDFDCFLADDQFDDFGLLDDIQGFEDNGPSALPDFDFADVEDLQLADSSFGFLDQLAPINISCPLKSFAAS

SEQ ID NO: 389 - protein, 123 aa, Arabidopsis thaliana
DSAWRLPVPESNDPDVIRRVAAEAAEMFRPVDLESGITVLPCAGDDVDLGFGSGSGSGSGSEERNSSSYGFGDYEEVSTTMMRLAEGPLMSPPRSYMEDMTPTNVYTEEEMCYEDMSLWSYRY

SEQ ID NO: 390 - protein, 134 aa, Arabidopsis thaliana
TCNNITSPKTTTNFSVSLTNTNIFEGNRVHVTEQSEDMKPTKSEEVMISLEDLENKKNIFRTFSFSNHEIENGVWKSNLFLGNFVEDLSPATSGSAITSEVLSAPAAVENSETADSYFSSLDNIIDFGQDWLWS

SEQ ID NO: 391 - protein, 109 aa, Arabidopsis thaliana
DSAWRLRIPESTCAKDIQKAAAEAALAFQDETCDTTTTNHGLDMEETMVEAIYTPEQSEGAFYMDEETMFGMPTLLDNMAEGMLLPPPSVQWNHNYDGEGDGDVSLWSY

SEQ ID NO: 392 - protein, 109 aa, Arabidopsis thaliana
DSAWRLRIPESTCAKEIQKAAAEAALNFQDEMCHMTTDAHGLDMEETLVEAIYTPEQSQDAFYMDEEAMLGMSSLLDNMAEGMLLPSPSVQWNYNFDVEGDDDVSLWSY

SEQ ID NO: 393 - protein, 181 aa, Arabidopsis thaliana
MQSTHISGGSSGGGGGGGGEVSRSGLSRIRSAPATWIETLLEEDEEEGLKPNLCLTELLTGNNNSGGVITSRDDSFEFLSSVEQGLYNHHQGGGFHRQNSSPADFLSGSGSGTDGYFSNFGIPANYDYLSTNVDISPTKRSRDMETQFSSQLKEEQMSGGISGMMDMNMDKIFEDSVPCRV

SEQ ID NO: 394 - protein, 124 aa, Arabidopsis thaliana
TSSSSHHLLDNLLDENTLLSPKSIQRVAAQAANSFNHFAPTSSAVSSPSDHDHHHDDGMQSLMGSFVDNHVSLMDSTSSWYDDHNGMFLFDNGAPFNYSPQLNSTTMLDEYFYEDADIPLWSFN

SEQ ID NO: 395 - protein, 200 aa, Arabidopsis thaliana
NGLGPRHGSQYGAPFKEEDWSDKEEEYTQNHLVAGPSKETSLAAKASHSYAPKDGLTGVISESCVSDVPPLTATVLPPLTSDVIAYNPFSSSPLLEVPQVSLDGGELNSMLDLFSVDNDDCLLFDDFDYHNEVRHPDGFVNKEAPVFLGDGNFSGMFDLSNDQVVELQDLIQSPTPHPPSPPAQASIPDDSRSNGQTKDD

SEQ ID NO: 396 - protein, 134 aa, Arabidopsis thaliana
NEIKTNTKIRKIPSEQTIGSGESSGLSSRVTSPSRDETMPFHSFANPVSTETDSSNIWISPEFILDSSKDYPQIQDVASQCFQQDFDFPIIGNQNMEFPASTSLDQNMDEFMQNGYWTNYGYDQTGLFGYSDFS

SEQ ID NO: 397 - protein, 237 aa, Arabidopsis thaliana
YTPTDVHTILTNPNLHSLIVSPYNNNQSFLPNSSPQFVIDHHPHYQNYHQPQQPKHTLPQTVLPAASFKTPVRHQSVDIQAFGNSPQNSSSNGSLSSSLDEENNFFFSLTSEEHNKSNNNSGYLDCIVPNHCLKPPPEATTTQNQAGASFTTPVASKASEPYGGFSNSYFEDGEMMMMNHHEFGSCDLSAMITNYGAAAASMSMEDYGMMEPQDLSSSSIAAFGDVVADTTGFYSVF

SEQ ID NO: 398 - protein, 117 aa, Arabidopsis thaliana
DNPPVISGGRNLSRSEIREAAARFANSAEDDSSGGAGYEIRQESASTSMDVDSEFLSMLPTVGSGNFASEFGLFPGFDDFSDEYSGDRFREQLSPTQDYYQLGEETYADGSMFLWNF

SEQ ID NO: 399 - protein, 103 aa, Arabidopsis thaliana
ELASSLPRPADSSSDSIRMAVHEATLCRTTEGTESAMQVDSSSSSNVAPTMVRLSPREIQAINESTLGSPTTMMHSTYDPMEFANDVEMNAWETYQSDFLWDP

SEQ ID NO: 400 - protein, 114 aa, Arabidopsis thaliana
DSAWRLRIPETTCPKEIQKAASEAAMAFQNETTTEGSKTAAEAEEAAGEGVREGERRAEEQNGGVFYMDDEALLGMPNFFENMAEGMLLPPPEVGWNHNDFDGVGDVSLWSFDE

SEQ ID NO: 401 - protein, 168 aa, Arabidopsis thaliana
PKSSSGNNTKTSLTANSGNPGGGSPSIDLALVYANFLNPKPDESILQENCDLATTDFLVDNPTGTSMDPSWSMDINDGHHDHYINPVEHIVEECGYNGLPPFPGEELLSLDTNGVWSDALLIGHNHVDVGVTPVQAVHEPVVHFADESNDSTNLLFGSWSPFDFTADG

SEQ ID NO: 402 - protein, 68 aa, Arabidopsis thaliana
HEYQMMKDGPNGSHENAVASSSSGYRGGGGGDDGREVIEFEYLDDSLLEELLDYGERSNQDNCNDANR

SEQ ID NO: 403 - protein, 227 aa, Arabidopsis thaliana
MIPNDDDDANSMKNYPLNDDDANSMKNYPLNDDDANSMENYPLRSIPTELSHTCSLIPPSLPNPSEAAADMSFNSELNQIMARPCDMLPANGGAVGHNPFLEPGFNCPETTDWIPSPLPHIYFPSGSPNLIMEDGVIDEIHKQSDLPLWYDDLITTDEDPLMSSILGDLLLDTNFNSASKVQQPSMQSQIQQPQAVLQQPSSCVELRPLDRTVSSNSNNNSNSNNAA